SEQUENCE LISTING (1 sequence)

SEQ ID NO: 1 - protein, 8 aa, Artificial Sequence
  synthetic peptide
SIINFEKL

(19) United States
(12) Patent Application Publication
(10) Pub. No.: US 2012/0258125 A1
(43) Pub. Date: Oct. 11, 2012

(54) METHOD TO IDENTIFY A NOVEL CLASS OF IMMUNOLOGIC ADJUVANTS

(75) Inventors: CAROL O. COWING, DEL MAR, CA (US); CHRISTOPHER COWING-ZITRON, DEL MAR, CA (US)

(73) Assignee: LANCELL, L.L.C., SAN DIEGO, CA (US)

(21) Appl. No.: 13/440,818

(22) Filed: Apr. 5, 2012

Related U.S. Application Data
(60) Provisional application No. 61/472,575, filed on Apr. 6, 2011.

(51) Int. Cl. A61K 39/00; A61P 37/04; A61K 31/125; A61K 31/225; C40B 30/04; C40B 40/06

(52) U.S. Cl. 424/184.1; 506/9; 506/16; 424/278.1

(57) ABSTRACT

Methods of identifying an adjuvant capable of activating dendritic cells including measuring expression level genes in skin of an animal prior to exposure to a test compound, wherein the genes are known to be upregulated or downregulated in the skin of the animal in response to topical application of dibutyl phthalate (DBP) to skin of said animal; exposing skin of an animal of the same species to the test compound; measuring expression level of the genes in the skin of the animal after exposure to the test compound; and comparing expression level of the genes measured before and after exposure to the test compound, wherein an increase or decrease in expression level of the genes following exposure to the test compound indicates that the test compound is capable of activating dendritic cells. Also included are compositions that induce dendritic cell migration and modulate expression level of genes in skin cells.

### CROSS-REFERENCE TO RELATED APPLICATIONS

This application claims benefit of U.S. Provisional Application No. 61/472,575 filed Apr. 6, 2011, which is hereby incorporated by reference in its entirety.

### STATEMENT REGARDING FEDERALLY SPONSORED R&D

This invention was made with government support under funds awarded by The National Institutes of Health (R44AI072925). The government has certain rights in the invention.

### FIELD OF THE INVENTION

Methods of identifying a novel class of immunologic adjuvants that activate dendritic cells.

### DESCRIPTION OF THE RELATED ART

Dendritic cells are found in virtually all mammalian tissues where they reside for long periods of time in an inactive state, provided the tissue is not perturbed by an infectious pathogen or by some other physical threat to the integrity of the organism. When a pathogen or physical danger appears, the nearby quiescent dendritic cells become activated. This activation involves release of the cells from the surrounding tissue and their migration to and through lymphatic vessels and into the draining lymph node. During this process, the dendritic cells undergo numerous changes in their gene expression and functional capacities that enable them to process molecules found in their environment at the time of their activation and display them in the antigen-binding sites of their MHC class I and II surface molecules. The cells also start to express high levels of chemokines, cytokines and co-stimulatory molecules that enable them to activate naive T cells in the draining lymph node to the antigens they brought with them from the traumatized tissue.

In the last two decades, many details of the mechanisms that activate dendritic cells have been elucidated. The general process whereby pathogens and danger signals are recognized and host cells respond to defend the organism has been termed “innate immunity.” Dendritic cells are key players in the defense of the host because their innate immune response to pathogens and danger signals results in activation of antigen-specific acquired immunity. Antigen-specific acquired immunity can provide the individual with a lifetime's worth of protection against individual infectious pathogens. Moreover, the efficacy of vaccines depends upon their ability to activate the innate immune response without risking infection by a pathogen. Immunologic adjuvants perform this function in modern vaccines that lack a viable, replicating pathogen capable of infecting the host.

A dominant system whereby dendritic cells and other cells involved in the innate immune response recognize pathogens or danger signals is the family of Toll-like receptors (TLR). TLRs are pattern recognition receptors that recognize molecular features common to a whole class of pathogens or danger signals (reviewed in Kawai, T. and Akira, S. 2010 “The role of pattern-recognition receptors in innate immunity: update on Toll-like receptors” Nature Immunology 11:373-383). There are about a dozen known TLRs. Thus, a limited number of receptors can recognize an unlimited array of potential pathogens, and a common system of gene activation pathways leads to a host defense response that eliminates the pathogen or counteracts the physical danger (TLR and Innate Immunity Pathways, SA Biosciences, 2010; on the world-wide-web at sabiosciences.com/pathwaymagazine/pathways7/toll-like-receptors-and-innate-immunity.php).

The mammalian transcriptional network activated by TLR ligands in primary dendritic cells has recently been identified (Amit, I. et al. 2009 “Unbiased reconstruction of a mammalian transcriptional network mediating pathogen responses” Science 326:257-263). An unbiased genome-wide mRNA expression analysis of 14,000 genes in the dendritic cell response to 5 TLR ligands over a 24-hour period identified 1,800 genes whose transcription was perturbed 1.7-fold or more by TLR activation. The 5 ligands used span the range of pathogens recognized by the TLR system, so the dendritic cell responses detected were broadly representative of dendritic cell activation by the dominant innate immune recognition system. From prior phenotypic and functional analyses, it is clear that the 24-hour period of ligand exposure was sufficient to detect all of the downstream activation events that result in the ability of dendritic cells to activate naive T and B cells.

We previously identified an initial group of small lipophilic molecules that can traverse the stratum corneum, activate skin dendritic cells and induce their migration to the draining lymph node (U.S. Pat. No. 6,210,672). For example, such lipophilic molecules may include dibutyl phthalate, dibutyl-D-tartarate, N,N-diethyl-toluamide, dibutylfumarate, di(2-ethylhexyl)fumarate, diisooctylmaleate, diethylhexylmaleate, diisooctylfumarate, benzoic acid, behenylmaleate, dioctylphthalate, dibutylmaleate, dioctymaleate, dibutylsuccinate, dioctylsuccinate, dinonylphthalate, diisononylphthalate, dimethylphthalate, diethylphthalate, dipropylphthalate, diphenylphthalate, dibenzylbutylphthalate, diethylmethylphthalate and camphor. The most effective of these, dibutylphthalate (DBP), results in a 10- to 100-fold increase in the number of activated dendritic cells in the draining node. This response occurs in the absence of antigen. We subsequently demonstrated that topical application of these molecules over the site of antigen delivery into the skin results in a potent immunologic adjuvant effect, enhancing both cellular and antibody immunity to the antigen (U.S. Pat. No. 7,229,621).

When DBP was applied to the skin of mice injected intradermally with adjuvant-free peptide (SIINFEKL) or protein (OVA) antigen, there was a dramatic increase in T cell and antibody responses. Peptide-bearing activated dendritic cells in the draining lymph node were increased 40-fold by topical DBP. Protective and therapeutic tumor-specific immunity were induced by topical DBP when antigen alone was ineffective. Topical DBP induced a 16-fold increase in the IgG1 Ab response to OVA adsorbed to alum and increased the IgG2c response 26-fold. Thus, DBP not only increased the total Ab but also balanced the isotypes produced. Consequently, when 60% of mice given a high dose of alum-adsorbed OVA succumbed to anaphylaxis, all of the mice survived when given the same dose but also treated with topical DBP.

We tested topical DBP as an adjuvant for a limiting dose of Fluzone vaccine injected i.d. in mice. None of the mice injected with Fluzone alone produced protective HAI titers, while all of the mice injected with Fluzone plus topical DBP had HAI titers>1:40; the difference in titers was highly significant (p<0.005). Serum antibodies of all IgG isotypes were highly significantly increased by topical DBP. More importantly, IgA Ab in bronchoalveolar lavage was highly significantly increased by topical DBP and remained undiminished 6 months after immunization. Using a split virion vaccine made from mouse-adapted PR8, mice survived a lethal dose of virus if given topical DBP with the vaccine but not when given the vaccine alone.

Multiple topical doses of DBP give no histologic evidence of inflammation in the skin. Using the known transdermal transport rate of DPB on human skin in vivo, the maximum absorbed dose resulting from topical use of DBP as an adjuvant would be no higher than the amount we ingest daily and five logs lower than the LOEL (lowest observed event level) in pregnant rats.

### SUMMARY OF THE INVENTION

Some embodiments relate to a method of identifying a candidate adjuvant capable of activating dendritic cells, the method comprising:

a) measuring expression level of a plurality of genes in skin of an animal prior to exposure to a test compound, wherein the plurality of genes are known to be upregulated or downregulated in the skin of the animal in response to topical application of dibutyl phthalate (DBP) to skin of said animal;

b) exposing skin of an animal of the same species to the test compound;

c) measuring expression level of the plurality of genes in the skin of the animal after exposure to the test compound; and

d) comparing expression level of the plurality of genes measured in steps (a) and (c), wherein an increase or decrease in expression level of the plurality of genes following exposure to the test compound by a pre-determined change in expression level indicates that the test compound is capable of activating dendritic cells.

In some embodiments, the pre-determined change in expression level is selected from the group consisting of: (a) an increase by a factor of at least 2; and (b) a decrease by a factor of at least 2.

In some embodiments, the plurality of genes is selected from the group of genes listed in Tables 1-5 and in Table 8, Nos. 1-215.

In some embodiments, the plurality of genes are significantly upregulated by Toll-like Receptor (TLR) stimulation of dendritic cells.

In some embodiments, the plurality of genes comprises early response gene(s).

In some embodiments, the increase in gene expression is measured using a weighted average.

In some embodiments, gene expression is measured using an array comprising a substrate and a plurality of polynucleotide probes affixed to the substrate.

In some embodiments, the array comprises a plurality of polynucleotide probes that are specifically complementary to said plurality of genes.

Some embodiments relate to a method of identifying a candidate immunological adjuvant capable of activating dendritic cells, the method comprising:

(a) identifying genes whose expression levels are upregulated or downregulated in skin of an animal in response to topical application of DBP and DBP analogs;

(b) quantifying the levels of activated dendritic cells in draining lymph nodes of said animal in response to topical application of DBP and DBP analogs;

(c) determining a model of activity of the DBP and DBP analogs, wherein a level of activated dendritic cells in draining lymph nodes measured in response to topical application of DBP and DBP analogs is correlated with the genes whose expression levels are upregulated or downregulated in response to topical application of DBP and DBP analogs; and

(d) determining whether a test compound is a candidate immunological adjuvant capable of activating dendritic cells on the basis of whether topical application of the test compound results in upregulation or downregulation of genes comparable to genes that show upregulation or downregulation in response to DBP or a DBP analog that leads to increased levels of activated dendritic cells in draining lymph nodes of said animal.

In some embodiments, the model of activity of the DBP and DBP analogs is selected from the group consisting of Bayesian additive regression trees (BART), multivariate adaptive regression splines (MARS), gradient-boosted generalized linear models (GLMs), and bagged generalized linear models.

In some embodiments, the genes whose expression levels are upregulated or downregulated are selected from the group of genes listed in:

(a) Table 8, Nos. 216-436 determined by the Wilcoxin model and the genes determined by the Wilcoxin model that are in common with genes listed as Nos. 1-215 of Table 8;

(b) Table 8, Nos. 437-794 determined by the Kendall model and the genes determined by the Kendall genes that are in common with genes listed as Nos. 1-215 of Table 8; and

(c) Table 9.

Some embodiments relate to an array comprising:

(a) a solid support; and

(b) a plurality of polynucleotide probes immobilized on said solid support,

wherein the plurality of polynucleotide probes are capable of hybridizing to at least 10 genes listed in Tables 1-5 and Table 8, optionally including one or more control probes.

In some embodiments, the array is a microarray.

In some embodiments of array, the plurality of polynucleotide probes is capable of hybridizing to at least 10 of the 384 genes listed in Table 9.

Some embodiments relate to a kit comprising the array and instructions for test compound screening and quantification of gene expression using the microarray.

Some embodiments relate to a method of monitoring the efficacy of a candidate adjuvant compound in a subject comprising:

a) measuring baseline expression of a plurality of genes known to be upregulated or downregulated in skin in response to topical application of DBP;

c) topically applying to the skin of said subject the candidate adjuvant compound,

d) measuring the expression of the plurality of genes after exposure of the to the candidate adjuvant compound, and

e) comparing expression levels of the plurality of genes before and after exposure to the candidate adjuvant compound, wherein a change in expression of any of the one or more of the plurality of genes by at least two-fold following exposure to the candidate adjuvant compound indicates that the compound is an effective adjuvant.

In some embodiments, the one or more genes are early response gene(s).

In some embodiments, the plurality of genes are significantly upregulated by Toll-like Receptor (TLR) stimulation.

In some embodiments, an expression level of the plurality of genes is known to be increased in activated dendritic cells.

Some embodiments relate to a composition comprising:

a lipophilic molecule having a molecular weight of less than 500 daltons that induces dendritic cell migration and modulates expression level of genes in skin cells, wherein at least 20% of genes whose expression level is increased or decreased by at least 2-fold by DBP are also increased or decreased, respectively, by at least 2-fold by said lipophilic molecule, wherein the lipophilic molecule is not DBP, and a pharmaceutically acceptable carrier.

Some embodiments relate to a vaccine comprising an antigen and a lipophilic molecule of less than 500 daltons, wherein the molecule induces dendritic cell migration and modulates expression level of genes in skin cells, wherein at least 20% of genes whose expression level is increased or decreased by at least 2-fold by DBP are also increased or decreased, respectively, by at least 2-fold by said lipophilic molecule, wherein the molecule is not DBP.

Some embodiments relate to a method of inducing an immune response in a subject comprising administering a vaccine disclosed herein to a subject.

### BRIEF DESCRIPTION OF THE DRAWINGS

FIG. 1. Topical Adjuvants Lack Toll-Like Receptor (TLR) Agonist Activity.

FIG. 2. 48 hr Response of Wild-Type and Caspase-1^{-/-} Mice to Topical FITC+DBP.

FIG. 3. Flowcytometric analysis of aryl hydrocarbon receptor −/− (Ahr−/−) response to topical DBP.

FIG. 4. IgG1 response to OVA with adjuvant.

FIG. 5. IgG2b response to OVA with adjuvant.

FIG. 6. IgG2c response to OVA with adjuvant.

FIG. 7. DBP Analogs Used—Nomenclature.

FIG. 8. Activity of DBP analogs plotted as a function of topical treatment with DBP analog vs. activated dendritic cells/lymph node 48 hours after treatment.

FIG. 9. Vaccination with influenza vaccine (Fluzone), with and without adjuvant.

FIG. 10. Vaccination with influenza vaccine (Fluzone), with and without dibutyl L tartrate (DBlT) adjuvant.

### DETAILED DESCRIPTION OF THE PREFERRED EMBODIMENT

It is desirable to identify additional compounds that act as immunological adjuvants in a manner similar to the initially identified group of small lipophilic molecules that we previously identified. However, in the absence of any knowledge of the receptor(s) for these topical adjuvants, identification of additional active molecules with previously utilized methodologies would be tedious and time consuming since previous assays were performed in live animals, followed by analysis of the cellular response in the draining lymph node 1-3 days later, or followed by subsequent measurement of an antigen-specific immune response.

Methods for automated high-throughput isolation of total RNA already exist (e.g., robotic systems such as the RNEASY 96 BIOROBOT 9640 by Qiagen), and rapid high-throughput PCR-based focused arrays can easily be constructed with a limited number of genes, e.g., 100-400 genes. Thus, the methodology currently exists to design and execute a rapid high-throughput screening to identify a novel class of immunologic adjuvants.

We disclose a simple method that can be used by anyone skilled in the art to screen large numbers of molecules rapidly for their potential to function as immunologic adjuvants in a manner similar to the molecules we have previously identified. This screening method enables the identification of molecules that have increased adjuvant activity, alter the nature of the subsequent immune response, and/or have decreased adverse effects.

Our immunologic adjuvants do not use the dominant pathogen and danger signal recognition systems. Yet, activation of dendritic cells by our small molecules results in a potent immunological adjuvant effect. These observations, combined with our previously disclosed data, indicate that we have identified members of a class of immunologic adjuvants that use an unidentified novel recognition system whereby inactive dendritic cells can become activated to enhance the immunogenicity of vaccine antigens.

In some embodiments, the immunological adjuvants are lipophilic molecules having a molecular weight of less than 500 daltons, wherein the molecules induce dendritic cell migration and modulate expression level of genes in skin cells in a manner similar to DBP.

Using our gene expression data and publicly known transcriptional networks, we designed simple and efficient screens to identify other molecules that have the functional properties of our immunologic adjuvants. The key elements for design of the disclosed method to identify immunologic adjuvants are:

1. Our topical adjuvants induce the same phenotypic changes in naïve, non-activated dendritic cells as those induced by TLR and NALP inflammasome ligands (U.S. Pat. Nos. 6,210,672 and 7,229,621).

2. The mammalian transcriptional network activated by TLR ligands in naïve primary dendritic cells has been identified (Amit, I. et al. 2009, supra).

3. Our topical adjuvants have no TLR agonist activity (see Example 1).

The preceding information enabled us to develop methods to identify the molecular signature characteristic of our previously identified topical adjuvants. In these methods, mammalian skin cells are exposed to our topical adjuvants in vivo, followed by extraction of total RNA at various time points thereafter, including the earliest time points that reveal transcription perturbed by our topical adjuvants. The extracted RNA is analyzed by whole genome array, (e.g., using the same chips as those used for TLR ligand-induced transcriptional analyses of the same mammalian species).

The present disclosure encompasses gene expression profiles produced in response to dibutylphthalate (DBP) exposure. We disclose that such gene expression profiles correlate with dendritic cell activity at a cellular level (Amit, I. et al. supra) and can be used in methods for screening potential dendritic cell activation agents. Additionally, the invention includes microarrays used to measure the expression of particular sets of genes. Referring to Tables 1-6 at the end of Example 5, we disclose the identities of genes that are upregulated in response to topical treatment of mice with DBP. Among these upregulated genes, we have identified 33 early response genes and 340 genes previously known to exhibit increased expression in activated dendritic cells. The sum of these early response genes and genes known to exhibit increased expression in activated dendritic cells is 373 genes, which can conveniently be surveyed by means of a probe array having (e.g., 16 rows and 24 columns, which is equal to 384 positions).

The invention encompasses a method for screening a test compound for dendritic cell activation properties, the method comprising: providing a cell or tissue, measuring expression by the cell or tissue of a plurality of genes selected from Tables 1-5 and Table 8, exposing the cell or tissue to the test compound, and re-measuring the expression by the cell or tissue of the plurality of genes, wherein the degree of change in expression of the plurality of genes corresponds to the degree of dendritic cell activation by the test compound. In certain embodiments, the degree of change in gene expression of the plurality of genes is measured using a weighted average. This method may employ screening any number of genes selected from Tables 1-5 and Table 8, for example, at least 10, 20, 50, 75, 100, 125, 150, 175 or 200 genes from Tables 1-5 and Table 8 may be screened. This method commonly employs an array (or “microarray”) comprising a substrate and a plurality of polynucleotide probes affixed to the substrate. The array generally comprises a plurality of polynucleotide probes that are specifically complementary to a plurality of genes as shown in Tables 1-5 and Table 8.

The invention further encompasses a method for monitoring dendritic cell activation in cell culture or in a subject during treatment. The method comprises taking a baseline reading of gene expression for at least one gene selected from a set of genes known to be up-regulated by DBP; administering DBP (or a derived or related compound, and then re-measuring the expression of at least one of the genes being monitored. In addition to prospective identification of new compounds, such a method may be useful for research to determine the efficacy of known compounds.

In some embodiments, the invention includes microarrays comprising a set of genes selected from the genes identified in this disclosure to be differentially regulated by DBP by at least two-fold.

In other embodiments, the invention includes microarrays comprising a set of genes selected from the genes identified by using general linear modeling methods (GLM).

The invention also includes methods of inducing an immune response in a subject against an antigen comprising administering the antigen to the subject along with an agent, wherein the agent differentially regulates the activity or expression of at least one, two, three, four, five, six or more genes selected from the genes identified in this disclosure to be differentially regulated by DBP by at least two-fold. The agent may be DBP or a related or derived compound, or an agent identified by the method of screening for agents disclosed herein, wherein the agent is identified on the basis of differentially regulating the activity or expression of at least one, two, three, four, five, six or more genes selected from the genes identified in this disclosure to be up-regulated by DBP by at least two-fold. In some embodiments, the agent may be identified on the basis of GLM methods, such as those disclosed herein.

In the present disclosure, microarray analysis was used to determine the changes in gene expression profiles of normal epithelial cells after exposure to DBP. Results of the microarray experiments disclosed herein are consistent with the dendritic cell activation property of DBP and help elucidate its molecular mechanism. Various genes found by this study to be up-regulated by DBP are known to play a role in dendritic cell activation (Amit, I. et al. 2009, supra). It is therefore reasoned that DBP activates dendritic cells by up-regulating various genes. Such genes are disclosed in this study to be up-regulated in the presence of DBP by greater than two-fold.

Monitoring the expression of these genes can be employed in a number of methods useful in therapy, in drug screening and in research into dendritic cell activating compounds. If a change in expression occurs in response to the administration of a drug (such as DBP) then the change in expression can reasonably be used as a quantitative marker that correlates with the degree of dendritic cell activation effectiveness of the drug treatment. Thus methods involving measurement of gene expression can be used to monitor efficacy of treatment, and to predict likely clinical outcomes. In drug screening, an animal or cell culture is exposed to a compound, and the expression of one or a plurality of genes is monitored to screen putative drug candidates. The greater the average change in gene expression of the genes in a particular panel (e.g., a panel of genes listed in Tables 1-5 and Table 8), the higher the score of the drug candidate. Such prioritization is routinely used by drug discovery companies. Gene expression profiles may be produced using arrays (microarrays) and quantitatively scored by measuring the average change in gene expression for a panel of genes in response to exposure to a set quantity of a compound for a set time. The score may be weighted by ascribing greater weight to specific genes. For example, a panel of genes may be selected to include the genes shown to be differentially regulated in this study. Particular weight may be given to the genes that are known activators of dendritic cells. Algorithms for scoring and weighting expression array results are well known in the art and one of skill could readily create or adapt an algorithm for use with the present methods.

By monitoring one or a plurality of the differentially regulated genes disclosed in this study before, during and after the administration of DBP or another agent, efficacy of treatment may be monitored, and clinical outcomes can be better predicted. Such monitoring may be used to determine appropriate treatment and drug dosages.

The invention further encompasses a method for monitoring dendritic cell activation in cell culture or in a subject during treatment. The method comprises taking a baseline reading of gene expression for at least one gene selected from a set of genes known to be differentially regulated by DBP; administering DBP (or a derived or related compound); and then re-measuring the expression of at least one of the genes being monitored. Such a method may be useful for research to determine the efficacy of various drugs, combinations of drugs and formulations to activate dendritic cells to induce an immune response during immunization of a subject. Such drugs may include DBP, optionally in combination with other adjuvants.

The invention also includes microarrays comprising at least one or a plurality of genes selected from genes shown in this disclosure to be differentially regulated by DBP by at least two-fold (the term “plurality” means two or more). In some embodiments, the microarrays comprise at least one or a plurality of genes identified on the basis of GLM methods, such as those disclosed herein. In certain embodiments, the microarray may include all of the genes identified herein, or it may include a subset. Such a microarray may be employed in the above methods for monitoring the gene expression profile of a subject (or cell culture) treated DBP (or a derived or related compound). By looking at changes in the gene expression profile, a qualitative and/or quantitative assessment can be deduced as to the degree to which genes are differentially regulated in response to a treatment, and therefore the effectiveness of a treatment may be determined.

The invention further includes methods for screening compounds for dendritic cell activation properties using the arrays described herein. Such methods involve exposure of cultured cells, tissues, organs or whole animals to a test compound, and the measurement of expression of a plurality of genes before and after exposure to the test compound. The microarray used may include probes for detecting any desired number of the genes disclosed herein as being differentially regulated in the presence of DBP. For example the array may include probes for detecting at least 2, 5, 10, 15, 20, 25, 30, 50, 75, 100, 125, 150, 175, 200, 250, 300, 350 or 384 such genes.

Microarrays are well known in the art and consist of a plurality of polynucleotides arranged regularly on a substrate such as paper, nylon or other type of membrane, filter, gel, polymer, chip, glass slide, or any other suitable support. The polynucleotides on the substrate bind complementary polynucleotides in a sample, thereby forming a hybridization complex and allowing sensitive and accurate detection. The polynucleotides may be cDNAs of gene open reading frames (or parts of genes) that bind specifically to complimentary mRNAs. Often the polynucleotides are short oligonucleotides of between about 6 and 25 bases in length. In some instances, the mRNAs of the sample may be used to create an amplified cDNA library (using PCR) and this library may then be screened using an array. In the present case, a microarray may include one or more polynucleotides or oligonucleotides derived from of the genes shown in this disclosure to be differentially regulated by DBP by at least two-fold.

In the present disclosure, the term “polynucleotide” refers to an oligonucleotide, nucleotide, or polynucleotide, and fragments thereof, and to DNA or RNA of genomic or synthetic origin which may be single- or double-stranded, and represent the sense or antisense strand.

The above methods may include exposure of a subject or cell culture, or ex-vivo or in vitro tissue or organ to DBP or related or derived compounds. In the present disclosure “related or derived compounds” include variations of DBP or compounds that are identified on the basis of differentially regulating the activity or expression of at least one, two, three, four, five, six or more genes selected from the genes identified in this disclosure to be differentially regulated by DBP by: (a) at least two-fold (the term “plurality” means two or more). In some embodiments, the agent may be identified on the basis of GLM methods, such as those disclosed herein.

The microarray data are validated by performing real-time reverse-transcription PCR on selected genes.

It is an object of this disclosure to identify the expression profiles which are characteristic to dendritic cell activation by DBP. In some embodiments, genes that show enhanced expression in response to DBP treatment are also know to exhibit increased expression during dendritic cell activation. It is further an object to use the expression profiles in assays to identify agents that can be used as adjuvants in dendritic cell activation in a manner similar to the mechanism of DBP.

In one aspect of the invention, the identification of genes that are differentially expressed in dendritic cells in response to treatment with DBP is provided, making possible the characterization of their temporal regulation and function in dendritic cell activation. Thus, expression profiles, nucleic acids and proteins are provided for differing states of dendritic cells, including resting and activated dendritic cells. Thus, the present invention makes possible the identification and characterization of targets useful in monitoring, rational drug design, and/or therapeutic intervention by activation of the immune system.

The invention provides methods of screening drug candidates. Such methods entail providing a cell that expresses an expression profile gene selected from the group of genes listed in Tables 1-5 and Table 8. A drug candidate is added to the cell. The effect of the drug candidate on the expression of the gene is then determined.

In some methods the level of expression in the absence of the drug candidate to the level of expression in the presence of the drug candidate is compared. In other methods, the cell expresses an expression profile gene set of at least one expression profile gene, and the effect of the drug candidate on the expression of the set is determined. In some such methods, the profile gene set comprises one or more genes selected from the genes presented in Tables 1-5 and Table 8, wherein expression of said one or more genes is altered as a result of the introduction of the drug candidate.

The invention further provides an array of probes. The array comprises a support bearing a plurality of nucleic acid probes complementary to a plurality of mRNAs fewer than 1000 in number, wherein the plurality of mRNA probes includes an mRNA expressed by a gene selected from the group consisting of genes listed in Tables 1-5 and Table 8. Some such arrays comprise a plurality of sets of probes wherein each set of probes is complementary to subsequences from an mRNA. In some arrays the probes are cDNA sequences.

### DEFINITIONS

The transitional term “comprising” is synonymous with “including,” “containing,” or “characterized by,” is inclusive or open-ended and does not exclude additional, unrecited elements or method steps.

The transitional phrase “consisting of” excludes any element, step, or ingredient not specified in the claim, but does not exclude additional components or steps that are unrelated to the invention such as impurities ordinarily associated therewith.

The transitional phrase “consisting essentially of” limits the scope of a claim to the specified materials or steps and those that do not materially affect the basic and novel characteristic(s) of the claimed invention.

“Dendritic cells” (DCs) are immune cells forming part of the mammalian immune system. They function as antigen-presenting cells and act as messengers between the innate and adaptive immunity. Some dendritic cells are present in tissues that are in contact with the external environment, such as the skin and the inner lining of the nose, lungs, stomach and intestines. For example, Langerhans cells are a specialized type of skin dendritic cell. Dendritic cells can also be found in an immature state in the blood. Once activated, they migrate to the lymph nodes where they interact with T cells and B cells to initiate and shape the adaptive immune response.

The term “stratum corneum” refers to a broad zone of 20 to 30 cell layers thick. The dead cell remnants which comprise the stratum corneum are almost completely filled with keratin fibrils and surrounded by highly ordered lipid bilayers. As long as the epidermis is unbroken, the heavily keratinized stratum corneum presents a formidable physical barrier to entry for most foreign substances. The mucous membranes which line the digestive, respiratory, urinary and reproductive tracts, provide a similar, but less formidable physical barrier, lacking the thick stratum corneum.

When a candidate molecule modulates expression level of genes in skin cells in a manner similar to DBP, at least 10%, 15%, 20%, 25%, 30%, 35%, 40%, 45%, 50%, 55%, 60%, 65%, 70%, 75%, 80%, 85%, 90%, 95% or 100% of the genes whose expression level is increased or decreased by at least 2-fold by DBP, as identified herein, are also increased or decreased, respectively, by at least 2-fold by said candidate molecule.

The epithelium of both skin and mucous membranes is richly populated with immature dendritic cells, called epidermal Langerhans cells and dermal dendritic cells. These phagocytic leukocytes are poised for capture of antigens which may enter the skin through physical breaches in the stratum corneum. After infection or physical trauma to the skin, signals are generated that induce Langerhans cells to leave the epidermis and migrate into the dermis. There, migrating epidermal Langerhans cells and dermal dendritic cells enter and migrate through afferent lymphatics to draining lymph nodes, carrying with them any antigens which had penetrated the protective stratum corneum (i.e., viral, bacterial, parasitic, allergic). Very small, lipophilic molecules may penetrate the intact stratum corneum. Some of these molecules can activate dendritic cells in the skin.

The term “patient” includes mammals, such as humans, domestic animals (e.g., dogs or cats), farm animals (cattle, horses, or pigs), monkeys, rabbits, rats, mice, and other laboratory animals.

The terms “nucleic acid” or “nucleic acid molecule” refer to a deoxyribonucleotide or ribonucleotide polymer in either single- or double-stranded form, and unless otherwise limited, can encompass known analogs of natural nucleotides that can function in a similar manner as naturally occurring nucleotides.

A polynucleotide probe is a single stranded nucleic acid capable of binding to a target nucleic acid of complementary sequence through one or more types of chemical bonds, usually through complementary base pairing, usually through hydrogen bond formation. A polynucleotide probe can include natural (i.e., A, G, C, or T) or modified bases (e.g., 7-deazaguanosine, inosine). Therefore, polynucleotide probes can be 5-10,000, 10-5,000, 10-500, 10-50, 10-25, 10-20, 15-25, and 15-20 bases long. Probe are typically about 10-50 bases long, and are often 15-20 bases. In its simplest embodiment, the array includes test probes (also referred to as polynucleotide probes) more than 5 bases long, preferably more than 10 bases long, and some more than 40 bases long. The probes can also be less than 50 bases long. In some cases, these polynucleotide probes can range from about 5 to about 45 or 5 to about 50 nucleotides long or from about 10 to about 40 nucleotides long, or from about 15 to about 40 nucleotides in length. The probes can also be about 20 or 25 nucleotides in length.

In addition, the bases in a polynucleotide probe can be joined by a linkage other than a phosphodiester bond, so long as it does not interfere with hybridization. Thus, polynucleotide probes can be peptide nucleic acids in which the constituent bases are joined by peptide bonds rather than phosphodiester linkages. The length of probes used as components of pools for hybridization to distal segments of a target sequence often increases as the spacing of the segments increased thereby allowing hybridization to be conducted under greater stringency to increase discrimination between matched and mismatched pools of probes.

Relatively short polynucleotide probes can be sufficient to specifically hybridize to and distinguish target sequences. Therefore, the polynucleotide probes can be less than 50 nucleotides in length, generally less than 46 nucleotides, more generally less than 41 nucleotides, most generally less than 36 nucleotides, preferably less than 31 nucleotides, more preferably less than 26 nucleotides, and most preferably less than 21 nucleotides in length. The probes can also be less than 16 nucleotides, less than 13 nucleotides in length, less than 9 nucleotides in length and less than 7 nucleotides in length.

Typically, arrays can have polynucleotides as short as 10 nucleotides or 15 nucleotides. In addition, 20 or 25 nucleotides can be used to specifically detect and quantify nucleic acid expression levels. Where ligation discrimination methods are used, the polynucleotide arrays can contain shorter polynucleotides. Arrays containing longer polynucleotides are also suitable. High density arrays can comprise greater than about 100, 1000, 16,000, 65,000, 250,000 or even greater than about 1,000,000 different polynucleotide probes.

For high throughput screening (e.g., of candidate molecules) by means of probe arrays, it us useful to define a limited number of number genes for survey of the effects of the compounds on gene expression. For example, a solid support with 384 probes (e.g., 16 rows×24 columns) may conveniently be used to survey the effects of a compound on a correspondingly limited number of genes.

The term “target nucleic acid” refers to a nucleic acid (often derived from a biological sample), to which the polynucleotide probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid can refer to the specific subsequence of a larger nucleic acid to which the probe is directed or to the overall sequence (e.g., gene or mRNA) whose expression level it is desired to detect. The difference in usage can be apparent from context.

“Subsequence” refers to a sequence of nucleic acids that comprise a part of a longer sequence of nucleic acids.

“Gene” refers to a unit of inheritable genetic material found in a chromosome, such as in a human chromosome. Each gene is composed of a linear chain of deoxyribonucleotides which can be referred to by the sequence of nucleotides forming the chain. Thus, “sequence” is used to indicate both the ordered listing of the nucleotides which form the chain, and the chain which has that sequence of nucleotides. The term “sequence” is used in the same way in referring to RNA chains, linear chains made of ribonucleotides. The gene includes regulatory and control sequences, sequences which can be transcribed into an RNA molecule, and can contain sequences with unknown function. Some of the RNA products (products of transcription from DNA) are messenger RNAs (mRNAs) which initially include ribonucleotide sequences (or sequence) which are translated into a polypeptide and ribonucleotide sequences which are not translated. The sequences which are not translated include control sequences, introns and sequences with unknowns function. It can be recognized that small differences in nucleotide sequence for the same gene can exist between different persons, or between normal cells and cancerous cells, without altering the identity of the gene.

“Gene expression pattern” means the set of genes of a specific tissue or cell type that are transcribed or “expressed” to form RNA molecules. Which genes are expressed in a specific cell line or tissue can depend on factors such as tissue or cell type, stage of development or the cell, tissue, or target organism and whether the cells are normal or transformed cells, such as cancerous cells. For example, a gene can be expressed at the embryonic or fetal stage in the development of a specific target organism and then become non-expressed as the target organism matures. Alternatively, a gene can be expressed in liver tissue but not in brain tissue of an adult human.

Specific hybridization refers to the binding, duplexing, or hybridizing of a molecule only to a particular nucleotide sequence under stringent conditions when that sequence is present in a complex mixture (e.g., total cellular) DNA or RNA. Stringent conditions are conditions under which a probe can hybridize to its target subsequence, but to no other sequences. Stringent conditions are sequence-dependent and are different in different circumstances. Longer sequences hybridize specifically at higher temperatures. Generally, stringent conditions are selected to be about 5° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target sequence hybridize to the target sequence at equilibrium. (As the target sequences are generally present in excess, at Tₘ, 50% of the probes are occupied at equilibrium). Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide or tetraalkyl ammonium salts. For example, conditions of 5.times.SSPE (750 mM NaCl, 50 mM Na Phosphate, 5 mM EDTA, pH 7.4) and a temperature of 25-30° C. are suitable for allele-specific probe hybridizations. (See Sambrook et al. in Molecular Cloning: A Laboratory Manual, Cold Spring Harbor Laboratory Press, New York, 2001).

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well-known in the art. Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, Proc Nat'l Acad Sci USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds 1995 supplement)).

One example of a useful algorithm is PILEUP. PILEUP uses a simplification of the progressive alignment method of Feng & Doolittle, J Mol Evol 35:351-360 (1987). The method used is similar to the method described by Higgins & Sharp, CABIOS 5:151-153 (1989). Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc Acids Res 12:387-395 (1984).

Another example of algorithms that are suitable for determining percent sequence identity and sequence similarity are the BLAST and the BLAST 2.0 algorithm, which are described in Altschul et al., J Mol Biol 215:403-410 (1990) and Altschul et al., Nucleic Acids Res 25:3389-3402 (1977)). Software for performing BLAST analyses is publicly available through the National Center for Biotechnology Information. The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSLUM62 scoring matrix (see Henikoff & Henikoff, Proc Natl Acad Sci USA 89:10915 (1989)).

The term “molecule” is used broadly to mean an organic or inorganic chemical such as a drug; a peptide, including a variant or modified peptide or peptide-like substance such as a peptidomimetic or peptoid; or a protein such as an antibody or a growth factor receptor or a fragment thereof, such as an Fᵥ, F_{c} or F_{ab} fragment of an antibody, which contains a binding domain. A molecule can be non-naturally occurring, produced as a result of in vitro methods, or can be naturally occurring, such as a protein or fragment thereof expressed from a cDNA library.

The term “specific binding” (and equivalent phrases) refers to the ability of a binding moiety (e.g., a receptor, antibody, ligand or anti-ligand) to bind preferentially to a particular target molecule (e.g., ligand or antigen) in the presence of a heterogeneous population of proteins and other biologics (i.e., without significant binding to other components present in a test sample). Typically, specific binding between two entities, such as a ligand and a receptor, means a binding affinity of at least about 10⁶ M⁻¹, and preferably at least about 10⁷, 10⁸, 10⁹, or 10¹⁰ M⁻¹.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe,” a “normalization control” probe, an expression level control probe and the like. A perfect match control or perfect match probe is, however, distinguished from a “mismatch control” or “mismatch probe.”

The terms “mismatch control” or “mismatch probe” refer to probes whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch can comprise one or more bases. While the mismatch(s) can be located anywhere in the mismatch probe, terminal mismatches are less desirable as terminal mismatch is less likely to prevent hybridization of the target sequence.

The term “probe set” comprises at least a plurality of genes perfectly matched with a known target sequence.

The terms “background” or “background signal intensity” refer to hybridization signals resulting from non-specific binding, or other interactions, between the labeled target nucleic acids and components of the polynucleotide array (e.g., the polynucleotide probes, control probes, or the array substrate). Background signals can also be produced by intrinsic fluorescence of the array components themselves. A single background signal can be calculated for the entire array, or a different background signal can be calculated for each region of the array. In some embodiments, background is calculated as the average hybridization signal intensity for the lowest 1% to 10% of the probes in the array, or region of the array. In expression monitoring arrays (i.e., where probes are preselected to hybridize to specific nucleic acids (genes), a different background signal can be calculated for each target nucleic acid. Where a different background signal is calculated for each target gene, the background signal is calculated for the lowest 1% to 10% of the probes for each gene. Where the probes to a particular gene hybridize well and thus appear to be specifically binding to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample such as bacterial genes where the sample is of mammalian origin). Background can also be calculated as the average signal intensity produced by regions of the array that lack any probes at all.

The term “quantifying” when used in the context of quantifying nucleic acid abundance or concentrations (e.g., transcription levels of a gene) can refer to absolute or to relative quantification. Absolute quantification can be accomplished by inclusion of known concentration(s) of one or more target nucleic acids (e.g., control nucleic acids or with known amounts of the target nucleic acids themselves) and referencing the hybridization intensity of unknowns with the known target nucleic acids (e.g., through generation of a standard curve). Alternatively, relative quantification can be accomplished by comparison of hybridization signals between two or more genes, or between two or more treatments to quantify the changes in hybridization intensity and, by implication, transcription level.

### Gene Expression Profiles

The present invention provides novel methods for screening for compositions which modulate dendritic cell activity. The expression levels of genes are determined for different cellular states of dendritic cells to provide expression profiles. A cell expression profile of a particular dendritic cell state can be a “fingerprint” of the state; while two states can have any particular gene similarly expressed, the evaluation of a number of genes simultaneously allows the generation of a gene expression profile that is unique to the state of the cell. By comparing expression profiles of dendritic cells in activated or resting states, information regarding which genes are important (including both up- and down-regulation of genes) in each of these states is obtained. This information can then be used in a number of ways. For example, the evaluation of a particular treatment regime can be evaluated: (e.g., does a particular drug act as an adjuvant in a particular patient. Furthermore, these gene expression profiles can be used in drug candidate screening to find drugs that mimic a particular expression profile; for example, screening can be done for drugs that induce dendritic cell activation in a manner similar to DBP. Accordingly, genes are identified and described which are differentially expressed within and among dendritic cells in different states, from which the expression profiles are generated as further described herein. For example, determinations of differentially expressed nucleic acids are provided herein for dendritic cells which are resting or activated.

“Differential expression,” or grammatical equivalents as used herein, refers to both qualitative as well as quantitative differences in the genes' temporal and/or cellular expression patterns within and among dendritic cells. Thus, a differentially expressed gene can qualitatively have its expression altered, including an activation or inactivation in, for example, resting or activated cells. Genes can be turned on or turned off in a particular state, relative to another state. Any comparison of two or more states can be made. Such a qualitatively regulated gene will exhibit an expression pattern within a state or cell type which can be detectable by standard techniques in one such state or cell type, but can be not detectable in both. Alternatively, the determination can be quantitative in that expression is increased or decreased; that is, the expression of the gene is either upregulated, resulting in an increased amount of transcript, or downregulated, resulting in a decreased amount of transcript. The degree to which expression differs need only be large enough to quantify using standard characterization techniques, for example, by using Affymetrix GENECHIP expression arrays (Lockhart, Nature Biotechnology, (1996) 14:1675-1680). Other methods include, but are not limited to, quantitative reverse transcriptase PCR, Northern analysis and RNase protection. Preferably the change or modulation in expression (i.e., upregulation or downregulation) is at least about 5%, more preferably at least about 10%, more preferably, at least about 20%, more preferably, at least about 30%, or more preferably by at least about 50%, or at least about 75%, and more preferably at least about 90%.

Any one, two, three, four, five, or ten or more genes can be evaluated. These genes include, but are not limited to genes listed in Tables 1-5 and Table 8). Generally, oligonucleotide sequences used in the evaluation of these genes are derived from their 3′ untranslated regions.

Differentially expressed genes can represent “expression profile genes,” which includes “target genes.” “Expression profile gene,” as used herein, refers to a differentially expressed gene whose expression pattern can be used in methods for identifying compounds useful in dendritic cell activation. In some instances, only a fragment of an expression profile gene is used, as further described below.

“Expression profile,” as used herein, refers to the pattern of gene expression generated from two up to all of the expression profile genes which exist for a given state. As outlined above, an expression profile is in a sense a “fingerprint” or “blueprint” of a particular cellular state; while two or more states have genes that are similarly expressed, the total expression profile of the state will be unique to that state. The gene expression profile obtained for a given dendritic cell state can be useful for a variety of applications, including evaluation of various treatment regimes. In addition, comparisons between the expression profiles of different dendritic cell states can be similarly informative. An expression profile can include genes which do not appreciably change between two states, so long as at least two genes which are differentially expressed are represented. The gene expression profile can also include at least one target gene, as defined below. Alternatively, the profile can include all of the genes which represent one or more states. Specific expression profiles are described below.

Gene expression profiles can be defined in several ways. For example, a gene expression profile can be the relative transcript level of any number of particular set of genes. Alternatively, a gene expression profile can be defined by comparing the level of expression of a variety of genes in one state to the level of expression of the same genes in another state. For example, genes can be either upregulated, downregulated, or remain substantially at the same level in both states.

### Target and Pathway Genes

In addition to expression profile genes, the present invention also provides target genes. “Target gene,” as used herein, refers to a differentially expressed expression profile gene whose expression is unique for a particular state, such that the presence or absence of the transcript of a target gene(s) can indicate the state the cell is in. A target gene can be completely unique to a particular state; the presence or absence of the gene is only seen in a particular cell state, or alternatively, cells in all other states express the gene but it is not seen in the first state. Alternatively, target genes can be identified as relevant to a comparison of two states, that is, the state is compared to another particular state or standard to determine the uniqueness of the target gene. Target genes can be used in the compound identification methods described herein.

It should be understood that a target gene for a first state can be an expression profile gene for a second state. The presence or absence of a particular target gene in one state can be diagnostic of the state; the same gene in a different state can be an expression profile gene.

### Sample Preparation

To measure the transcription level (and thereby the expression level) of a gene or genes, a nucleic acid sample comprising mRNA transcript(s) of the gene or genes, or nucleic acids derived from the mRNA transcript(s) is provided. A nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample. Thus, suitable samples include mRNA transcripts of the gene or genes, cDNA reverse transcribed from the mRNA, cRNA transcribed from the cDNA, DNA amplified from the genes, RNA transcribed from amplified DNA, and the like.

In some methods, a nucleic acid sample is the total mRNA isolated from a biological sample. The term “biological sample,” as used herein, refers to a sample obtained from an organism or from components (e.g., cells) or an organism. The sample can be of any biological tissue or fluid. Frequently the sample is from a patient. Such samples include sputum, blood, blood cells (e.g., white cells), tissue or fine needle biopsy samples, urine, peritoneal fluid, and pleural fluid, or cells therefrom. Biological samples can also include sections of tissues such as frozen sections taken for histological purposes. Often two samples are provided for purposes of comparison. The samples can be, for example, from different cell or tissue types, from different species, from different individuals in the same species or from the same original sample subjected to two different treatments (e.g., drug-treated and control).

Generation of cDNAs

Methods of isolation and purification of nucleic acids are widely known in the art. The total nucleic acid can be isolated from a given sample using, for example, an acid guanidinium-phenol-choloroform extraction method and poly A⁺ mRNA is isolated by oligo dT column chromatography or by using (dT)ₙ magnetic beads.

The sample mRNA can be reverse transcribed with a reverse transcriptase and a primer consisting of oligo dT and a sequence encoding the phage T7 promoter to provide single stranded DNA template. The second DNA strand is polymerized using a DNA polymerase. Methods of in vitro polymerization are well known (see, e.g., Sambrook, supra).

After amplification, the nucleic acids are typically cleaved into smaller fragments. Cleavage can be achieved by DNaseI digestion, restriction enzyme digestion, or sonication. Nucleic acids are typically labeled. Label can be introduced during amplification either by linkage to one of the primers or by one of the nucleotides being incorporated. Alternatively, labeling can be effected after amplification and cleavage by end-labeling. Detectable labels suitable for use in the present invention include any composition detectable by spectroscopic, photochemical, biochemical, immunochemical, electrical, optical or chemical means.

In general, nucleic acid probes comprising the expression profile genes, including differentially expressed genes and target genes, can be attached to a solid support, generally in an array format, to allow for gene expression monitoring. “Gene” in this context includes full length genes and fragments thereof, and can comprise either the coding strand or its complement, and can be a portion of a gene, a regulatory sequence, genomic DNA, cDNA, RNA including mRNA and rRNA.

In some cases, the differentially expressed nucleic acid can be a fragment, or expressed sequence tag (EST). Once a differentially expressed nucleic acid which is not a full length gene is identified, it can be cloned and, if necessary, its constituent parts recombined to form an entire fall length or mature differentially expressed nucleic acid. Using methods described herein and known in the art, it can be used to identify the full length clone. Wherein the full length nucleic acid has a signal peptide and/or transmembrane region(s), it can be modified to exclude one or more of these regions so as to encode a peptide in its mature soluble form. Once isolated from its natural source, e.g., contained within a plasmid or other vector or excised therefrom as a linear nucleic acid segment, the recombinant differentially expressed nucleic acid can be further-used as a probe to identify and isolate other differentially expressed nucleic acid acids. It can also be used as a “precursor” nucleic acid to make modified or variant differentially expressed nucleic acid acids and proteins. Where two or more nucleic acids overlap, the overlapping portion(s) of one of the overlapping nucleic acids can be omitted and the nucleic acids combined for example by ligation to form a longer linear differentially expressed nucleic acid so as to, for example, encode the full length or mature peptide. The same applies to the amino acid sequences of differentially expressed polypeptides in that they can be combined so as to form one contiguous peptide.

It should be noted that the nucleic acid probes used herein need not be identical to the wild-type genes listed in Tables 1-5 and Table 8. Nucleic acids having sequence identity with differentially expressed nucleic acids preferably have about 65% or 75%, more preferably greater than about 80%, even more preferably greater than about 85% and most preferably greater than 90% sequence identity. In some embodiments the sequence identity will be as high as about 93 to 95 or 98%. Sequence identity will be determined using standard techniques known in the art, including, but not limited to, the local sequence identity algorithm of Smith & Waterman (supra), by the sequence identity alignment algorithm of Needleman & Wunsch, J. (supra), by the search for similarity method of Pearson & Lipman, (supra), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al, (supra).

The PCR method of amplification is described in PCR Technology: Principles and Applications for DNA Amplification (ed. H. A. Erlich, Freeman Press, NY, N.Y., 1992); and PCR Protocols: A Guide to Methods and Applications (eds. Innis, et al., Academic Press, San Diego, Calif., 1990). Nucleic acids in a target sample are usually labeled in the course of amplification by inclusion of one or more labeled nucleotides in the amplification mix. Labels can also be attached to amplification products after amplification e.g., by end-labeling. The amplification product can be RNA or DNA depending on the enzyme and substrates used in the amplification reaction.

Other suitable amplification methods include the ligase chain reaction (LCR) (see Wu and Wallace, Genomics 4: 560 (1989), Landegren et al., Science 241: 1077 (1988), transcription amplification (Kwoh et al., Proc Nall Acad Sci U.S.A 86: 1173 (1989)), and self-sustained sequence replication (Guatelli et al., Proc Nat'l Acad Sci U.S.A 87: 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

A variety of labels can be incorporated into target nucleic acids in the course of amplification or after amplification. Suitable labels include fluorescein or biotin, the latter being detected by staining with phycoerythrin-streptavidin after hybridization. In some methods, hybridization of target nucleic acids is compared with control nucleic acids. Optionally, such hybridizations can be performed simultaneously using different labels are used for target and control samples. Control and target samples can be diluted, if desired, prior to hybridization to equalize fluorescence intensities.

### Supports

Supports can be made of a variety of materials, such as glass, silica, plastic, nylon or nitrocellulose. Supports are preferably rigid and have a planar surface. Supports typically have from 1-10,000,000 discrete spatially addressable regions, or cells. Supports having 10-1,000,000 or 100-100,000 or 200-500 cells are common. In some supports, all cells are occupied by pooled mixtures of probes. In other supports, some cells are occupied by pooled mixtures of probes, and other cells are occupied, at least to the degree of purity obtainable by synthesis methods, by a single type of polynucleotide.

The location and sequence of each different polynucleotide probe in the array is generally known. Moreover, the large number of different probes can occupy a relatively small area providing a high density array having a probe density of generally greater than about 60, more generally greater than about 100, and most generally greater than about 500 different polynucleotide probes per cm². The small surface area of the array (often less than about 10 cm², preferably less than about 5 cm² more preferably less than about 2 cm², and most preferably less than about 1.6 cm²) permits the use of small sample volumes and extremely uniform hybridization conditions

### Synthesis of Probe Arrays

Arrays of probes can be synthesized in a step-by-step manner on a support or can be attached in presynthesized form. A preferred method of synthesis entails the use of light to direct the synthesis of polynucleotide probes in high-density, miniaturized arrays. Algorithms for design of masks to reduce the number of synthesis cycles may be utilized. Arrays can also be synthesized in a combinatorial fashion by delivering monomers to cells of a support by mechanically constrained flowpaths. Arrays can also be synthesized by spotting monomers reagents on to a support using an ink jet printer.

After hybridization of control and target samples to an array containing one or more probe sets as described above and optional washing to remove unbound and nonspecifically bound probe, the hybridization intensity for the respective samples is determined for each probe in the array. For fluorescent labels, hybridization intensity can be determined by, for example, a scanning confocal microscope in photon counting mode. Some types of label provide a signal that can be amplified by enzymatic methods.

### Design of Arrays

One type of array detects the presence and/or levels of particular mRNA sequences that are known in advance. In these arrays, polynucleotide probes can be selected to hybridize to particular preselected subsequences of mRNA gene sequence. Such expression monitoring arrays can include a plurality of probes for each mRNA to be detected. For analysis of mRNA nucleic acids, the probes are designed to be complementary to the region of the mRNA that is incorporated into the nucleic acids (i.e., the 3′ end). The array can also include one or more control probes.

### Control Probes

Arrays can contain control probes in addition to the probes described above. Normalization controls are typically perfectly complementary to one or more labeled reference polynucleotides that are added to the nucleic acid sample. The signals obtained from the normalization controls after hybridization provide a control for variations in hybridization conditions, label intensity, reading and analyzing efficiency and other factors that can cause the signal of a perfect hybridization to vary between arrays. Signals (e.g., fluorescence intensity) read from all other probes in the array can be divided by the signal (e.g., fluorescence intensity) from the control probes thereby normalizing the measurements.

Virtually any probe can serve as a normalization control. However, hybridization efficiency can vary with base composition and probe length. Normalization probes can be selected to reflect the average length of the other probes present in the array, however, they can also be selected to cover a range of lengths. The normalization control(s) can also be selected to reflect the (average) base composition of the other probes in the array. However one or a fewer normalization probes can be used and they can be selected such that they hybridize well (i.e., no secondary structure) and do not match any target-specific probes.

Normalization probes can be localized at any position in the array or at multiple positions throughout the array to control for spatial variation in hybridization efficiently. The normalization controls can be located at the corners or edges of the array as well as in the middle of the array.

Expression level controls can be probes that hybridize specifically with constitutively expressed genes in the biological sample. Expression level controls can be designed to control for the overall health and metabolic activity of a cell. Examination of the covariance of an expression level control with the expression level of the target nucleic acid can indicate whether measured changes or variations in expression level of a gene is due to changes in transcription rate of that gene or to general variations in health of the cell. Thus, for example, when a cell is in poor health or lacking a critical metabolite the expression levels of both an active target gene and a constitutively expressed gene are expected to decrease. The converse can also be true. Thus where the expression levels of both an expression level control and the target gene appear to both decrease or to both increase, the change can be attributed to changes in the metabolic activity of the cell as a whole, not to differential expression of the target gene in question. Conversely, where the expression levels of the target gene and the expression level control do not covary, the variation in the expression level of the target gene can be attributed to differences in regulation of that gene and not to overall variations in the metabolic activity of the cell.

Virtually any constitutively expressed gene can provide a suitable target for expression level controls. Typically expression level control probes can have sequences complementary to subsequences of constitutively expressed genes including, but not limited to the β-actin gene, the transferrin receptor gene, the GAPDH gene, and the like.

### Methods of Detection

In one method of detection, mRNA or nucleic acid derived therefrom, typically in denatured form, are applied to an array. The component strands of the nucleic acids hybridize to complementary probes, which are identified by detecting label. Optionally, the hybridization signal of matched probes can be compared with that of corresponding mismatched or other control probes. Binding of mismatched probe serves as a measure of background and can be subtracted from binding of matched probes. A significant difference in binding between a perfectly matched probes and a mismatched probes signifies that the nucleic acid to which the matched probes are complementary is present. Binding to the perfectly matched probes is typically at least 1.2, 1.5, 2, 5 or 10 or 20 times higher than binding to the mismatched probes.

In a variation of the above method, nucleic acids are not labeled but are detected by template-directed extension of a probe hybridized to a nucleic acid strand with the nucleic acid strand serving as a template. The probe is extended with a labeled nucleotide, and the position of the label indicates, which probes in the array have been extended. By performing multiple rounds of extension using different bases bearing different labels, it is possible to determine the identity of additional bases in the tag than are determined through complementarity with the probe to which the tag is hybridized.

### Analysis of Hybridization Patterns

The position of label is detected for each probe in the array using a reader. For customized arrays, the hybridization pattern can then be analyzed to determine the presence and/or relative amounts or absolute amounts of known mRNA species in samples being analyzed. Comparison of the expression patterns of two samples is useful for identifying mRNAs and their corresponding genes that are differentially expressed between the two samples. Expression monitoring can be used to monitor expression of various genes in response to a candidate drug.

### Screening for Dendritic Cell Activity Modulators

### Candidate Bioactive Agents

Having identified a number of suitable expression profiles in response to DBP stimulation of dendritic cells, the information is used in a wide variety of ways. In a preferred method, the expression profiles can be used in conjunction with high throughput screening techniques, to allow monitoring for expression profile genes after treatment with a candidate agent. In a preferred method, the candidate agents are added to cells.

The term “candidate bioactive agent” or “drug candidate” or grammatical equivalents as used herein describes any molecule, e.g., protein, oligopeptide, small organic molecule, polysaccharide, polynucleotide, to be tested for bioactive agents that are capable of directly or indirectly activating dendritic cells. In preferred methods, the bioactive agents modulate the expression profiles, or expression profile nucleic acids provided herein. Generally, a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents can be subjected to directed or random chemical modifications, such as acylation, alkylation, esterification, amidification to produce structural analogs. In some methods, the candidate bioactive agents are organic chemical moieties.

### Drug Screening Methods

Several different drug screening methods can be accomplished to identify drugs or bioactive agents that modulate dendritic cell activity. One such method is the screening of candidate agents that can induce a particular expression profile, thus preferably generating the associated phenotype. Candidate agents that can mimic or produce an expression profile similar to an expression profile as shown herein is expected to result in activation of dendritic cells. Thus, candidate agents can be determined that mimic the DBP induced expression profile in dendritic cells.

In other methods, after having identified the differentially expressed genes important in any one state, candidate agent screening can be run to alter the expression of individual genes. For example, particularly in the case of target genes whose presence or absence is unique between two states, screening for modulators of the target gene expression can be done.

In other methods, screening can be done to alter the biological function of the expression product of the differentially expressed gene. Again, having identified the importance of a gene in a particular state, screening for agents that bind and/or modulate the biological activity of the gene product can be performed.

Thus, screening of candidate agents that modulate dendritic cell activity either at the level of gene expression or protein level can be accomplished.

In some methods, a candidate agent can be administered to naïve dendritic cells, to determine if an associated dendritic cell activity expression profile is induced. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) can be put into a viral construct such as a retroviral construct and added to the cell, such that expression of the peptide agent is accomplished.

Once the candidate agent has been administered to the cells, the cells can be washed if desired and allowed to incubate under preferably physiological conditions for some period of time. The cells are then harvested and a new gene expression profile is generated, as outlined herein.

For example, dendritic cells can be screened for agents that activate the cells. A change in at least one gene of the expression profile indicates that the agent has an effect on dendritic cell activity. In a preferred method, an activated dendritic cell profile is induced or maintained, before, during, and/or after stimulation with antigen. By defining such a signature for dendritic cell activation, screens for new drugs that mimic the phenotype can be devised. With this approach, the drug target need not be known and need not be represented in the original expression screening platform, nor does the level of transcript for the target protein need to change.

Dendritic cell activation by DBP or other immunological adjuvants identified by the methods disclosed herein may occur in the skin of a subject or in any other organ where dendritic cells are located.

In some preferred methods, screens can be done on individual genes and/or gene products. After having identified a particular differentially expressed gene as important in a particular state, screening of modulators of either the expression of the gene or the gene product itself can be completed.

### Utilization of Identified Agents as Adjuvants

Agents that elicit a gene expression response in dendritic cells similar to the response induced by DBP are tested for their ability to act as adjuvants, whereby an immune response to an antigen given in combination with the agent is enhanced.

### Animal Models

In a preferred method, nucleic acids which encode differentially expressed proteins or their modified forms can also be used to generate either transgenic animals, including “knock-in” and “knock out” animals which, in turn, are useful in the development and screening of therapeutically useful reagents. A non-human transgenic animal (e.g., a mouse or rat) is an animal having cells that contain a transgene, which transgene is introduced into the animal or an ancestor of the animal at a prenatal, e.g., an embryonic stage. A transgene is a DNA which is integrated into the genome of a cell from which a transgenic animal develops, and can include both the addition of all or part of a gene or the deletion of all or part of a gene. In some methods, cDNA encoding a differentially expressed protein can be used to clone genomic DNA encoding a differentially expressed protein in accordance with established techniques and the genomic sequences used to generate transgenic animals that contain cells which either express (or overexpress) or suppress the desired DNA. Typically, particular cells would be targeted for a differentially expressed protein transgene incorporation with tissue-specific enhancers. Transgenic animals that include a copy of a transgene encoding a differentially expressed protein introduced into the germ line of the animal at an embryonic stage can be used to examine the effect of increased expression of the desired nucleic acid.

Similarly, non-human homologues of a differentially expressed protein can be used to construct a transgenic animal comprising a differentially expressed protein “knock out” animal which has a defective or altered gene encoding a differentially expressed protein as a result of homologous recombination between the endogenous gene encoding a differentially expressed protein and altered genomic DNA encoding a differentially expressed protein introduced into an embryonic cell of the animal. For example, cDNA encoding a differentially expressed protein can be used to clone genomic DNA encoding a differentially expressed protein in accordance with established techniques. A portion of the genomic DNA encoding a differentially expressed protein can be deleted or replaced with another gene, such as a gene encoding a selectable marker which can be used to monitor integration.

In order to overexpress a target gene sequence, the coding portion of the target gene sequence can be ligated to a regulatory sequence which is capable of driving gene expression in the animal and cell type of interest. Such regulatory regions will be well known to those of skill in the art, and can be utilized in the absence of undue experimentation.

For under expression of an endogenous target gene sequence, such a sequence can be isolated and engineered such that when reintroduced into the genome of the animal of interest, the endogenous target gene alleles will be inactivated. Preferably, the engineered target gene sequence is introduced via gene targeting such that the endogenous target sequence is disrupted upon integration of the engineered target sequence into the animal's genome.

Animals of any species, including, but not limited to, mice, rats, rabbits, guinea pigs, pigs, micro-pigs, goats, and non-human primates, e.g., baboons, monkeys, and chimpanzees can be used to generate animal models for the study of dendritic cell activation.

### Pharmaceutical Compositions and Methods of Administration

DBP or variants and derivatives thereof, or novel compounds identified by the prospective screening methods disclosed herein can be incorporated into pharmaceutical compositions suitable for administration. Such compositions typically comprise the active ingredient and a pharmaceutically acceptable carrier. Methods of formulation and delivery of peptide drugs are well known in the art.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

In some preferred methods, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. Formulations for injection can be presented in unit dosage form, e.g., in ampules or in multidose containers, with an added preservative. The compositions are formulated as sterile, substantially isotonic and in fall compliance with all Good Manufacturing Practice (GMP) regulations of the U.S. Food and Drug Administration.

Injection solutions and suspensions can be prepared from sterile powders, granules, and tablets of the kind previously described. Cells transduced by the packaged nucleic acid as described above in the context of ex vivo therapy can also be administered intravenously or parenterally as described above.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

### Kits

The present invention provides for kits for screening dendritic cell activity modulators. For example, some kits comprise probe arrays as described herein. Optional additional components of the kit include, for example, other restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions.

Usually, the kits of the present invention also contain instructions for carrying out the methods.

### Example 1

### Topical Adjuvants Lack TLR Agonist Activity

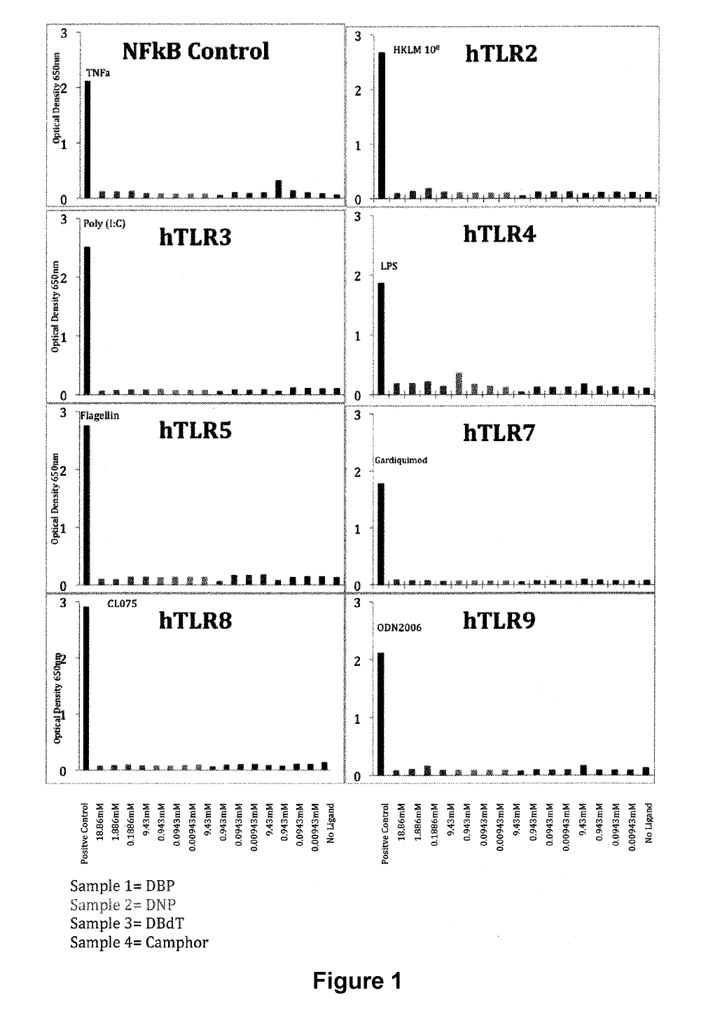

We tested our topical adjuvants for their ability to act via human Toll Like Receptors (hTLR2, hTLR3, hTLR4, hTLR5, hTLR7, hTLR8 and hTLR9). To our surprise, when we tested our topical adjuvants for TLR agonist activity, they had none (FIG. 1). Thus, our topically active molecules had no effect in a TLR ligand screen.

The test sample stock solutions were 1) 1.886 M DBP in 50% DMSO v/v, 2) 0.943 M DNP in 60% DMSO v/v, 3) 0.943 M DBdT in 100% DMSO, and 4) 0.943 M camphor in 100% DMSO. TLR stimulation was tested by assessing NF-κB activation in HEK293 cells expressing a given TLR (Invivogen). The secreted alkaline phosphatase reporter is under the control of a promoter inducible by the transcription factor NF-κB. This reporter gene allows the monitoring of signaling through the TLR, based on the activation of NF-κB. In a 96-well plate (200 μL total volume) containing the appropriate cells (25,000-50,000 cells/well), 20 μL of test sample or the positive control ligands was added to the wells. After a 16-20 hr incubation, the OD was read at 650 nm on a Beckman Coulter AD 340C Absorbance Detector.

The activity of each sample was tested on seven different human TLRs (TLR2, 3, 4, 5, 7, 8 and 9). Each test sample was tested at 1/100, 1/1000, 1/10,000 and 1/100,000 corresponding to a final concentration of DBP at 18.86 mM, 1.886 mM, 0.1886 mM and 0.01886 mM; DNP at 9.43 mM, 0.943 mM, 0.0943 mM and 0.00943 mM; DBdT at 9.43 mM, 0.943 mM, 0.0943 mM and 0.00943 mM; and camphor at 9.43 mM, 0.943 mM, 0.0943 mM and 0.00943 mM. The test samples were compared to the control ligands listed below. The assays were performed in triplicate and are reported as OD values.

The positive control ligands were: hTLR2: HKLM (heat-killed Listeria monocytogenes) at 108 cells/ml; hTLR3: Poly(I:C) at 1 μg/mL; hTLR4: E. coli K12 LPS at 100 ng/ml; hTLR5: S. typhimurium flagellin at 100 ng/ml; hTLR7: Gardiquimod at 1 μg/mL; hTLR8: CL075 at 1 μg/mL; hTLR9: CpG ODN 2006 at 100 ng/ml; and NF-κB Control cells: TNFα at 100 ng/ml.

### Example 2

### Response of Wild-type and Caspase^{-/-} Mice to Topical FITC and DBP

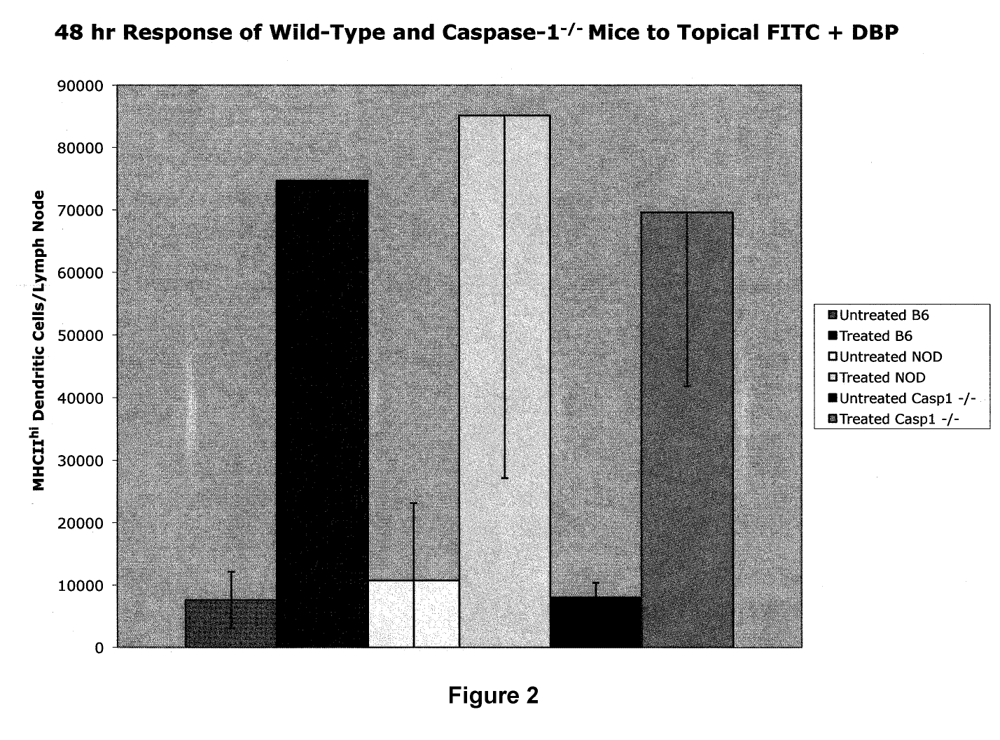

We also tested the relevance of a second dendritic cell activation system that intersects with TLR, the NALP inflammasome (reviewed in Franchi, L. et al. 2009 “The inflammasome: a Caspase-1-activation platform that regulates immune responses and disease pathogenesis” Nature Immunology 10:241-247). We determined that dendritic cell activation by our adjuvants does not depend on Caspase 1, an essential enzyme for the release of functional IL1-beta upon activation of NALP 1 and NALP 3 inflammasomes (FIG. 2). The dendritic cell response to DBP was undiminished in Casp1^{-/-} mice, indicating that active IL-1β is not required.

Mice were treated topically on the shaved abdominal skin with FITC dissolved in acetone (5 mg/ml) combined with an equal volume of DBP. Two days later, the skin draining inguinal lymph node cells were stained for immunofluorescent flow cytometry using monoclonal antibodies specific for CD19, CD11c, CD40, I-Ab, CD80, and CD86 obtained from BD Pharmingen, and detected using a Becton Dickenson FACSCalibur followed by analysis of the data using FlowJo software.

### Example 3

### Role of Aryl Hydrocarbon Receptor in DBP Activation of Skin Dendritic Cells

The aryl hydrocarbon receptor (AhR), also known as the dioxin receptor, is a transcription factor that mediates the response to the potentially toxic effects of environmental aromatic hydrocarbons. Ahr is expressed in murine and human monocytes and epidermal Langerhans cells and in murine dendritic epidermal T cells (DETC). Langerhans cell maturation and contact hypersensitivity to FITC plus DBP in C57BL/6 mice is reported to be impaired in AhR−/− mice (Jux, B. et al. 2009 J Immunol 182: 6709-6717). AhR was also found to be critical for homeostasis of DETC in mouse skin (Kadow, S. et al. 2011 J Immunol 187: 3104-3110) and AhR agonist signaling has been reported to interfere with the development of human monocytes and Langerhans cells (Platzer, B. et al. 2009 J

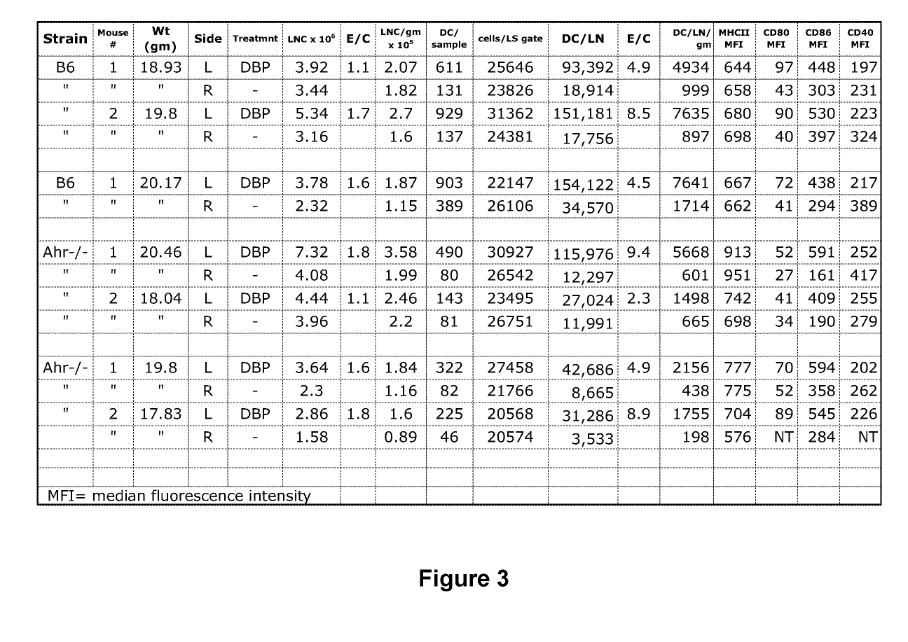

Immunol 183: 66-74). It seemed likely, therefore, that the AhR would be required for the activation of skin dendritic cells by topical DBP. To our surprise, we found no evidence for any impairment in the response to DBP in AhR−/− mice (see FIG. 3, Ahr−/− response to topical DBP).

If one measures the number of dendritic cells per lymph node per gram body weight in Ahr−/− mice vs. C57BL/6 wild type mice, there is a deficit in AhR−/−: 1,203 (SD=445) vs 476 (SD=208) p=0.016. Similarly, the absolute number of activated dendritic cells in the draining lymph node/gram body weight 48 hours after topical DBP in B6 mice vs Ahr−/− mice was 6,737 (SD1,561) vs 2,769 (SD1,951) p=0.017. However, the E/C was 5.97 in B6 mice vs 6.375 in Ahr−/− mice. Moreover, the level of the activation markers MHC class II, CD80, CD86 and CD40 expressed on the membrane of dendritic cells induced by DBP to migrate to the draining lymph node was not significantly different in B6 and Ahr−/− mice. These data demonstrate that the absence of AhR during development leads to a reduction in the number of skin derived dendritic cells, consistent with the findings of Platzer et al in humans, but there was no impairment in the ability of those dendritic cells to respond to topical DBP. Thus, the AhR transcription factor is not required for the activation of skin dendritic cells by topical DBP. By contrast, the skin dendritic cell response to topical dinitrofluorobenzene was severely impaired in AhR−/− mice.

### Example 4

### The Cutaneous Lymphoid Stress Response

The cutaneous lymphoid stress response has recently been elucidated (Strid, J. et al. 2011 Science 334: 1293-1297). TCRγδ intraepithelial lymphocytes (IEL) and dendritic epidermal T cells (DETC) express the NK cell activating receptor NKG2D and respond to endogenous NKG2D ligands. Murine NKG2D endogenous ligands include retinoic acid early inducible cDNA clone 1 (Rae-1) and the H60 family of glycoproteins. When physicochemical damage is perceived, epithelial cells respond by up-regulation of these endogenous NKG2D ligands. Antigens encountered at the same time as cutaneous epithelial stress were shown to induce strong primary and secondary atopic immune responses in mice.

Adjuvant and endotoxin-free ovalbumin (OVA) patches applied to the shaved skin of transgenic mice, acutely induced to express Rae-1 only in keratinocytes, produced strong IgG1 and IgE anti-OVA but negligible IgG2a anti-OVA. Total soluble serum IgE was also elevated. Thus NKG2D ligands expressed in the skin act as an adjuvant for antigens delivered to the skin resulting in an allergic response comprised mainly of IgG1 and IgE. While IgG1 responses were made in the absence of DETC, DETC were absolutely required for the antigen-specific and total IgE response. Mild tissue abrasion caused by tape-stripping B6 mouse skin also induced Rae-1 expression in the epidermis and resulted in morphological rearrangements of DETC and epidermal Langerhans cells. Tape stripping has been shown to induce the migration of Langerhans cells. This resulted in DETC-dependent I113, and I125 up-regulation within 2 hr in the epidermis and a subsequent increase in systemic IgE. Thus, mild cutaneous abrasion provokes the initial events of lymphoid stress-surveillance.

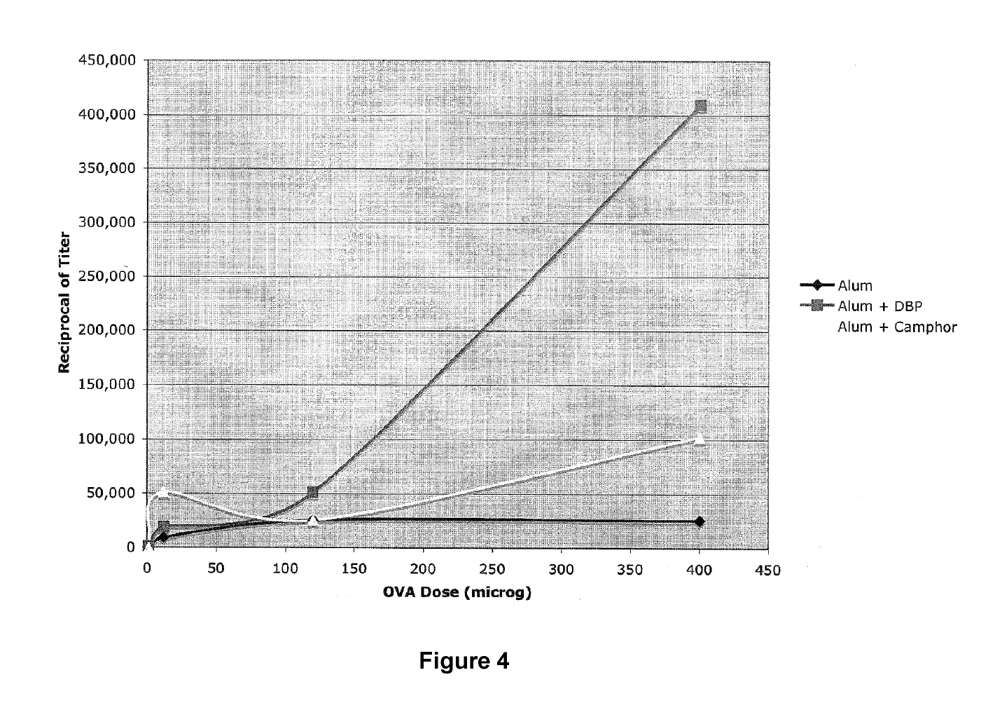

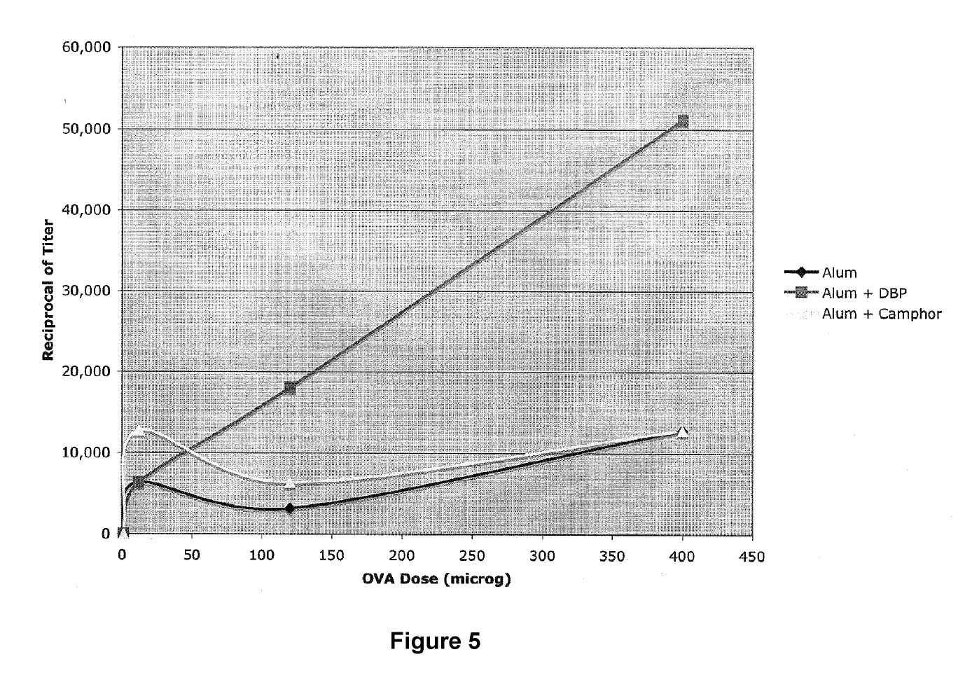

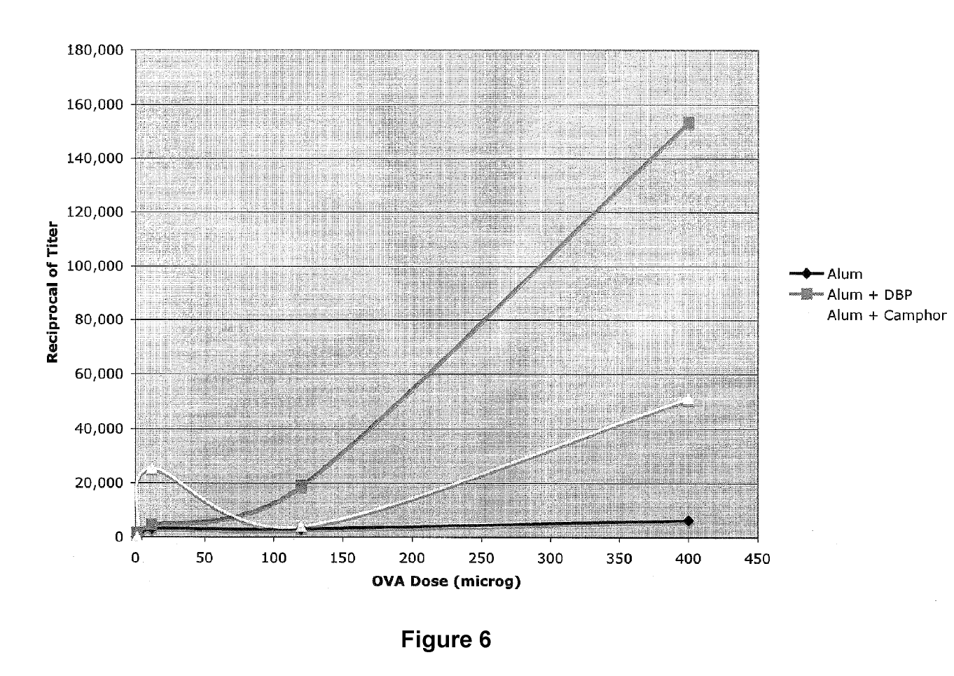

The cutaneous lymphoid stress response bears a strong resemblance to the effects of topical DBP. DBP is required for the induction of contact hypersensitivity to FITC in mice, a form of allergic response. Topical DBP induces the migration of Langerhans cells from the epidermis to draining lymph nodes. And, topical DBP acts as an adjuvant for antigens injected into the skin, including endotoxin-free OVA. One might expect, therefore, that topical DBP would cause the same effects as tape stripping or occlusive skin patch immunization. To our surprise, none of the effects that characterize the DETC-dependent cutaneous lymphoid stress-surveillance response are found in the response to DBP. Specifically, topical application of DBP to the ear skin of C57B1/6 mice induced no statistically significant increase in the expression of Rae-1, H60a, KLrk1 (encodes NKG2D), I1-I3 or I1-25. Expression of the most relevant gene, Rae-1, was actually diminished by topical DBP. Moreover, when endotoxin-free OVA was injected intradermally into B6 mice followed by topical application of DBP, antigen-specific IgG1, IgG2b and IgG2c antibodies were significantly elevated (see FIGS. 4, 5 and 6) but antigen-specific IgE was undetectable.

The adjuvant activity of topical DBP has nothing in common with the adjuvant activity characteristic of endogenous NKG2D-ligands that links lymphoid stress-surveillance to atopy. In fact, topical application of DBP in the absence of any exogenous (foreign) antigen results in no histologic evidence of contact hypersensitivity even after multiple applications (see DBP+CHS). These data fully distinguish our prototypic topical adjuvant, DBP, from the effects of a diverse group of adjuvants that depend on some form of physical or chemical disruption of the stratum corneum, including occlusive skin patches (U.S. Pat. Nos. 6,797,276 and 7,378,097) and tape stripping.

### Example 5

### Identification of Cellular Genes Modulated by DBP Showing≦2-Fold Increased Expression

Mice: C57BL/6J female mice obtained from The Jackson Laboratory were used between 8 and 12 weeks of age.

Treatment: Mice were untreated or treated topically on the dorsal side of each ear with 15 mL of dibutylphthalate (DBP). At the designated time points after treatment, the animals were euthanized and the ears washed with 70% isopropanol. Full thickness skin was obtained from the dorsal side of both ears and placed immediately in liquid nitrogen. The time between euthanasia and placement of both skin samples in liquid nitrogen was less than 1 minute. Each sample for RNA extraction contained two dorsal ear skin samples obtained from a single mouse both of whose ears received the same treatment.

RNA Extraction: While in liquid nitrogen, the skin was pulverized with a motorized mortar and pestle (CryoGrinder), transferred by inversion of the mortar directly into 1.5 ml cryogenic vials (Nalgene) and stored in liquid nitrogen until RNA extraction. Total RNA from both ears was extracted by homogenization (Pro200) in TRIzol Reagent (Invitrogen) with 7.5 μg of RNase-free glycogen added to the mixture, according to the manufacturer's directions. The RNA was purified on a solid phase RNeasy MinElute Cleanup column (Qiagen) and eluted with 17 μL of RNase-free water. The samples were stored at −20° C. until use.

Quantification of RNA: RNA yields were quantified by UV absorption using a NanoDrop NG-1000 spectrophotometer, where 1 AU 260 equals 40 μg RNA/ml. Purity was estimated by the 260 and 280 nm absorbance ratio; values between 1.7 and 2.1 were considered acceptable.

Assessment of RNA Quality: The quality of sample RNA was assessed using a RNA 6000 LABCHIP Kit on an Agilent 2100 Bioanalyzer. The Bioanalyzer determines the integrity/purity of the RNA samples and permits the screening of total and mRNA sample degradation and ribosomal RNA contamination. An RNA 6000 ladder was also loaded on a specified well and used as a reference for data analysis and a built-in quality control measure. The quality and integrity of the RNA was determined through visual inspection of each electropherogram. In general, only samples with a RIN (RNA integrity number) of 7 or greater, as determined by the Bioanalyzer, were used for gene arrays. This quality control analysis was repeated following the labeling procedure to ensure that the probes were efficiently labeled and had the correct size before hybridization to the chips. All samples that were run on genome wide arrays were obtained as independent duplicates from two individual mice.

Microarray Data Interpretation: Gene expression was compared between untreated and treated total RNA samples. “Transition” data, which represent the change in expression level for a given gene, are presented as a “weighted difference.” Changes in gene expression level were considered to be significant when the adjusted p values were less than 5.0. The “gene expression signature” arbitrarily includes only those genes that were transcribed with a weighted increase of 2.0 or greater, which was sustained over the subsequent time points tested. Genes whose expression was transiently increased by DBP were excluded; this occurred in less than 20 of the 13,373 unique genes analyzed. This decision was based on the assumption that genes that were significantly down-regulated by DBP treatment were, for the most part, a prerequisite of genes whose expression was increased or whose down-regulation was necessary for skin dendritic cells to release from and exit the skin, and therefore the latter were subsumed under the former.

The weighted difference is a metric that takes into account both the variability between different time-points and the variability between replicates at the same time-point. The calculations are performed for each time-point transition, i.e., 30 minutes vs. 1 hour, 1 hour vs. 2 hours, etc. There are two steps to the calculation: the calculation of a modified Euclidean difference between the two time-points, and the calculation of the Euclidean difference between the replicates within each time-point. The first measure is an unweighted difference, while the final metric given in the spreadsheet “weights” this difference by taking into account the second measure.

In a Euclidean difference calculation, each data point is considered as a vector in a multi-dimensional space, and the actual straight-line distance between the data points is calculated. This assumes that the data points have the same dimensions and can be compared pairwise, i.e., the first samples in the two time-points compared to each other plus the second two samples compared to each other. For the current dataset this would amount to treating each time-point as a two-dimensional vector and calculating the length of the line connecting them. We wished to consider not just ordered pairwise comparisons, but every possible pairwise contrast, i.e. the second sample in the first time-point contrasted with the first sample in the second time-point. To calculate this modified Euclidean difference between time-points, each pairing of a single sample from each of the two time-points is considered; for each pairing, the squared difference in expression is calculated. For the current dataset in which there are two replicates for each time-point, this amounts to four comparisons. We calculate the sum of the squared differences for each pairing, and take the square root. This is the modified Euclidean distance between the two time-points. This measures the absolute distance between the two time-points in a four-dimensional space, giving a fuller picture of the difference between the two time-points.

A caveat for an unweighted distance measure such as calculated in the first step is that a large difference can be observed between time-points as the result of a single outlier in a single time-point; that is, large variance within a single time-point can create the illusion of a significant difference between two time-points, despite the difference being in fact less significant given poor reproducibility of the data within the variable time-point. Thus we must weight the initial difference calculation, taking into account the variability between the replicates within each time-point. To do this, we take as one vector the first replicates in the two time-points, and take the second replicates as a second vector, calculating the true Euclidean distance between the two. This amounts to taking the squared difference between the two replicates in the first time-point plus the squared difference between the two replicates in the second time-point. We multiply this by two in order to balance the two differences in the sum for the second step with the four differences in the first sum, and take the square root. This measures the absolute difference between the replicates within each time-point.

The final metric given is the ratio of the modified Euclidean distance between the two time-points divided by the absolute Euclidean distance between the replicates within the two time-points. This “weighted difference” measures the variability between the two time-points while adjusting for the effects of within-time-point variabilty.

It is recognized that transcription factors responsible for the “gene expression signature” described herein may not be transcriptionally activated to the extent that would include them in this arbitrary definition of “gene expression signature”. However, there are methods well known in the field that enable identification of transcription factors that may regulate expression of the genes included in the “gene expression signature”. Transcription factor candidates identified by these methods can be confirmed or excluded by techniques well known in the field. Such techniques include the use of: gene knock-out mice, gene knock-in cells, small interfering RNA administered in vivo, short hairpin RNA administered ex vivo, etc.

Tables 1-5 below summarize genes that exhibit at least a 2-fold increase in expression following DBP administration. Table 1 lists early response genes that show an increased level of expression within 15 minutes after DBP application and Tables 2-5 list genes whose expression is increased greater than 2-fold at the 15-30 minute, 30 minute-1 hr, 1-2 hr and 2-4 hr time intervals.

TABLE 1A
Genes showing ≧2-fold increased expression 15 minutes after DBP application, with corresponding expression changes at 15-30 min, 30 min-1 hr, 1-2 hr and 2-4 hr time intervals.

| | | | | Change in Gene Expression (Weighted Difference) | | | | | | | | | |
|---|---|---|---|---|---|---|---|---|---|---|---|---|---|
| | | | Universal | | First | 15 min to | 15 min to | | 30 min to | | | | |
| | Universal | Universal Gene | Gene Refseq | | 15 min | 30 min | 30 min | 30 min to 1 hr | 1 hr | 1 hr to 2 hr | 1 hr to 2 hr | 2 hr to 4 hr | 2 hr to 4 hr |
| # | Symbol | Name | ID | First 15 min | P-value | Transition | P-value | Transition | P-value | Transition | P-value | Transition | P-value |
| 1 | Btg2 | B-cell | NM_007570 | 61.86531439 | 0 | 5.99403178 | 0.004496403 | 2.925919685 | 0.033217176 | −0.010048642 | 0.971504047 | 1.009020311 | 0.10982464 |
| | | translocation | | | | | | | | | | | |
| | | gene 2, anti- | | | | | | | | | | | |
| | | proliferative | | | | | | | | | | | |
| 2 | Myc | myelocytomatosis | NM_010849 | 24.16257556 | 0.00022482 | 2.987068599 | 0.017423561 | 1.894949254 | 0.06986286 | −0.014487234 | 0.959195144 | 1.296465811 | 0.074415468 |
| | | oncogene | | | | | | | | | | | |
| 3 | Mkks | McKusick- | NM_001141946 | 11.96527418 | 0.000786871 | −2.532575587 | 0.024674011 | 0.281068558 | 0.53939973 | −0.165672391 | 0.650011241 | −0.240590713 | 0.486398381 |
| | | Kaufman | | | | | | | | | | | |
| | | syndrome protein | | | | | | | | | | | |
| 4 | Cysltr1 | cysteinyl | NM_021476 | 7.655821061 | 0.001854766 | −1.813058309 | 0.041760342 | 0.066808148 | 0.847740558 | −1.79399909 | 0.066546763 | −0.804907722 | 0.149786421 |
| | | leukotriene | | | | | | | | | | | |
| | | receptor 1 | | | | | | | | | | | |
| 5 | Prf1 | perforin 1 (pore | NM_011073 | 7.177260282 | 0.002248201 | −0.05377361 | 0.818963579 | 0.518246744 | 0.353080036 | −0.311682249 | 0.472122302 | −0.168160292 | 0.588860162 |
| | | forming protein) | | | | | | | | | | | |
| 6 | Frk | fyn-related kinase | NM_001159544 | 6.498095791 | 0.002810252 | −5.361480682 | 0.005789119 | 3.984952636 | 0.020121403 | −0.267212115 | 0.520571043 | −0.04940198 | 0.846616457 |
| 7 | Jun | Jun oncogene | NM_010591 | 6.095048476 | 0.003091277 | 1.717160424 | 0.045863309 | 0.723354141 | 0.256351169 | −0.001533195 | 0.995391187 | 0.997070789 | 0.111904227 |
| 8 | Atp2c2 | ATPase, Ca++ | NM_026922 | 5.814962673 | 0.003372302 | 0.175422063 | 0.561432104 | −0.00870375 | 0.977630396 | 0.3309572 | 0.45368705 | −0.002189451 | 0.99274955 |
| | | transporting, | | | | | | | | | | | |
| | | type 2C, member | | | | | | | | | | | |
| | | 2 | | | | | | | | | | | |
| 9 | Ier3 | immediate early | NM_133662 | 5.580988846 | 0.003540917 | 12.73334953 | 0.001405126 | 7.145350667 | 0.006744604 | 0.77859953 | 0.207565198 | 4.521244361 | 0.00814973 |
| | | response 3 | | | | | | | | | | | |
| 10 | Nr4a1 | nuclear receptor | NM_010444 | 5.480483423 | 0.003765737 | 5.842232424 | 0.004889838 | 1.556174971 | 0.095155126 | −0.473370693 | 0.344874101 | 4.571957632 | 0.00792491 |
| | | subfamily 4, | | | | | | | | | | | |
| | | group A, member | | | | | | | | | | | |
| | | 1 | | | | | | | | | | | |
| 11 | Gimap7 | GTPase, IMAP | NM_146167 | 4.789013414 | 0.005002248 | −0.224869142 | 0.488084532 | −0.004048929 | 0.989826888 | 0.133133799 | 0.703630845 | 0.032047883 | 0.900123651 |
| | | family member 7 | | | | | | | | | | | |
| 12 | Junb | Jun-B oncogene | NM_008416 | 4.500293456 | 0.005508094 | 3.923739037 | 0.010735162 | 2.771391807 | 0.036926709 | −0.07540189 | 0.813343076 | 0.911865318 | 0.126180306 |
| 13 | Madcam1 | mucosal vascular | NM_013591 | 4.390072685 | 0.005789119 | 0.282376488 | 0.421931205 | 0.046002632 | 0.891917716 | −0.045974971 | 0.878540917 | −0.010812661 | 0.965321493 |
| | | addressin cell | | | | | | | | | | | |
| | | adhesion | | | | | | | | | | | |
| | | molecule 1 | | | | | | | | | | | |
| 14 | Zfp36 | zinc finger | NM_011756 | 4.190382972 | 0.006575989 | 10.69712874 | 0.001742356 | 2.800470573 | 0.036252248 | 0.10246334 | 0.759948291 | 2.637714303 | 0.021751349 |
| | | protein 36 | | | | | | | | | | | |
| 15 | Gbp8 | guanylate- | NM_029509 | 4.072945064 | 0.006857014 | 0.569774492 | 0.222234712 | 0.104836859 | 0.778214928 | −1.039112135 | 0.145683453 | −0.042227499 | 0.86763714 |
| | | binding protein 8 | | | | | | | | | | | |
| 16 | Med17 | mediator | NM_144933 | 3.948920511 | 0.00747527 | −0.229473778 | 0.483138489 | 0.062005716 | 0.857407824 | 0.107266784 | 0.750730665 | −2.25887E−05 | 0.999943795 |
| | | complex subunit | | | | | | | | | | | |
| | | 17 | | | | | | | | | | | |
| 17 | Akr1c18 | aldo-keto | NM_134066 | 3.655366792 | 0.008599371 | −0.611886405 | 0.204867356 | 1.114466239 | 0.153720773 | −0.247100557 | 0.542659622 | −0.085625911 | 0.753035072 |
| | | reductase family | | | | | | | | | | | |
| | | 1, member C18 | | | | | | | | | | | |
| 18 | Egr1 | early growth | NM_007913 | 3.56132877 | 0.008936601 | 4.966501406 | 0.006744604 | 5.495107787 | 0.012196493 | −0.21265539 | 0.586668165 | 2.347908188 | 0.026641187 |
| | | response 1 | | | | | | | | | | | |
| 19 | N6amt2 | N-6 adenine- | NM_026526 | 3.34708852 | 0.010004496 | −0.799750689 | 0.147650629 | 0.083296434 | 0.816546763 | −0.077911528 | 0.807947392 | −0.028239036 | 0.911870504 |
| | | specific DNA | | | | | | | | | | | |
| | | methyltransferase | | | | | | | | | | | |
| | | 2 (putative) | | | | | | | | | | | |
| 20 | Slc39a8 | solute carrier | NM_001135149 | 3.193071961 | 0.011297212 | 25.26582088 | 0.000505845 | 2.303891389 | 0.050415917 | 1.188451972 | 0.121065647 | 2.100573294 | 0.032430306 |
| | | family 39 (metal | | | | | | | | | | | |
| | | ion transporter), | | | | | | | | | | | |
| | | member 8 | | | | | | | | | | | |
| 21 | Sik1 | salt inducible | NM_010831 | 3.071787659 | 0.012084083 | 2.596613582 | 0.02332509 | 0.724085231 | 0.256126349 | 0.00776877 | 0.977742806 | 1.009360042 | 0.10982464 |
| | | kinase 1 | | | | | | | | | | | |
| 22 | LOC100046066 | similar to FAST | XM_001475471 | 2.503208024 | 0.018547662 | −2.103790537 | 0.033835432 | 2.144166053 | 0.057104317 | −0.857700079 | 0.188230665 | −0.146427632 | 0.626686151 |
| | | kinase domains 1 | | | | | | | | | | | |
| 23 | Gem | GTP binding | NM_010276 | 2.491134556 | 0.018660072 | 2.697376669 | 0.021301709 | 3.043801823 | 0.03102518 | 0.64836594 | 0.253540917 | 1.54623663 | 0.055699191 |
| | | protein (gene | | | | | | | | | | | |
| | | overexpressed in | | | | | | | | | | | |
| | | skeletal muscle) | | | | | | | | | | | |
| 24 | Cdh10 | cadherin 10 | NM_009865 | 2.487449212 | 0.018772482 | 0.197931801 | 0.526022932 | 0.238400045 | 0.587286421 | −0.151907939 | 0.672493255 | −0.008005196 | 0.974089478 |
| 25 | Tmed3 | transmembrane | NM_025360 | 2.370322108 | 0.020683453 | −0.499305241 | 0.256351169 | −0.195276448 | 0.642816996 | −0.257512421 | 0.529957284 | −0.101344914 | 0.714871853 |
| | | emp24 domain | | | | | | | | | | | |
| | | containing 3 | | | | | | | | | | | |
| 26 | Il10ra | interleukin 10 | NM_008348 | 2.325225048 | 0.021526529 | 2.225926115 | 0.03113759 | 0.615170648 | 0.301427608 | 0.132574794 | 0.704136691 | 0.125391786 | 0.666872752 |
| | | receptor, alpha | | | | | | | | | | | |
| 27 | Cox17 | cytochrome c | NM_001017429 | 2.31782868 | 0.021582734 | −0.657229003 | 0.188511691 | −0.025119811 | 0.938343076 | 0.160402659 | 0.658217176 | −0.125510431 | 0.666479317 |
| | | oxidase, subunit | | | | | | | | | | | |
| | | XVII assembly | | | | | | | | | | | |
| | | protein homolog | | | | | | | | | | | |
| | | (yeast) | | | | | | | | | | | |
| 28 | Mndal | myeloid nuclear | NM_001170853 | 2.310591378 | 0.021638939 | 1.805144373 | 0.042041367 | 0.327198977 | 0.493311601 | 0.573326476 | 0.28928732 | 0.095476993 | 0.728417266 |
| | | differentiation | | | | | | | | | | | |
| | | antigen like | | | | | | | | | | | |
| 29 | P2ry1 | purinergic | NM_008772 | 2.278856274 | 0.022144784 | −2.115717623 | 0.033385791 | 1.470228475 | 0.102799011 | −0.517065906 | 0.319694245 | −0.249432023 | 0.474201888 |
| | | receptor P2Y, G- | | | | | | | | | | | |
| | | protein coupled 1 | | | | | | | | | | | |
| 30 | Acot5 | acyl-CoA | NM_145444 | 2.250930441 | 0.022650629 | 1.22527489 | 0.078630845 | 1.878421314 | 0.07076214 | 12.14525981 | 0.001629946 | 2.190611463 | 0.029844874 |
| | | thioesterase 5 | | | | | | | | | | | |
| 31 | Ccr4 | chemokine (C-C | NM_009916 | 2.160176658 | 0.025123651 | −0.287022578 | 0.416985162 | 0.205497348 | 0.628765737 | −0.030959967 | 0.915580036 | 0.039958245 | 0.875168615 |
| | | motif) receptor 4 | | | | | | | | | | | |
| 32 | Cish | cytokine | NM_009895 | 2.01064125 | 0.028608363 | 1.724933544 | 0.045694694 | 1.825330763 | 0.074303058 | 0.933421117 | 0.167828237 | 0.720415473 | 0.17254946 |
| | | inducible SH2- | | | | | | | | | | | |
| | | containing | | | | | | | | | | | |
| | | protein | | | | | | | | | | | |
| 33 | Egr2 | early growth | NM_010118 | 2.002974924 | 0.028833183 | 1.229035585 | 0.078293615 | 2.325723855 | 0.049797662 | −0.105702574 | 0.753484712 | 0.593442976 | 0.217850719 |
| | | response 2 | | | | | | | | | | | |

TABLE 1B
Subset of genes in Table 1A that are also significantly activated via Toll-Like Receptor (TLR) stimulation in dendritic cells
(Amit, I. et al. 2009, supra).

| | | | | | Stimulation of | | | |
|---|---|---|---|---|---|---|---|---|
| | Universal | Universal Gene | Universal Gene | Stimulation of | TLR7 by | Stimulation of | Stimulation of | Stimulation of |
| # | Symbol | Name | Refseq ID | TLR9 by CpG | GRD | TLR4 by LPS | TLR2 by Pam | TLR3 by PiC |
| 1 | Btg2 | B-cell | NM_007570 | | | | | |
| | | translocation gene | | | | | | |
| | | 2, anti- | | | | | | |
| | | proliferative | | | | | | |
| 2 | Myc | myelocytomatosis | NM_010849 | | | | | |
| | | oncogene | | | | | | |
| 3 | Mkks | McKusick- | NM_001141946 | | | | | |
| | | Kaufman | | | | | | |
| | | syndrome protein | | | | | | |
| 4 | Cysltr1 | cysteinyl | NM_021476 | | | | | |
| | | leukotriene | | | | | | |
| | | receptor 1 | | | | | | |
| 5 | Prf1 | perforin 1 (pore | NM_011073 | | | + | + | |
| | | forming protein) | | | | | | |
| 6 | Frk | fyn-related kinase | NM_001159544 | + | | + | | + |
| 7 | Jun | Jun oncogene | NM_010591 | | | + | + | + |
| 8 | Atp2c2 | ATPase, Ca++ | NM_026922 | | | | | |
| | | transporting, type | | | | | | |
| | | 2C, member 2 | | | | | | |
| 9 | Ier3 | immediate early | NM_133662 | + | | + | | + |
| | | response 3 | | | | | | |
| 10 | Nr4a1 | nuclear receptor | NM_010444 | + | | | | |
| | | subfamily 4, | | | | | | |
| | | group A, member 1 | | | | | | |
| 11 | Gimap7 | GTPase, IMAP | NM_146167 | | | | | + |
| | | family member 7 | | | | | | |
| 12 | Junb | Jun-B oncogene | NM_008416 | | | + | + | + |
| 13 | Madcam1 | mucosal vascular | NM_013591 | | | | | |
| | | addressin cell | | | | | | |
| | | adhesion | | | | | | |
| | | molecule 1 | | | | | | |
| 14 | Zfp36 | zinc finger | NM_011756 | | + | | | |
| | | protein 36 | | | | | | |
| 15 | Gbp8 | guanylate-binding | NM_029509 | | | | | |
| | | protein 8 | | | | | | |
| 16 | Med17 | mediator complex | NM_144933 | + | | | + | + |
| | | subunit 17 | | | | | | |
| 17 | Akr1c18 | aldo-keto | NM_134066 | | | | | |
| | | reductase family | | | | | | |
| | | 1, member C18 | | | | | | |
| 18 | Egr1 | early growth | NM_007913 | | | | | |
| | | response 1 | | | | | | |
| 19 | N6amt2 | N-6 adenine- | NM_026526 | | + | | | |
| | | specific DNA | | | | | | |
| | | methyltransferase | | | | | | |
| | | 2 (putative) | | | | | | |
| 20 | Slc39a8 | solute carrier | NM_001135149 | | | | | + |
| | | family 39 (metal | | | | | | |
| | | ion transporter), | | | | | | |
| | | member 8 | | | | | | |
| 21 | Sik1 | salt inducible | NM_010831 | | | | + | |
| | | kinase 1 | | | | | | |
| 22 | LOC100046066 | similar to FAST | XM_001475471 | | | | | |
| | | kinase domains 1 | | | | | | |
| 23 | Gem | GTP binding | NM_010276 | + | | | + | + |
| | | protein (gene | | | | | | |
| | | overexpressed in | | | | | | |
| | | skeletal muscle) | | | | | | |
| 24 | Cdh10 | cadherin 10 | NM_009865 | + | | + | | |
| 25 | Tmed3 | transmembrane | NM_025360 | | | + | | |
| | | emp24 domain | | | | | | |
| | | containing 3 | | | | | | |
| 26 | Il10ra | interleukin 10 | NM_008348 | | | | | |
| | | receptor, alpha | | | | | | |
| 27 | Cox17 | cytochrome c | NM_001017429 | + | | + | + | |
| | | oxidase, subunit | | | | | | |
| | | XVII assembly | | | | | | |
| | | protein homolog | | | | | | |
| | | (yeast) | | | | | | |
| 28 | Mndal | myeloid nuclear | NM_001170853 | | | | + | + |
| | | differentiation | | | | | | |
| | | antigen like | | | | | | |
| 29 | P2ry1 | purinergic | NM_008772 | | | | | |
| | | receptor P2Y, G- | | | | | | |
| | | protein coupled 1 | | | | | | |
| 30 | Acot5 | acyl-CoA | NM_145444 | | | | + | |
| | | thioesterase 5 | | | | | | |
| 31 | Ccr4 | chemokine (C-C | NM_009916 | | + | + | | |
| | | motif) receptor 4 | | | | | | |
| 32 | Cish | cytokine | NM_009895 | | | | | + |
| | | inducible SH2- | | | | | | |
| | | containing protein | | | | | | |
| 33 | Egr2 | early growth | NM_010118 | | | + | | + |
| | | response 2 | | | | | | |

TABLE 2A
Genes showing ≧2-fold increased expression 15-30 minutes after DBP application, with corresponding expression changes at 30 min-1 hr, 1-2 hr and 2-4 hr time intervals.

| | | | | Change in Gene Expression (Weighted Difference) | | | | | | | |
|---|---|---|---|---|---|---|---|---|---|---|---|
| | | | | 15 min to | 15 min to | | | | | | |
| | | | | 30 min | 30 min | 30 min to 1 hr | 30 min to 1 hr | 1 hr to 2 hr | 1 hr to 2 hr | 2 hr to 4 hr | 2 hr to 4 hr |
| # | Universal Symbol | Universal Gene Name | Universal Gene Refseq ID | Transition | P-value | Transition | P-value | Transition | P-value | Transition | P-value |
| 1 | H6pd | hexose-6- | NM_173371 | 50.660567 | 0.00011241 | −0.1323903 | 0.73341951 | 0.0768132 | 0.810308 | 0.02160376 | 0.93086781 |
| | | phosphate | | | | | | | | | |
| | | dehydrogenase | | | | | | | | | |
| | | (glucose 1- | | | | | | | | | |
| | | dehydrogenase) | | | | | | | | | |
| 2 | Il6 | interleukin 6 | NM_031168 | 46.9897323 | 0.00011241 | 50.8425683 | 5.6205E−05 | 98.2862155 | 5.6205E−05 | 11.6653974 | 0.00101169 |
| 3 | Samhd1 | SAM domain and | NM_001139520 | 38.8539426 | 0.00016862 | 0.0721158 | 0.83751124 | 0.3780169 | 0.41378147 | 0.03573701 | 0.88764613 |
| | | HD domain, 1 | | | | | | | | | |
| 4 | Ccl20 | chemokine (C-C | NM_001159738 | 42.371726 | 0.00016862 | 30.2573915 | 0.00022482 | 7.5461823 | 0.0044402 | 13.8685395 | 0.00073067 |
| | | motif) ligand 20 | | | | | | | | | |
| 5 | Hdc | histidine | NM_008230 | 38.9552826 | 0.00016862 | −0.1574406 | 0.69463804 | 1.27753148 | 0.10943121 | 0.93319771 | 0.12185252 |
| | | decarboxylase | | | | | | | | | |
| 6 | LOC100047619 | similar to solute | XR_033736 | 33.7422613 | 0.00028103 | 0.02020511 | 0.95059577 | 0.97294706 | 0.15883543 | 1.23980565 | 0.07936151 |
| | | carrier family 7 | | | | | | | | | |
| | | (cationic amino | | | | | | | | | |
| | | acid transporter, | | | | | | | | | |
| | | y+ system), | | | | | | | | | |
| | | member 5 | | | | | | | | | |
| 7 | Slc7a5 | solute carrier | NM_011404 | 33.7422613 | 0.00028103 | 0.02020511 | 0.95059577 | 0.97294706 | 0.15883543 | 1.23980565 | 0.07936151 |
| | | family 7 (cationic | | | | | | | | | |
| | | amino acid | | | | | | | | | |
| | | transporter, y+ | | | | | | | | | |
| | | system), member 5 | | | | | | | | | |
| 8 | Cxcl1 | chemokine (C—X—C | NM_008176 | 30.1601552 | 0.00044964 | 15.1093962 | 0.00151754 | 3.27743733 | 0.02529227 | 9.9247383 | 0.00146133 |
| | | motif) ligand 1 | | | | | | | | | |
| 9 | Iigp1 | interferon | NM_001146275 | 24.1038373 | 0.00050585 | −0.0296957 | 0.93002473 | 0.38326113 | 0.41012815 | 0.05527114 | 0.83042941 |
| | | inducible GTPase 1 | | | | | | | | | |
| 10 | Slc39a8 | solute carrier | NM_001135149 | 25.2658209 | 0.00050585 | 2.30389139 | 0.05041592 | 1.18845197 | 0.12106565 | 2.10057329 | 0.03243031 |
| | | family 39 (metal | | | | | | | | | |
| | | ion transporter), | | | | | | | | | |
| | | member 8 | | | | | | | | | |
| 11 | Ccl2 | chemokine (C-C | NM_011333 | 21.4075718 | 0.00067446 | 4.01115821 | 0.0200652 | 4.62046134 | 0.01247752 | 6.32609048 | 0.00410297 |
| | | motif) ligand 2 | | | | | | | | | |
| 12 | Ccl1 | chemokine (C-C | NM_011329 | 23.1761517 | 0.00067446 | 3.5936137 | 0.02354991 | 1.43753398 | 0.09375 | 0.71191361 | 0.17513489 |
| | | motif) ligand 1 | | | | | | | | | |
| 13 | Icam1 | intercellular | NM_010493 | 19.2243223 | 0.00073067 | 6.99699648 | 0.00719424 | 5.47276099 | 0.00904901 | 7.67026655 | 0.00297887 |
| | | adhesion | | | | | | | | | |
| | | molecule 1 | | | | | | | | | |
| 14 | Cxcl16 | chemokine (C—X—C | NM_023158 | 19.2452177 | 0.00073067 | 5.26271307 | 0.01320818 | 3.64120451 | 0.01939074 | 1.82170157 | 0.04181655 |
| | | motif) ligand 16 | | | | | | | | | |
| 15 | Clic4 | chloride | NM_013885 | 21.1258808 | 0.00073067 | 6.13588003 | 0.00966727 | 3.49886826 | 0.0212455 | 3.64361412 | 0.01174685 |
| | | intracellular | | | | | | | | | |
| | | channel 4 | | | | | | | | | |
| | | (mitochondrial) | | | | | | | | | |
| 16 | Cd83 | CD83 antigen | NM_009856 | 18.1580884 | 0.00078687 | 7.50922652 | 0.00623876 | 7.48079843 | 0.00460881 | 9.95963671 | 0.00140513 |
| 17 | Dusp2 | dual specificity | NM_010090 | 17.4255573 | 0.00078687 | 5.17045251 | 0.01360162 | 3.84605067 | 0.01719874 | 5.62986843 | 0.00483363 |
| | | phosphatase 2 | | | | | | | | | |
| 18 | Irf1 | interferon | NM_001159396 | 16.949717 | 0.00084308 | 2.67627275 | 0.03962455 | 6.1187518 | 0.00741906 | 6.00349211 | 0.00432779 |
| | | regulatory factor 1 | | | | | | | | | |
| 19 | Ccl7 | chemokine (C-C | NM_013654 | 15.074359 | 0.00095549 | 2.33140886 | 0.04974146 | 3.79223618 | 0.017817 | 5.50173481 | 0.00505845 |
| | | motif) ligand 7 | | | | | | | | | |
| 20 | Ccl12 | chemokine (C-C | NM_011331 | 14.4331203 | 0.00095549 | 3.05139765 | 0.03085656 | 7.98700883 | 0.00404676 | 10.4997414 | 0.00123651 |
| | | motif) ligand 12 | | | | | | | | | |
| 21 | Cidec | cell death- | NM_178373 | 14.5810161 | 0.00095549 | −0.0993663 | 0.78810701 | 0.4236447 | 0.3783161 | 0.2631277 | 0.45767761 |
| | | inducing DFFA- | | | | | | | | | |
| | | like effector c | | | | | | | | | |
| 22 | Egln3 | EGL nine | NM_028133 | 14.9711442 | 0.00095549 | 0.76368675 | 0.24291817 | 2.1063939 | 0.0513714 | 0.54821569 | 0.2377473 |
| | | homolog 3 (C. elegans) | | | | | | | | | |
| 23 | F7 | coagulation | NM_010172 | 15.3847719 | 0.00095549 | 0.35080549 | 0.47127923 | 0.38379633 | 0.40967851 | −0.0105462 | 0.96610836 |
| | | factor VII | | | | | | | | | |
| 24 | Angptl4 | angiopoietin-like 4 | NM_020581 | 14.1394415 | 0.0011241 | 1.59971218 | 0.09127698 | 3.43838172 | 0.02259442 | 3.11203559 | 0.01607464 |
| 25 | Ier3 | immediate early | NM_133662 | 12.7333495 | 0.00140513 | 7.14535067 | 0.0067446 | 0.77859953 | 0.2075652 | 4.52124436 | 0.00814973 |
| | | response 3 | | | | | | | | | |
| 26 | Zc3h12a | zinc finger CCCH | NM_153159 | 12.3784909 | 0.00151754 | 4.27125949 | 0.01809802 | 0.03134441 | 0.91484937 | 0.7117596 | 0.1751911 |
| | | type containing | | | | | | | | | |
| | | 12A | | | | | | | | | |
| 27 | Clec7a | C-type lectin | NM_020008 | 12.5882959 | 0.00151754 | 2.9263444 | 0.03321718 | 5.72189875 | 0.00831835 | 1.413746 | 0.06491682 |
| | | domain family 7, | | | | | | | | | |
| | | member a | | | | | | | | | |
| 28 | Ccl3 | chemokine (C-C | NM_011337 | 12.3765496 | 0.00151754 | 1.98463517 | 0.06519784 | 7.07158877 | 0.00522707 | 9.28151753 | 0.00179856 |
| | | motif) ligand 3 | | | | | | | | | |
| 29 | Pqlc1 | PQ loop repeat | NM_001164420 | 11.3964952 | 0.00162995 | 1.38038426 | 0.1138152 | 3.01953548 | 0.02922662 | 9.18503591 | 0.00179856 |
| | | containing 1 | | | | | | | | | |
| 30 | Serpine1 | serine (or | NM_008871 | 11.6300307 | 0.00162995 | 12.5739677 | 0.002192 | 4.43850027 | 0.01348921 | 3.74756861 | 0.01129721 |
| | | cysteine) | | | | | | | | | |
| | | peptidase | | | | | | | | | |
| | | inhibitor, clade E, | | | | | | | | | |
| | | member 1 | | | | | | | | | |
| 31 | Il1b | interleukin 1 beta | NM_008361 | 11.7085917 | 0.00162995 | 2.86561752 | 0.03484712 | 11.1468343 | 0.00207959 | 10.0351689 | 0.00140513 |
| 32 | Cyp2e1 | cytochrome P450, | NM_021282 | 10.3733796 | 0.00174236 | −2.1471569 | 0.05699191 | 0.55764873 | 0.29636915 | 0.21384798 | 0.52147032 |
| | | family 2, | | | | | | | | | |
| | | subfamily e, | | | | | | | | | |
| | | polypeptide 1 | | | | | | | | | |
| 33 | 1810011010Rik | RIKEN cDNA | NM_026931 | 10.5114013 | 0.00174236 | 6.03779268 | 0.00989209 | 1.25804078 | 0.11139838 | 1.59317099 | 0.05260791 |
| | | 1810011010 gene | | | | | | | | | |
| 34 | Zfp36 | zinc finger | NM_011756 | 10.6971287 | 0.00174236 | 2.80047057 | 0.03625225 | 0.10246334 | 0.75994829 | 2.6377143 | 0.02175135 |
| | | protein 36 | | | | | | | | | |
| 35 | Mmp12 | matrix | NM_008605 | 10.5938185 | 0.00174236 | 1.80304557 | 0.07570818 | 1.01833358 | 0.14922437 | 0.00512224 | 0.98291367 |
| | | metallopeptidase | | | | | | | | | |
| | | 12 | | | | | | | | | |
| 36 | Ccl17 | chemokine (C-C | NM_011332 | 10.6930711 | 0.00174236 | 4.43143245 | 0.01691772 | 3.76277949 | 0.01798561 | 2.3795327 | 0.02596673 |
| | | motif) ligand 17 | | | | | | | | | |
| 37 | Ifi202b | interferon | NM_008327 | 9.73500327 | 0.00179856 | 3.38443256 | 0.02551709 | 2.13360846 | 0.05035971 | 1.95644914 | 0.03748876 |
| | | activated gene | | | | | | | | | |
| | | 202B | | | | | | | | | |
| 38 | Bcl3 | B-cell | NM_033601 | 9.84654364 | 0.00179856 | 6.33731345 | 0.0089366 | 15.8103466 | 0.00073067 | 10.9418141 | 0.00118031 |
| | | leukemia/lymphoma 3 | | | | | | | | | |
| 39 | Gpr65 | G-protein coupled | NM_008152 | 9.90520859 | 0.00179856 | 0.22919639 | 0.59908948 | 1.04166745 | 0.14529002 | 1.24731384 | 0.07863085 |
| | | receptor 65 | | | | | | | | | |
| 40 | Slc39a14 | solute carrier | NM_001135151 | 9.56623875 | 0.00191097 | 0.93150362 | 0.19244604 | 0.6314831 | 0.26124101 | 0.36841815 | 0.35420414 |
| | | family 39 (zinc | | | | | | | | | |
| | | transporter), | | | | | | | | | |
| | | member 14 | | | | | | | | | |
| 41 | Saa3 | serum amyloid A 3 | NM_011315 | 9.55832414 | 0.00191097 | 5.09773571 | 0.01388264 | 13.3179235 | 0.00134892 | 3.65816029 | 0.01174685 |
| 42 | Mefv | Mediterranean | NM_001161790 | 8.87429253 | 0.00213579 | 3.19154688 | 0.02849595 | 0.8242427 | 0.19553732 | 0.44882895 | 0.29445818 |
| | | fever | | | | | | | | | |
| 43 | Spp1 | secreted | NM_009263 | 8.68154735 | 0.0022482 | −0.281227 | 0.53928732 | 0.75506909 | 0.2142536 | 0.07909554 | 0.76798561 |
| | | phosphoprotein 1 | | | | | | | | | |
| 44 | Nfkbiz | nuclear factor of | NM_001159394 | 8.62216885 | 0.00230441 | 1.68112883 | 0.08492581 | −0.093318 | 0.77725944 | 0.62176522 | 0.20689074 |
| | | kappa light | | | | | | | | | |
| | | polypeptide gene | | | | | | | | | |
| | | enhancer in B- | | | | | | | | | |
| | | cells inhibitor, | | | | | | | | | |
| | | zeta | | | | | | | | | |
| 45 | Epgn | epithelial | NM_053087 | 7.85430609 | 0.00258543 | 1.45180137 | 0.10459757 | 2.29261298 | 0.04507644 | 14.7995621 | 0.00067446 |
| | | mitogen | | | | | | | | | |
| 46 | Mt1 | metallothionein 1 | NM_013602 | 7.72301081 | 0.00264164 | 2.85975997 | 0.03507194 | 1.31968042 | 0.10426034 | 1.6245562 | 0.05142761 |
| 47 | Nfkb1 | nuclear factor of | NM_008689 | 7.79503808 | 0.00264164 | 0.97351356 | 0.18221673 | 0.22673564 | 0.56755845 | 0.78652956 | 0.15450764 |
| | | kappa light | | | | | | | | | |
| | | polypeptide gene | | | | | | | | | |
| | | enhancer in B- | | | | | | | | | |
| | | cells 1, p105 | | | | | | | | | |
| 48 | Cebpd | CCAAT/enhancer | NM_007679 | 7.71535747 | 0.00269784 | 1.22851624 | 0.1350607 | 1.08000767 | 0.13742131 | 0.91806087 | 0.12505621 |
| | | binding protein | | | | | | | | | |
| | | (C/EBP), delta | | | | | | | | | |
| 49 | Acer2 | alkaline | NM_139306 | 7.11535396 | 0.00325989 | 0.61755155 | 0.3002473 | 1.07257539 | 0.13871403 | 0.5597188 | 0.23252023 |
| | | ceramidase 2 | | | | | | | | | |
| 50 | Coro2a | coronin, actin | NM_001164804 | 7.17388509 | 0.00325989 | −0.6558152 | 0.28181205 | 0.6234436 | 0.26483813 | 0.29395399 | 0.42159397 |
| | | binding protein | | | | | | | | | |
| | | 2A | | | | | | | | | |
| 51 | Best2 | bestrophin 2 | NM_001130193 | 7.22971747 | 0.00325989 | −0.5194498 | 0.35251799 | 0.18909687 | 0.61572617 | 0.03274307 | 0.89821268 |
| 52 | Ccl24 | chemokine (C-C | NM_019577 | 6.97715318 | 0.0033161 | 1.19614286 | 0.1395571 | 3.64763706 | 0.01922212 | 2.44736531 | 0.02478642 |
| | | motif) ligand 24 | | | | | | | | | |
| 53 | Ltb4r1 | leukotriene B4 | NM_008519 | 7.05393371 | 0.0033161 | 1.14680171 | 0.14793165 | 0.09739975 | 0.76950315 | 1.1531927 | 0.08936601 |
| | | receptor 1 | | | | | | | | | |
| 54 | Tnfrsf1a | tumor necrosis | NM_011609 | 6.91597494 | 0.0033723 | 0.77898844 | 0.2376911 | 0.1333243 | 0.703125 | 0.23706202 | 0.49050135 |
| | | factor receptor | | | | | | | | | |
| | | superfamily, | | | | | | | | | |
| | | member 1a | | | | | | | | | |
| 55 | Dusp6 | dual specificity | NM_026268 | 6.81365029 | 0.00342851 | 4.38788749 | 0.01714254 | 0.88288791 | 0.18092401 | 2.81472178 | 0.01944694 |
| | | phosphatase 6 | | | | | | | | | |
| 56 | LOC100044068 | similar to | XM_001473873 | 6.84456115 | 0.00342851 | 3.0257189 | 0.03119379 | 2.69763506 | 0.03479092 | 2.16273071 | 0.03085656 |
| | | interferon- | | | | | | | | | |
| | | inducible Ifi202b | | | | | | | | | |
| 57 | Cxcl2 | chemokine (C—X—C | NM_009140 | 6.74609007 | 0.00348471 | 0.28801384 | 0.5323741 | 6.66026522 | 0.00590153 | 11.9170126 | 0.00095549 |
| | | motif) ligand 2 | | | | | | | | | |
| 58 | Adam8 | a disintegrin and | NM_007403 | 6.7055836 | 0.00354092 | 3.40517197 | 0.02529227 | 7.99930794 | 0.00404676 | 4.28349907 | 0.00916142 |
| | | metallopeptidase | | | | | | | | | |
| | | domain 8 | | | | | | | | | |
| 59 | Alox12e | arachidonate | NM_145684 | 6.4927174 | 0.00399056 | 1.37302275 | 0.11505171 | 0.2942215 | 0.48971448 | −0.0074003 | 0.97633768 |
| | | lipoxygenase, | | | | | | | | | |
| | | epidermal | | | | | | | | | |
| 60 | Cd86 | CD86 antigen | NM_019388 | 6.33708619 | 0.00404676 | 6.82774927 | 0.00741906 | 3.14941814 | 0.02697842 | 3.54352797 | 0.01281475 |
| 61 | Scd3 | stearoyl- | NM_024450 | 6.41855514 | 0.00404676 | 0.46140248 | 0.3873089 | 0.09563483 | 0.77270683 | −0.0783056 | 0.76989658 |
| | | coenzyme A | | | | | | | | | |
| | | desaturase 3 | | | | | | | | | |
| 62 | Rela | v-rel | NM_009045 | 6.20530525 | 0.00421538 | 0.2150874 | 0.61617581 | 0.40179256 | 0.39422212 | 0.30399283 | 0.41215153 |
| | | reticuloendotheliosis | | | | | | | | | |
| | | viral | | | | | | | | | |
| | | oncogene | | | | | | | | | |
| | | homolog A | | | | | | | | | |
| | | (avian) | | | | | | | | | |
| 63 | Tph1 | tryptophan | NM_001136084 | 6.17605687 | 0.00427158 | 0.19822575 | 0.63832059 | 0.94536107 | 0.16552383 | 0.16518565 | 0.59301933 |
| | | hydroxylase 1 | | | | | | | | | |
| 64 | Ccl11 | chemokine (C-C | NM_011330 | 6.17467356 | 0.00427158 | 0.24737144 | 0.57508993 | 1.66293625 | 0.07492131 | 1.33899465 | 0.07064973 |
| | | motif) ligand 11 | | | | | | | | | |
| 65 | Plin4 | perilipin 4 | NM_020568 | 6.08235041 | 0.0044402 | −0.5392633 | 0.3415018 | 0.1377402 | 0.69525629 | 0.04290508 | 0.86550135 |
| 66 | Ptx3 | pentraxin related | NM_008987 | 6.06198358 | 0.0044402 | 4.40558721 | 0.01703013 | 41.4831921 | 0.00011241 | 5.39750438 | 0.00528327 |
| | | gene | | | | | | | | | |
| 67 | Ralgds | ral guanine | NM_001145834 | 6.00440461 | 0.0044964 | 1.05874761 | 0.16417491 | 0.21038921 | 0.58908498 | 0.76384751 | 0.15973471 |
| | | nucleotide | | | | | | | | | |
| | | dissociation | | | | | | | | | |
| | | stimulator | | | | | | | | | |
| 68 | Btg2 | B-cell | NM_007570 | 5.99403178 | 0.0044964 | 2.92591969 | 0.03321718 | −0.0100486 | 0.97150405 | 1.00902031 | 0.10982464 |
| | | translocation | | | | | | | | | |
| | | gene 2, anti- | | | | | | | | | |
| | | proliferative | | | | | | | | | |
| 69 | Pyy | peptide YY | NM_145435 | 5.95583133 | 0.00460881 | 4.37949069 | 0.01731115 | 4.67010147 | 0.01208408 | 2.58685115 | 0.0223134 |
| 70 | Rab20 | RAB20, member | NM_011227 | 5.93418114 | 0.00466502 | 1.63598213 | 0.08835432 | 4.51604257 | 0.01309577 | 3.5981169 | 0.0122527 |
| | | RAS oncogene | | | | | | | | | |
| | | family | | | | | | | | | |
| 71 | C330016010Rik | RIKEN cDNA | NM_145974 | 5.89011971 | 0.00477743 | 0.25999081 | 0.56120728 | 0.0367146 | 0.9028777 | 0.16895266 | 0.58773606 |
| | | C330016010 gene | | | | | | | | | |
| 72 | Zfp593 | zinc finger | NM_024215 | 5.86625108 | 0.00483363 | 1.96587703 | 0.06598471 | 0.77274397 | 0.20891412 | 1.80120839 | 0.04277203 |
| | | protein 593 | | | | | | | | | |
| 73 | LOC100048724 | similar to fatty | XM_001481056 | 5.845032 | 0.00488984 | 0.46012888 | 0.38809577 | 0.1762198 | 0.63404901 | −0.0978682 | 0.72262815 |
| | | acid desaturase | | | | | | | | | |
| 74 | Nr4a1 | nuclear receptor | NM_010444 | 5.84223242 | 0.00488984 | 1.55617497 | 0.09515513 | −0.4733707 | 0.3448741 | 4.57195763 | 0.00792491 |
| | | subfamily 4, | | | | | | | | | |
| | | group A, member 1 | | | | | | | | | |
| 75 | Marcksl1 | MARCKS-like 1 | NM_010807 | 5.82282966 | 0.00500225 | 1.33217474 | 0.11994155 | 2.12811128 | 0.05058453 | 8.90695041 | 0.00185477 |
| 76 | Hamp2 | hepcidin | NM_183257 | 5.73739636 | 0.00505845 | −0.6185737 | 0.29985387 | −0.0815016 | 0.80013489 | −0.4057136 | 0.32295414 |
| | | antimicrobial | | | | | | | | | |
| | | peptide 2 | | | | | | | | | |
| 77 | Fcer1a | Fc receptor, IgE, | NM_010184 | 5.73183159 | 0.00511466 | 0.82197454 | 0.22307779 | 1.30112847 | 0.10667716 | 0.15162088 | 0.61690647 |
| | | high affinity I, | | | | | | | | | |
| | | alpha polypeptide | | | | | | | | | |
| 78 | LOC100044927 | similar to TNF- | XM_001473344 | 5.68811053 | 0.00522707 | 3.21118781 | 0.02827113 | 13.8550031 | 0.0011241 | 63.0500222 | 0 |
| | | stimulated gene 6 | | | | | | | | | |
| | | protein | | | | | | | | | |
| 79 | Anxa3 | annexin A3 | NM_013470 | 5.59545957 | 0.00533948 | −0.501131 | 0.36375899 | 0.2035989 | 0.59644784 | 0.02061236 | 0.93362185 |
| 80 | Ccl4 | chemokine (C-C | NM_013652 | 5.58590142 | 0.00533948 | 1.48904639 | 0.10139388 | 2.01143383 | 0.05558678 | 1.06096756 | 0.10116906 |
| | | motif) ligand 4 | | | | | | | | | |
| 81 | Gpr84 | G protein-coupled | NM_030720 | 5.59997638 | 0.00533948 | −2.1769182 | 0.05569919 | 1.50018783 | 0.08801709 | 0.20689997 | 0.53046313 |
| | | receptor 84 | | | | | | | | | |
| 82 | Rgs1 | regulator of G- | NM_015811 | 5.52164798 | 0.00545189 | 3.62873132 | 0.02310027 | 0.93157967 | 0.16833408 | 1.58171898 | 0.05316996 |
| | | protein signaling 1 | | | | | | | | | |
| 83 | Defb1 | defensin beta 1 | NM_007843 | 5.46091834 | 0.0056205 | 0.4984581 | 0.36566996 | 0.77564244 | 0.20835207 | 0.2292619 | 0.50129272 |
| 84 | Nfkbia | nuclear factor of | NM_010907 | 5.41137712 | 0.00573291 | 2.49660173 | 0.04417716 | 3.01809178 | 0.02922662 | 3.98460234 | 0.0100045 |
| | | kappa light | | | | | | | | | |
| | | polypeptide gene | | | | | | | | | |
| | | enhancer in B- | | | | | | | | | |
| | | cells inhibitor, | | | | | | | | | |
| | | alpha | | | | | | | | | |
| 85 | Gsdmc | gasdermin C | NM_031378 | 5.43066543 | 0.00573291 | 5.76942767 | 0.01084757 | 0.09582681 | 0.7723134 | −0.0089433 | 0.97071718 |
| 86 | Ch25h | cholesterol 25- | NM_009890 | 5.40912727 | 0.00578912 | 3.44331554 | 0.02501124 | 3.76590989 | 0.01792941 | 7.6351804 | 0.00303507 |
| | | hydroxylase | | | | | | | | | |
| 87 | Casp4 | caspase 4, | NM_007609 | 5.29167121 | 0.00590153 | 1.99060348 | 0.06514164 | 1.47420945 | 0.09015288 | 1.71821518 | 0.04704362 |
| | | apoptosis-related | | | | | | | | | |
| | | cysteine | | | | | | | | | |
| | | peptidase | | | | | | | | | |
| 88 | LOC100044206 | hypothetical | XM_001471596 | 5.29167121 | 0.00590153 | 1.99060348 | 0.06514164 | 1.47420945 | 0.09015288 | 1.71821518 | 0.04704362 |
| | | Protein | | | | | | | | | |
| | | LOC100044206 | | | | | | | | | |
| 89 | LOC100046186 | similar to | XM_001475752 | 5.23802358 | 0.00607014 | 1.04728418 | 0.16597347 | 0.83731147 | 0.19278327 | 0.29848136 | 0.41704137 |
| | | receptor activity | | | | | | | | | |
| | | modifying | | | | | | | | | |
| | | protein 3 | | | | | | | | | |
| 90 | Ramp3 | receptor | NM_019511 | 5.23802358 | 0.00607014 | 1.04728418 | 0.16597347 | 0.83731147 | 0.19278327 | 0.29848136 | 0.41704137 |
| | | (calcitonin) | | | | | | | | | |
| | | activity | | | | | | | | | |
| | | modifying | | | | | | | | | |
| | | protein 3 | | | | | | | | | |
| 91 | Tnfaip2 | tumor necrosis | NM_009396 | 5.19518242 | 0.00618255 | 6.31814794 | 0.00899281 | 5.14278983 | 0.0100607 | 5.64776754 | 0.00483363 |
| | | factor, alpha- | | | | | | | | | |
| | | induced protein 2 | | | | | | | | | |
| 92 | Ankrd57 | ankyrin repeat | NM_172939 | 5.0553538 | 0.00651978 | 2.82535477 | 0.03591502 | 0.69500165 | 0.23504946 | 1.18041957 | 0.08661196 |
| | | domain 57 | | | | | | | | | |
| 93 | Mat2a | methionine | NM_145569 | 4.95212012 | 0.0067446 | −0.0096436 | 0.9754384 | 0.34561335 | 0.43997302 | 0.8446544 | 0.14101844 |
| | | adenosyltransferase | | | | | | | | | |
| | | II, alpha | | | | | | | | | |
| 94 | Egr1 | early growth | NM_007913 | 4.96650141 | 0.0067446 | 5.49510779 | 0.01219649 | −0.2126554 | 0.58666817 | 2.34790819 | 0.02664119 |
| | | response 1 | | | | | | | | | |
| 95 | Slfn2 | schlafen 2 | NM_011408 | 4.9352757 | 0.0067446 | 0.78324807 | 0.23673561 | 2.67951833 | 0.03535297 | 1.31096085 | 0.07312275 |
| 96 | Ccr7 | chemokine (C-C | NM_007719 | 4.88311562 | 0.00680081 | 0.23459852 | 0.59223246 | 0.01766164 | 0.95025854 | 0.05622961 | 0.82750674 |
| | | motif) receptor 7 | | | | | | | | | |
| 97 | Pmaip1 | phorbol-12- | NM_021451 | 4.77120933 | 0.00708183 | 3.37088162 | 0.02574191 | 4.08404899 | 0.01568121 | 8.41677488 | 0.0022482 |
| | | myristate-13- | | | | | | | | | |
| | | acetate-induced | | | | | | | | | |
| | | protein 1 | | | | | | | | | |
| 98 | Tnfaip6 | tumor necrosis | NM_009398 | 4.76766094 | 0.00708183 | 1.52642027 | 0.09790917 | 8.02345848 | 0.00399056 | 7.24962887 | 0.00325989 |
| | | factor alpha | | | | | | | | | |
| | | induced protein 6 | | | | | | | | | |
| 99 | Il4i1 | interleukin 4 | NM_010215 | 4.61978333 | 0.00736286 | 5.74368474 | 0.01107239 | 7.85163456 | 0.00410297 | 0.95668071 | 0.11848022 |
| | | induced 1 | | | | | | | | | |
| 100 | Nup62-ili41 | Nup62-Il4i1 | NM_001171024 | 4.61978333 | 0.00736286 | 5.74368474 | 0.01107239 | 7.85163456 | 0.00410297 | 0.95668071 | 0.11848022 |
| | | protein | | | | | | | | | |
| 101 | Mt2 | metallothionein 2 | NM_008630 | 4.58986149 | 0.00747527 | 1.76473976 | 0.07863085 | 1.45966223 | 0.091558 | 3.26779046 | 0.01489433 |
| 102 | Batf3 | basic leucine | NM_030060 | 4.57425362 | 0.00758768 | 0.35656651 | 0.46599595 | 0.27702573 | 0.50927383 | 0.000413 | 0.99848246 |
| | | zipper | | | | | | | | | |
| | | transcription | | | | | | | | | |
| | | factor, ATF-like 3 | | | | | | | | | |
| 103 | Nr4a2 | nuclear receptor | NM_001139509 | 4.51727932 | 0.0078125 | 0.67106861 | 0.27630396 | 0.11490736 | 0.73617356 | 3.39369087 | 0.01393885 |
| | | subfamily 4, | | | | | | | | | |
| | | group A, member 2 | | | | | | | | | |
| 104 | Plek | pleckstrin | NM_019549 | 4.49463175 | 0.00792491 | 2.07058379 | 0.06075764 | 3.68222379 | 0.01910971 | 2.3550479 | 0.02647257 |
| 105 | Phlda1 | pleckstrin | NM_009344 | 4.50695393 | 0.00792491 | 1.71521141 | 0.0814411 | 1.62229545 | 0.07790018 | 3.90126828 | 0.01045414 |
| | | homology-like | | | | | | | | | |
| | | domain, family A, | | | | | | | | | |
| | | member 1 | | | | | | | | | |
| 106 | Pfkfb3 | 6-phosphofructo- | NM_133232 | 4.44926086 | 0.00809353 | −0.061901 | 0.85752023 | 0.67544194 | 0.24263714 | 0.59524615 | 0.216558 |
| | | 2- | | | | | | | | | |
| | | kinase/fructose- | | | | | | | | | |
| | | 2,6- | | | | | | | | | |
| | | biphosphatase 3 | | | | | | | | | |
| 107 | Slc10a6 | solute carrier | NM_029415 | 4.39614022 | 0.00837455 | 4.66496744 | 0.01528777 | 1.70221325 | 0.07239209 | 0.68788824 | 0.18317221 |
| | | family 10 | | | | | | | | | |
| | | (sodium/bile acid | | | | | | | | | |
| | | cotransporter | | | | | | | | | |
| | | family), member 6 | | | | | | | | | |
| 108 | Havcr2 | hepatitis A virus | NM_134250 | 4.3688207 | 0.00854317 | 9.63334987 | 0.00382194 | 12.4077268 | 0.00151754 | 6.3485387 | 0.00410297 |
| | | cellular receptor 2 | | | | | | | | | |
| 109 | Ms4a4b | membrane- | NM_021718 | 4.34089254 | 0.00859937 | −0.1005784 | 0.78625225 | −0.0608294 | 0.84588579 | −0.2999093 | 0.41558004 |
| | | spanning 4- | | | | | | | | | |
| | | domains, | | | | | | | | | |
| | | subfamily A, | | | | | | | | | |
| | | member 4B | | | | | | | | | |
| 110 | Pex13 | peroxisomal | NM_023651 | 4.33390796 | 0.00859937 | 0.13843169 | 0.72363984 | 0.20757884 | 0.59228867 | 0.36211541 | 0.35875674 |
| | | biogenesis factor | | | | | | | | | |
| | | 13 | | | | | | | | | |
| 111 | Bhlhe40 | basic helix-loop- | NM_011498 | 4.27114365 | 0.00899281 | 1.63188267 | 0.08857914 | 1.6988707 | 0.0725045 | 61.5798556 | 0 |
| | | helix family, | | | | | | | | | |
| | | member e40 | | | | | | | | | |
| 112 | Maff | v-maf | NM_010755 | 4.25694238 | 0.00910522 | 1.57189806 | 0.09352518 | 3.56359487 | 0.02040243 | 1.94341521 | 0.03782599 |
| | | musculoaponeurotic | | | | | | | | | |
| | | fibrosarcoma | | | | | | | | | |
| | | oncogene family, | | | | | | | | | |
| | | protein F (avian) | | | | | | | | | |
| 113 | Ptges | prostaglandin E | NM_022415 | 4.18747914 | 0.00949865 | 2.67215191 | 0.03968076 | 2.28046149 | 0.04530126 | 2.4867706 | 0.02399955 |
| | | synthase | | | | | | | | | |
| 114 | Il7r | interleukin 7 | NM_008372 | 4.17932999 | 0.00955486 | 0.79891003 | 0.23094649 | 0.627431 | 0.26326439 | 0.07574108 | 0.77529227 |
| | | receptor | | | | | | | | | |
| 115 | Ttr | transthyretin | NM_013697 | 4.1290499 | 0.00972347 | −0.1842554 | 0.65647482 | 0.35103995 | 0.43553282 | 0.32481388 | 0.39472797 |
| 116 | Map3k6 | mitogen-activated | NM_016693 | 4.13054921 | 0.00972347 | −0.4044428 | 0.42772032 | 0.85605003 | 0.18873651 | 0.49287303 | 0.267817 |
| | | protein kinase | | | | | | | | | |
| | | kinase kinase 6 | | | | | | | | | |
| 117 | Mafk | v-maf | NM_010757 | 4.06754785 | 0.0100045 | 0.37254051 | 0.45318121 | 0.06496886 | 0.83515063 | 0.12942949 | 0.65894784 |
| | | musculoaponeurotic | | | | | | | | | |
| | | fibrosarcoma | | | | | | | | | |
| | | oncogene family, | | | | | | | | | |
| | | protein K (avian) | | | | | | | | | |
| 118 | Clec4e | C-type lectin | NM_019948 | 4.05505054 | 0.01017311 | 3.10159607 | 0.02984487 | 21.6527479 | 0.00039344 | 3.0198837 | 0.01697392 |
| | | domain family 4, | | | | | | | | | |
| | | member e | | | | | | | | | |
| 119 | Cfd | complement | NM_013459 | 3.99183535 | 0.01062275 | −0.3847738 | 0.44351394 | 0.50381288 | 0.32790018 | 0.02732386 | 0.91417491 |
| | | factor D (adipsin) | | | | | | | | | |
| 120 | B4galt1 | UDP- | NM_022305 | 3.98573457 | 0.01062275 | 0.5722498 | 0.32340378 | 0.67590875 | 0.24224371 | 0.47550564 | 0.27804631 |
| | | Gal:betaGlcNAc | | | | | | | | | |
| | | beta 1,4- | | | | | | | | | |
| | | galactosyltransferase, | | | | | | | | | |
| | | polypeptide 1 | | | | | | | | | |
| 121 | Gpr133 | G protein-coupled | NM_001081342 | 3.94541878 | 0.01062275 | −0.1416785 | 0.71773831 | 0.08178043 | 0.79946043 | 0.05685675 | 0.82565198 |
| | | receptor 133 | | | | | | | | | |
| 122 | Junb | Jun-B oncogene | NM_008416 | 3.92373904 | 0.01073516 | 2.77139181 | 0.03692671 | −0.0754019 | 0.81334308 | 0.91186532 | 0.12618031 |
| 123 | Zkscan6 | zinc finger with | NM_026107 | 3.92704251 | 0.01073516 | 0.13796239 | 0.72408948 | 0.39089513 | 0.40366457 | 0.18876024 | 0.55614883 |
| | | KRAB and SCAN | | | | | | | | | |
| | | domains 6 | | | | | | | | | |
| 124 | Uap1 | UDP-N- | NM_133806 | 3.8780438 | 0.01095998 | 0.19584521 | 0.64186151 | 2.03682032 | 0.05446268 | 3.76225238 | 0.0111286 |
| | | acetylglucosamine | | | | | | | | | |
| | | pyrophosphorylase 1 | | | | | | | | | |
| 125 | Pde4b | phosphodiesterase | NM_019840 | 3.81200545 | 0.01140962 | 2.15899652 | 0.05648606 | 5.64070642 | 0.00848696 | 17.0448274 | 0.00044964 |
| | | 4B, cAMP | | | | | | | | | |
| | | specific | | | | | | | | | |
| 126 | Myd88 | myeloid | NM_010851 | 3.79473227 | 0.01152203 | 4.83805855 | 0.01438849 | 0.62147791 | 0.26551259 | 0.84650172 | 0.14073741 |
| | | differentiation | | | | | | | | | |
| | | primary response | | | | | | | | | |
| | | gene 88 | | | | | | | | | |
| 127 | Rhod | ras homolog gene | NM_007485 | 3.77727519 | 0.01174685 | 0.27580764 | 0.54502023 | 1.59662113 | 0.07975495 | 1.25014896 | 0.07851844 |
| | | family, member D | | | | | | | | | |
| 128 | Il1rl1 | interleukin 1 | NM_001025602 | 3.77525741 | 0.01174685 | 0.07467543 | 0.83234038 | 0.71491085 | 0.22757419 | 0.06251477 | 0.81058903 |
| | | receptor-like 1 | | | | | | | | | |
| 129 | Gbp2 | guanylate | NM_010260 | 3.73967899 | 0.01185926 | 0.06470337 | 0.85167491 | 0.82760087 | 0.19491906 | 0.29513981 | 0.42035746 |
| | | binding protein 2 | | | | | | | | | |
| 130 | Fkbp5 | FK506 binding | NM_010220 | 3.73526454 | 0.01185926 | −0.1955722 | 0.64214254 | 0.66845848 | 0.24533498 | 0.48420085 | 0.27281924 |
| | | protein 5 | | | | | | | | | |
| 131 | Bcl6b | B-cell | NM_007528 | 3.71320111 | 0.01197167 | 0.16854283 | 0.67800135 | 1.22223002 | 0.11623201 | 0.82677141 | 0.14484038 |
| | | CLL/lymphoma 6, | | | | | | | | | |
| | | member B | | | | | | | | | |
| 132 | Sema4d | sema domain, | NM_013660 | 3.71806759 | 0.01197167 | 3.4304472 | 0.02517986 | 0.36281031 | 0.42507869 | 0.12119283 | 0.67485387 |
| | | immunoglobulin | | | | | | | | | |
| | | domain (Ig), | | | | | | | | | |
| | | transmembrane | | | | | | | | | |
| | | domain (TM) and | | | | | | | | | |
| | | short cytoplasmic | | | | | | | | | |
| | | domain, | | | | | | | | | |
| | | (semaphorin) 4D | | | | | | | | | |
| 133 | Gch1 | GTP | NM_008102 | 3.69279437 | 0.01197167 | 1.28299674 | 0.1261241 | 5.26290024 | 0.00961106 | 6.94987782 | 0.00359712 |
| | | cyclohydrolase 1 | | | | | | | | | |
| 134 | Tlr2 | toll-like receptor 2 | NM_011905 | 3.65677595 | 0.01219649 | 1.87226237 | 0.07115558 | 2.86925116 | 0.03169964 | 3.05442226 | 0.0167491 |
| 135 | Myo1g | myosin IG | NM_178440 | 3.65416613 | 0.0122527 | 4.7595168 | 0.01483813 | 0.99299921 | 0.15439523 | 0.01184753 | 0.96178058 |
| 136 | Osmr | oncostatin M | NM_011019 | 3.64649525 | 0.0122527 | 1.32271927 | 0.12089703 | 1.00073264 | 0.15270908 | 0.7578233 | 0.16085881 |
| | | receptor | | | | | | | | | |
| 137 | Ifrd1 | interferon-related | NM_013562 | 3.64027281 | 0.0123089 | 1.11813383 | 0.15270908 | 1.42060161 | 0.09543615 | 1.33804857 | 0.07081835 |
| | | developmental | | | | | | | | | |
| | | regulator 1 | | | | | | | | | |
| 138 | Ippk | inositol 1,3,4,5,6- | NM_199056 | 3.63984716 | 0.0123089 | 0.43030174 | 0.40866682 | 0.49028325 | 0.33599371 | 0.30472197 | 0.41136466 |
| | | pentakisphosphate | | | | | | | | | |
| | | 2-kinase | | | | | | | | | |
| 139 | Irf5 | interferon | NM_012057 | 3.62541877 | 0.01242131 | 1.97880483 | 0.06542266 | 0.14253456 | 0.6875 | 0.28870537 | 0.42727068 |
| | | regulatory factor 5 | | | | | | | | | |
| 140 | Cytip | cytohesin 1 | NM_139200 | 3.61715217 | 0.01247752 | 2.49335034 | 0.04440198 | 0.74421961 | 0.21734487 | 0.59367404 | 0.21773831 |
| | | interacting | | | | | | | | | |
| | | protein | | | | | | | | | |
| 141 | Rgs16 | regulator of G- | NM_011267 | 3.58927 | 0.01253372 | 0.4528537 | 0.39321043 | 0.81166724 | 0.19964029 | 1.36220813 | 0.06879496 |
| | | protein signaling | | | | | | | | | |
| | | 16 | | | | | | | | | |
| 142 | LOC640502 | similar to UDP-N- | XM_917484 | 3.58325899 | 0.01253372 | 0.2173708 | 0.61314074 | 2.02941663 | 0.0546875 | 3.0504132 | 0.0167491 |
| | | acetylhexosamine | | | | | | | | | |
| | | pyrophosphorylase | | | | | | | | | |
| 143 | Ifngr1 | interferon gamma | NM_010511 | 3.57954461 | 0.01258993 | −0.0549409 | 0.87247077 | 0.23825409 | 0.55367581 | 0.11360387 | 0.68957959 |
| | | receptor 1 | | | | | | | | | |
| 144 | Socs2 | suppressor of | NM_001168655 | 3.52240809 | 0.01270234 | 1.86342177 | 0.07149281 | 0.58589293 | 0.2823179 | 0.84233253 | 0.14135567 |
| | | cytokine | | | | | | | | | |
| | | signaling 2 | | | | | | | | | |
| 145 | Stc1 | stanniocalcin 1 | NM_009285 | 3.5153317 | 0.01275854 | −0.2373619 | 0.58863534 | 0.77195691 | 0.20913894 | 0.21490263 | 0.5201214 |
| 146 | Adcy8 | adenylate cyclase 8 | NM_009623 | 3.5176503 | 0.01275854 | 0.08266991 | 0.81761466 | −0.0732097 | 0.81800809 | −0.0088017 | 0.97127923 |
| 147 | Pdzklip1 | PDZK1 | NM_001164557 | 3.50242833 | 0.01287095 | 0.64062447 | 0.28911871 | 0.11402383 | 0.73808453 | 0.00086616 | 0.99690872 |
| | | interacting | | | | | | | | | |
| | | protein 1 | | | | | | | | | |
| 148 | Ehf | ets homologous | NM_007914 | 3.49402096 | 0.01292716 | 2.91680635 | 0.03332959 | 0.00372868 | 0.98909622 | 0.47128507 | 0.28080036 |
| | | factor | | | | | | | | | |
| 149 | Hmgcs2 | 3-hydroxy-3- | NM_008256 | 3.48786158 | 0.01298336 | 0.01068485 | 0.9732464 | 0.18143164 | 0.6260679 | −0.0054321 | 0.98223921 |
| | | methylglutaryl- | | | | | | | | | |
| | | Coenzyme A | | | | | | | | | |
| | | synthase 2 | | | | | | | | | |
| 150 | Ubr2 | ubiquitin protein | NM_146078 | 3.48363679 | 0.01298336 | −0.122992 | 0.74898831 | 0.06583453 | 0.83301484 | 0.00875844 | 0.97133543 |
| | | ligase E3 | | | | | | | | | |
| | | component n- | | | | | | | | | |
| | | recognin 2 | | | | | | | | | |
| 151 | Rassf1 | Ras association | NM_019713 | 3.47722936 | 0.01303957 | 3.263729 | 0.02748426 | 1.56033724 | 0.08295863 | 2.32248243 | 0.02692221 |
| | | (RalGDS/AF-6) | | | | | | | | | |
| | | domain family | | | | | | | | | |
| | | member 1 | | | | | | | | | |
| 152 | Tnf | tumor necrosis | NM_013693 | 3.47583332 | 0.01303957 | 1.17664638 | 0.14253597 | 1.95537048 | 0.05834083 | 1.05303462 | 0.10229317 |
| | | factor | | | | | | | | | |
| 153 | Niacr1 | niacin receptor 1 | NM_030701 | 3.46899256 | 0.01320818 | 1.17076 | 0.14354766 | 0.71332544 | 0.22813624 | 1.13318775 | 0.09200764 |
| 154 | Gadd45b | growth arrest | NM_008655 | 3.45075276 | 0.01326439 | 1.68421841 | 0.08447617 | 3.00720998 | 0.02928282 | 5.18167323 | 0.00578912 |
| | | and DNA-damage- | | | | | | | | | |
| | | inducible 45 beta | | | | | | | | | |
| 155 | Dusp8 | dual specificity | NM_008748 | 3.43115891 | 0.0133768 | 1.79615193 | 0.07627023 | 1.59248316 | 0.08003597 | 2.36036228 | 0.02630396 |
| | | phosphatase 8 | | | | | | | | | |
| 156 | Asgr2 | asialoglycoprotein | NM_007493 | 3.43351819 | 0.0133768 | 0.96802097 | 0.1830598 | 0.13049691 | 0.70784622 | −0.0340011 | 0.89371628 |
| | | receptor 2 | | | | | | | | | |
| 157 | Acaa1b | acetyl-Coenzyme | NM_146230 | 3.43332118 | 0.0133768 | 0.65678402 | 0.28147482 | −0.1088767 | 0.7477518 | −0.014793 | 0.95222572 |
| | | A acyltransferase | | | | | | | | | |
| | | 1B | | | | | | | | | |
| 158 | Lat2 | linker for | NM_020044 | 3.41363763 | 0.01348921 | 0.14632263 | 0.71127473 | 0.49191528 | 0.33413894 | 0.0562507 | 0.82750674 |
| | | activation of T | | | | | | | | | |
| | | cells family, | | | | | | | | | |
| | | member 2 | | | | | | | | | |
| 159 | Ereg | epiregulin | NM_007950 | 3.3941229 | 0.01354541 | 2.40236525 | 0.04665018 | 0.97567471 | 0.15821718 | 1.36266635 | 0.06873876 |
| 160 | LOC100045163 | similar to RIKEN | XM_001473518 | 3.39368277 | 0.01354541 | 0.21494035 | 0.61645683 | 0.13464308 | 0.70042716 | 0.0308805 | 0.90349595 |
| | | cDNA | | | | | | | | | |
| | | 1100001H23 gene | | | | | | | | | |
| 161 | Plbd1 | phospholipase B | NM_025806 | 3.39368277 | 0.01354541 | 0.21494035 | 0.61645683 | 0.13464308 | 0.70042716 | 0.0308805 | 0.90349595 |
| | | domain | | | | | | | | | |
| | | containing 1 | | | | | | | | | |
| 162 | Slc5a1 | solute carrier | NM_019810 | 3.37973229 | 0.01360162 | 7.30817939 | 0.00663219 | 3.47131647 | 0.02191996 | 0.8131135 | 0.14787545 |
| | | family 5 | | | | | | | | | |
| | | (sodium/glucose | | | | | | | | | |
| | | cotransporter), | | | | | | | | | |
| | | member 1 | | | | | | | | | |
| 163 | Ptpn22 | protein tyrosine | NM_008979 | 3.35449558 | 0.01377023 | 0.32642369 | 0.49404227 | 0.19720539 | 0.60560926 | 0.05941868 | 0.81840153 |
| | | phosphatase, | | | | | | | | | |
| | | non-receptor type | | | | | | | | | |
| | | 22 (lymphoid) | | | | | | | | | |
| 164 | LOC100044257 | hypothetical | XM_001471785 | 3.34345715 | 0.01388264 | 0.00773053 | 0.97948516 | 0.84811345 | 0.19047887 | 0.22461549 | 0.50725045 |
| | | protein | | | | | | | | | |
| | | LOC100044257 | | | | | | | | | |
| 165 | Otud7a | OTU domain | NM_130880 | 3.34345715 | 0.01388264 | 0.00773053 | 0.97948516 | 0.84811345 | 0.19047887 | 0.22461549 | 0.50725045 |
| | | containing 7A | | | | | | | | | |
| 166 | Zfp295 | zinc finger | NM_001081684 | 3.34088675 | 0.01388264 | 0.79429853 | 0.23263264 | 1.30877108 | 0.10555306 | 7.21010695 | 0.00325989 |
| | | protein 295 | | | | | | | | | |
| 167 | Nnat | neuronatin | NM_010923 | 3.32416957 | 0.01393885 | −0.2115294 | 0.62078462 | 0.10913599 | 0.74735836 | −0.0532227 | 0.83666817 |
| 168 | Ptger2 | prostaglandin E | NM_008964 | 3.30373675 | 0.01416367 | −0.0222784 | 0.94548112 | 0.52642148 | 0.31486061 | 0.10853346 | 0.70048336 |
| | | receptor 2 | | | | | | | | | |
| | | (subtype EP2) | | | | | | | | | |
| 169 | Mlxip | MLX interacting | NM_133917 | 3.29003595 | 0.01427608 | −0.1885049 | 0.6506857 | 0.23312933 | 0.56013939 | 0.33169351 | 0.38888264 |
| | | protein | | | | | | | | | |
| 170 | Xdh | xanthine | NM_011723 | 3.23922021 | 0.01489433 | −0.124574 | 0.74657149 | 0.335247 | 0.44947167 | 0.07091116 | 0.78771358 |
| | | dehydrogenase | | | | | | | | | |
| 171 | Psors1c2 | psoriasis | NM_020576 | 3.24030204 | 0.01489433 | −0.2074397 | 0.62584308 | 1.82591346 | 0.06457959 | 0.0641786 | 0.80665468 |
| | | susceptibility 1 | | | | | | | | | |
| | | candidate 2 | | | | | | | | | |
| | | (human) | | | | | | | | | |
| 172 | Lep | leptin | NM_008493 | 3.24023439 | 0.01489433 | −0.0525912 | 0.87713579 | 0.23268406 | 0.56081385 | 0.31812607 | 0.39967401 |
| 173 | Pdcd1lg2 | programmed cell | NM_021396 | 3.22025397 | 0.01500674 | 1.13104321 | 0.15057329 | 0.52677343 | 0.3147482 | 0.05555579 | 0.82981115 |
| | | death 1 ligand 2 | | | | | | | | | |
| 174 | Slc3a2 | solute carrier | NM_001161413 | 3.23117977 | 0.01500674 | 0.88739004 | 0.20413669 | 1.04788504 | 0.14360387 | 1.12305004 | 0.09341277 |
| | | family 3 | | | | | | | | | |
| | | (activators of | | | | | | | | | |
| | | dibasic and | | | | | | | | | |
| | | neutral amino | | | | | | | | | |
| | | acid transport), | | | | | | | | | |
| | | member 2 | | | | | | | | | |
| 175 | Igtp | interferon gamma | NM_018738 | 3.23158404 | 0.01500674 | −0.0980382 | 0.79029901 | 0.08490195 | 0.79378372 | −0.0610718 | 0.81401754 |
| | | induced GTPase | | | | | | | | | |
| 176 | Sla | src-like adaptor | NM_001029841 | 3.21968946 | 0.01500674 | 0.03510876 | 0.91861511 | 0.19311547 | 0.61100495 | 0.09633098 | 0.72605665 |
| 177 | Nts | neurotensin | NM_024435 | 3.20975438 | 0.01506295 | 0.43381098 | 0.40630621 | 1.95402793 | 0.05850944 | 1.15790767 | 0.08897257 |
| 178 | Azin1 | antizyme | NM_001102458 | 3.21074226 | 0.01506295 | 0.83863576 | 0.21886241 | 2.69713549 | 0.03479092 | 1.18782523 | 0.08554406 |
| | | inhibitor 1 | | | | | | | | | |
| 179 | Ets2 | E26 avian | NM_011809 | 3.20673315 | 0.01523156 | 7.92277271 | 0.0055643 | 0.45316371 | 0.35673336 | 2.44563912 | 0.02484263 |
| | | leukemia | | | | | | | | | |
| | | oncogene 2, 3′ | | | | | | | | | |
| | | domain | | | | | | | | | |
| 180 | Cd209c | CD209c antigen | NM_130903 | 3.19117889 | 0.01534397 | 1.88453999 | 0.07059353 | 0.03734344 | 0.90130396 | 0 | 1 |
| 181 | Srgn | serglycin | NM_011157 | 3.17565516 | 0.01545638 | 0.79528176 | 0.23235162 | 1.95424069 | 0.05850944 | 5.35372956 | 0.00545189 |
| 182 | Arc | activity regulated | NM_018790 | 3.16393498 | 0.01551259 | 0.87485488 | 0.2076214 | 0.71643839 | 0.22706835 | 0.72546856 | 0.17120054 |
| | | cytoskeletal- | | | | | | | | | |
| | | associated | | | | | | | | | |
| | | protein | | | | | | | | | |
| 183 | Tslp | thymic stromal | NM_021367 | 3.1501285 | 0.01573741 | 2.77667493 | 0.03692671 | 1.21356892 | 0.11758094 | 2.59356419 | 0.02220099 |
| | | lymphopoietin | | | | | | | | | |
| 184 | LOC68395 | histocompatibility | XM_903697 | 3.14717692 | 0.01573741 | 0.35465999 | 0.46790692 | −0.1428804 | 0.68688174 | −0.0325584 | 0.89877473 |
| | | 2, Q region | | | | | | | | | |
| | | locus 6-like | | | | | | | | | |
| 185 | Bcar1 | breast cancer | NM_009954 | 3.08729989 | 0.01658049 | 2.25088239 | 0.05238309 | 0.79450888 | 0.20301259 | 1.77088974 | 0.04423336 |
| | | anti-estrogen | | | | | | | | | |
| | | resistance 1 | | | | | | | | | |
| 186 | Ifitm6 | interferon | NM_001033632 | 3.03390499 | 0.01691772 | 0.19298321 | 0.64557104 | 3.3562544 | 0.02360612 | 0.65793883 | 0.19340153 |
| | | induced | | | | | | | | | |
| | | transmembrane | | | | | | | | | |
| | | protein 6 | | | | | | | | | |
| 187 | Cyr61 | cysteine rich | NM_010516 | 3.02357158 | 0.01703013 | 0.35081271 | 0.47122302 | −0.8338162 | 0.19362635 | 2.69841698 | 0.02073966 |
| | | protein 61 | | | | | | | | | |
| 188 | Oasl2 | 2′-5′ | NM_011854 | 3.00776029 | 0.01719874 | 1.30374624 | 0.12314523 | −0.2886921 | 0.49505396 | −0.1273398 | 0.66232014 |
| | | oligoadenylate | | | | | | | | | |
| | | synthetase-like 2 | | | | | | | | | |
| 189 | Map3k8 | mitogen-activated | NM_007746 | 2.96851051 | 0.01776079 | 0.76916715 | 0.24128822 | 0.60642193 | 0.27163894 | 2.37950136 | 0.02596673 |
| | | protein kinase | | | | | | | | | |
| | | kinase kinase 8 | | | | | | | | | |
| 190 | Traf1 | TNF receptor- | NM_009421 | 2.94642649 | 0.01804182 | 1.10526035 | 0.15580036 | 1.1683535 | 0.12438174 | 0.65388234 | 0.19491906 |
| | | associated factor 1 | | | | | | | | | |
| 191 | Dok2 | docking protein 2 | NM_010071 | 2.95258152 | 0.01804182 | 1.70258119 | 0.08250899 | 1.02110799 | 0.14877473 | 0.13212196 | 0.65332734 |
| 192 | Retnla | resistin like alpha | NM_020509 | 2.93926584 | 0.01826664 | 0.08727138 | 0.80968975 | 0.47078218 | 0.34611061 | −0.0100758 | 0.96695144 |
| 193 | Trim25 | tripartite motif- | NM_009546 | 2.93880497 | 0.01826664 | 0.27554906 | 0.54524505 | 1.19000158 | 0.12084083 | 1.96622326 | 0.03732014 |
| | | containing 25 | | | | | | | | | |
| 194 | Klf13 | Kruppel-like | NM_021366 | 2.90995726 | 0.01860387 | −0.752572 | 0.2466839 | 0.36138771 | 0.42614658 | 0.29411804 | 0.42131295 |
| | | factor 13 | | | | | | | | | |
| 195 | Ifi204 | interferon | NM_008329 | 2.89303607 | 0.01910971 | −0.8077482 | 0.22796763 | 3.05970174 | 0.02838354 | 0.84139756 | 0.1416929 |
| | | activated gene | | | | | | | | | |
| | | 204 | | | | | | | | | |
| 196 | St3gal5 | ST3 beta- | NM_001035228 | 2.8798431 | 0.01933453 | 0.00548104 | 0.98606115 | 0.61900026 | 0.26618705 | 0.39093267 | 0.33391412 |
| | | galactoside alpha- | | | | | | | | | |
| | | 2,3- | | | | | | | | | |
| | | sialyltransferase 5 | | | | | | | | | |
| 197 | Pck1 | phosphoenolpyruvate | NM_011044 | 2.86139005 | 0.01955935 | −0.0415267 | 0.90265288 | 0.81433046 | 0.19851619 | 0.39827523 | 0.32868705 |
| | | carboxykinase 1, | | | | | | | | | |
| | | cytosolic | | | | | | | | | |
| 198 | Lta | lymphotoxin A | NM_010735 | 2.84791448 | 0.01967176 | 7.85389739 | 0.00567671 | 0.33214615 | 0.45211331 | 0.03600102 | 0.88669065 |
| 199 | Picalm | phosphatidylinositol | NM_146194 | 2.80888113 | 0.0201214 | 1.10879795 | 0.15484487 | 0.00124658 | 0.99634667 | 0.78772137 | 0.15428282 |
| | | binding | | | | | | | | | |
| | | clathrin assembly | | | | | | | | | |
| | | protein | | | | | | | | | |
| 200 | Per1 | period homolog 1 | NM_001159367 | 2.78389102 | 0.02023381 | 1.59431277 | 0.09161421 | 0.02673331 | 0.92586556 | 0.2754017 | 0.44272707 |
| | | (Drosophila) | | | | | | | | | |
| 201 | Serpina3n | serine (or | NM_009252 | 2.78139233 | 0.02023381 | −0.4468412 | 0.39714478 | 1.39291477 | 0.09796538 | 0.45266028 | 0.29220998 |
| | | cysteine) | | | | | | | | | |
| | | peptidase | | | | | | | | | |
| | | inhibitor, clade | | | | | | | | | |
| | | A, member 3N | | | | | | | | | |
| 202 | Lce3a | late cornified | NM_001039594 | 2.7527024 | 0.02051484 | 0.63398158 | 0.29209757 | 1.00999583 | 0.15085432 | −0.0366748 | 0.88494829 |
| | | envelope 3A | | | | | | | | | |
| 203 | 2310016C08Rik | RIKEN cDNA | NM_023516 | 2.74635497 | 0.02057104 | 3.06919162 | 0.03040692 | 34.4555498 | 0.00011241 | 6.7025142 | 0.00376574 |
| | | 2310016C08 gene | | | | | | | | | |
| 204 | Cd44 | CD44 antigen | NM_001039150 | 2.73488076 | 0.02085207 | 1.65434311 | 0.08672437 | 0.93039304 | 0.16872752 | 0.91404098 | 0.12595549 |
| 205 | Arid5a | AT rich | NM_001172205 | 2.72829805 | 0.02090827 | 0.80120398 | 0.23010342 | 1.24486335 | 0.11302833 | 4.08447325 | 0.00983588 |
| | | interactive | | | | | | | | | |
| | | domain 5A | | | | | | | | | |
| | | (MRF1-like) | | | | | | | | | |
| 206 | Gem | GTP binding | NM_010276 | 2.69737667 | 0.02130171 | 3.04380182 | 0.03102518 | 0.64836594 | 0.25354092 | 1.54623663 | 0.05569919 |
| | | protein (gene | | | | | | | | | |
| | | overexpressed in | | | | | | | | | |
| | | skeletal muscle) | | | | | | | | | |
| 207 | Inpp5d | inositol | NM_001110192 | 2.70118366 | 0.02130171 | 0.13171891 | 0.73431879 | 0.17212194 | 0.64000674 | 0.05099183 | 0.84167041 |
| | | polyphosphate-5- | | | | | | | | | |
| | | phosphatase D | | | | | | | | | |
| 208 | Rel | reticuloendotheliosis | NM_009044 | 2.7053087 | 0.02130171 | 2.30829334 | 0.05035971 | 1.22815327 | 0.11544514 | 1.79253734 | 0.04322167 |
| | | oncogene | | | | | | | | | |
| 209 | Klf9 | Kuppel-like | NM_010638 | 2.71384146 | 0.02130171 | −0.5363519 | 0.34330036 | 0.23464574 | 0.55834083 | 0.27787682 | 0.44070369 |
| | | factor 9 | | | | | | | | | |
| 210 | Nlrp3 | NLR family, pyrin | NM_145827 | 2.68529939 | 0.02152653 | −0.6333254 | 0.2924348 | 0.64989113 | 0.25286646 | 0.1733182 | 0.57874326 |
| | | domain | | | | | | | | | |
| | | containing 3 | | | | | | | | | |
| 211 | Pard6b | par-6 | NM_021409 | 2.68834179 | 0.02152653 | 3.78666011 | 0.02175135 | 0.82047429 | 0.19716727 | 1.62302629 | 0.05142761 |
| | | (partitioning | | | | | | | | | |
| | | defective 6) | | | | | | | | | |
| | | homolog beta (C. elegans) | | | | | | | | | |
| 212 | Ccr1 | chemokine (C-C | NM_009912 | 2.67942996 | 0.02158273 | 0.4255944 | 0.41232014 | 1.19122872 | 0.12061601 | 0.48026261 | 0.27529227 |
| | | motif) receptor 1 | | | | | | | | | |
| 213 | Ccl9 | chemokine (C-C | NM_011338 | 2.67232531 | 0.02180755 | 0.92216732 | 0.19480665 | 3.56212053 | 0.02045863 | 1.44542536 | 0.0625 |
| | | motif) ligand 9 | | | | | | | | | |
| 214 | Stat5a | signal transducer | NM_001164062 | 2.66126899 | 0.02191996 | 0.46352155 | 0.38573516 | 0.21365472 | 0.58543165 | 0.94381371 | 0.12011016 |
| | | and activator of | | | | | | | | | |
| | | transcription 5A | | | | | | | | | |
| 215 | Nfkbie | nuclear factor of | NM_008690 | 2.63639266 | 0.0223696 | 3.43135484 | 0.02517986 | 0.62597452 | 0.26388264 | 2.05425247 | 0.03383543 |
| | | kappa light | | | | | | | | | |
| | | polypeptide gene | | | | | | | | | |
| | | enhancer in B- | | | | | | | | | |
| | | cells inhibitor, | | | | | | | | | |
| | | epsilon | | | | | | | | | |
| 216 | Stk17b | serine/threonine | NM_133810 | 2.63629462 | 0.0223696 | 1.14182574 | 0.14883094 | 1.19713423 | 0.11999775 | 1.05561702 | 0.10189973 |
| | | kinase 17b | | | | | | | | | |
| | | (apoptosis- | | | | | | | | | |
| | | inducing) | | | | | | | | | |
| 217 | Il2rg | interleukin 2 | NM_013563 | 2.6314745 | 0.0223696 | 0.5312369 | 0.34611061 | 0.25491839 | 0.53338579 | 0.20778869 | 0.529058 |
| | | receptor, gamma | | | | | | | | | |
| | | chain | | | | | | | | | |
| 218 | Socs3 | suppressor of | NM_007707 | 2.62914582 | 0.02248201 | 2.70919548 | 0.03866906 | 2.54842159 | 0.03861286 | 2.71069194 | 0.02045863 |
| | | cytokine | | | | | | | | | |
| | | signaling 3 | | | | | | | | | |
| 219 | Cpa1 | carboxypeptidase | NM_025350 | 2.61114077 | 0.02281924 | 0.34604618 | 0.47616906 | −0.0243733 | 0.93244155 | −0.035007 | 0.88961331 |
| | | A1 | | | | | | | | | |
| 220 | Hmgcr | 3-hydroxy-3- | NM_008255 | 2.61204957 | 0.02281924 | 0.8714108 | 0.20880171 | 0.96334065 | 0.16119604 | 2.4082112 | 0.02546088 |
| | | methylglutaryl- | | | | | | | | | |
| | | Coenzyme A | | | | | | | | | |
| | | reductase | | | | | | | | | |
| 221 | Rasa3 | RAS p21 protein | NM_009025 | 2.61418781 | 0.02281924 | 0.49165942 | 0.36870504 | −0.049748 | 0.87117806 | 0.1976996 | 0.54260342 |
| | | activator 3 | | | | | | | | | |
| 222 | Sdhaf1 | succinate | NM_001033140 | 2.59906842 | 0.02315647 | −0.2188705 | 0.61167941 | 0.58522857 | 0.28254272 | 0.17232096 | 0.58059802 |
| | | dehydrogenase | | | | | | | | | |
| | | complex | | | | | | | | | |
| | | assembly factor 1 | | | | | | | | | |
| 223 | Sik1 | salt inducible | NM_010831 | 2.59661358 | 0.02332509 | 0.72408523 | 0.25612635 | 0.00776877 | 0.97774281 | 1.00936004 | 0.10982464 |
| | | kinase 1 | | | | | | | | | |
| 224 | Tnfrsf12a | tumor necrosis | NM_001161746 | 2.59284904 | 0.02338129 | 2.59761799 | 0.04164793 | 1.39699314 | 0.09734712 | 2.53219746 | 0.02287545 |
| | | factor receptor | | | | | | | | | |
| | | superfamily, | | | | | | | | | |
| | | member 12a | | | | | | | | | |
| 225 | Tnfaip3 | tumor necrosis | NM_001166402 | 2.58282093 | 0.02366232 | 1.4344221 | 0.10656475 | 4.20114812 | 0.01500674 | 4.75734859 | 0.00713804 |
| | | factor, alpha- | | | | | | | | | |
| | | induced protein 3 | | | | | | | | | |
| 226 | Gpr97 | G protein-coupled | NM_173036 | 2.5546788 | 0.02399955 | −0.5774172 | 0.3203125 | 0.47504617 | 0.34408723 | 0.13053541 | 0.65714928 |
| | | receptor 97 | | | | | | | | | |
| 227 | Jmjd6 | jumonji domain | NM_033398 | 2.54864601 | 0.02422437 | −0.1493575 | 0.70694694 | 0.09085657 | 0.78209308 | 0.05297404 | 0.83723022 |
| | | containing 6 | | | | | | | | | |
| 228 | Dedd2 | death effector | NM_207677 | 2.5249399 | 0.02489883 | 0.11569103 | 0.76039793 | 0.40296011 | 0.39349146 | 0.17717343 | 0.57317896 |
| | | domain- | | | | | | | | | |
| | | containing DNA | | | | | | | | | |
| | | binding protein 2 | | | | | | | | | |
| 229 | Ear2 | eosinophil- | NM_007895 | 2.51267459 | 0.02501124 | 0.74288111 | 0.24943795 | 0.15592691 | 0.66647932 | −0.1042129 | 0.70936376 |
| | | associated, | | | | | | | | | |
| | | ribonuclease A | | | | | | | | | |
| | | family, member 2 | | | | | | | | | |
| 230 | Ptk6 | PTK6 protein | NM_009184 | 2.51187568 | 0.02501124 | 0.94903177 | 0.18721897 | 0.13064975 | 0.70734038 | 0.08683949 | 0.74988759 |
| | | tyrosine kinase 6 | | | | | | | | | |
| 231 | Dnajb4 | DnaJ (Hsp40) | NM_025926 | 2.51669777 | 0.02501124 | 0.20665751 | 0.62713579 | 0.16525409 | 0.6506295 | 0.40898958 | 0.32104317 |
| | | homolog, | | | | | | | | | |
| | | subfamily B, | | | | | | | | | |
| | | member 4 | | | | | | | | | |
| 232 | Nsg1 | neuron specific | NM_010942 | 2.49310541 | 0.02540468 | −0.0094565 | 0.97583183 | 0.33845179 | 0.44677383 | 0.08979089 | 0.74353642 |
| | | gene family | | | | | | | | | |
| | | member 1 | | | | | | | | | |
| 233 | Gucy2c | guanylate cyclase | NM_001127318 | 2.49041476 | 0.0256295 | 1.81285074 | 0.07503372 | −0.1971992 | 0.60560926 | 0.18902317 | 0.55598022 |
| | | 2c | | | | | | | | | |
| 234 | Sele | selectin, | NM_011345 | 2.47785833 | 0.02574191 | 0.66566599 | 0.27827113 | 1.82843638 | 0.06435477 | 2.27398368 | 0.02810252 |
| | | endothelial cell | | | | | | | | | |
| 235 | Ppp1r15a | protein | NM_008654 | 2.45217361 | 0.02613534 | 0.27556077 | 0.54524505 | 0.25073811 | 0.53810701 | 0.27513774 | 0.44289568 |
| | | phosphatase 1, | | | | | | | | | |
| | | regulatory | | | | | | | | | |
| | | (inhibitor) | | | | | | | | | |
| | | subunit 15A | | | | | | | | | |
| 236 | Mettl7b | methyltransferase | NM_027853 | 2.44506501 | 0.02619155 | 0.59156034 | 0.31311826 | 0.024846 | 0.93098022 | 0.28631344 | 0.4296875 |
| | | like 7B | | | | | | | | | |
| 237 | Glipr1 | GLI pathogenesis- | NM_028608 | 2.43483208 | 0.02658498 | 0.83120923 | 0.22088579 | −0.081862 | 0.79917941 | 0.20239569 | 0.53692671 |
| | | related 1 (glioma) | | | | | | | | | |
| 238 | Kctd4 | potassium | NM_026214 | 2.43155862 | 0.02664119 | −0.3525619 | 0.4698179 | 0.57795382 | 0.28630845 | −0.2432698 | 0.48252023 |
| | | channel | | | | | | | | | |
| | | tetramerisation | | | | | | | | | |
| | | domain | | | | | | | | | |
| | | containing 4 | | | | | | | | | |
| 239 | Rnase1 | ribonuclease, | NM_011271 | 2.3932731 | 0.02748426 | 0.74822607 | 0.24797662 | 0.00740529 | 0.97830486 | 0.00790113 | 0.97453912 |
| | | RNase A family, 1 | | | | | | | | | |
| | | (pancreatic) | | | | | | | | | |
| 240 | Ier2 | immediate early | NM_010499 | 2.38660683 | 0.02759667 | 14.5249742 | 0.00168615 | −0.2604754 | 0.52686601 | 0.41841689 | 0.31435477 |
| | | response 2 | | | | | | | | | |
| 241 | Ccl22 | chemokine (C-C | NM_009137 | 2.36489757 | 0.02821493 | 1.50670752 | 0.09993255 | 0.24021959 | 0.55103417 | 0.04723487 | 0.85302383 |
| | | motif) ligand 22 | | | | | | | | | |
| 242 | Odc1 | ornithine | NM_013614 | 2.35855218 | 0.02832734 | −1.953296 | 0.06665917 | 0.94339171 | 0.16580486 | 0.32040463 | 0.39832509 |
| | | decarboxylase, | | | | | | | | | |
| | | structural 1 | | | | | | | | | |
| 243 | Csrp1 | cysteine and | NM_007791 | 2.34090057 | 0.02866457 | 0.86935422 | 0.20947617 | 1.10975375 | 0.13213804 | 1.55676691 | 0.05530576 |
| | | glycine-rich | | | | | | | | | |
| | | protein 1 | | | | | | | | | |
| 244 | Pvr | poliovirus | NM_027514 | 2.33716983 | 0.02872077 | 0.24033061 | 0.58464478 | 1.32552446 | 0.10397932 | 2.14131464 | 0.03119379 |
| | | receptor | | | | | | | | | |
| 245 | Aqp3 | aquaporin 3 | NM_016689 | 2.31420784 | 0.02922662 | 1.57259951 | 0.09346897 | 0.5440975 | 0.30457509 | 0.85105648 | 0.13966951 |
| 246 | Cd2 | CD2 antigen | NM_013486 | 2.3086761 | 0.02933903 | 1.42284155 | 0.1083071 | −0.0103072 | 0.97060477 | −0.0090921 | 0.97015513 |
| 247 | Rnf19b | ring finger | NM_029219 | 2.30156654 | 0.02939523 | 0.62641742 | 0.2957509 | 2.09551538 | 0.05198966 | 1.83378019 | 0.04142311 |
| | | protein 19B | | | | | | | | | |
| 248 | Ece1 | endothelin | NM_199307 | 2.29901327 | 0.02945144 | −0.0211646 | 0.94772932 | 0.69064415 | 0.23701664 | 0.30768986 | 0.40861061 |
| | | converting | | | | | | | | | |
| | | enzyme 1 | | | | | | | | | |
| 249 | Gnl3 | guanine | NM_153547 | 2.297145 | 0.02950764 | 0.36101958 | 0.46256745 | 0.36478387 | 0.42356115 | 0.75564175 | 0.16153327 |
| | | nucleotide | | | | | | | | | |
| | | binding protein- | | | | | | | | | |
| | | like 3 (nucleolar) | | | | | | | | | |
| 250 | Ccrn4l | CCR4 carbon | NM_009834 | 2.28491907 | 0.03001349 | 5.14477788 | 0.01371403 | 3.09887676 | 0.02776529 | 3.45513957 | 0.01360162 |
| | | catabolite | | | | | | | | | |
| | | repression 4-like | | | | | | | | | |
| | | (S. cerevisiae) | | | | | | | | | |
| 251 | LOC100047134 | similar to carbon | XM_001477322 | 2.28491907 | 0.03001349 | 5.14477788 | 0.01371403 | 3.09887676 | 0.02776529 | 3.45513957 | 0.01360162 |
| | | catabolite | | | | | | | | | |
| | | repression 4 | | | | | | | | | |
| | | protein homolog | | | | | | | | | |
| 252 | Dclre1b | DNA cross-link | NM_001025312 | 2.26060842 | 0.03040692 | 0.25315447 | 0.5692446 | −0.0644145 | 0.83638714 | 0.06907488 | 0.79294065 |
| | | repair 1B, PSO2 | | | | | | | | | |
| | | homolog (S. cerevisiae) | | | | | | | | | |
| 253 | 1600029D21Rik | RIKEN cDNA | NM_029639 | 2.25439228 | 0.03051933 | 5.66907819 | 0.01129721 | 1.8300822 | 0.06435477 | 2.48633407 | 0.02399955 |
| | | 1600029D21 gene | | | | | | | | | |
| 254 | LOC100048365 | similar to keratin | XM_001480092 | 2.24904464 | 0.03057554 | 0.43585493 | 0.40456385 | 4.99621941 | 0.01056655 | 0.13065378 | 0.65692446 |
| | | associated | | | | | | | | | |
| | | protein 6-1 | | | | | | | | | |
| 255 | Il10ra | interleukin 10 | NM_008348 | 2.22592612 | 0.03113759 | 0.61517065 | 0.30142761 | 0.13257479 | 0.70413669 | 0.12539179 | 0.66687275 |
| | | receptor, alpha | | | | | | | | | |
| 256 | Csrp2 | cysteine and | NM_007792 | 2.2045347 | 0.03141862 | −0.3733639 | 0.4520009 | 0.86235351 | 0.18671313 | 0.17799026 | 0.57216727 |
| | | glycine-rich | | | | | | | | | |
| | | protein 2 | | | | | | | | | |
| 257 | LOC100045085 | similar to Anxa3 | XM_001473636 | 2.19252706 | 0.03153103 | −0.4376169 | 0.40355216 | 0.26026309 | 0.52697842 | 0.01730956 | 0.94385117 |
| 258 | F2rl1 | coagulation | NM_007974 | 2.19830877 | 0.03153103 | 0.60221906 | 0.30727293 | 0.53004742 | 0.31272482 | 1.97725664 | 0.03709532 |
| | | factor II | | | | | | | | | |
| | | (thrombin) | | | | | | | | | |
| | | receptor-like 1 | | | | | | | | | |
| 259 | Akap12 | A kinase (PRKA) | NM_031185 | 2.18746708 | 0.03175585 | 0.58879014 | 0.31424236 | 2.5328955 | 0.0390625 | 2.09620357 | 0.03248651 |
| | | anchor protein | | | | | | | | | |
| | | (gravin) 12 | | | | | | | | | |
| 260 | Phlda2 | pleckstrin | NM_009434 | 2.18384785 | 0.03186826 | 1.74384597 | 0.07975495 | 0.28515183 | 0.49881969 | 0.13220044 | 0.65310252 |
| | | homology-like | | | | | | | | | |
| | | domain, family A, | | | | | | | | | |
| | | member 2 | | | | | | | | | |
| 261 | Card14 | caspase | NM_130886 | 2.1835209 | 0.03192446 | 1.09839989 | 0.15714928 | 0.35820333 | 0.42901304 | 0.617183 | 0.20846448 |
| | | recruitment | | | | | | | | | |
| | | domain family, | | | | | | | | | |
| | | member 14 | | | | | | | | | |
| 262 | 1700017B05Rik | RIKEN cDNA | NM_028820 | 2.16154904 | 0.03243031 | −0.0991281 | 0.78844424 | 0.61699087 | 0.26703013 | 0.72118624 | 0.17226844 |
| | | 1700017B05 gene | | | | | | | | | |
| 263 | Bsdc1 | BSD domain | NM_133889 | 2.15231245 | 0.03276754 | 0.94139816 | 0.18952338 | 0.18555888 | 0.62078462 | 0.09079075 | 0.74123201 |
| | | containing 1 | | | | | | | | | |
| 264 | Il1r1 | interleukin 1 | NM_001123382 | 2.14729678 | 0.03287995 | 0.11125886 | 0.76596223 | 0.09015603 | 0.78332959 | 0.18057586 | 0.56896358 |
| | | receptor, type I | | | | | | | | | |
| 265 | LOC100048342 | similar to | XM_001480046 | 2.14683783 | 0.03287995 | 0.5410926 | 0.34071493 | 0.44065368 | 0.36561376 | 0.76238449 | 0.15984712 |
| | | mKIAA1547 | | | | | | | | | |
| | | protein | | | | | | | | | |
| 266 | Tle3 | transducin-like | NM_001083927 | 2.14683783 | 0.03287995 | 0.5410926 | 0.34071493 | 0.44065368 | 0.36561376 | 0.76238449 | 0.15984712 |
| | | enhancer of split | | | | | | | | | |
| | | 3, homolog of | | | | | | | | | |
| | | Drosophila E(spl) | | | | | | | | | |
| 267 | Parp14 | poly (ADP-ribose) | NM_001039530 | 2.13510091 | 0.03310477 | 0.62498056 | 0.296875 | 0.32882231 | 0.45548561 | 0.10543114 | 0.70734038 |
| | | polymerase | | | | | | | | | |
| | | family, member | | | | | | | | | |
| | | 14 | | | | | | | | | |
| 268 | Rac2 | RAS-related C3 | NM_009008 | 2.13113017 | 0.03321718 | 0.03701855 | 0.91361286 | −0.0131486 | 0.96324191 | −0.1057452 | 0.70660971 |
| | | botulinum | | | | | | | | | |
| | | substrate 2 | | | | | | | | | |
| 269 | Dgat1 | diacylglycerol 0- | NM_010046 | 2.12756617 | 0.03327338 | 0.94980313 | 0.18699415 | 1.47502415 | 0.08992806 | 2.84212134 | 0.01944694 |
| | | acyltransferase 1 | | | | | | | | | |
| 270 | Ltb4r2 | leukotriene B4 | NM_020490 | 2.12229255 | 0.03332959 | 0.38976648 | 0.43935477 | 0.90149349 | 0.17592176 | 0.23163059 | 0.49876349 |
| | | receptor 2 | | | | | | | | | |
| 271 | Stk40 | serine/threonine | NM_001145827 | 2.11604694 | 0.03338579 | 0.6823252 | 0.27147032 | 0.98649737 | 0.15585656 | 2.01267489 | 0.03540917 |
| | | kinase 40 | | | | | | | | | |
| 272 | Slc6a6 | solute carrier | NM_009320 | 2.10677052 | 0.03372302 | −0.1828965 | 0.65866682 | 0.10933801 | 0.74707734 | 0.04394716 | 0.86207284 |
| | | family 6 | | | | | | | | | |
| | | (neurotransmitter | | | | | | | | | |
| | | transporter, | | | | | | | | | |
| | | taurine), member 6 | | | | | | | | | |
| 273 | Ppif | peptidylprolyl | NM_134084 | 2.1063667 | 0.03372302 | −0.0147004 | 0.96329811 | 0.95093042 | 0.16417491 | 0.50111878 | 0.26292716 |
| | | isomerase F | | | | | | | | | |
| | | (cyclophilin F) | | | | | | | | | |
| 274 | Hp | haptoglobin | NM_017370 | 2.10532106 | 0.03377923 | −0.4456469 | 0.39770683 | 0.93449278 | 0.16760342 | 0.33333537 | 0.38742131 |
| 275 | Slpi | secretory | NM_011414 | 2.1028078 | 0.03383543 | 1.9742402 | 0.06564748 | 9.09967716 | 0.00325989 | 2.0609433 | 0.03366682 |
| | | leukocyte | | | | | | | | | |
| | | peptidase | | | | | | | | | |
| | | inhibitor | | | | | | | | | |
| 276 | Per2 | period homolog 2 | NM_011066 | 2.09758259 | 0.03383543 | −1.0617781 | 0.16350045 | 0.37265004 | 0.41749101 | 0.18985926 | 0.55446268 |
| | | (Drosophila) | | | | | | | | | |
| 277 | Thrsp | thyroid hormone | NM_009381 | 2.08641772 | 0.03389164 | −0.38949 | 0.43957959 | 0.46245564 | 0.35055081 | 0.36652213 | 0.35544065 |
| | | responsive | | | | | | | | | |
| | | SPOT14 homolog | | | | | | | | | |
| | | (Rattus) | | | | | | | | | |
| 278 | Tsc22d3 | TSC22 domain | NM_001077364 | 2.09207093 | 0.03389164 | 0.18152609 | 0.66007194 | 0.05459139 | 0.85959982 | 0.18867275 | 0.55620504 |
| | | family, member 3 | | | | | | | | | |
| 279 | 1810055G02Rik | RIKEN cDNA | NM_028077 | 2.08893945 | 0.03389164 | −0.142971 | 0.71644559 | 0.71934171 | 0.22555081 | 1.54168857 | 0.05609263 |
| | | 1810055G02 gene | | | | | | | | | |
| 280 | Sell | selectin, | NM_001164059 | 2.0800956 | 0.03394784 | 0.38502956 | 0.44334532 | 0.5974804 | 0.27636016 | 0.02686296 | 0.91535522 |
| | | lymphocyte | | | | | | | | | |
| 281 | LOC100046643 | similar to sprouty 1 | XM_001476559 | 2.07423653 | 0.03400405 | 0.51024728 | 0.35763264 | 1.56252207 | 0.0825652 | 0.81218486 | 0.14793165 |
| 282 | Spry1 | sprouty homolog | NM_011896 | 2.07423653 | 0.03400405 | 0.51024728 | 0.35763264 | 1.56252207 | 0.0825652 | 0.81218486 | 0.14793165 |
| | | 1 (Drosophila) | | | | | | | | | |
| 283 | Plekhb2 | pleckstrin | NM_145516 | 2.0760573 | 0.03400405 | 0.39914505 | 0.43232914 | 0.13741498 | 0.69570594 | 0.61603519 | 0.2086893 |
| | | homology | | | | | | | | | |
| | | domain | | | | | | | | | |
| | | containing, | | | | | | | | | |
| | | family B | | | | | | | | | |
| | | (evectins) | | | | | | | | | |
| | | member 2 | | | | | | | | | |
| 284 | Ctla2b | cytotoxic T | NM_001145801 | 2.0749751 | 0.03400405 | 0.01012455 | 0.97442671 | 0.60167892 | 0.27433678 | 0.23956448 | 0.48741007 |
| | | lymphocyte- | | | | | | | | | |
| | | associated | | | | | | | | | |
| | | protein 2 beta | | | | | | | | | |
| 285 | Tgm2 | transglutaminase | NM_009373 | 2.07285047 | 0.03406025 | 0.1126016 | 0.76393885 | 0.97618492 | 0.15810477 | 0.93793069 | 0.12123426 |
| | | 2, C polypeptide | | | | | | | | | |
| 286 | Tubb2a | tubulin, beta 2A | NM_009450 | 2.05611344 | 0.03445369 | 0.22439866 | 0.60454137 | 0.34033002 | 0.44469424 | 0.87642931 | 0.13433004 |
| 287 | Cyp4a10 | cytochrome P450, | NM_010011 | 2.05692908 | 0.03445369 | 1.42554426 | 0.10785746 | 0.21614181 | 0.58183453 | 0.09366533 | 0.73403777 |
| | | family 4, | | | | | | | | | |
| | | subfamily a, | | | | | | | | | |
| | | polypeptide 10 | | | | | | | | | |
| 288 | Cxcl13 | chemokine (C—X—C | NM_018866 | 2.05176262 | 0.03450989 | 0.94081688 | 0.19002923 | 0.89958494 | 0.17642761 | 0.17399706 | 0.57790018 |
| | | motif) ligand 13 | | | | | | | | | |
| 289 | Tgif1 | TGFB-induced | NM_001164074 | 2.03815924 | 0.03495953 | 1.328257 | 0.12022257 | −0.084979 | 0.79367131 | 0.37639334 | 0.34717851 |
| | | factor homeobox 1 | | | | | | | | | |
| 290 | Gyk | glycerol kinase | NM_008194 | 2.02623036 | 0.03529676 | 0.93884091 | 0.19064748 | 0.74494118 | 0.21700764 | 1.95341687 | 0.03760117 |
| 291 | Gzmb | granzyme B | NM_013542 | 2.02212312 | 0.03552158 | 3.4560186 | 0.0245616 | 3.44476144 | 0.02253822 | 1.4754938 | 0.06036421 |
| 292 | 5730408K05Rik | RIKEN cDNA | NR_027866 | 2.0055359 | 0.0356902 | 0.51316764 | 0.35555306 | 0.42372758 | 0.3783161 | 1.34087416 | 0.07053732 |
| | | 5730408K05 gene | | | | | | | | | |
| 293 | Pvrl1 | poliovirus | NM_021424 | 2.00842209 | 0.0356902 | 0.98053436 | 0.18053058 | 0.32521501 | 0.45953237 | 0.21088736 | 0.5245616 |
| | | receptor-related 1 | | | | | | | | | |
| 294 | Abcc3 | ATP-binding | NM_029600 | 2.01030497 | 0.0356902 | 1.66027511 | 0.08621853 | 0.4252139 | 0.37713579 | 0.11200848 | 0.6931205 |
| | | cassette, sub- | | | | | | | | | |
| | | family C | | | | | | | | | |
| | | (CFTR/MRP), | | | | | | | | | |
| | | member 3 | | | | | | | | | |
| 295 | Hmox1 | heme oxygenase | NM_010442 | 2.00218455 | 0.03585881 | −0.0349675 | 0.91872752 | 0.27788615 | 0.50803732 | 0.1595527 | 0.60397932 |
| | | (decycling) 1 | | | | | | | | | |
| 296 | Plk3 | polo-like kinase 3 | NM_013807 | 2.00300366 | 0.03585881 | 1.56792893 | 0.09397482 | 2.84676262 | 0.03203687 | 3.38687981 | 0.01399505 |
| | | (Drosophila) | | | | | | | | | |
| 297 | Adap2 | ArfGAP with dual | NM_172133 | 2.00194741 | 0.03585881 | −0.0531711 | 0.87578687 | 0.03687362 | 0.90259667 | 0.01311355 | 0.95711556 |
| | | PH domains 2 | | | | | | | | | |

TABLE 2B
Subset of genes in Table 2A that are also significantly activated via Toll-Like
Receptor (TLR) stimulation in dendritic cells (Amit, I. et al. 2009, supra).

| | | | | Stimulation | Stimulation | Stimulation | Stimulation | Stimulation |
|---|---|---|---|---|---|---|---|---|
| | Universal | Universal Gene | Universal Gene | of TLR9 | of TLR7 | of TLR4 | of TLR2 | of TLR3 |
| | Symbol | Name | Refseq ID | by CpG | by GRD | by LPS | by Pam | by PiC |
| 1 | H6pd | hexose-6-phosphate | NM_173371 | | | | | |
| | | dehydrogenase (glucose 1- | | | | | | |
| | | dehydrogenase) | | | | | | |
| 2 | Il6 | interleukin 6 | NM_031168 | | | | | |
| 3 | Samhd1 | SAM domain and HD domain, | NM_01139520 | | + | + | | + |
| | | 1 | | | | | | |
| 4 | Ccl20 | chemokine (C-C motif) ligand | NM_01159738 | | | | | |
| | | 20 | | | | | | |
| 5 | Hdc | histidine decarboxylase | NM_008230 | + | | | | |
| 6 | LOC100047619 | similar to solute carrier | XR_033736 | + | + | | + | |
| | | family 7 (cationic amino acid | | | | | | |
| | | transporter, y+ system), | | | | | | |
| | | member 5 | | | | | | |
| 7 | Slc7a5 | solute carrier family 7 | NM_011404 | + | + | | + | |
| | | (cationic amino acid | | | | | | |
| | | transporter, y+ system), | | | | | | |
| | | member 5 | | | | | | |
| 8 | Cxcl1 | chemokine (C-X-C motif) | NM_008176 | + | + | + | | |
| | | ligand 1 | | | | | | |
| 9 | Iigp1 | interferon inducible GTPase 1 | NM_001146275 | | | | | + |
| 10 | Slc39a8 | solute carrier family 39 | NM_001135149 | | | | | + |
| | | (metal ion transporter), | | | | | | |
| | | member 8 | | | | | | |
| 11 | Ccl2 | chemokine (C-C motif) ligand | NM_011333 | | | | + | + |
| | | 2 | | | | | | |
| 12 | Ccl1 | chemokine (C-C motif) ligand | NM_011329 | | | | | |
| | | 1 | | | | | | |
| 13 | Icam1 | intercellular adhesion | NM_010493 | | + | | + | |
| | | molecule 1 | | | | | | |
| 14 | Cxcl16 | chemokine (C-X-C motif) | NM_023158 | | | | | |
| | | ligand 16 | | | | | | |
| 15 | Clic4 | chloride intracellular channel | NM_013885 | | | | | |
| | | 4 (mitochondrial) | | | | | | |
| 16 | Cd83 | CD83 antigen | NM_009856 | | | | | + |
| 17 | Dusp2 | dual specificity phosphatase | NM_010090 | | | | | |
| | | 2 | | | | | | |
| 18 | Irf1 | interferon regulatory factor 1 | NM_001159396 | | | | | + |
| 19 | Ccl7 | chemokine (C-C motif) ligand | NM_013654 | | | | | + |
| | | 7 | | | | | | |
| 20 | Ccl12 | chemokine (C-C motif) ligand | NM_011331 | | | | | |
| | | 12 | | | | | | |
| 21 | Cidec | cell death-inducing DFFA-like | NM_178373 | | | | | |
| | | effector c | | | | | | |
| 22 | Egln3 | EGL nine homolog 3 | NM_028133 | | + | | | |
| | | (C. elegans) | | | | | | |
| 23 | F7 | coagulation factor VII | NM_010172 | | | + | | |
| 24 | Angptl4 | angiopoietin-like 4 | NM_020581 | | | | | |
| 25 | Ier3 | immediate early response 3 | NM_133662 | + | | + | | + |
| 26 | Zc3h12a | zinc finger CCCH type | NM_153159 | + | | | + | + |
| | | containing 12A | | | | | | |
| 27 | Clec7a | C-type lectin domain family 7, | NM_020008 | + | | | + | |
| | | member a | | | | | | |
| 28 | Ccl3 | chemokine (C-C motif) ligand | NM_011337 | | | | | |
| | | 3 | | | | | | |
| 29 | Pqlc1 | PQ loop repeat containing 1 | NM_001164420 | + | + | + | + | |
| 30 | Serpine1 | serine (or cysteine) peptidase | NM_008871 | | + | | | |
| | | inhibitor, clade E, member 1 | | | | | | |
| 31 | Il1b | interleukin 1 beta | NM_008361 | | | | | |
| 32 | Cyp2e1 | cytochrome P450, family 2, | NM_021282 | + | | + | + | + |
| | | subfamily e, polypeptide 1 | | | | | | |
| 33 | 1810011O10Rik | RIKEN cDNA 1810011O10 | NM_026931 | | | | + | + |
| | | gene | | | | | | |
| 34 | Zfp36 | zinc finger protein 36 | NM_011756 | | + | | | |
| 35 | Mmp12 | matrix metallopeptidase 12 | NM_008605 | | | | | |
| 36 | Ccl17 | chemokine (C-C motif) ligand | NM_011332 | | | | | |
| | | 17 | | | | | | |
| 37 | Ifi202b | interferon activated gene | NM_008327 | | | | | + |
| | | 202B | | | | | | |
| 38 | Bcl3 | B-cell leukemia/lymphoma 3 | NM_033601 | | | | | |
| 39 | Gpr65 | G-protein coupled receptor | NM_008152 | | | | | |
| | | 65 | | | | | | |
| 40 | Slc39a14 | solute carrier family 39 (zinc | NM_001135151 | | | | | + |
| | | transporter), member 14 | | | | | | |
| 41 | Saa3 | serum amyloid A 3 | NM_011315 | | | | | |
| 42 | Mefv | Mediterranean fever | NM_001161790 | | | | | |
| 43 | Spp1 | secreted phosphoprotein 1 | NM_009263 | + | + | + | + | |
| 44 | Nfkbiz | nuclear factor of kappa light | NM_001159394 | | + | | | |
| | | polypeptide gene enhancer in | | | | | | |
| | | B-cells inhibitor, zeta | | | | | | |
| 45 | Epgn | epithelial mitogen | NM_053087 | | | | | |
| 46 | Mt1 | metallothionein 1 | NM_013602 | | | + | | |
| 47 | Nfkb1 | nuclear factor of kappa light | NM_008689 | + | | | + | |
| | | polypeptide gene enhancer in | | | | | | |
| | | B-cells 1, p105 | | | | | | |
| 48 | Cebpd | CCAAT/enhancer binding | NM_007679 | | | | | |
| | | protein (C/EBP), delta | | | | | | |
| 49 | Acer2 | alkaline ceramidase 2 | NM_139306 | + | + | + | | + |
| 50 | Coro2a | coronin, actin binding | NM_001164804 | | | | | |
| | | protein 2A | | | | | | |
| 51 | Best2 | bestrophin 2 | NM_001130193 | | | | + | |
| 52 | Ccl24 | chemokine (C-C motif) ligand | NM_019577 | | | | + | + |
| | | 24 | | | | | | |
| 53 | Ltb4r1 | leukotriene B4 receptor 1 | NM_008519 | + | + | + | + | |
| 54 | Tnfrsf1a | tumor necrosis factor | NM_011609 | | + | | | + |
| | | receptor superfamily, | | | | | | |
| | | member 1a | | | | | | |
| 55 | Dusp6 | dual specificity phosphatase | NM_026268 | | | | | |
| | | 6 | | | | | | |
| 56 | LOC100044068 | similar to interferon- | XM_001473873 | + | + | + | + | |
| | | inducible Ifi202b | | | | | | |
| 57 | Cxcl2 | chemokine (C-X-C motif) | NM_009140 | | | | | |
| | | ligand 2 | | | | | | |
| 58 | Adam8 | a disintegrin and | NM_007403 | | | | | + |
| | | metallopeptidase domain 8 | | | | | | |
| 59 | Alox12e | arachidonate lipoxygenase, | NM_145684 | | | | + | |
| | | epidermal | | | | | | |
| 60 | Cd86 | CD86 antigen | NM_019388 | + | | | | + |
| 61 | Scd3 | stearoyl-coenzyme A | NM_024450 | | | | | + |
| | | desaturase 3 | | | | | | |
| 62 | Rela | v-rel reticuloendotheliosis | NM_009045 | + | | | | |
| | | viral oncogene homolog A | | | | | | |
| | | (avian) | | | | | | |
| 63 | Tph1 | tryptophan hydroxylase 1 | NM_001136084 | + | | | | |
| 64 | Ccl11 | chemokine (C-C motif) ligand | NM_011330 | + | | + | | |
| | | 11 | | | | | | |
| 65 | Plin4 | perilipin 4 | NM_020568 | | | | + | |
| 66 | Ptx3 | pentraxin related gene | NM_008987 | | | | + | |
| 67 | Ralgds | ral guanine nucleotide | NM_001145834 | | | | | + |
| | | dissociation stimulator | | | | | | |
| 68 | Btg2 | B-cell translocation gene 2, | NM_007570 | | | | | |
| | | anti-proliferative | | | | | | |
| 69 | Pyy | peptide YY | NM_145435 | + | | | | |
| 70 | Rab20 | RAB20, member RAS | NM_011227 | | + | | | |
| | | oncogene family | | | | | | |
| 71 | C330016O10Rik | RIKEN cDNA C330016O10 | NM_145974 | + | + | + | + | + |
| | | gene | | | | | | |
| 72 | Zfp593 | zinc finger protein 593 | NM_024215 | | + | | + | |
| 73 | LOC100048724 | similar to fatty acid | XM_001481056 | | | | | + |
| | | desaturase | | | | | | |
| 74 | Nr4a1 | nuclear receptor subfamily 4, | NM_010444 | + | | | | |
| | | group A, member 1 | | | | | | |
| 75 | Marcksl1 | MARCKS-like 1 | NM_010807 | | | | | |
| 76 | Hamp2 | hepcidin antimicrobial | NM_183257 | | | | + | |
| | | peptide 2 | | | | | | |
| 77 | Fcer1a | Fc receptor, IgE, high affinity | NM_010184 | | | + | | |
| | | I, alpha polypeptide | | | | | | |
| 78 | LOC100044927 | similar to TNF-stimulated | XM_001473344 | + | | | + | + |
| | | gene 6 protein | | | | | | |
| 79 | Anxa3 | annexin A3 | NM_013470 | | | | + | + |
| 80 | Ccl4 | chemokine (C-C motif) ligand | NM_013652 | | | | | + |
| | | 4 | | | | | | |
| 81 | Gpr84 | G protein-coupled receptor | NM_030720 | | | | | + |
| | | 84 | | | | | | |
| 82 | Rgs1 | regulator of G-protein | NM_015811 | | | | | |
| | | signaling 1 | | | | | | |
| 83 | Defb1 | defensin beta 1 | NM_007843 | | | | | |
| 84 | Nfkbia | nuclear factor of kappa light | NM_010907 | | | + | | + |
| | | polypeptide gene enhancer in | | | | | | |
| | | B-cells inhibitor, alpha | | | | | | |
| 85 | Gsdmc | gasdermin C | NM_031378 | + | | | | |
| 86 | Ch25h | cholesterol 25-hydroxylase | NM_009890 | | + | | + | + |
| 87 | Casp4 | caspase 4, apoptosis-related | NM_007609 | | + | | | |
| | | cysteine peptidase | | | | | | |
| 88 | LOC100044206 | hypothetical protein | XM_001471596 | | + | | | |
| | | LOC100044206 | | | | | | |
| 89 | LOC100046186 | similar to receptor activity | XM_001475752 | | | + | | |
| | | modifying protein 3 | | | | | | |
| 90 | Ramp3 | receptor (calcitonin) activity | NM_019511 | | | + | | |
| | | modifying protein 3 | | | | | | |
| 91 | Tnfaip2 | tumor necrosis factor, alpha- | NM_009396 | + | + | + | + | + |
| | | induced protein 2 | | | | | | |
| 92 | Ankrd57 | ankyrin repeat domain 57 | NM_172939 | | | | | |
| 93 | Mat2a | methionine | NM_145569 | + | + | + | + | + |
| | | adenosyltransferase II, alpha | | | | | | |
| 94 | Egr1 | early growth response 1 | NM_007913 | | | | | |
| 95 | Slfn2 | schlafen 2 | NM_011408 | | + | + | | |
| 96 | Ccr7 | chemokine (C-C motif) | NM_007719 | | | | | |
| | | receptor 7 | | | | | | |
| 97 | Pmaip1 | phorbol-12-myristate-13- | NM_021451 | + | + | | + | + |
| | | acetate-induced protein 1 | | | | | | |
| 98 | Tnfaip6 | tumor necrosis factor alpha | NM_009398 | | | | + | + |
| | | induced protein 6 | | | | | | |
| 99 | Il4i1 | interleukin 4 induced 1 | NM_010215 | | | | | |
| 100 | Nup62-il4i1 | Nup62-Il4i1 protein | NM_001171024 | | | | | |
| 101 | Mt2 | metallothionein 2 | NM_008630 | + | + | | | + |
| 102 | Batf3 | basic leucine zipper | NM_030060 | | + | + | | |
| | | transcription factor, ATF-like | | | | | | |
| | | 3 | | | | | | |
| 103 | Nr4a2 | nuclear receptor subfamily 4, | NM_001139509 | | | | | |
| | | group A, member 2 | | | | | | |
| 104 | Plek | pleckstrin | NM_019549 | | | + | | + |
| 105 | Phlda1 | pleckstrin homology-like | NM_009344 | | | + | | |
| | | domain, family A, member 1 | | | | | | |
| 106 | Pfkfb3 | 6-phosphofructo-2- | NM_133232 | + | + | + | + | |
| | | kinase/fructose-2,6- | | | | | | |
| | | biphosphatase 3 | | | | | | |
| 107 | Slc10a6 | solute carrier family 10 | NM_029415 | | | | | |
| | | (sodium/bile acid | | | | | | |
| | | cotransporter family), | | | | | | |
| | | member 6 | | | | | | |
| 108 | Havcr2 | hepatitis A virus cellular | NM_134250 | | | | | |
| | | receptor 2 | | | | | | |
| 109 | Ms4a4b | membrane-spanning 4- | NM_021718 | | | | + | |
| | | domains, subfamily A, | | | | | | |
| | | member 4B | | | | | | |
| 110 | Pex13 | peroxisomal biogenesis factor | NM_023651 | + | + | + | | |
| | | 13 | | | | | | |
| 111 | Bhlhe40 | basic helix-loop-helix family, | NM_011498 | | + | + | | |
| | | member e40 | | | | | | |
| 112 | Maff | v-maf musculoaponeurotic | NM_010755 | | | | | |
| | | fibrosarcoma oncogene | | | | | | |
| | | family, protein F (avian) | | | | | | |
| 113 | Ptges | prostaglandin E synthase | NM_022415 | + | | + | + | + |
| 114 | Il7r | interleukin 7 receptor | NM_008372 | | | + | + | |
| 115 | Ttr | transthyretin | NM_013697 | | | | | |
| 116 | Map3k6 | mitogen-activated protein | NM_016693 | | | | | |
| | | kinase kinase kinase 6 | | | | | | |
| 117 | Mafk | v-maf musculoaponeurotic | NM_010757 | | | | + | |
| | | fibrosarcoma oncogene | | | | | | |
| | | family, protein K (avian) | | | | | | |
| 118 | Clec4e | C-type lectin domain family 4, | NM_019948 | | | | + | |
| | | member e | | | | | | |
| 119 | Cfd | complement factor D | NM_013459 | | | | | |
| | | (adipsin) | | | | | | |
| 120 | B4galt1 | UDP-Gal: betaGlcNAc beta 1,4- | NM_022305 | | + | | | |
| | | galactosyltransferase, | | | | | | |
| | | polypeptide 1 | | | | | | |
| 121 | Gpr133 | G protein-coupled receptor | NM_001081342 | | | | + | |
| | | 133 | | | | | | |
| 122 | Junb | Jun-B oncogene | NM_008416 | | | + | + | + |
| 123 | Zkscan6 | zinc finger with KRAB and | NM_026107 | | | + | + | |
| | | SCAN domains 6 | | | | | | |
| 124 | Uap1 | UDP-N-acetylglucosamine | NM_133806 | | | | + | |
| | | pyrophosphorylase 1 | | | | | | |
| 125 | Pde4b | phosphodiesterase 4B, cAMP | NM_019840 | | + | | | + |
| | | specific | | | | | | |
| 126 | Myd88 | myeloid differentiation | NM_010851 | | | | | |
| | | primary response gene 88 | | | | | | |
| 127 | Rhod | ras homolog gene family, | NM_007485 | | + | | | |
| | | member D | | | | | | |
| 128 | Il1rl1 | interleukin 1 receptor-like 1 | NM_001025602 | | | | | |
| 129 | Gbp2 | guanylate binding protein 2 | NM_010260 | + | | + | | + |
| 130 | Fkbp5 | FK506 binding protein 5 | NM_010220 | | | + | + | + |
| 131 | Bcl6b | B-cell CLL/lymphoma 6, | NM_007528 | + | | + | + | + |
| | | member B | | | | | | |
| 132 | Sema4d | sema domain, | NM_013660 | + | | + | | + |
| | | immunoglobulin domain (Ig), | | | | | | |
| | | transmembrane domain (TM) | | | | | | |
| | | and short cytoplasmic | | | | | | |
| | | domain, (semaphorin) 4D | | | | | | |
| 133 | Gch1 | GTP cyclohydrolase 1 | NM_008102 | | + | + | | |
| 134 | Tlr2 | toll-like receptor 2 | NM_011905 | | | | + | + |
| 135 | Myo1g | myosin IG | NM_178440 | | + | | | |
| 136 | Osmr | oncostatin M receptor | NM_011019 | + | + | | | |
| 137 | Ifrd1 | interferon-related | NM_013562 | | | + | + | + |
| | | developmental regulator 1 | | | | | | |
| 138 | Ippk | inositol 1,3,4,5,6- | NM_199056 | | | + | | + |
| | | pentakisphosphate 2-kinase | | | | | | |
| 139 | Irf5 | interferon regulatory factor 5 | NM_012057 | | | | | |
| 140 | Cytip | cytohesin 1 interacting | NM_139200 | | | | | |
| | | protein | | | | | | |
| 141 | Rgs16 | regulator of G-protein | NM_011267 | | + | + | | + |
| | | signaling 16 | | | | | | |
| 142 | LOC640502 | similar to UDP-N- | XM_917484 | | | | + | |
| | | acetylhexosamine | | | | | | |
| | | pyrophosphorylase | | | | | | |
| 143 | Ifngr1 | interferon gamma receptor 1 | NM_010511 | + | | | + | |
| 144 | Socs2 | suppressor of cytokine | NM_001168655 | | | | + | |
| | | signaling 2 | | | | | | |
| 145 | Stc1 | stanniocalcin 1 | NM_009285 | | | | | + |
| 146 | Adcy8 | adenylate cyclase 8 | NM_009623 | | | | | |
| 147 | Pdzk1ip1 | PDZK1 interacting protein 1 | NM_001164557 | | + | | | + |
| 148 | Ehf | ets homologous factor | NM_007914 | + | | + | | |
| 149 | Hmgcs2 | 3-hydroxy-3-methylglutaryl- | NM_008256 | | | | | + |
| | | Coenzyme A synthase 2 | | | | | | |
| 150 | Ubr2 | ubiquitin protein ligase E3 | NM_146078 | | | | | |
| | | component n-recognin 2 | | | | | | |
| 151 | Rassf1 | Ras association (RalGDS/AF-6) | NM_019713 | + | | | | + |
| | | domain family member 1 | | | | | | |
| 152 | Tnf | tumor necrosis factor | NM_013693 | | | | | |
| 153 | Niacr1 | niacin receptor 1 | NM_030701 | | | | | |
| 154 | Gadd45b | growth arrest and DNA- | NM_008655 | | | | | |
| | | damage-inducible 45 beta | | | | | | |
| 155 | Dusp8 | dual specificity phosphatase | NM_008748 | | | | | + |
| | | 8 | | | | | | |
| 156 | Asgr2 | asialoglycoprotein receptor 2 | NM_007493 | | | | | |
| 157 | Acaa1b | acetyl-Coenzyme A | NM_146230 | | | | + | |
| | | acyltransferase 1B | | | | | | |
| 158 | Lat2 | linker for activation of T cells | NM_020044 | | | | | |
| | | family, member 2 | | | | | | |
| 159 | Ereg | epiregulin | NM_007950 | | | | | + |
| 160 | LOC100045163 | similar to RIKEN cDNA | XM_001473518 | + | | | | + |
| | | 1100001H23 gene | | | | | | |
| 161 | Plbd1 | phospholipase B domain | NM_025806 | + | | | | + |
| | | containing 1 | | | | | | |
| 162 | Slc5a1 | solute carrier family 5 | NM_019810 | + | + | + | + | + |
| | | (sodium/glucose | | | | | | |
| | | cotransporter), member 1 | | | | | | |
| 163 | Ptpn22 | protein tyrosine phosphatase, | NM_008979 | | | | | |
| | | non-receptor type 22 | | | | | | |
| | | (lymphoid) | | | | | | |
| 164 | LOC100044257 | hypothetical protein | XM_001471785 | | | | | |
| | | LOC100044257 | | | | | | |
| 165 | Otud7a | OTU domain containing 7A | NM_130880 | | | | | |
| 166 | Zfp295 | zinc finger protein 295 | NM_001081684 | | | | | |
| 167 | Nnat | neuronatin | NM_010923 | | | + | | |
| 168 | Ptger2 | prostaglandin E receptor 2 | NM_008964 | + | | | | |
| | | (subtype EP2) | | | | | | |
| 169 | Mlxip | MLX interacting protein | NM_133917 | | | | | |
| 170 | Xdh | xanthine dehydrogenase | NM_011723 | | | | | |
| 171 | Psors1c2 | psoriasis susceptibility 1 | NM_020576 | | | | | |
| | | candidate 2 (human) | | | | | | |
| 172 | Lep | leptin | NM_008493 | | | | | |
| 173 | Pdcd1lg2 | programmed cell death 1 | NM_021396 | + | + | | | + |
| | | ligand 2 | | | | | | |
| 174 | Slc3a2 | solute carrier family 3 | NM_001161413 | + | | + | | + |
| | | (activators of dibasic and | | | | | | |
| | | neutral amino acid | | | | | | |
| | | transport), member 2 | | | | | | |
| 175 | IgtP | interferon gamma induced | NM_018738 | + | | + | + | + |
| | | GTPase | | | | | | |
| 176 | Sla | src-like adaptor | NM_001029841 | | | | | |
| 177 | Nts | neurotensin | NM_024435 | | | | | |
| 178 | Azin1 | antizyme inhibitor 1 | NM_001102458 | | | | | |
| 179 | Ets2 | E26 avian leukemia oncogene | NM_011809 | | + | + | + | |
| | | 2, 3′ domain | | | | | | |
| 180 | Cd209c | CD209c antigen | NM_130903 | | | | | |
| 181 | Srgn | serglycin | NM_011157 | | | | + | |
| 182 | Arc | activity regulated | NM_018790 | | | | | |
| | | cytoskeletal-associated | | | | | | |
| | | protein | | | | | | |
| 183 | Tslp | thymic stromal | NM_021367 | | | | | |
| | | lymphopoietin | | | | | | |
| 184 | LOC68395 | histocompatibility 2, Q region | XM_903697 | | | | | |
| | | locus 6-like | | | | | | |
| 185 | Bcar1 | breast cancer anti-estrogen | NM_009954 | | + | + | + | + |
| | | resistance 1 | | | | | | |
| 186 | Ifitm6 | interferon induced | NM_001033632 | | | | | |
| | | transmembrane protein 6 | | | | | | |
| 187 | Cyr61 | cysteine rich protein 61 | NM_010516 | | | | + | + |
| 188 | Oasl2 | 2′-5′ oligoadenylate | NM_011854 | | | | | |
| | | synthetase-like 2 | | | | | | |
| 189 | Map3k8 | mitogen-activated protein | NM_007746 | + | | | | |
| | | kinase kinase kinase 8 | | | | | | |
| 190 | Traf1 | TNF receptor-associated | NM_009421 | | | | | + |
| | | factor 1 | | | | | | |
| 191 | Dok2 | docking protein 2 | NM_010071 | | | + | + | |
| 192 | Retnla | resistin like alpha | NM_020509 | | | | | |
| 193 | Trim25 | tripartite motif-containing 25 | NM_009546 | | | | | |
| 194 | Klf13 | Kruppel-like factor 13 | NM_021366 | | | | | |
| 195 | Ifi204 | interferon activated gene 204 | NM_008329 | + | | + | | |
| 196 | St3gal5 | ST3 beta-galactoside alpha- | NM_001035228 | | | | + | |
| | | 2,3-sialyltransferase 5 | | | | | | |
| 197 | Pck1 | phosphoenolpyruvate | NM_011044 | | | | | + |
| | | carboxykinase 1, cytosolic | | | | | | |
| 198 | Lta | lymphotoxin A | NM_010735 | | | | + | + |
| 199 | Picalm | phosphatidylinositol binding | NM_146194 | + | + | + | + | + |
| | | clathrin assembly protein | | | | | | |
| 200 | Per1 | period homolog 1 | NM_001159367 | | | + | | |
| | | (Drosophila) | | | | | | |
| 201 | Serpina3n | serine (or cysteine) peptidase | NM_009252 | | | | | |
| | | inhibitor, clade A, member | | | | | | |
| | | 3N | | | | | | |
| 202 | Lce3a | late cornified envelope 3A | NM_001039594 | | | | | |
| 203 | 2310016C08Rik | RIKEN cDNA 2310016C08 | NM_023516 | | + | | | |
| | | gene | | | | | | |
| 204 | Cd44 | CD44 antigen | NM_001039150 | | + | | + | + |
| 205 | Arid5a | AT rich interactive domain | NM_001172205 | | | | | |
| | | 5A (MRF1-like) | | | | | | |
| 206 | Gem | GTP binding protein (gene | NM_010276 | + | | | + | + |
| | | overexpressed in skeletal | | | | | | |
| | | muscle) | | | | | | |
| 207 | Inpp5d | inositol polyphosphate-5- | NM_001110192 | | | | | |
| | | phosphatase D | | | | | | |
| 208 | Rel | reticuloendotheliosis | NM_009044 | | | | + | |
| | | oncogene | | | | | | |
| 209 | Klf9 | Kruppel-like factor 9 | NM_010638 | | | | | |
| 210 | Nlrp3 | NLR family, pyrin domain | NM_145827 | | + | | | |
| | | containing 3 | | | | | | |
| 211 | Pard6b | par-6 (partitioning defective | NM_021409 | | | | | |
| | | 6) homolog beta (C. elegans) | | | | | | |
| 212 | Ccr1 | chemokine (C-C motif) | NM_009912 | | | | | |
| | | receptor 1 | | | | | | |
| 213 | Ccl9 | chemokine (C-C motif) ligand | NM_011338 | | | | | |
| | | 9 | | | | | | |
| 214 | Stat5a | signal transducer and | NM_001164062 | | | | | |
| | | activator of transcription 5A | | | | | | |
| 215 | Nfkbie | nuclear factor of kappa light | NM_008690 | + | + | + | + | + |
| | | polypeptide gene enhancer in | | | | | | |
| | | B-cells inhibitor, epsilon | | | | | | |
| 216 | Stk17b | serine/threonine kinase 17b | NM_133810 | | | | + | |
| | | (apoptosis-inducing) | | | | | | |
| 217 | Il2rg | interleukin 2 receptor, | NM_013563 | | | | | |
| | | gamma chain | | | | | | |
| 218 | Socs3 | suppressor of cytokine | NM_007707 | | | | + | + |
| | | signaling 3 | | | | | | |
| 219 | Cpa1 | carboxypeptidase A1 | NM_025350 | | + | + | | |
| 220 | Hmgcr | 3-hydroxy-3-methylglutaryl- | NM_008255 | | | | + | |
| | | Coenzyme A reductase | | | | | | |
| 221 | Rasa3 | RAS p21 protein activator 3 | NM_009025 | | | | | |
| 222 | Sdhaf1 | succinate dehydrogenase | NM_001033140 | | + | + | + | + |
| | | complex assembly factor 1 | | | | | | |
| 223 | Sik1 | salt inducible kinase 1 | NM_010831 | | | | + | |
| 224 | Tnfrsf12a | tumor necrosis factor | NM_001161746 | | + | + | | + |
| | | receptor superfamily, | | | | | | |
| | | member 12a | | | | | | |
| 225 | Tnfaip3 | tumor necrosis factor, alpha- | NM_001166402 | | | + | + | + |
| | | induced protein 3 | | | | | | |
| 226 | Gpr97 | G protein-coupled receptor | NM_173036 | | | | | |
| | | 97 | | | | | | |
| 227 | Jmjd6 | jumonji domain containing 6 | NM_033398 | + | | + | | + |
| 228 | Dedd2 | death effector domain- | NM_207677 | | + | + | + | |
| | | containing DNA binding | | | | | | |
| | | protein 2 | | | | | | |
| 229 | Ear2 | eosinophil-associated, | NM_007895 | + | + | + | | + |
| | | ribonuclease A family, | | | | | | |
| | | member 2 | | | | | | |
| 230 | Ptk6 | PTK6 protein tyrosine kinase | NM_009184 | + | + | | + | |
| | | 6 | | | | | | |
| 231 | Dnajb4 | DnaJ (Hsp40) homolog, | NM_025926 | | | | | |
| | | subfamily B, member 4 | | | | | | |
| 232 | Nsg1 | neuron specific gene family | NM_010942 | | | + | | |
| | | member 1 | | | | | | |
| 233 | Gucy2c | guanylate cyclase 2c | NM_001127318 | | | | + | + |
| 234 | Sele | selectin, endothelial cell | NM_011345 | | | + | + | + |
| 235 | Ppp1r15a | protein phosphatase 1, | NM_008654 | + | | | | |
| | | regulatory (inhibitor) subunit | | | | | | |
| | | 15A | | | | | | |
| 236 | Mettl7b | methyltransferase like 7B | NM_027853 | | + | + | | + |
| 237 | Glipr1 | GLI pathogenesis-related 1 | NM_028608 | + | | + | + | |
| | | (glioma) | | | | | | |
| 238 | Kctd4 | potassium channel | NM_026214 | | | | | |
| | | tetramerisation domain | | | | | | |
| | | containing 4 | | | | | | |
| 239 | Rnase1 | ribonuclease, RNase A family, | NM_011271 | | | + | + | |
| | | 1 (pancreatic) | | | | | | |
| 240 | Ier2 | immediate early response 2 | NM_010499 | | | | | |
| 241 | Ccl22 | chemokine (C-C motif) ligand | NM_009137 | | | | | + |
| | | 22 | | | | | | |
| 242 | Odc1 | ornithine decarboxylase, | NM_013614 | | + | + | + | + |
| | | structural 1 | | | | | | |
| 243 | Csrp1 | cysteine and glycine-rich | NM_007791 | | | | | + |
| | | protein 1 | | | | | | |
| 244 | Pvr | poliovirus receptor | NM_027514 | | | + | | |
| 245 | Aqp3 | aquaporin 3 | NM_016689 | | | + | + | + |
| 246 | Cd2 | CD2 antigen | NM_013486 | | | | | |
| 247 | Rnf19b | ring finger protein 19B | NM_029219 | | | | | + |
| 248 | Ece1 | endothelin converting | NM_199307 | | | | | |
| | | enzyme 1 | | | | | | |
| 249 | Gnl3 | guanine nucleotide binding | NM_153547 | | + | | | |
| | | protein-like 3 (nucleolar) | | | | | | |
| 250 | Ccrn4l | CCR4 carbon catabolite | NM_009834 | | + | + | | + |
| | | repression 4-like | | | | | | |
| | | (S. cerevisiae) | | | | | | |
| 251 | LOC100047134 | similar to carbon catabolite | XM_001477322 | | + | + | | + |
| | | repression 4 protein homolog | | | | | | |
| 252 | Dclre1b | DNA cross-link repair 1B, | NM_001025312 | | | | | |
| | | PSO2 homolog (S. cerevisiae) | | | | | | |
| 253 | 1600029D21Rik | RIKEN cDNA 1600029D21 | NM_029639 | | | | | |
| | | gene | | | | | | |
| 254 | LOC100048365 | similar to keratin associated | XM_001480092 | | | | | |
| | | protein 6-1 | | | | | | |
| 255 | Il10ra | interleukin 10 receptor, | NM_008348 | | | | | |
| | | alpha | | | | | | |
| 256 | Csrp2 | cysteine and glycine-rich | NM_007792 | | | | | |
| | | protein 2 | | | | | | |
| 257 | LOC100045085 | similar to Anxa3 | XM_001473636 | | | | | |
| 258 | F2rl1 | coagulation factor II | NM_007974 | + | | | + | |
| | | (thrombin) receptor-like 1 | | | | | | |
| 259 | Akap12 | A kinase (PRKA) anchor | NM_031185 | | | + | + | |
| | | protein (gravin) 12 | | | | | | |
| 260 | Phlda2 | pleckstrin homology-like | NM_009434 | | | | | |
| | | domain, family A, member 2 | | | | | | |
| 261 | Card14 | caspase recruitment domain | NM_130886 | | | | | + |
| | | family, member 14 | | | | | | |
| 262 | 1700017B05Rik | RIKEN cDNA 1700017B05 | NM_028820 | | | | | |
| | | gene | | | | | | |
| 263 | Bsdc1 | BSD domain containing 1 | NM_133889 | | + | | | + |
| 264 | Il1r1 | interleukin 1 receptor, type I | NM_001123382 | | | + | + | + |
| 265 | LOC100048342 | similar to mKIAA1547 protein | XM_001480046 | | + | | | |
| 266 | Tle3 | transducin-like enhancer of | NM_001083927 | | + | | | |
| | | split 3, homolog of | | | | | | |
| | | Drosophila E(spl) | | | | | | |
| 267 | Parp14 | poly (ADP-ribose) polymerase | NM_001039530 | | | + | | + |
| | | family, member 14 | | | | | | |
| 268 | Rac2 | RAS-related C3 botulinum | NM_009008 | | | + | | |
| | | substrate 2 | | | | | | |
| 269 | Dgat1 | diacylglycerol O- | NM_010046 | | | | | + |
| | | acyltransferase 1 | | | | | | |
| 270 | Ltb4r2 | leukotriene B4 receptor 2 | NM_020490 | | | | | |
| 271 | Stk40 | serine/threonine kinase 40 | NM_001145827 | | + | | + | |
| 272 | Slc6a6 | solute carrier family 6 | NM_009320 | | | | | |
| | | (neurotransmitter | | | | | | |
| | | transporter, taurine), | | | | | | |
| | | member 6 | | | | | | |
| 273 | Ppif | peptidylprolyl isomerase F | NM_134084 | | | | + | |
| | | (cyclophilin F) | | | | | | |
| 274 | Hp | haptoglobin | NM_017370 | | | | | |
| 275 | Slpi | secretory leukocyte peptidase | NM_011414 | | | | | + |
| | | inhibitor | | | | | | |
| 276 | Per2 | period homolog 2 | NM_011066 | | + | | | |
| | | (Drosophila) | | | | | | |
| 277 | Thrsp | thyroid hormone responsive | NM_009381 | | + | + | | |
| | | SPOT14 homolog (Rattus) | | | | | | |
| 278 | Tsc22d3 | TSC22 domain family, | NM_001077364 | | | | | |
| | | member 3 | | | | | | |
| 279 | 1810055G02Rik | RIKEN cDNA 1810055G02 | NM_028077 | | | | | |
| | | gene | | | | | | |
| 280 | Sell | selectin, lymphocyte | NM_001164059 | | + | + | + | + |
| 281 | LOC100046643 | similar to sprouty 1 | XM_001476559 | | | + | | |
| 282 | Spry1 | sprouty homolog 1 | NM_011896 | | | + | | |
| | | (Drosophila) | | | | | | |
| 283 | Plekhb2 | pleckstrin homology domain | NM_145516 | + | | | + | |
| | | containing, family B | | | | | | |
| | | (evectins) member 2 | | | | | | |
| 284 | Ctla2b | cytotoxic T lymphocyte- | NM_001145801 | | | | | |
| | | associated protein 2 beta | | | | | | |
| 285 | Tgm2 | transglutaminase 2, C | NM_009373 | | | | | |
| | | polypeptide | | | | | | |
| 286 | Tubb2a | tubulin, beta 2A | NM_009450 | | | | + | + |
| 287 | Cyp4a10 | cytochrome P450, family 4, | NM_010011 | | + | | | |
| | | subfamily a, polypeptide 10 | | | | | | |
| 288 | Cxcl13 | chemokine (C-X-C motif) | NM_018866 | | | | | |
| | | ligand 13 | | | | | | |
| 289 | Tgif1 | TGFB-induced factor | NM_001164074 | | | | | + |
| | | homeobox 1 | | | | | | |
| 290 | Gyk | glycerol kinase | NM_008194 | + | | + | | + |
| 291 | Gzmb | granzyme B | NM_013542 | + | + | + | + | |
| 292 | 5730408K05Rik | RIKEN cDNA 5730408K05 | NR_027866 | + | | + | | + |
| | | gene | | | | | | |
| 293 | Pvrl1 | poliovirus receptor-related 1 | NM_021424 | | + | + | + | |
| 294 | Abcc3 | ATP-binding cassette, sub- | NM_029600 | | | | | |
| | | family C (CFTR/MRP), | | | | | | |
| | | member 3 | | | | | | |
| 295 | Hmox1 | heme oxygenase (decycling) | NM_010442 | | | + | | + |
| | | 1 | | | | | | |
| 296 | Plk3 | polo-like kinase 3 | NM_013807 | + | + | + | | |
| | | (Drosophila) | | | | | | |
| 297 | Adap2 | ArfGAP with dual PH domains | NM_172133 | | | | | |
| | | 2 | | | | | | |

TABLE 3
Genes showing ≧2-fold increased expression 30 minutes to 1 hour after DBP application, with corresponding expression
changes at 1-2 hr and 2-4 hr time intervals.

| | | | | Change in Gene Expression (Weighted Difference) | | | |
|---|---|---|---|---|---|---|---|
| | | | | 30 min to | 30 min to | | 1 hr to |
| | Universal | Universal Gene | Universal Gene | 1 hr | 1 hr P- | 1 hr to 2 hr | 2 hr P- |
| # | Symbol | Name | Refseq ID | Transition | value | Transition | value |
| 1 | Riok2 | RIO kinase 2 (yeast) | NM_025934 | 21.6415603 | 0.00067446 | 0.52987812 | 0.31289344 |
| 2 | Trim16 | tripartite motif- | NM_053169 | 16.375814 | 0.00129272 | 2.68040358 | 0.03524056 |
| | | containing 16 | | | | | |
| 3 | Has2 | hyaluronan synthase 2 | NM_008216 | 13.9822557 | 0.00174236 | −0.0074131 | 0.97819245 |
| 4 | Xcl1 | chemokine (C motif) | NM_008510 | 13.50186 | 0.00185477 | −0.1460012 | 0.68176709 |
| | | ligand 1 | | | | | |
| 5 | Fos | FBJ osteosarcoma | NM_010234 | 13.3784483 | 0.00191097 | −0.025132 | 0.93030576 |
| | | oncogene | | | | | |
| 6 | Zfp820 | zinc finger protein | NM_029281 | 12.0616225 | 0.00230441 | −0.2190941 | 0.57778777 |
| | | 820 | | | | | |
| 7 | Pvrl2 | poliovirus receptor- | NM_001159724 | 10.0872623 | 0.00354092 | 1.66278546 | 0.07492131 |
| | | related 2 | | | | | |
| 8 | Klra20 | killer cell lectin-like | NM_053150 | 9.99178954 | 0.00359712 | −0.0096013 | 0.97274056 |
| | | receptor subfamily | | | | | |
| | | A, member 20 | | | | | |
| 9 | Krtap6-1 | keratin associated | NM_010672 | 7.58940073 | 0.00607014 | 6.3046581 | 0.00685701 |
| | | protein 6-1 | | | | | |
| 10 | 6330416L07Rik | RIKEN cDNA | NM_176962 | 6.62309169 | 0.00792491 | 0.56751293 | 0.29215378 |
| | | 6330416L07 gene | | | | | |
| 11 | Gm10229 | predicted gene | XM_001474256 | 6.57967734 | 0.00803732 | 5.79618154 | 0.00809353 |
| | | 10229 | | | | | |
| 12 | Enox2 | ecto-NOX disulfide- | NM_145951 | 6.52399412 | 0.00809353 | −0.3385345 | 0.44677383 |
| | | thiol exchanger 2 | | | | | |
| 13 | Expi | extracellular | NM_007969 | 6.50352418 | 0.00814973 | 0.133497 | 0.70250674 |
| | | proteinase inhibitor | | | | | |
| 14 | Fam64a | family with sequence | NM_144526 | 6.25086602 | 0.00933004 | −1.0509019 | 0.14304182 |
| | | similarity 64, | | | | | |
| | | member A | | | | | |
| 15 | Krtap15 | keratin associated | NM_013713 | 5.79063193 | 0.01084757 | 10.5103331 | 0.0022482 |
| | | protein 15 | | | | | |
| 16 | Klf10 | Kruppel-like factor | NM_013692 | 5.69958541 | 0.01124101 | 0.84251695 | 0.19171538 |
| | | 10 | | | | | |
| 17 | Il12rb2 | interleukin 12 | NM_008354 | 5.65275728 | 0.01135342 | 0.88494678 | 0.18030576 |
| | | receptor, beta 2 | | | | | |
| 18 | Krtap16-10 | keratin associated | NM_183296 | 5.3276603 | 0.01292716 | 7.32468153 | 0.00483363 |
| | | protein 16-10 | | | | | |
| 19 | Krtap16-8 | keratin associated | NM_130856 | 5.30039537 | 0.01309577 | 7.02047155 | 0.00545189 |
| | | protein 16-8 | | | | | |
| 20 | Il2 | interleukin 2 | NM_008366 | 5.1268197 | 0.01371403 | −0.357578 | 0.42957509 |
| 21 | Krtap16-5 | keratin associated | NM_130857 | 4.97485192 | 0.01416367 | 7.54470731 | 0.0044402 |
| | | protein 16-5 | | | | | |
| 22 | Prss22 | protease, serine, 22 | NM_133731 | 4.66744578 | 0.01523156 | 0.73953789 | 0.21908723 |
| 23 | Trp53inp1 | transformation | NM_021897 | 4.54057146 | 0.01618705 | 0.34187618 | 0.44317671 |
| | | related protein 53 | | | | | |
| | | inducible nuclear | | | | | |
| | | protein 1 | | | | | |
| 24 | Krt25 | keratin 25 | NM_133730 | 4.39134397 | 0.01714254 | 9.64882464 | 0.00286646 |
| 25 | Tchh | trichohyalin | NM_001163098 | 4.35197049 | 0.01759218 | 12.2943658 | 0.00157374 |
| 26 | Zcchc18 | zinc finger, CCHC | NM_001035509 | 4.28419496 | 0.01798561 | 0.03450383 | 0.90765513 |
| | | domain containing | | | | | |
| | | 18 | | | | | |
| 27 | Krtap16-7 | keratin associated | NM_028621 | 4.24631928 | 0.01837905 | 7.11949077 | 0.00517086 |
| | | protein 16-7 | | | | | |
| 28 | Eif1a | eukaryotic | NM_010120 | 4.22902245 | 0.01854766 | 3.8969793 | 0.01686151 |
| | | translation initiation | | | | | |
| | | factor 1A | | | | | |
| 29 | Zscan12 | zinc finger and SCAN | NM_016684 | 4.16481488 | 0.01905351 | −0.4377773 | 0.36758094 |
| | | domain containing | | | | | |
| | | 12 | | | | | |
| 30 | Id1 | inhibitor of DNA | NM_010495 | 4.03460696 | 0.01989658 | −0.5035777 | 0.32801259 |
| | | binding 1 | | | | | |
| 31 | Wbp5 | WW domain binding | NM_011712 | 4.02911146 | 0.02000899 | −0.0659248 | 0.83267761 |
| | | protein 5 | | | | | |
| 32 | Krtap6-2 | keratin associated | NM_010673 | 3.89774182 | 0.02079586 | 6.77114239 | 0.00567671 |
| | | protein 6-2 | | | | | |
| 33 | Krtap8-1 | keratin associated | NM_010675 | 3.8371282 | 0.0211893 | 6.82619962 | 0.0055643 |
| | | protein 8-1 | | | | | |
| 34 | Ik | IK cytokine | NM_011879 | 3.83932583 | 0.0211893 | −0.0538758 | 0.86128597 |
| 35 | 2010109K11Rik | RIKEN cDNA | NM_001162903 | 3.83047285 | 0.0212455 | 3.41887705 | 0.02281924 |
| | | 2010109K11 gene | | | | | |
| 36 | Gadd45a | growth arrest and | NM_007836 | 3.72281042 | 0.02208858 | 0.80263649 | 0.20160746 |
| | | DNA-damage- | | | | | |
| | | inducible 45 alpha | | | | | |
| 37 | Mmp1b | matrix | NM_032007 | 3.71952001 | 0.02208858 | −0.1315585 | 0.70587905 |
| | | metallopeptidase 1b | | | | | |
| | | (interstitial | | | | | |
| | | collagenase) | | | | | |
| 38 | Zfp764 | zinc finger protein | NM_001167832 | 3.6894216 | 0.02259442 | −0.3605099 | 0.42704586 |
| | | 764 | | | | | |
| 39 | Slc26a5 | solute carrier family | NM_030727 | 3.67324898 | 0.02265063 | 0.23669253 | 0.55603642 |
| | | 26, member 5 | | | | | |
| 40 | Stambpl1 | STAM binding | NM_029682 | 3.61036715 | 0.02321268 | 0.69723809 | 0.23403777 |
| | | protein like 1 | | | | | |
| 41 | St5 | suppression of | NM_001001326 | 3.56899028 | 0.02371853 | 0.09067627 | 0.78259892 |
| | | tumorigenicity 5 | | | | | |
| 42 | Acot4 | acyl-CoA thioesterase 4 | NM_134247 | 3.5301706 | 0.02394335 | 0.11052882 | 0.74488534 |
| 43 | Map3k14 | mitogen-activated | NM_016896 | 3.52035547 | 0.02411196 | 1.38362847 | 0.09886466 |
| | | protein kinase | | | | | |
| | | kinase kinase 14 | | | | | |
| 44 | Krt27 | keratin 27 | NM_010666 | 3.30527402 | 0.02652878 | 10.8601872 | 0.00213579 |
| 45 | Lefty1 | left right | NM_010094 | 3.29222129 | 0.02664119 | 0.14925361 | 0.67698966 |
| | | determination factor 1 | | | | | |
| 46 | Krtap4-16 | keratin associated | NM_001013823 | 3.21219237 | 0.02827113 | 7.86936284 | 0.00410297 |
| | | protein 4-16 | | | | | |
| 47 | Krtap8-2 | keratin associated | NM_010676 | 3.18505161 | 0.02849595 | 5.46087208 | 0.00910522 |
| | | protein 8-2 | | | | | |
| 48 | Cxcl14 | chemokine (C—X—C | NM_019568 | 3.17650255 | 0.02872077 | −0.1254758 | 0.71745728 |
| | | motif) ligand 14 | | | | | |
| 49 | Fastkd1 | FAST kinase domains 1 | NM_177244 | 3.15302322 | 0.02883318 | −0.5126072 | 0.3225607 |
| 50 | Gtpbp5 | GTP binding protein 5 | NM_001083328 | 3.09405383 | 0.03001349 | −0.4452098 | 0.3626911 |
| 51 | Acsl4 | acyl-CoA synthetase | NM_001033600 | 3.05190004 | 0.03080036 | −0.0448811 | 0.88146358 |
| | | long-chain family | | | | | |
| | | member 4 | | | | | |
| 52 | Tyrp1 | tyrosinase-related | NM_031202 | 3.05016176 | 0.03085656 | 0.61422971 | 0.26792941 |
| | | protein 1 | | | | | |
| 53 | Plscr1 | phospholipid | NM_011636 | 3.01343278 | 0.03141862 | 0.23465597 | 0.55828462 |
| | | scramblase 1 | | | | | |
| 54 | Med21 | mediator complex | NM_025315 | 2.92180268 | 0.03321718 | −0.0109105 | 0.96875 |
| | | subunit 21 | | | | | |
| 55 | A030005K14Rik | RIKEN cDNA | XR_002331 | 2.90250845 | 0.033442 | 6.08260421 | 0.00753147 |
| | | A030005K14 gene | | | | | |
| 56 | Krtap16-1 | keratin associated | NM_130870 | 2.87572137 | 0.03445369 | 6.36721169 | 0.0066884 |
| | | protein 16-1 | | | | | |
| 57 | Cd207 | CD207 antigen | NM_144943 | 2.8578155 | 0.03512815 | −0.0350785 | 0.90591277 |
| 58 | Zfp654 | zinc finger protein | NM_028059 | 2.8373896 | 0.03557779 | −0.2298275 | 0.56418615 |
| | | 654 | | | | | |
| 59 | AY026312 | cDNA sequence | NM_133359 | 2.75410849 | 0.03703912 | 5.32705365 | 0.00927383 |
| | | AY026312 | | | | | |
| 60 | Zfp7 | zinc finger protein 7 | NM_145916 | 2.74122858 | 0.03726394 | −0.1005709 | 0.76393885 |
| 61 | Ear11 | eosinophil- | NM_053113 | 2.73713967 | 0.03748876 | 0.36852056 | 0.42046987 |
| | | associated, | | | | | |
| | | ribonuclease A | | | | | |
| | | family, member 11 | | | | | |
| 62 | Cyp27b1 | cytochrome P450, | NM_010009 | 2.68744216 | 0.03951214 | 2.60539143 | 0.03760117 |
| | | family 27, subfamily | | | | | |
| | | b, polypeptide 1 | | | | | |
| 63 | Sox4 | SRY-box containing | NM_009238 | 2.68540095 | 0.03956835 | −0.1669257 | 0.64860612 |
| | | gene 4 | | | | | |
| 64 | Cd40 | CD40 antigen | NM_011611 | 2.66003324 | 0.03990558 | 4.05879207 | 0.01573741 |
| 65 | Fam43a | family with sequence | NM_177632 | 2.65157365 | 0.04001799 | 0.89720535 | 0.17710207 |
| | | similarity 43, | | | | | |
| | | member A | | | | | |
| 66 | Pdlim4 | PDZ and LIM domain 4 | NM_019417 | 2.63888777 | 0.0401866 | −0.0535226 | 0.86229766 |
| 67 | Tnfrsf18 | tumor necrosis | NM_009400 | 2.64028359 | 0.0401866 | −0.011021 | 0.96841277 |
| | | factor receptor | | | | | |
| | | superfamily, | | | | | |
| | | member 18 | | | | | |
| 68 | Leng1 | leukocyte receptor | NM_027203 | 2.62962308 | 0.04035522 | −0.0869246 | 0.78990558 |
| | | cluster (LRC) | | | | | |
| | | member 1 | | | | | |
| 69 | Apaf1 | apoptotic peptidase | NM_001042558 | 2.61778035 | 0.04114209 | 0.43891912 | 0.36662545 |
| | | activating factor 1 | | | | | |
| 70 | S100a9 | S100 calcium | NM_009114 | 2.61069123 | 0.0413107 | −0.0174751 | 0.950933 |
| | | binding protein A9 | | | | | |
| | | (calgranulin B) | | | | | |
| 71 | Krt33a | keratin 33A | NM_027983 | 2.60663957 | 0.04159173 | 8.33275258 | 0.00370953 |
| 72 | Hbegf | heparin-binding | NM_010415 | 2.59984619 | 0.04164793 | 0.30500182 | 0.47841727 |
| | | EGF-like growth | | | | | |
| | | factor | | | | | |
| 73 | Krt17 | keratin 17 | NM_010663 | 2.59481252 | 0.04176034 | 0.11648545 | 0.7321268 |
| 74 | Gcm2 | glial cells missing | NM_008104 | 2.5656754 | 0.04249101 | −0.2010201 | 0.59948291 |
| | | homolog 2 | | | | | |
| | | (Drosophila) | | | | | |
| 75 | Krtap14 | keratin associated | NM_013707 | 2.55339146 | 0.04254721 | 6.84314805 | 0.0055643 |
| | | protein 14 | | | | | |
| 76 | Ovol1 | OVO homolog-like 1 | NM_019935 | 2.506194 | 0.04395234 | 0.27440772 | 0.51281475 |
| | | (Drosophila) | | | | | |
| 77 | Sass6 | spindle assembly 6 | NM_028349 | 2.49633945 | 0.04423336 | −0.8207983 | 0.19688624 |
| | | homolog (C. elegans) | | | | | |
| 78 | Atf3 | activating | NM_007498 | 2.48444574 | 0.044683 | 0.52372201 | 0.31660297 |
| | | transcription factor 3 | | | | | |
| 79 | Krt71 | keratin 71 | NM_019956 | 2.46938747 | 0.04496403 | 7.59662267 | 0.00427158 |
| 80 | Zdhhc20 | zinc finger, DHHC | NM_029492 | 2.46658474 | 0.04496403 | −0.0323704 | 0.91310701 |
| | | domain containing | | | | | |
| | | 20 | | | | | |
| 81 | Birc3 | baculoviral IAP | NM_007464 | 2.39168001 | 0.04704362 | 2.07636354 | 0.05272032 |
| | | repeat-containing 3 | | | | | |
| 82 | Erlin1 | ER lipid raft | NM_001164359 | 2.38026342 | 0.04777428 | 0.13865639 | 0.69373876 |
| | | associated 1 | | | | | |
| 83 | Errfi1 | ERBB receptor | NM_133753 | 2.33905483 | 0.04974146 | 0.85979033 | 0.18761241 |
| | | feedback inhibitor 1 | | | | | |

| | | Change in Gene Expression | | Activated via Toll-Like Receptor (TLR) stimulation | | | | |
|---|---|---|---|---|---|---|---|---|
| | | (Weighted Difference) | | in dendritic cells (Amit, I. et al. 2009, supra) | | | | |
| | | | 2 hr to | Stimulation | Stimulation | Stimulation | Stimulation | Stimulation |
| | Universal | 2 hr to 4 hr | 4 hr P- | of TLR9 by | of TLR7 by | of TLR4 by | of TLR2 by | of TLR3 by |
| # | Symbol | Transition | value | CpG | GRD | LPS | Pam | PiC |
| 1 | Riok2 | 0.52490714 | 0.25050585 | | | | | |
| 2 | Trim16 | 1.58196085 | 0.05316996 | | | | | + |
| 3 | Has2 | 0.16801257 | 0.58908498 | | | + | | + |
| 4 | Xcl1 | 0.01834031 | 0.94031025 | + | + | | | + |
| 5 | Fos | 0.29587339 | 0.4196268 | + | + | + | + | + |
| 6 | Zfp820 | −1.3399156 | 0.07059353 | | | + | | |
| 7 | Pvrl2 | 0.9469571 | 0.11977293 | | + | + | + | + |
| 8 | Klra20 | −0.0655371 | 0.80350719 | | + | | | |
| 9 | Krtap6-1 | 0.21685274 | 0.51776079 | | | | + | + |
| 10 | 6330416L07Rik | −0.1190954 | 0.67839478 | | | | | |
| 11 | Gm10229 | 0.18959854 | 0.55508094 | + | + | + | | + |
| 12 | Enox2 | −0.063231 | 0.80856565 | | | | | + |
| 13 | Expi | −0.1280731 | 0.66091502 | + | + | + | | + |
| 14 | Fam64a | −0.2127607 | 0.52248201 | | | | + | |
| 15 | Krtap15 | 0.25519187 | 0.46712005 | + | + | + | + | + |
| 16 | Klf10 | 0.98609583 | 0.11336556 | | | | | + |
| 17 | Il12rb2 | 0.00873419 | 0.97139164 | + | + | + | + | |
| 18 | Krtap16-10 | 0.3124944 | 0.40450764 | + | + | | + | + |
| 19 | Krtap16-8 | 0.27510252 | 0.44289568 | | | | | + |
| 20 | Il2 | −0.0174799 | 0.94323291 | | | | | |
| 21 | Krtap16-5 | 0.29255147 | 0.42311151 | | | | | |
| 22 | Prss22 | 0.99343844 | 0.11224146 | + | | | | + |
| 23 | Trp53inp1 | 0.09958063 | 0.71908723 | | | | + | |
| 24 | Krt25 | 0.14640688 | 0.62674236 | | | | + | |
| 25 | Tchh | 0.32193083 | 0.3973134 | | | + | | + |
| 26 | Zcchc18 | −0.0384112 | 0.88000225 | + | | | + | |
| 27 | Krtap16-7 | 0.35383978 | 0.36696268 | | | | | |
| 28 | Eif1a | 3.6044127 | 0.0122527 | | + | | | + |
| 29 | Zscan12 | −0.0807248 | 0.76472572 | | | | | |
| 30 | Id1 | 0.03436732 | 0.89242356 | + | | + | | + |
| 31 | Wbp5 | 0.00969689 | 0.96830036 | | | | + | |
| 32 | Krtap6-2 | 0.43652907 | 0.3024393 | | | + | | |
| 33 | Krtap8-1 | 0.11256704 | 0.6919402 | | | | + | |
| 34 | Ik | −0.0132016 | 0.95689074 | | | | | |
| 35 | 2010109K11Rik | 0.17220085 | 0.58087905 | | + | + | + | |
| 36 | Gadd45a | 1.03917996 | 0.10448516 | | | | | |
| 37 | Mmp1b | −0.030825 | 0.90366457 | + | | + | + | |
| 38 | Zfp764 | −0.0572887 | 0.82430306 | | | | | |
| 39 | Slc26a5 | −0.0691975 | 0.7923786 | | | | | |
| 40 | Stambpl1 | 0.54213183 | 0.24038894 | + | | + | | + |
| 41 | St5 | 0.19599 | 0.54524505 | | | | | + |
| 42 | Acot4 | 0.06896219 | 0.79333408 | | + | + | | + |
| 43 | Map3k14 | 2.10194534 | 0.03243031 | | | | | |
| 44 | Krt27 | 0.32079248 | 0.39821268 | + | + | + | | + |
| 45 | Lefty1 | 0.31478638 | 0.40248426 | | | | | + |
| 46 | Krtap4-16 | 0.29083587 | 0.42496628 | + | | | | |
| 47 | Krtap8-2 | 0.26981226 | 0.44969649 | + | | + | | |
| 48 | Cxcl14 | −0.0231961 | 0.92654002 | | | | | |
| 49 | Fastkd1 | −0.0273581 | 0.91417491 | + | | + | | |
| 50 | Gtpbp5 | −0.3883518 | 0.33571268 | | | | | |
| 51 | Acsl4 | 0.1173122 | 0.68244155 | | | | | |
| 52 | Tyrp1 | −0.6692568 | 0.189692 | + | | + | | + |
| 53 | Plscr1 | 0.76471623 | 0.15967851 | | | | | |
| 54 | Med21 | −0.0076312 | 0.97560701 | + | | | + | + |
| 55 | A030005K14Rik | 0.20188181 | 0.53765737 | | | | | |
| 56 | Krtap16-1 | 0.30059992 | 0.41501799 | + | | | | |
| 57 | Cd207 | 0.21106508 | 0.52433678 | | | | + | |
| 58 | Zfp654 | −0.0340479 | 0.89354766 | | | | | + |
| 59 | AY026312 | 0.22575502 | 0.50607014 | + | + | + | + | + |
| 60 | Zfp7 | 0.46625305 | 0.28355441 | | | | + | |
| 61 | Ear11 | 0.26912861 | 0.45059577 | | | | | |
| 62 | Cyp27b1 | 1.74545738 | 0.0458071 | | + | | + | |
| 63 | Sox4 | −0.2758152 | 0.44216502 | | | | + | |
| 64 | Cd40 | 0.75902478 | 0.16063399 | | | | | |
| 65 | Fam43a | 1.52931915 | 0.05676709 | | | | | |
| 66 | Pdlim4 | 0.01016135 | 0.96667041 | | | | | + |
| 67 | Tnfrsf18 | 0.43938615 | 0.30058453 | + | + | + | + | + |
| 68 | Leng1 | 0.00325425 | 0.98904002 | | | | | |
| 69 | Apaf1 | −0.0001967 | 0.99943795 | | + | | | |
| 70 | S100a9 | 0.4473778 | 0.29513264 | | | + | + | + |
| 71 | Krt33a | 0.12953176 | 0.65883543 | | | | | |
| 72 | Hbegf | 1.98771403 | 0.03647707 | | + | | | |
| 73 | Krt17 | 0.19801289 | 0.54232239 | | | | | |
| 74 | Gcm2 | −0.019744 | 0.93592626 | | | | | |
| 75 | Krtap14 | 0.13550484 | 0.64703237 | + | + | + | | + |
| 76 | Ovol1 | 0.0264436 | 0.91642311 | + | | + | + | + |
| 77 | Sass6 | −0.4522843 | 0.2924348 | | | | | |
| 78 | Atf3 | 1.86912119 | 0.04024281 | | | | | |
| 79 | Krt71 | 0.06916942 | 0.79265962 | + | | + | | + |
| 80 | Zdhhc20 | 0.00324145 | 0.98915243 | | | | | |
| 81 | Birc3 | 4.05907162 | 0.00994829 | | + | + | | |
| 82 | Erlin1 | 0.04868084 | 0.84953912 | + | | | | |
| 83 | Errfi1 | 0.50190492 | 0.26253372 | + | + | + | + | + |

TABLE 4
Genes showing ≧2-fold increased expression 1-2 hours after DBP application, with corresponding expression change at a 2-4 hr time interval.

| | | | | Change in Gene Expression | | | | Activated via Toll-Like Receptor (TLR) stimulation | | | | |
|---|---|---|---|---|---|---|---|---|---|---|---|---|
| | | | | (Weighted Difference) | | | | in dendritic cells (Amit, I. et al. 2009, supra). | | | | |
| | Universal | Universal Gene | Universal Gene | 1 hr to 2 hr | 1 hr to 2 hr | 2 hr to 4 hr | 2 hr to 4 hr | Stimulation of | Stimulation of | Stimulation of | Stimulation of | Stimulation of |
| # | Symbol | Name | Refseq ID | Transition | P-value | Transition | P-value | TLR9 by CpG | TLR7 by GRD | TLR4 by LPS | TLR2 by Pam | TLR3 by PiC |
| 1 | Gjb2 | gap junction | NM_008125 | 80.6245784 | 5.6205E−05 | 1.18839298 | 0.08554406 | + | | + | + | + |
| | | protein, beta 2 | | | | | | | | | | |
| 2 | Tfpi2 | tissue factor | NM_009364 | 21.0917704 | 0.00056205 | 0.34512465 | 0.37488759 | | | | | |
| | | pathway inhibitor 2 | | | | | | | | | | |
| 3 | Timp1 | tissue inhibitor of | NM_001044384 | 14.7377459 | 0.00095549 | 1.86294221 | 0.04052383 | | | | | |
| | | metalloproteinase 1 | | | | | | | | | | |
| 4 | Plin2 | perilipin 2 | NM_007408 | 11.1824713 | 0.00207959 | 4.45245874 | 0.00831835 | | | | | + |
| 5 | Arl5a | ADP-ribosylation | NM_182994 | 10.8324979 | 0.00213579 | 23.2966619 | 0.00028103 | | | | | |
| | | factor-like 5A | | | | | | | | | | |
| 6 | Thbs2 | thrombospondin 2 | NM_011581 | 9.52606014 | 0.00297887 | −0.292623 | 0.4229991 | + | | | | |
| 7 | Tubb6 | tubulin, beta 6 | NM_026473 | 9.09376144 | 0.00325989 | 7.75043441 | 0.00286646 | | | | | |
| 8 | Wfdc12 | WAP four-disulfide | NM_138684 | 8.74770049 | 0.00354092 | 0.21526276 | 0.51950315 | | | + | + | |
| | | core domain 12 | | | | | | | | | | |
| 9 | Klk6 | kallikrein related- | NM_001164696 | 8.13690812 | 0.00382194 | 1.15591678 | 0.08908498 | | | | | |
| | | peptidase 6 | | | | | | | | | | |
| 10 | Krtap4-2 | keratin associated | NM_026807 | 7.85418617 | 0.00410297 | 0.87642863 | 0.13433004 | | | | | |
| | | protein 4-2 | | | | | | | | | | |
| 11 | Eif2s2 | eukaryotic | NM_026030 | 7.6458927 | 0.00415917 | 0.36369577 | 0.35768885 | | | | | + |
| | | translation initiation | | | | | | | | | | |
| | | factor 2, subunit 2 | | | | | | | | | | |
| | | (beta) | | | | | | | | | | |
| 12 | Slc30a4 | solute carrier family | NM_011774 | 7.64070948 | 0.00415917 | 1.4572962 | 0.06165692 | + | | | | + |
| | | 30 (zinc | | | | | | | | | | |
| | | transporter), | | | | | | | | | | |
| | | member 4 | | | | | | | | | | |
| 13 | Adamts4 | a disintegrin-like | NM_172845 | 7.82945661 | 0.00415917 | 1.70764639 | 0.04743705 | | | + | + | |
| | | and | | | | | | | | | | |
| | | metallopeptidase | | | | | | | | | | |
| | | (reprolysin type) | | | | | | | | | | |
| | | with | | | | | | | | | | |
| | | thrombospondin | | | | | | | | | | |
| | | type 1 motif, 4 | | | | | | | | | | |
| 14 | Krtap5-2 | keratin associated | NM_027844 | 7.50408513 | 0.00455261 | 0.33435962 | 0.38652203 | + | + | + | | |
| | | protein 5-2 | | | | | | | | | | |
| 15 | Ptpla | protein tyrosine | NM_001012396 | 7.3254256 | 0.00483363 | 0.08314299 | 0.75910522 | | | | | |
| | | phosphatase-like | | | | | | | | | | |
| | | (proline instead of | | | | | | | | | | |
| | | catalytic arginine), | | | | | | | | | | |
| | | member a | | | | | | | | | | |
| 16 | 2310033E01Rik | RIKEN cDNA | NM_001037143 | 7.04214777 | 0.00533948 | 0.22579026 | 0.50607014 | | + | + | + | |
| | | 2310033E01 gene | | | | | | | | | | |
| 17 | Slc34a2 | solute carrier family | NM_011402 | 6.7788935 | 0.00567671 | 0.71297518 | 0.17491007 | | | | | |
| | | 34 (sodium | | | | | | | | | | |
| | | phosphate), member 2 | | | | | | | | | | |
| 18 | Krtap28-13 | keratin associated | XM_896507 | 6.68213439 | 0.00590153 | 0.21809404 | 0.51635567 | + | | | | + |
| | | protein 28-13 | | | | | | | | | | |
| 19 | Krt86 | keratin 86 | NM_010667 | 6.69121599 | 0.00590153 | 0.25751143 | 0.46453462 | | | | | |
| 20 | C230052I12Rik | RIKEN cDNA | NM_178643 | 6.59889523 | 0.00612635 | −0.1696823 | 0.58616232 | + | + | | | |
| | | C230052I12 gene | | | | | | | | | | |
| 21 | Krtap3-3 | keratin associated | NM_025524 | 6.56125843 | 0.00623876 | 0.12272831 | 0.67221223 | | | + | | |
| | | protein 3-3 | | | | | | | | | | |
| 22 | Gm11567 | predicted gene | NM_001101613 | 6.42854181 | 0.00651978 | 0.14705618 | 0.62589928 | | | | + | + |
| | | 11567 | | | | | | | | | | |
| 23 | Krtap1-5 | keratin associated | NM_027157 | 6.37151666 | 0.0066884 | 0.26800415 | 0.45194469 | | | + | | + |
| | | protein 1-5 | | | | | | | | | | |
| 24 | Slc15a3 | solute carrier family | NM_023044 | 6.29761548 | 0.00691322 | 0.77486421 | 0.15703687 | + | + | + | + | + |
| | | 15, member 3 | | | | | | | | | | |
| 25 | Tmem56 | transmembrane | NM_178936 | 6.09101325 | 0.00747527 | 0.8224762 | 0.14596448 | | + | | | |
| | | protein 56 | | | | | | | | | | |
| 26 | Krt34 | keratin 34 | NM_027563 | 6.0219745 | 0.00758768 | 0.11242995 | 0.69227743 | + | + | + | + | + |
| 27 | Krtap16-9 | keratin associated | NM_130876 | 5.89890128 | 0.0078125 | 0.44964688 | 0.29389613 | | | | | |
| | | protein 16-9 | | | | | | | | | | |
| 28 | Ptgir | prostaglandin I | NM_008967 | 5.63466244 | 0.00848696 | 2.06016151 | 0.03366682 | | | | | |
| | | receptor (IP) | | | | | | | | | | |
| 29 | 2310008H09Rik | RIKEN cDNA | NM_001168218 | 5.55409846 | 0.00882419 | −0.0380008 | 0.88112635 | | | | | |
| | | 2310008H09 gene | | | | | | | | | | |
| 30 | 1110032A04Rik | RIKEN cDNA | NM_001164210 | 5.57388923 | 0.00882419 | 1.19434688 | 0.0848696 | | + | + | | |
| | | 1110032A04 gene | | | | | | | | | | |
| 31 | LOC100048309 | similar to interferon | XM_001479990 | 5.52002834 | 0.0089366 | 1.04591523 | 0.1037545 | + | | + | | |
| | | activated gene 204 | | | | | | | | | | |
| 32 | Krtap3-1 | keratin associated | NM_023511 | 5.36476077 | 0.00927383 | 0.16242063 | 0.59858363 | + | | + | | |
| | | protein 3-1 | | | | | | | | | | |
| 33 | Gm10228 | predicted gene | XM_001474297 | 5.28918355 | 0.00955486 | 0.11574149 | 0.68603867 | | | | | |
| | | 10228 | | | | | | | | | | |
| 34 | LOC100047762 | similar to Aspartate | XM_001478835 | 5.1064135 | 0.01011691 | 0.94036243 | 0.12095324 | | | + | | |
| | | aminotransferase, | | | | | | | | | | |
| | | cytoplasmic | | | | | | | | | | |
| | | (Transaminase A) | | | | | | | | | | |
| | | (Glutamate | | | | | | | | | | |
| | | oxaloacetate | | | | | | | | | | |
| | | transaminase 1) | | | | | | | | | | |
| 35 | Cd14 | CD14 antigen | NM_009841 | 5.11840806 | 0.01011691 | 1.04201208 | 0.10420414 | | | | | |
| 36 | Ms4a6d | membrane-spanning | NM_026835 | 5.09123936 | 0.01017311 | 0.69055631 | 0.18210432 | + | | | + | |
| | | 4-domains, | | | | | | | | | | |
| | | subfamily A, | | | | | | | | | | |
| | | member 6D | | | | | | | | | | |
| 37 | Ifit1 | interferon-induced | NM_008331 | 5.09251174 | 0.01017311 | 0.03896494 | 0.87842851 | | | | | |
| | | protein with | | | | | | | | | | |
| | | tetratricopeptide | | | | | | | | | | |
| | | repeats 1 | | | | | | | | | | |
| 38 | Sepx1 | selenoprotein X 1 | NM_013759 | 5.06651134 | 0.01034173 | 0.40114664 | 0.32660746 | | | | | |
| 39 | Cxcr2 | chemokine (C—X—C | NM_009909 | 4.85742713 | 0.0111286 | 0.99718582 | 0.11190423 | | | | | |
| | | motif) receptor 2 | | | | | | | | | | |
| 40 | LOC100048304 | hypothetical protein | XM_001479976 | 4.41097574 | 0.01365782 | −0.3709812 | 0.35212455 | + | + | + | + | + |
| | | LOC100048304 | | | | | | | | | | |
| 41 | 2310042E22Rik | RIKEN cDNA | NM_025634 | 4.4241317 | 0.01365782 | 0.25464411 | 0.46801933 | + | | + | | |
| | | 2310042E22 gene | | | | | | | | | | |
| 42 | Hsd17b2 | hydroxysteroid (17- | NM_008290 | 4.29625235 | 0.01461331 | 0.0686635 | 0.79383993 | + | + | + | + | + |
| | | beta) dehydrogenase 2 | | | | | | | | | | |
| 43 | Sprr1b | small proline-rich | NM_009265 | 4.11889249 | 0.01545638 | 0.0903382 | 0.74235612 | | | | | |
| | | protein 1B | | | | | | | | | | |
| 44 | A030005L19Rik | RIKEN cDNA | XM_896385 | 4.08420706 | 0.01568121 | 0.27716853 | 0.44143435 | | | | | |
| | | A030005L19 gene | | | | | | | | | | |
| 45 | LOC100046232 | similar to | XM_001475817 | 4.06268329 | 0.01573741 | 2.14104734 | 0.03119379 | | | | | |
| | | NFIL3/E4BP4 | | | | | | | | | | |
| | | transcription factor | | | | | | | | | | |
| 46 | Nfil3 | nuclear factor, | NM_017373 | 4.06268329 | 0.01573741 | 2.14104734 | 0.03119379 | | | | | |
| | | interleukin 3, | | | | | | | | | | |
| | | regulated | | | | | | | | | | |
| 47 | Crym | crystallin, mu | NM_016669 | 3.99134628 | 0.01613085 | 0.27437351 | 0.44396358 | | | | | |
| 48 | Klrb1b | killer cell lectin-like | NM_030599 | 3.91332499 | 0.0167491 | 1.22715232 | 0.08116007 | | | + | + | |
| | | receptor subfamily | | | | | | | | | | |
| | | B member 1B | | | | | | | | | | |
| 49 | Elovl7 | ELOVL family | NM_029001 | 3.87356119 | 0.01691772 | 0.4257536 | 0.3091277 | + | | | | + |
| | | member 7, | | | | | | | | | | |
| | | elongation of long | | | | | | | | | | |
| | | chain fatty acids | | | | | | | | | | |
| | | (yeast) | | | | | | | | | | |
| 50 | Krtap16-4 | keratin associated | NM_130873 | 3.87196929 | 0.01691772 | 0.56072091 | 0.23190198 | | + | + | | + |
| | | protein 16-4 | | | | | | | | | | |
| 51 | Myo5b | myosin VB | NM_201600 | 3.87608828 | 0.01691772 | −0.005423 | 0.98223921 | | | + | | |
| 52 | AI848100 | expressed sequence | NM_172645 | 3.80597313 | 0.01764838 | −4.5892458 | 0.0078125 | | | | | + |
| | | AI848100 | | | | | | | | | | |
| 53 | Tcfec | transcription factor | NM_031198 | 3.80617339 | 0.01764838 | 0.62899927 | 0.20396808 | + | + | | | |
| | | EC | | | | | | | | | | |
| 54 | Fam134b | family with | NM_001034851 | 3.7747341 | 0.01792941 | 1.43925961 | 0.06294964 | + | | + | + | + |
| | | sequence similarity | | | | | | | | | | |
| | | 134, member B | | | | | | | | | | |
| 55 | S100a3 | S100 calcium | NM_011310 | 3.77534455 | 0.01792941 | 0.16797661 | 0.58908498 | + | + | + | + | |
| | | binding protein A3 | | | | | | | | | | |
| 56 | Slc22a23 | solute carrier family | NM_001033167 | 3.71660113 | 0.01866007 | −0.0025908 | 0.99128822 | | | + | | + |
| | | 22, member 23 | | | | | | | | | | |
| 57 | Clec4d | C-type lectin domain | NM_001163161 | 3.70420272 | 0.01871628 | 0.36805413 | 0.35454137 | | | | | |
| | | family 4, member d | | | | | | | | | | |
| 58 | Cln8 | ceroid- | NM_012000 | 3.69152434 | 0.0189411 | 0.40334817 | 0.32492131 | | | | | |
| | | lipofuscinosis, | | | | | | | | | | |
| | | neuronal 8 | | | | | | | | | | |
| 59 | Krt31 | keratin 31 | NM_010659 | 3.67601182 | 0.01916592 | 0.16240632 | 0.59863984 | | | | | |
| 60 | Got1 | glutamate | NM_010324 | 3.63007248 | 0.01955935 | 0.83520848 | 0.14354766 | | | + | | + |
| | | oxaloacetate | | | | | | | | | | |
| | | transaminase 1, | | | | | | | | | | |
| | | soluble | | | | | | | | | | |
| 61 | Irg1 | immunoresponsive | NM_008392 | 3.61605653 | 0.01972797 | 1.10961891 | 0.09509892 | | | + | | + |
| | | gene 1 | | | | | | | | | | |
| 62 | Ocln | occludin | NM_008756 | 3.54485101 | 0.02073966 | 1.28363431 | 0.07559577 | | | | | + |
| 63 | Dleu2 | deleted in | NR_028264 | 3.51103946 | 0.02113309 | 0.80814514 | 0.14871853 | | | + | + | + |
| | | lymphocytic | | | | | | | | | | |
| | | leukemia, 2 | | | | | | | | | | |
| 64 | Rybp | RING1 and YY1 | NM_019743 | 3.50236068 | 0.0211893 | −0.012644 | 0.95897032 | | | | | |
| | | binding protein | | | | | | | | | | |
| 65 | Gabpb1 | GA repeat binding | NM_010249 | 3.48728086 | 0.02141412 | −0.1514677 | 0.61701888 | | | | | |
| | | protein, beta 1 | | | | | | | | | | |
| 66 | Fam89a | family with | NM_001081120 | 3.46541035 | 0.02191996 | 1.12812295 | 0.09234487 | | | | | |
| | | sequence similarity | | | | | | | | | | |
| | | 89, member A | | | | | | | | | | |
| 67 | LOC100047808 | hypothetical protein | XM_001478908 | 3.46541035 | 0.02191996 | 1.12812295 | 0.09234487 | | | | | |
| | | LOC100047808 | | | | | | | | | | |
| 68 | Larp1b | La | NM_001040399 | 3.45261023 | 0.0223134 | 0.41450263 | 0.31722122 | | | | + | |
| | | ribonucleoprotein | | | | | | | | | | |
| | | domain family, | | | | | | | | | | |
| | | member 1B | | | | | | | | | | |
| 69 | Ly6g6d | lymphocyte antigen | NM_033478 | 3.41650281 | 0.02281924 | 0.05778192 | 0.82345998 | | | + | + | |
| | | 6 complex, locus | | | | | | | | | | |
| | | G6D | | | | | | | | | | |
| 70 | Cyp1a1 | cytochrome P450, | NM_001136059 | 3.37878377 | 0.02304406 | 1.56299527 | 0.05446268 | | | + | | |
| | | family 1, subfamily | | | | | | | | | | |
| | | a, polypeptide 1 | | | | | | | | | | |
| 71 | LOC100047340 | hypothetical protein | XM_001477942 | 3.35661906 | 0.02360612 | 1.39150403 | 0.06665917 | | | | | + |
| | | LOC100047340 | | | | | | | | | | |
| 72 | Rcc2 | regulator of | NM_173867 | 3.35661906 | 0.02360612 | 1.39150403 | 0.06665917 | | | | | + |
| | | chromosome | | | | | | | | | | |
| | | condensation 2 | | | | | | | | | | |
| 73 | Stx19 | syntaxin 19 | NM_026588 | 3.33428857 | 0.02388714 | 0.05085325 | 0.84212005 | + | | | | + |
| 74 | Krt33b | keratin 33B | NM_013570 | 3.21245345 | 0.02613534 | 0.19503861 | 0.54642536 | + | + | | | |
| 75 | Dnase1l3 | deoxyribonuclease | NM_007870 | 3.16535884 | 0.02669739 | 0.37799869 | 0.34532374 | | | + | | |
| | | 1-like 3 | | | | | | | | | | |
| 76 | Krtap5-4 | keratin associated | NM_015809 | 3.16762579 | 0.02669739 | 0.29847059 | 0.41704137 | | | | | |
| | | protein 5-4 | | | | | | | | | | |
| 77 | LOC100044239 | hypothetical protein | XM_001471771 | 3.04383828 | 0.02860836 | 1.16495629 | 0.08829811 | | | | + | |
| | | LOC100044239 | | | | | | | | | | |
| 78 | Clec4n | C-type lectin domain | NM_020001 | 2.98537036 | 0.02967626 | 1.14346893 | 0.09077113 | | | | | |
| | | family 4, member n | | | | | | | | | | |
| 79 | Hacl1 | 2-hydroxyacyl-CoA | NM_019975 | 2.98503165 | 0.02967626 | 0.70325721 | 0.17805755 | | | | | |
| | | lyase 1 | | | | | | | | | | |
| 80 | Upp1 | uridine | NM_001159401 | 2.86526243 | 0.03175585 | 0.43019714 | 0.30654227 | + | + | | | |
| | | phosphorylase 1 | | | | | | | | | | |
| 81 | Arrdc4 | arrestin domain | NM_001042592 | 2.8160644 | 0.0323741 | 1.43881868 | 0.06306205 | | | + | | + |
| | | containing 4 | | | | | | | | | | |
| 82 | Plaur | plasminogen | NM_011113 | 2.81588233 | 0.0323741 | 1.27660058 | 0.07638264 | | | | + | |
| | | activator, urokinase | | | | | | | | | | |
| | | receptor | | | | | | | | | | |
| 83 | Sult2b1 | sulfotransferase | NM_017465 | 2.79903757 | 0.03265513 | 0.58776161 | 0.21970549 | + | + | + | + | + |
| | | family, cytosolic, | | | | | | | | | | |
| | | 2B, member 1 | | | | | | | | | | |
| 84 | Ms4a4c | membrane-spanning | NM_029499 | 2.7118821 | 0.03467851 | 1.23346308 | 0.080317 | | | | | |
| | | 4-domains, | | | | | | | | | | |
| | | subfamily A, | | | | | | | | | | |
| | | member 4C | | | | | | | | | | |
| 85 | Pppde1 | PPPDE peptidase | NM_024282 | 2.68421521 | 0.03512815 | 0.31927666 | 0.39894335 | | | | | + |
| | | domain containing 1 | | | | | | | | | | |
| 86 | Csf2rb | colony stimulating | NM_007780 | 2.685042 | 0.03512815 | 0.54425406 | 0.23977068 | | | | | |
| | | factor 2 receptor, | | | | | | | | | | |
| | | beta, low-affinity | | | | | | | | | | |
| | | (granulocyte- | | | | | | | | | | |
| | | macrophage) | | | | | | | | | | |
| 87 | 2010109I03Rik | RIKEN cDNA | NM_025929 | 2.67174323 | 0.0356902 | 0.92811659 | 0.12314523 | | | | + | |
| | | 2010109I03 gene | | | | | | | | | | |
| 88 | Slc15a1 | solute carrier family | NM_053079 | 2.66832773 | 0.03591502 | 1.1746413 | 0.08723022 | + | + | + | + | + |
| | | 15 (oligopeptide | | | | | | | | | | |
| | | transporter), | | | | | | | | | | |
| | | member 1 | | | | | | | | | | |
| 89 | Gabrd | gamma- | NM_008072 | 2.65918709 | 0.03613984 | 0.59033328 | 0.21914344 | + | + | | + | |
| | | aminobutyric acid | | | | | | | | | | |
| | | (GABA) A receptor, | | | | | | | | | | |
| | | subunit delta | | | | | | | | | | |
| 90 | Cd274 | CD274 antigen | NM_021893 | 2.64564632 | 0.03664568 | 0.21170979 | 0.52371853 | | | | + | |
| 91 | Tbc1d10a | TBC1 domain family, | NM_134023 | 2.63679783 | 0.03692671 | 1.31989931 | 0.07244829 | | | | | |
| | | member 10a | | | | | | | | | | |
| 92 | Krtap12-1 | keratin associated | NM_010670 | 2.62742828 | 0.03698291 | 0.15268911 | 0.61544514 | + | + | + | + | + |
| | | protein 12-1 | | | | | | | | | | |
| 93 | Tnfsf9 | tumor necrosis | NM_009404 | 2.60848673 | 0.03754496 | 1.07891615 | 0.09841502 | + | | + | + | + |
| | | factor (ligand) | | | | | | | | | | |
| | | superfamily, | | | | | | | | | | |
| | | member 9 | | | | | | | | | | |
| 94 | Chi3l3 | chitinase 3-like 3 | NM_009892 | 2.59156881 | 0.03782599 | 0.72718218 | 0.17052608 | + | | + | + | |
| 95 | Samd8 | sterile alpha motif | NM_026283 | 2.56636009 | 0.03833183 | 0.84118196 | 0.1417491 | | + | | | |
| | | domain containing 8 | | | | | | | | | | |
| 96 | Gprc5d | G protein-coupled | NM_053118 | 2.35976379 | 0.04333408 | 0.12088743 | 0.67547212 | | | | | |
| | | receptor, family C, | | | | | | | | | | |
| | | group 5, member D | | | | | | | | | | |
| 97 | Cwh43 | cell wall biogenesis | NM_181323 | 2.35269594 | 0.0435027 | 0.01097054 | 0.96487185 | | | | | |
| | | 43 C-terminal | | | | | | | | | | |
| | | homolog (S. cerevisiae) | | | | | | | | | | |
| 98 | Slc25a33 | solute carrier family | NM_027460 | 2.29793582 | 0.04479541 | 1.11929261 | 0.09425585 | + | | + | | + |
| | | 25, member 33 | | | | | | | | | | |
| 99 | Saa1 | serum amyloid A 1 | NM_009117 | 2.29826459 | 0.04479541 | 0.64690748 | 0.19733588 | | + | | | |
| 100 | Alas1 | aminolevulinic acid | NM_020559 | 2.2965094 | 0.04496403 | 1.07747256 | 0.09847122 | | | | | + |
| | | synthase 1 | | | | | | | | | | |
| 101 | Slc9a3r1 | solute carrier family 9 | NM_012030 | 2.28561789 | 0.04524505 | 4.48288699 | 0.00831835 | | | | | |
| | | (sodium/hydrogen | | | | | | | | | | |
| | | exchanger), member | | | | | | | | | | |
| | | 3 regulator 1 | | | | | | | | | | |
| 102 | 2010011I20Rik | RIKEN cDNA | NM_025912 | 2.26122916 | 0.04591951 | −0.1009756 | 0.71582734 | | + | + | + | + |
| | | 2010011I20 gene | | | | | | | | | | |
| 103 | Cldn4 | claudin 4 | NM_009903 | 2.25692176 | 0.04603192 | 0.95323507 | 0.11892986 | | | | + | |
| 104 | Sprr2h | small proline-rich | NM_011474 | 2.25222871 | 0.04603192 | −0.0607334 | 0.81491682 | | | | | |
| | | protein 2H | | | | | | | | | | |
| 105 | Gjb6 | gap junction | NM_001010937 | 2.24273425 | 0.04636915 | −0.0348524 | 0.88995054 | | + | | + | |
| | | protein, beta 6 | | | | | | | | | | |
| 106 | LOC100044180 | hypothetical protein | XM_001471623 | 2.22019028 | 0.04715603 | −0.1482775 | 0.62337005 | | | | | |
| | | LOC100044180 | | | | | | | | | | |
| 107 | Atp13a3 | ATPase type 13A3 | NM_001128094 | 2.21538049 | 0.04726844 | 1.83754643 | 0.04142311 | + | + | + | + | + |
| 108 | Crem | cAMP responsive | NM_001110850 | 2.2036957 | 0.04771808 | 1.16398419 | 0.08835432 | | | + | | + |
| | | element modulator | | | | | | | | | | |
| 109 | Clqtnf3 | Clq and tumor | NM_030888 | 2.19887414 | 0.04788669 | −0.6050404 | 0.21251124 | + | | | | + |
| | | necrosis factor | | | | | | | | | | |
| | | related protein 3 | | | | | | | | | | |
| 110 | Il1f10 | interleukin 1 family, | NM_153077 | 2.18476351 | 0.04839254 | 0.13990055 | 0.63882644 | | | | + | |
| | | member 10 | | | | | | | | | | |
| 111 | Krt6a | keratin 6A | NM_008476 | 2.16225545 | 0.04917941 | 0.97929188 | 0.11460207 | | | | | |
| 112 | Rdh9 | retinol | NM_153133 | 2.15196229 | 0.04951664 | 0.13363459 | 0.65051709 | | | | | |
| | | dehydrogenase 9 | | | | | | | | | | |

TABLE 5
Genes showing ≧2-fold increased expression at 2-4 hours after DBP application.

| | | | | | | Activated via Toll-Like Receptor (TLR) stimulation in | | | | |
|---|---|---|---|---|---|---|---|---|---|---|
| | | | | Change in | | dendritic cells (Amit, I. et al. 2009, supra) | | | | |
| | | | | Gene Expression | | Stim- | Stim- | Stim- | Stim- | Stim- |
| | | | | (Weighted Difference) | | ulation | ulation | ulation | ulation | ulation |
| | Universal | Universal | Universal | 2 hr to 4 hr | 2 hr to 4 hr | of TLR9 | of TLR7 | of TLR4 | of TLR2 | of TLR3 |
| # | Symbol | Gene Name | Gene Refseq ID | Transition | P-value | by CpG | by GRD | by LPS | by Pam | by PiC |
| 1 | Kcnk1 | potassium | NM_008430 | 32.7968417 | 0.00016862 | | | | + | + |
| | | channel, | | | | | | | | |
| | | subfamily K, | | | | | | | | |
| | | member 1 | | | | | | | | |
| 2 | Myl2 | myosin, light | NM_010861 | 12.1981662 | 0.00095549 | | | | | |
| | | polypeptide 2, | | | | | | | | |
| | | regulatory, | | | | | | | | |
| | | cardiac, slow | | | | | | | | |
| 3 | Myh3 | myosin, heavy | NM_001099635 | 8.0121673 | 0.00258543 | | | | | |
| | | polypeptide 3, | | | | | | | | |
| | | skeletal muscle, | | | | | | | | |
| | | embryonic | | | | | | | | |
| 4 | Mtap7d1 | microtubule- | NM_001145970 | 6.99020153 | 0.00359712 | | | | | |
| | | associated | | | | | | | | |
| | | protein 7 | | | | | | | | |
| | | domain | | | | | | | | |
| | | containing 1 | | | | | | | | |
| 5 | Prss23 | protease, serine, | NM_029614 | 6.98262963 | 0.00359712 | + | | + | + | |
| | | 23 | | | | | | | | |
| 6 | Rai12 | retinoic acid | NM_018740 | 6.68284377 | 0.00376574 | | | | + | + |
| | | induced 12 | | | | | | | | |
| 7 | Tmem51 | transmembrane | NM_145402 | 6.1038808 | 0.00432779 | + | + | + | + | |
| | | protein 51 | | | | | | | | |
| 8 | Gart | phosphoribosyl | NM_010256 | 5.26757151 | 0.00578912 | | | | | |
| | | glycinamide | | | | | | | | |
| | | formyltransferase | | | | | | | | |
| 9 | Fosl1 | fos-like antigen 1 | NM_010235 | 4.43246666 | 0.00837455 | + | + | + | + | + |
| 10 | Irf4 | interferon | NM_013674 | 3.71470542 | 0.01152203 | | | | | |
| | | regulatory | | | | | | | | |
| | | factor 4 | | | | | | | | |
| 11 | Maml1 | mastermind like | NM_175334 | 3.56351987 | 0.01258993 | | | | | + |
| | | 1 (Drosophila) | | | | | | | | |
| 12 | Tnni1 | troponin I, | NM_001112702 | 3.47010747 | 0.013433 | | | + | + | + |
| | | skeletal, slow 1 | | | | | | | | |
| 13 | Areg | amphiregulin | NM_009704 | 3.40055397 | 0.01393885 | | | | + | |
| 14 | Pxmp4 | peroxisomal | NM_021534 | 3.3622076 | 0.01421987 | | | | | |
| | | membrane | | | | | | | | |
| | | protein 4 | | | | | | | | |
| 15 | Tnip1 | TNFAIP3 | NM_021327 | 3.26696838 | 0.01489433 | | | | | |
| | | interacting | | | | | | | | |
| | | protein 1 | | | | | | | | |
| 16 | Lcn5 | lipocalin 5 | NM_001042630 | 3.18313058 | 0.01568121 | | | + | | |
| 17 | Dlx4 | distal-less | NM_007867 | 3.05032674 | 0.0167491 | + | | | + | |
| | | homeobox 4 | | | | | | | | |
| 18 | Tnnt1 | troponin T1, | NM_011618 | 2.94642724 | 0.01854766 | | | + | + | + |
| | | skeletal, slow | | | | | | | | |
| 19 | Il10 | interleukin 10 | NM_010548 | 2.93162311 | 0.01888489 | | | | | |
| 20 | Il20 | interleukin 20 | NM_021380 | 2.87592292 | 0.01922212 | + | + | + | + | |
| 21 | Has1 | hyaluronan | NM_008215 | 2.53950068 | 0.02265063 | | | | | + |
| | | synthase1 | | | | | | | | |
| 22 | Rassf5 | Ras association | NM_018750 | 2.51865958 | 0.02332509 | + | | | | + |
| | | (RalGDS/AF-6) | | | | | | | | |
| | | domain family | | | | | | | | |
| | | member 5 | | | | | | | | |
| 23 | Trib1 | tribbles | NM_144549 | 2.43195379 | 0.02517986 | | | | | |
| | | homolog 1 | | | | | | | | |
| | | (Drosophila) | | | | | | | | |
| 24 | Ddx21 | DEAD (Asp-Glu- | NM_019553 | 2.41260051 | 0.02540468 | + | + | + | | + |
| | | Ala-Asp) box | | | | | | | | |
| | | polypeptide 21 | | | | | | | | |
| 25 | Dusp14 | dual specificity | NM_019819 | 2.29074458 | 0.02782149 | | | + | | + |
| | | phosphatase 14 | | | | | | | | |
| 26 | Ctgf | connective | NM_010217 | 2.28979124 | 0.02782149 | | | | | |
| | | tissue growth | | | | | | | | |
| | | factor | | | | | | | | |
| 27 | Slc25a25 | solute carrier | NM_001164357 | 2.2852384 | 0.0278777 | | + | | | + |
| | | family 25 | | | | | | | | |
| | | (mitochondrial | | | | | | | | |
| | | carrier, | | | | | | | | |
| | | phosphate | | | | | | | | |
| | | carrier), | | | | | | | | |
| | | member 25 | | | | | | | | |
| 28 | Tnfaip8l1 | tumor necrosis | NM_025566 | 2.28046503 | 0.0279339 | + | | + | + | + |
| | | factor, alpha- | | | | | | | | |
| | | induced protein | | | | | | | | |
| | | 8-like 1 | | | | | | | | |
| 29 | Tiparp | TCDD-inducible | NM_178892 | 2.28135713 | 0.0279339 | | | | | |
| | | poly(ADP- | | | | | | | | |
| | | ribose) | | | | | | | | |
| | | polymerase | | | | | | | | |
| 30 | Fbxo42 | F-box protein | NM_172518 | 2.2674251 | 0.02815872 | | + | + | + | + |
| | | 42 | | | | | | | | |
| 31 | Map3k11 | mitogen- | NM_022012 | 2.24861928 | 0.02855216 | + | | + | | + |
| | | activated | | | | | | | | |
| | | protein kinase | | | | | | | | |
| | | kinase kinase | | | | | | | | |
| | | 11 | | | | | | | | |
| 32 | Zfp326 | zinc finger | NM_018759 | 2.16438827 | 0.03080036 | | | | | |
| | | protein 326 | | | | | | | | |
| 33 | Fam115c | family with | NM_146174 | 2.16283978 | 0.03085656 | | | | | |
| | | sequence | | | | | | | | |
| | | similarity 115, | | | | | | | | |
| | | member C | | | | | | | | |
| 34 | LOC100046410 | similar to | XM_001476169 | 2.1343764 | 0.03136241 | | + | | | |
| | | palmdelphin | | | | | | | | |
| 35 | Palmd | palmdelphin | NM_023245 | 2.1343764 | 0.03136241 | | + | | | |
| 36 | Nedd4l | neural | NM_001114386 | 2.06707826 | 0.0334982 | | | + | | + |
| | | precursor cell | | | | | | | | |
| | | expressed, | | | | | | | | |
| | | developmentally | | | | | | | | |
| | | down- | | | | | | | | |
| | | regulated gene | | | | | | | | |
| | | 4-like | | | | | | | | |

TABLE 6
DBP-Activated Gene Expression (≧2x Increased Expression)

| Time | | DBP-Induced | TLR-Induced | % Shared | DBP Unique |
|---|---|---|---|---|---|
| 15 | min | 33* | 21 | 64 | 12 |
| 30 | min | 297 | 190^{#} | 64 | 107 |
| 1 | hr | 83 | 57^{#} | 69 | 26 |
| 2 | hr | 112 | 70^{#} | 63 | 42 |
| 4 | hr | 36 | 23^{#} | 64 | 13 |

| | | | | |
|---|---|---|---|---|
| Total: | 561 (33*) | 361 (340^{#}) | 64 | 200 |

*Early response genes
^{#}Known dendritic cell activation genes
Early Response Genes + DC activation genes = 373

The genes that were upregulated by DBP, and by the various TLR agonists, as reported by Amit et al., can be assumed to be “dendritic cell activation genes.” Additional genes whose regulation was altered 2-fold or more by DBP may be acting on other cell types in the skin. All of our gene expression data were obtained using full thickness skin in vivo. Therefore, the alteration in gene expression induced by topical DBP that was not reported in isolated dendritic cells by Amit et al. may be gene expression induced by DBP in other cell types in the skin. Those genes may also be relevant for inducing the activation and migration of dendritic cells out of other tissues or organs.

### Example 6

### Identification of Cellular Genes Modulated by DBP on the Basis of a log₂-Fold Change in Gene Expression and by Two General Linear Modeling Methods

### Overview

We have developed novel methods to identify the mechanism(s) of action of a class of immunological adjuvants exemplified by DBP. Drug mechanism of action is a complex process which may occur on many levels, but a common property of drug action is that, for a drug to have a strong physiological effect, it must do so by manipulating gene expression, either directly or indirectly. Based upon the large scale effects of DBP on cell fate and migration, we have developed a model of its action, by determining how it affects gene expression globally. Our novel method for creating this model utilizes recent advances is biological assays, machine learning, and statistics.

Given the existence of approximately 30,000 genes in the average mammalian genome, it is a difficult problem to determine the exact genetic basis for the mechanism of action of a drug, both in terms of the collection of the necessary data and the complexity of analysing such data. Microarray technology provides a powerful tool for the first step in this process, the data acquisition, by allowing the simultaneous quantification of the expression of many genes. Microarrays work by binding a sample of RNA to sequence-specific, fluorescent probes on a solid support (e.g., a glass slide), and then measuring the intensity of fluorescence at each probe, as a reporter for the quantity of specific transcripts in the initial sample. Microarrays produce a vast amount of data, the processing of which, while not trivial, is rather standardized. We describe our method of processing in the next section.

This mass of data produced by microarrays thus places the central technical demands on the analysis, rather than the collection, of necessary information to formulate a model of drug mechanism of action. The model we describe is of this form: given a global quantification of the change in gene expression in a sample following drug treatment, our model outputs a prediction of draining lymph-node counts of activated dendritic cells, the measurable variable we have found to be most accurate in predicting adjuvant activity. In order for a model of drug action to be useful, it must meet certain heuristic requirements: 1) it must accurately follow known data; 2) it must reliably predict new data; 3) it must be of a manageable size and complexity to enable implementation. Within the framework of our model, these requirements are met as follows. 1) In the training phase, our model is defined based upon the gene expression and draining lymph-node counts for four of eight analogues measured. 2) In the test phase, our model is applied to the gene expression of the four remaining analogues, and the predicted draining lymph-node counts are compared to the measured values. 3) Rather than use the full set of genes on a microarray chip, we select a limited subset of the greatest predictive power, which can fit on a much easier to use, and much cheaper, qPCR plate. The model-building phase is described below, and is implemented using many recent advances in machine learning and statistics.

### Microarrays

To obtain the gene expression data, we used two microarrays made by Affymetrix: (1) GENECHIP MOUSE GENOME 430A 2.0 ARRAY, which contains 22,600 probe sets to analyze the expression level of over 14,000 genes in the mouse genome, and (2) GENE CHIP MOUSE GENOME 430 2.0 ARRAY, which contains 45,000 probe sets to analyze the expression level of 39,000 transcripts and variants from over 34,000 genes in the mouse genome. Thus, we measured and compared the expression levels of essentially all genes in the mouse genome. The general liner model (GLM) analyses discussed below used the combined data obtained from both kinds of arrays.

### Microarray Data Processing

The raw output from a microarray chip is a list of several fluorescent intensities measured for each gene assayed. There are multiple measurements because, for each gene there are multiple complementary probes, called a probe set, which tile the gene sequence, thereby increasing specificity. There are four steps in the processing of microarray data that must be followed in order to generate data that may reliably be used as a measure of gene expression: 1) quality control (QC); 2) background correction; 3) normalization; and 4) probe set summarization. Following step 1, we included an extra step to combine the data from multiple chip models. All microarray processing was done using the statistical program R, with the following packages: ‘affy’, ‘affyPLM’, ‘multtest’, ‘stats’, ‘robustbase’. Raw microarray data in the form of CEL files were imported into R ExpressionSet data frame objects using the package ‘affy’.

### Quality Control

We performed four types of QC on the microarrays analysed. All three steps were designed with two goals in mind: determining the quality of individual chips/samples, and assessing the feasibility of combining all the chips/samples into a single dataset. The first step was an estimation of RNA degradation for each sample, for each chip. This determines whether the individual samples were of reasonable quality for the chip model used. Different chip models have different tolerances for RNA degradation, and thus one must analyse RNA degradation relative to the chip used. The second quality control step was the generation of a whiskered boxplot of raw signal intensities for each chip/sample side-by-side. The third step was to generate histograms of signal intensity for each chip/sample, while the fourth was to generate MA plots relative to the median amongst all chips. These three steps help in determining whether the overall distributions of signal intensities are comparable amongst the chips, which is necessary for reliable combination of multiple microarray datasets. The last QC step was to regenerate the fluorescent images of the microarrays in order to visually inspect the chips for deleterious spatial artifacts. The first and last steps were performed using the R package ‘affyPLM’, while the second, third, and fourth steps were done using the R package ‘affy’. In all cases we found all of the chips to be of sufficient quality, and of comparable signal intensity distributions, as to allow combination into a single dataset.

### Chip Combination

We used two different chip models, with one chip containing a superset of the probes of the other chip. We explored both chip models because we were unsure if the smaller chip covered enough of the genome to give a complete model of the mechanism of action of DBP. We show below that it does. In order to combine the two chip models, we manually mapped probes of identical sequence on the two chips, and selected only those probe sets that matched perfectly between the two chip models. We then extracted the signal intensities for all chips at those probe sets and combined them into a single ExpressionSet object.

### Background Correction, Normalization, Summarization

Raw microarray data is very noisy, owing to unavoidable technical artifacts in the fluorescence signals. The data must therefore be processed in order to derive reliable measures of gene expression. In the first part of this processing, one determines an estimate of the background signal present, and subtracts this background to derive true signal intensities. A common algorithm for this step is the Robust Multichip Average (RMA) algorithms, which computes a common background signal estimate from multiple chips, this multichip estimate being more resistant to outliers (more robust) than single chip estimates. We used the ‘affy’ implementation of RMA to background correct the chips. Once the background signal has been subtracted, in order to make chips comparable, one must normalize the resulting signals, as some chips may be inherently brighter than others, or may have localized regions of increased brightness, which may not correlate with gene expression. The method that we used to normalize the data was based upon the quantile normalization algorithm as implemented in ‘affy’, which seeks to normalize entire chips to each other without distorting the statistical properties of individual probe sets, by equalizing select quantiles of signal intensity (i.e. scaling all probe sets on all chips such that their 75th percentiles of signal intensity are equal). Finally, we used Tukey's median polish method to summarize the multiple probes within each probe set to a single value which represents the best estimate of expression of each gene in each sample given the values across the probe set. Once again, this was done using the R package ‘affy’.

### Output Data

The output data from the summarization step was on a log-2 scale, with a single value for each gene and each sample/chip. The log transformation was applied because it is considered a more reliable measure of gene expression than raw summarized values. This was transformed into an R data frame which was used as the input variables for the machine learning phase.

### Model Building

We have taken a machine learning approach to building a model of drug activity. In machine learning, one uses complex algorithms to find patterns and correlations in large datasets, starting from a basic hypothesis as to the form of the relationship between variables. We have tried four algorithms in an attempt to build a model of the activity of DBP and its analogues: Bayesian additive regression trees (BART), multivariate adaptive regression splines (MARS), gradient-boosted generalized linear models (GLMs), and bagged generalized linear models. Each will be described below, following a description of the general framework.

### Framework: Supervised Learning

The general class of algorithms we have used are called supervised learning. In supervised learning, one is given a dataset of input variables measured in a collection of samples, and an output response variable measured in those samples, with the goal of explaining the variation in the response variable based upon the variation in the input variable. In this analysis, the input variables are the measured gene expression, and the output variables are the mean draining lymph node content of activated dendritic cells for each analogue.

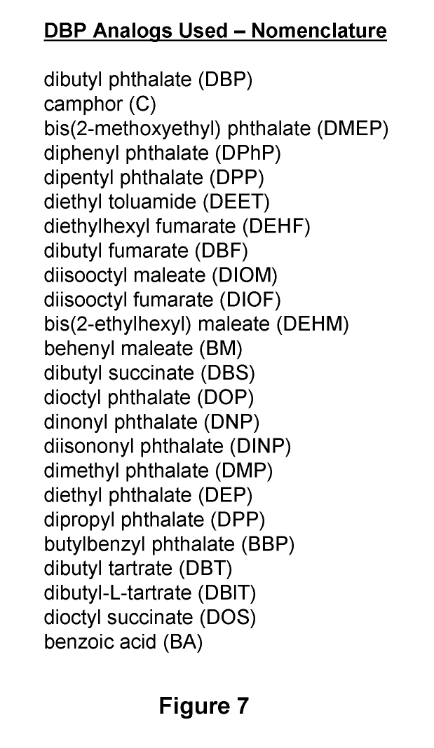

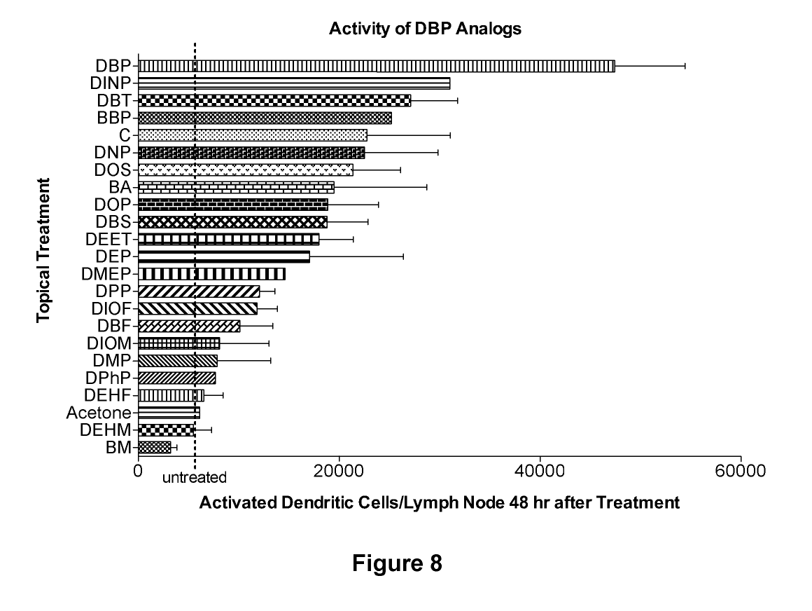

Supervised learning proceeds in two steps: training and testing. In the training phase, one uses a subset of samples to develop the model, applying the specific machine learning algorithm to construct the best possible fit of the model type to the known data. In the test phase, one then applies this fitted model to new data samples, in an attempt to predict the response variable. We have partitioned our microarray data into two groups for this purpose. The training set consisted of all the microarrays for DBP, untreated, DEET, and DMP, while the test set consisted of Acetone, DEHM, DEP, and dibutyl L tartrate (DBlT). This partitioning was chosen in order to provide both a training and test set that covered a broad range of counts of activated dendritic cells in draining lymph nodes. A listing of DBP analogs initially tested is shown in FIG. 7, and corresponding activities of the DBP analogs are shown in FIG. 8.

A goal of the methods disclosed herein is to develop an easy, affordable screening method for novel DBP analogues. To that end, given the high price of microarrays and the difficulty in their preparation, we attempted to limit the number of genes considered as input variables. This also has the positive effect of making the supervised learning algorithms more efficient. We chose to limit genes to subsets of 384, enough to fit on a custom qPCR plate, using the following three criteria in the selection process:
(1) absolute value of log-2 fold change in DBP versus untreated, see Table 8, #s 1-215, “log₂-fold change (LFC)”;
(2) p-value of differential expression between DBP and untreated as determined by the Wilcoxon rank-sum test, see Table 8, #s 216-436, “Wilcoxon”; and
(3) the Kendall tau correlation coefficient between gene expression and draining lymph node counts in DBP versus untreated, see Table 8, #s 437-794 “Kendall.”

The Wilcoxon test was performed using the R package ‘multtest’, and the Kendall tau was computed using the R package ‘stats’. For all three criteria, we chose the 384 most significant genes for use in the supervised learning. We applied each algorithm to each gene set in turn to test their predictive capacity.

### BART

A reference for the algorithm of BART is Chipman, H. A. et al. 2009 “BART: Bayesian Additive Regression Trees” Annals of Applied Statistics 4(1): 266-298). The specific implementation used was in the R package ‘BayesTree’. We used the command ‘bart’ with the following input parameters: ‘x.train’ was the training set gene expression, ‘y.train’ was the training set draining lymph node counts, ‘x.test’ was the test set gene expression, ‘sigest’ was set to NA, ‘sigdf’ was set to 3, ‘sigquant’ was set to 0.90, ‘k’ was set to 2.0, ‘power’ was set to 2.0, ‘base’ was set to 0.95, ‘ntree’ was set to 20000, ‘ndpost’ was set to 100000, and ‘nskip’ was set to 10000.

### MARS

A reference for MARS is the book ‘The Elements of Statistical Learning: Data Mining, Inference, and Prediction, by Hastie, T., Tibshirani, R., and Friedman, J., Second Edition, (2009) Springer Science+Business Media, LLC. The specific implementation used was the R package ‘earth’. The command used in training was ‘earth’, with the following input parameters: ‘x’ was the training set gene expression, ‘y’ was the training set draining lymph node counts, ‘weights’ was set to NULL, ‘wp’ was set to NULL, ‘scale.y’ was set to FALSE, subset was set to NULL, ‘glm’ was set to list(family=poisson)', ‘ncross’ was set to 100, and ‘nfold’ was set to 100. The ‘predict’ method of the resulting object was used with the test set gene expression as the ‘newdata’ argument to generate predictions.

### Gradient-Boosted Generalized Linear Models

A reference for gradient boosting and generalized linear models is the book ‘The Elements of Statistical Learning: Data Mining, Inference, and Prediction, by Hastie, T., Tibshirani, R., and Friedman, J., Second Edition, (2009) Springer Science+Business Media, LLC. The specific implementation used was in the R package ‘mboost’. The command used was ‘glmboose, with the following input parameters: ‘x’ was set to the training set gene expression, ‘y’ was set to the training set draining lymph node counts, and ‘control’ was set to ‘boost_control(mstop=100000)’. The ‘predict’ method of the resulting object was used with the test set gene expression as the ‘newdata’ argument to generate predictions.

### Bagged Generalized Linear Models

A reference for bagging and generalized linear models is the book ‘The Elements of Statistical Learning: Data Mining, Inference, and Prediction, by Hastie, T., Tibshirani, R., and Friedman, J., Second Edition, (2009) Springer Science+Business Media, LLC. The implementation of the individual GLMs was done using the R package ‘stats’. The specific command was ‘glm’ with the following inputs: ‘x’ was set to the training set gene expression, ‘y’ was set to the training set draining lymph node counts, and ‘family’ was set to ‘poisson’. The implementation of bagging was hand-coded, using 10000 rounds of glm with random subsets of between 10 and 15 genes each. The ‘predict’ method of the resulting objects was used with the test set gene expression as the ‘newdata’ argument to generate predictions.

### Predictions

The best predictor out of the tested combinations of algorithm and gene set was using MARS with the genes selected for log-2 fold change, shown in Table 7 as ‘Earth_Abs_LFC’, with a mean standard error (MSE) of 7.04%. Another notable performer was ‘Bart_Wilcoxon’, the application of BART to the genes selected for Wilcoxon p-value, with an MSE of 11.3%. These accuracies are easily sufficient to be used for a screen for novel DBP analogues, as proposed herein.

TABLE 7

| Predictor | MSE |
|---|---|
| Bagging_Abs_LFC | 0.1760 |
| Bagging_Wilcoxon | 0.2322 |
| Bagging_KT | 12.8291 |
| Bart_KT | 0.1953 |
| Bart_Abs_LFC | 0.6234 |
| Bart_Wilcoxon | 0.1129 |
| Earth_KT | 0.2581 |
| Earth_Abs_LFC | 0.0704 |
| Earth_Wilcoxon | 0.2109 |
| Glmboost_KT | 0.2367 |
| Glmboost_Abs_LFC | 0.1800 |
| Glmboost_Wilcoxon | 0.3891 |

### CONCLUSIONS

A traditional view has been that a prospective test of a predictor is the most valid way to test a model. However, when vast quantities of data are utilized, such as genome-wide expression data or predictions of linear B cell epitopes that antibodies might recognize in the universe of globular proteins, “machine learning” algorithms are necessary to obtain a simple pattern that enables a prediction of a single function out of countless different possibilities. Existing data is used to obtain such an algorithm.

Here, we divided a data set into two approximately equal halves, where both halves contain samples covering a broad spectrum of outcomes. A first set was termed the “training” set. Using the GLM methods described herein, we input: (a) all of the raw data (e.g., expression levels of 39,000 unique transcripts) contained in the training set, and (b) the outcome of each individual member (e.g., number of activated dendritic cells in the draining lymph nodes after 48 hr) into the computer program, which found the best correlation between gene expression levels and outcome level. We took the best fit predictor and applied it to the gene expression data from the individual members of the “test” set, and the algorithm determined the expected number of activated dendritic cells in the draining lymph nodes in each case. The predicted outcomes were 77.5% accurate when compared to the experimentally obtained data for the test set. In bioinformatics, that level of accuracy is considered a useful predictive algorithm.

The bioinformatics approaches we used here employ known data sets to determine the predictive value of a particular analysis. Because machine learning is an ongoing process, the more data sets that are input into a predictive system, the more accurate the predictions will become.

Many of the genes that we have identified have been included in various commercially available arrays. Indeed, genome wide arrays covering ˜30,000 genes are designed to include probes for as many genes as possible and their intent is to include the entire expressed genome of a given species. However, such arrays are too large and currently too expensive to constitute a useful screen for any one desired drug outcome. In contrast, we have identified a gene expression signature induced by DBP that is small enough in terms of number of genes to be a useful screen for molecules having adjuvant activity similar to DBP. Based on the mean standard errors generated by our analyses, we have indeed identified a small enough gene set whose differential expression characterizes the adjuvant effect of DBP. As the adjuvant effect of DBP is unique in terms of known mechanisms of action, as well as in its ability to be used independently of a vaccine, which no other known immunologic adjuvant can do, the gene expression signature induced by DBP that we have identified here is of significant value.

TABLE 8
Summary of Genes Identified by ≧2-Fold Expression Changes and by General Linear Models
(“Wilcoxon” and “Kendall”)

| | | | | ≧2-Fold | | |
|---|---|---|---|---|---|---|
| # | Gene Name | Symbol | Refseq_ID | change | Wilcoxon | Kendall |
| 1 | mucolipin 2 | Mcoln2 | NM_001005846_at | Yes | Yes | No |
| 2 | zinc finger and BTB | Zbtb16 | NM_001033324_at | Yes | No | Yes |
| | domain containing 16 | | | | | |
| 3 | CD44 antigen | Cd44 | NM_001039150_at | Yes | No | No |
| 4 | CD44 antigen | Cd44 | NM_001039151_at | Yes | No | No |
| 5 | tissue inhibitor of | Timp1 | NM_001044384_at | Yes | Yes | No |
| | metalloproteinase 1 | | | | | |
| 6 | cAMP responsive element | Crem | NM_001110850_at | Yes | Yes | No |
| | modulator | | | | | |
| 7 | cAMP responsive element | Crem | NM_001110853_at | Yes | Yes | No |
| | modulator | | | | | |
| 8 | cAMP responsive element | Crem | NM_001110854_at | Yes | Yes | No |
| | modulator | | | | | |
| 9 | cAMP responsive element | Crem | NM_001110855_at | Yes | Yes | No |
| | modulator | | | | | |
| 10 | cAMP responsive element | Crem | NM_001110856_at | Yes | Yes | No |
| | modulator | | | | | |
| 11 | cAMP responsive element | Crem | NM_001110857_at | Yes | Yes | No |
| | modulator | | | | | |
| 12 | cAMP responsive element | Crem | NM_001110858_at | Yes | Yes | No |
| | modulator | | | | | |
| 13 | cAMP responsive element | Crem | NM_001110859_at | Yes | Yes | No |
| | modulator | | | | | |
| 14 | solute carrier family 39 | Slc39a8 | NM_001135149_at | Yes | Yes | Yes |
| | (metal ion transporter), | | | | | |
| | member 8 | | | | | |
| 15 | solute carrier family 39 | Slc39a8 | NM_001135150_at | Yes | Yes | Yes |
| | (metal ion transporter), | | | | | |
| | member 8 | | | | | |
| 16 | nuclear receptor | Nr4a2 | NM_001139509_at | Yes | Yes | No |
| | subfamily 4, group A, | | | | | |
| | member 2 | | | | | |
| 17 | nuclear factor of kappa | Nfkbiz | NM_001159394_at | Yes | Yes | No |
| | light polypeptide gene | | | | | |
| | enhancer in B-cells | | | | | |
| | inhibitor, zeta | | | | | |
| 18 | nuclear factor of kappa | Nfkbiz | NM_001159395_at | Yes | Yes | No |
| | light polypeptide gene | | | | | |
| | enhancer in B-cells | | | | | |
| | inhibitor, zeta | | | | | |
| 19 | interferon regulatory | Irf1 | NM_001159396_at | Yes | Yes | No |
| | factor 1 | | | | | |
| 20 | chemokine (C-C motif) | Ccl20 | NM_001159738_at | Yes | Yes | No |
| | ligand 20 | | | | | |
| 21 | tumor necrosis factor | Tnfrsf12a | NM_001161746_at | Yes | Yes | No |
| | receptor superfamily, | | | | | |
| | member 12a | | | | | |
| 22 | trichohyalin | Tchh | NM_001163098_at | Yes | Yes | No |
| 23 | C-type lectin domain | Clec4d | NM_001163161_at | Yes | Yes | No |
| | family 4, member d | | | | | |
| 24 | solute carrier family 25 | Slc25a25 | NM_001164357_at | Yes | No | No |
| | (mitochondrial carrier, | | | | | |
| | phosphate carrier), | | | | | |
| | member 25 | | | | | |
| 25 | solute carrier family 25 | Slc25a25 | NM_001164358_at | Yes | No | No |
| | (mitochondrial carrier, | | | | | |
| | phosphate carrier), | | | | | |
| | member 25 | | | | | |
| 26 | kallikrein related- | Klk6 | NM_001164696_at | Yes | Yes | No |
| | peptidase 6 | | | | | |
| 27 | kallikrein related- | Klk6 | NM_001164697_at | Yes | Yes | No |
| | peptidase 6 | | | | | |
| 28 | kallikrein related- | Klk6 | NM_001164698_at | Yes | Yes | No |
| | peptidase 6 | | | | | |
| 29 | tumor necrosis factor, | Tnfaip3 | NM_001166402_at | Yes | Yes | No |
| | alpha-induced protein 3 | | | | | |
| 30 | suppressor of cytokine | Socs2 | NM_001168655_at | Yes | No | No |
| | signaling 2 | | | | | |
| 31 | suppressor of cytokine | Socs2 | NM_001168656_at | Yes | No | No |
| | signaling 2 | | | | | |
| 32 | suppressor of cytokine | Socs2 | NM_001168657_at | Yes | No | No |
| | signaling 2 | | | | | |
| 33 | Nup62-Il4i1 protein | Nup62-il4i1 | NM_001171024_at | Yes | Yes | No |
| 34 | AT rich interactive | Arid5a | NM_001172205_at | Yes | Yes | No |
| | domain 5A (MRF1-like) | | | | | |
| 35 | AT rich interactive | Arid5a | NM_001172206_at | Yes | Yes | No |
| | domain 5A (MRF1-like) | | | | | |
| 36 | CD44 antigen | Cd44 | NM_001177785_at | Yes | No | No |
| 37 | CD44 antigen | Cd44 | NM_001177786_at | Yes | No | No |
| 38 | CD44 antigen | Cd44 | NM_001177787_at | Yes | No | No |
| 39 | spermine oxidase | Smox | NM_001177833_at | Yes | Yes | No |
| 40 | spermine oxidase | Smox | NM_001177834_at | Yes | Yes | No |
| 41 | spermine oxidase | Smox | NM_001177835_at | Yes | Yes | No |
| 42 | spermine oxidase | Smox | NM_001177836_at | Yes | Yes | No |
| 43 | spermine oxidase | Smox | NM_001177837_at | Yes | Yes | No |
| 44 | spermine oxidase | Smox | NM_001177838_at | Yes | Yes | No |
| 45 | spermine oxidase | Smox | NM_001177839_at | Yes | Yes | No |
| 46 | spermine oxidase | Smox | NM_001177840_at | Yes | Yes | No |
| 47 | phosphodiesterase 4B, | Pde4b | NM_001177980_at | Yes | Yes | No |
| | cAMP specific | | | | | |
| 48 | phosphodiesterase 4B, | Pde4b | NM_001177981_at | Yes | Yes | No |
| | cAMP specific | | | | | |
| 49 | phosphodiesterase 4B, | Pde4b | NM_001177982_at | Yes | Yes | No |
| | cAMP specific | | | | | |
| 50 | RIKEN cDNA 2310016C08 | 2310016C08Rik | NM_001190461_at | Yes | Yes | No |
| | gene | | | | | |
| 51 | a disintegrin and | Adam8 | NM_007403_at | Yes | Yes | Yes |
| | metallopeptidase domain 8 | | | | | |
| 52 | baculoviral IAP repeat- | Birc3 | NM_007464_at | Yes | No | No |
| | containing 3 | | | | | |
| 53 | activating transcription | Atf3 | NM_007498_at | Yes | Yes | No |
| | factor 3 | | | | | |
| 54 | B-cell translocation gene | Btg2 | NM_007570_at | Yes | Yes | Yes |
| | 2, anti-proliferative | | | | | |
| 55 | caspase 4, apoptosis- | Casp4 | NM_007609_at | Yes | Yes | No |
| | related cysteine | | | | | |
| | peptidase | | | | | |
| 56 | CCAAT/enhancer binding | Cebpd | NM_007679_at | Yes | Yes | No |
| | protein (C/EBP), delta | | | | | |
| 57 | suppressor of cytokine | Socs2 | NM_007706_at | Yes | No | No |
| | signaling 2 | | | | | |
| 58 | suppressor of cytokine | Socs3 | NM_007707_at | Yes | Yes | No |
| | signaling 3 | | | | | |
| 59 | mitogen-activated | Map3k8 | NM_007746_at | Yes | No | No |
| | protein kinase kinase | | | | | |
| | kinase 8 | | | | | |
| 60 | colony stimulating factor | Csf2rb | NM_007780_at | Yes | No | Yes |
| | 2 receptor, beta, low- | | | | | |
| | affinity (granulocyte- | | | | | |
| | macrophage) | | | | | |
| 61 | early growth response 1 | Egr1 | NM_007913_at | Yes | Yes | No |
| 62 | ets homologous factor | Ehf | NM_007914_at | Yes | Yes | No |
| 63 | epiregulin | Ereg | NM_007950_at | Yes | Yes | No |
| 64 | fibroblast growth factor | Fgfbp1 | NM_008009_at | Yes | No | No |
| | binding protein 1 | | | | | |
| 65 | GTP cyclohydrolase 1 | Gch1 | NM_008102_at | Yes | Yes | No |
| 66 | gap junction protein, | Gjb2 | NM_008125_at | Yes | Yes | No |
| | beta 2 | | | | | |
| 67 | G-protein coupled | Gpr65 | NM_008152_at | Yes | Yes | No |
| | receptor 65 | | | | | |
| 68 | chemokine ( C—X—C motif) | Cxcl1 | NM_008176_at | Yes | Yes | No |
| | ligand 1 | | | | | |
| 69 | hyaluronan synthase 2 | Has2 | NM_008216_at | Yes | Yes | No |
| 70 | hyaluronan synthase 3 | Has3 | NM_008217_at | Yes | Yes | No |
| 71 | histidine decarboxylase | Hdc | NM_008230_at | Yes | Yes | Yes |
| 72 | interferon activated gene | Ifi202b | NM_008327_at | Yes | Yes | No |
| | 202B | | | | | |
| 73 | interleukin 1 beta | Il1b | NM_008361_at | Yes | Yes | No |
| 74 | interleukin 7 receptor | Il7r | NM_008372_at | Yes | No | Yes |
| 75 | interferon regulatory | Irf1 | NM_008390_at | Yes | Yes | No |
| | factor 1 | | | | | |
| 76 | immunoresponsive gene 1 | Irg1 | NM_008392_at | Yes | Yes | No |
| 77 | Jun-B oncogene | Junb | NM_008416_at | Yes | Yes | No |
| 78 | keratin 6A | Krt6a | NM_008476_at | Yes | Yes | Yes |
| 79 | lipocalin 2 | Lcn2 | NM_008491_at | Yes | Yes | No |
| 80 | metallothionein 2 | Mt2 | NM_008630_at | Yes | No | No |
| 81 | nuclear factor of kappa | Nfkbie | NM_008690_at | Yes | Yes | No |
| | light polypeptide gene | | | | | |
| | enhancer in B-cells | | | | | |
| | inhibitor, epsilon | | | | | |
| 82 | natriuretic peptide type B | Nppb | NM_008726_at | Yes | Yes | No |
| 83 | dual specificity | Dusp8 | NM_008748_at | Yes | No | No |
| | phosphatase 8 | | | | | |
| 84 | serine (or cysteine) | Serpine1 | NM_008871_at | Yes | Yes | No |
| | peptidase inhibitor, | | | | | |
| | clade E, member 1 | | | | | |
| 85 | pentraxin related gene | Ptx3 | NM_008987_at | Yes | Yes | No |
| 86 | reticuloendotheliosis | Rel | NM_009044_at | Yes | Yes | No |
| | oncogene | | | | | |
| 87 | S100 calcium binding | S100a9 | NM_009114_at | Yes | Yes | No |
| | protein A9 (calgranulin | | | | | |
| | B) | | | | | |
| 88 | chemokine ( C—X—C motif) | Cxcl2 | NM_009140_at | Yes | Yes | No |
| | ligand 2 | | | | | |
| 89 | solute carrier family 1 | Slc1a1 | NM_009199_at | Yes | Yes | No |
| | (neuronal/epithelial | | | | | |
| | high affinity glutamate | | | | | |
| | transporter, system | | | | | |
| | Xag), member 1 | | | | | |
| 90 | small proline-rich | Sprr1a | NM_009264_at | Yes | Yes | No |
| | protein 1A | | | | | |
| 91 | small proline-rich | Sprr1b | NM_009265_at | Yes | Yes | No |
| | protein 1B | | | | | |
| 92 | stanniocalcin 1 | Stc1 | NM_009285_at | Yes | No | Yes |
| 93 | pleckstrin homology-like | Phlda1 | NM_009344_at | Yes | Yes | No |
| | domain, family A, | | | | | |
| | member 1 | | | | | |
| 94 | tumor necrosis factor, | Tnfaip2 | NM_009396_at | Yes | Yes | No |
| | alpha-induced protein 2 | | | | | |
| 95 | tumor necrosis factor, | Tnfaip3 | NM_009397_at | Yes | Yes | No |
| | alpha-induced protein 3 | | | | | |
| 96 | tumor necrosis factor | Tnfaip6 | NM_009398_at | Yes | Yes | No |
| | alpha induced protein 6 | | | | | |
| 97 | TNF receptor-associated | Traf1 | NM_009421_at | Yes | No | No |
| | factor 1 | | | | | |
| 98 | amphiregulin | Areg | NM_009704_at | Yes | Yes | No |
| 99 | CCR4 carbon catabolite | Ccrn4l | NM_009834_at | Yes | Yes | No |
| | repression 4-like (S. cerevisiae) | | | | | |
| 100 | CD14 antigen | Cd14 | NM_009841_at | Yes | Yes | No |
| 101 | CD44 antigen | Cd44 | NM_009851_at | Yes | No | No |
| 102 | CD83 antigen | Cd83 | NM_009856_at | Yes | Yes | No |
| 103 | cholesterol 25- | Ch25h | NM_009890_at | Yes | Yes | No |
| | hydroxylase | | | | | |
| 104 | cytokine inducible SH2- | Cish | NM_009895_at | Yes | No | No |
| | containing protein | | | | | |
| 105 | chemokine ( C—X—C motif) | Cxcr2 | NM_009909_at | Yes | Yes | No |
| | receptor 2 | | | | | |
| 106 | cytochrome P450, family | Cyp27b1 | NM_010009_at | Yes | Yes | No |
| | 27, subfamily b, | | | | | |
| | polypeptide 1 | | | | | |
| 107 | diacylglycerol O- | Dgat1 | NM_010046_at | Yes | No | No |
| | acyltransferase 1 | | | | | |
| 108 | interleukin 4 induced 1 | Il4i1 | NM_010215_at | Yes | Yes | No |
| 109 | FK506 binding protein 5 | Fkbp5 | NM_010220_at | Yes | No | Yes |
| 110 | fos-like antigen 1 | Fosl1 | NM_010235_at | Yes | Yes | No |
| 111 | GTP binding protein | Gem | NM_010276_at | Yes | Yes | No |
| | (gene overexpressed in | | | | | |
| | skeletal muscle) | | | | | |
| 112 | heparin-binding EGF-like | Hbegf | NM_010415_at | Yes | Yes | No |
| | growth factor | | | | | |
| 113 | nuclear receptor | Nr4a1 | NM_010444_at | Yes | Yes | No |
| | subfamily 4, group A, | | | | | |
| | member 1 | | | | | |
| 114 | intercellular adhesion | Icam1 | NM_010493_at | Yes | Yes | No |
| | molecule 1 | | | | | |
| 115 | immediate early | Ier2 | NM_010499_at | Yes | No | No |
| | response 2 | | | | | |
| 116 | cysteine rich protein 61 | Cyr61 | NM_010516_at | Yes | Yes | No |
| 117 | Jun oncogene | Jun | NM_010591_at | Yes | No | No |
| 118 | keratin 27 | Krt27 | NM_010666_at | Yes | Yes | No |
| 119 | v-maf | Maff | NM_010755_at | Yes | Yes | No |
| | musculoaponeurotic | | | | | |
| | fibrosarcoma oncogene | | | | | |
| | family, protein F (avian) | | | | | |
| 120 | C-type lectin domain | Clec4d | NM_010819_at | Yes | Yes | No |
| | family 4, member d | | | | | |
| 121 | nuclear factor of kappa | Nfkbia | NM_010907_at | Yes | No | No |
| | light polypeptide gene | | | | | |
| | enhancer in B-cells | | | | | |
| | inhibitor, alpha | | | | | |
| 122 | plasminogen activator, | Plaur | NM_011113_at | Yes | No | No |
| | urokinase receptor | | | | | |
| 123 | kallikrein related- | Klk6 | NM_011177_at | Yes | Yes | No |
| | peptidase 6 | | | | | |
| 124 | RAB20, member RAS | Rab20 | NM_011227_at | Yes | Yes | No |
| | oncogene family | | | | | |
| 125 | serum amyloid A 3 | Saa3 | NM_011315_at | Yes | Yes | No |
| 126 | chemokine (C-C motif) | Ccl1 | NM_011329_at | Yes | Yes | No |
| | ligand 1 | | | | | |
| 127 | chemokine (C-C motif) | Ccl12 | NM_011331_at | Yes | Yes | No |
| | ligand 12 | | | | | |
| 128 | chemokine (C-C motif) | Ccl17 | NM_011332_at | Yes | Yes | No |
| | ligand 17 | | | | | |
| 129 | chemokine (C-C motif) | Ccl2 | NM_011333_at | Yes | Yes | No |
| | ligand 2 | | | | | |
| 130 | chemokine (C-C motif) | Ccl3 | NM_011337_at | Yes | Yes | No |
| | ligand 3 | | | | | |
| 131 | chemokine (C-C motif) | Ccl9 | NM_011338_at | Yes | Yes | No |
| | ligand 9 | | | | | |
| 132 | solute carrier family 34 | Slc34a2 | NM_011402_at | Yes | Yes | No |
| | (sodium phosphate), | | | | | |
| | member 2 | | | | | |
| 133 | solute carrier family 7 | Slc7a5 | NM_011404_at | Yes | Yes | Yes |
| | (cationic amino acid | | | | | |
| | transporter, y+ system), | | | | | |
| | member 5 | | | | | |
| 134 | schlafen 2 | Slfn2 | NM_011408_at | Yes | Yes | No |
| 135 | schlafen 4 | Slfn4 | NM_011410_at | Yes | Yes | No |
| 136 | secretory leukocyte | Slpi | NM_011414_at | Yes | Yes | No |
| | peptidase inhibitor | | | | | |
| 137 | basic helix-loop-helix | Bhlhe40 | NM_011498_at | Yes | No | No |
| | family, member e40 | | | | | |
| 138 | tissue inhibitor of | Timp1 | NM_011593_at | Yes | Yes | No |
| | metalloproteinase 1 | | | | | |
| 139 | phospholipid scramblase | Plscr1 | NM_011636_at | Yes | No | No |
| 140 | zinc finger protein 36 | Zfp36 | NM_011756_at | Yes | Yes | No |
| 141 | toll-like receptor 2 | Tlr2 | NM_011905_at | Yes | Yes | No |
| 142 | interferon activated gene | Ifi202b | NM_011940_at | Yes | Yes | No |
| | 202B | | | | | |
| 143 | cAMP responsive element | Crem | NM_013498_at | Yes | Yes | No |
| | modulator | | | | | |
| 144 | granzyme B | Gzmb | NM_013542_at | Yes | Yes | No |
| 145 | metallothionein 1 | Mt1 | NM_013602_at | Yes | No | No |
| 146 | nuclear receptor | Nr4a2 | NM_013613_at | Yes | Yes | No |
| | subfamily 4, group A, | | | | | |
| | member 2 | | | | | |
| 147 | chemokine (C-C motif) | Ccl7 | NM_013654_at | Yes | Yes | No |
| | ligand 7 | | | | | |
| 148 | tumor necrosis factor | Tnf | NM_013693_at | Yes | Yes | No |
| 149 | pyruvate dehydrogenase | Pdk4 | NM_013743_at | Yes | Yes | No |
| | kinase, isoenzyme 4 | | | | | |
| 150 | tumor necrosis factor | Tnfrsf12a | NM_013749_at | Yes | Yes | No |
| | receptor superfamily, | | | | | |
| | member 12a | | | | | |
| 151 | breast cancer anti- | Bcar3 | NM_013867_at | Yes | No | No |
| | estrogen resistance 3 | | | | | |
| 152 | chloride intracellular | Clic4 | NM_013885_at | Yes | No | No |
| | channel 4 | | | | | |
| | (mitochondrial) | | | | | |
| 153 | regulator of G-protein | Rgs1 | NM_015811_at | Yes | Yes | No |
| | signaling 1 | | | | | |
| 154 | mitogen-activated | Map3k6 | NM_016693_at | Yes | No | No |
| | protein kinase kinase | | | | | |
| | kinase 6 | | | | | |
| 155 | keratin 35 | Krt35 | NM_016880_at | Yes | No | No |
| 156 | chemokine (C-C motif) | Ccl20 | NM_016960_at | Yes | Yes | No |
| | ligand 20 | | | | | |
| 157 | nuclear factor, | Nfil3 | NM_017373_at | Yes | Yes | No |
| | interleukin 3, regulated | | | | | |
| 158 | activity regulated | Arc | NM_018790_at | Yes | Yes | No |
| | cytoskeletal-associated | | | | | |
| | protein | | | | | |
| 159 | CD86 antigen | Cd86 | NM_019388_at | Yes | Yes | No |
| 160 | interleukin 1 family, | Il1f6 | NM_019450_at | Yes | Yes | No |
| | member 6 | | | | | |
| 161 | receptor (calcitonin) | Ramp3 | NM_019511_at | Yes | No | Yes |
| | activity modifying | | | | | |
| | protein 3 | | | | | |
| 162 | pleckstrin | Plek | NM_019549_at | Yes | Yes | No |
| 163 | solute carrier family 5 | Slc5a1 | NM_019810_at | Yes | Yes | No |
| | (sodium/glucose | | | | | |
| | cotransporter), member 1 | | | | | |
| 164 | phosphodiesterase 4B, | Pde4b | NM_019840_at | Yes | Yes | No |
| | cAMP specific | | | | | |
| 165 | C-type lectin domain | Clec4e | NM_019948_at | Yes | Yes | No |
| | family 4, member e | | | | | |
| 166 | keratin 71 | Krt71 | NM_019956_at | Yes | Yes | No |
| 167 | C-type lectin domain | Clec7a | NM_020008_at | Yes | Yes | No |
| | family 7, member a | | | | | |
| 168 | angiopoietin-like 4 | Angptl4 | NM_020581_at | Yes | Yes | Yes |
| 169 | thymic stromal | Tslp | NM_021367_at | Yes | Yes | No |
| | lymphopoietin | | | | | |
| 170 | interleukin 20 | Il20 | NM_021380_at | Yes | Yes | No |
| 171 | phorbol-12-myristate-13- | Pmaip1 | NM_021451_at | Yes | Yes | No |
| | acetate-induced protein 1 | | | | | |
| 172 | prostaglandin E synthase | Ptges | NM_022415_at | Yes | No | No |
| 173 | solute carrier family 15, | Slc15a3 | NM_023044_at | Yes | No | No |
| | member 3 | | | | | |
| 174 | chemokine ( C—X—C motif) | Cxcl16 | NM_023158_at | Yes | Yes | No |
| | ligand 16 | | | | | |
| 175 | RIKEN cDNA 2310016C08 | 2310016C08Rik | NM_023516_at | Yes | Yes | No |
| | gene | | | | | |
| 176 | zinc finger protein 593 | Zfp593 | NM_024215_at | Yes | No | No |
| 177 | stefin A3 | Stfa3 | NM_025288_at | Yes | Yes | No |
| 178 | solute carrier family 39 | Slc39a8 | NM_026228_at | Yes | Yes | Yes |
| | (metal ion transporter), | | | | | |
| | member 8 | | | | | |
| 179 | dual specificity | Dusp6 | NM_026268_at | Yes | Yes | No |
| | phosphatase 6 | | | | | |
| 180 | tubulin, beta 6 | Tubb6 | NM_026473_at | Yes | Yes | No |
| 181 | mucolipin 2 | Mcoln2 | NM_026656_at | Yes | Yes | No |
| 182 | RIKEN cDNA 1810011010 | 1810011010Rik | NM_026931_at | Yes | Yes | No |
| | gene | | | | | |
| 183 | ring finger protein 19B | Rnf19b | NM_029219_at | Yes | No | Yes |
| 184 | solute carrier family 10 | Slc10a6 | NM_029415_at | Yes | No | No |
| | (sodium/bile acid | | | | | |
| | cotransporter family), | | | | | |
| | member 6 | | | | | |
| 185 | ATP-binding cassette, | Abcc3 | NM_029600_at | Yes | No | Yes |
| | sub-family C | | | | | |
| | (CFTR/MRP), member 3 | | | | | |
| 186 | RIKEN cDNA 1600029D21 | 1600029D21Rik | NM_029639_at | Yes | Yes | No |
| | gene | | | | | |
| 187 | killer cell lectin-like | Klrb1b | NM_030599_at | Yes | No | Yes |
| | receptor subfamily B | | | | | |
| | member 1B | | | | | |
| 188 | nuclear factor of kappa | Nfkbiz | NM_030612_at | Yes | Yes | No |
| | light polypeptide gene | | | | | |
| | enhancer in B-cells | | | | | |
| | inhibitor, zeta | | | | | |
| 189 | Jun dimerization protein 2 | Jdp2 | NM_030887_at | Yes | No | No |
| 190 | interleukin 6 | Il6 | NM_031168_at | Yes | Yes | No |
| 191 | A kinase (PRKA) anchor | Akap12 | NM_031185_at | Yes | No | No |
| | protein (gravin) 12 | | | | | |
| 192 | B-cell | Bcl3 | NM_033601_at | Yes | Yes | No |
| | leukemia/lymphoma 3 | | | | | |
| 193 | epithelial mitogen | Epgn | NM_053087_at | Yes | Yes | No |
| 194 | calcitonin-related | Calcb | NM_054084_at | Yes | No | No |
| | polypeptide, beta | | | | | |
| 195 | OTU domain containing | Otud7a | NM_130880_at | Yes | No | Yes |
| | 7A | | | | | |
| 196 | immediate early | Ier3 | NM_133662_at | Yes | Yes | No |
| | response 3 | | | | | |
| 197 | keratin 25 | Krt25 | NM_133730_at | Yes | Yes | No |
| 198 | protease, serine, 22 | Prss22 | NM_133731_at | Yes | Yes | No |
| 199 | UDP-N-acetylglucosamine | Uap1 | NM_133806_at | Yes | No | No |
| | pyrophosphorylase 1 | | | | | |
| 200 | RIKEN cDNA 2010002N04 | 2010002N04Rik | NM_134133_at | Yes | Yes | No |
| | gene | | | | | |
| 201 | hepatitis A virus cellular | Havcr2 | NM_134250_at | Yes | Yes | Yes |
| | receptor 2 | | | | | |
| 202 | cytohesin 1 interacting | Cytip | NM_139200_at | Yes | No | No |
| | protein | | | | | |
| 203 | CD207 antigen | Cd207 | NM_144943_at | Yes | Yes | No |
| 204 | peptide YY | Pyy | NM_145435_at | Yes | Yes | Yes |
| 205 | acyl-CoA thioesterase 5 | Acot5 | NM_145444_at | Yes | Yes | No |
| 206 | spermine oxidase | Smox | NM_145533_at | Yes | Yes | No |
| 207 | AT rich interactive | Arid5a | NM_145996_at | Yes | Yes | No |
| | domain 5A (MRF1-like) | | | | | |
| 208 | solute carrier family 25 | Slc25a25 | NM_146118_at | Yes | No | No |
| | (mitochondrial carrier, | | | | | |
| | phosphate carrier), | | | | | |
| | member 25 | | | | | |
| 209 | zinc finger CCCH type | Zc3h12a | NM_153159_at | Yes | Yes | No |
| | containing 12A | | | | | |
| 210 | dual specificity | Dusp7 | NM_153459_at | Yes | No | No |
| | phosphatase 7 | | | | | |
| 211 | a disintegrin-like and | Adamts4 | NM_172845_at | Yes | Yes | No |
| | metallopeptidase | | | | | |
| | (reprolysin type) with | | | | | |
| | thrombospondin type 1 | | | | | |
| | motif, 4 | | | | | |
| 212 | thymic stromal | Tslp | NR_033206_at | Yes | Yes | No |
| | lymphopoietin | | | | | |
| 213 | AT rich interactive | Arid5a | NR_033310_at | Yes | Yes | No |
| | domain 5A (MRF1-like) | | | | | |
| 214 | interferon-activable | LOC100044068 | XM_001473873_at | Yes | Yes | No |
| | protein 202-like | | | | | |
| 215 | c-C motif chemokine 12- | LOC100504977 | XM_003085794_at | Yes | Yes | No |
| | like | | | | | |
| 216 | NA | NA | AFFX- | No | Yes | No |
| | | | 18SRNAMur/X00686_3_at | | | |
| 217 | NA | NA | AFFX- | No | Yes | No |
| | | | 18SRNAMur/X00686_5_at | | | |
| 218 | NA | NA | AFFX- | No | Yes | No |
| | | | 18SRNAMur/X00686_M_at | | | |
| 219 | NA | NA | AFFX-BioB-3_at | No | Yes | No |
| 220 | NA | NA | AFFX-BioB-5_at | No | Yes | No |
| 221 | NA | NA | AFFX-BioB-M_at | No | Yes | No |
| 222 | NA | NA | AFFX-BioC-3_at | No | Yes | No |
| 223 | NA | NA | AFFX-BioC-5_at | No | Yes | No |
| 224 | NA | NA | AFFX-BioDn-3_at | No | Yes | No |
| 225 | NA | NA | AFFX-BioDn-5_at | No | Yes | No |
| 226 | NA | NA | AFFX-r2-Bs-lys-3_at | No | Yes | No |
| 227 | NA | NA | AFFX-r2-Bs-lys-5_at | No | Yes | No |
| 228 | NA | NA | AFFX-r2-Bs-lys-M_at | No | Yes | No |
| 229 | NA | NA | AFFX-r2-Bs-thr-5_s_at | No | Yes | No |
| 230 | NA | NA | AFFX-r2-Ec-bioB-3_at | No | Yes | No |
| 231 | NA | NA | AFFX-r2-Ec-bioB-5_at | No | Yes | No |
| 232 | NA | NA | AFFX-r2-Ec-bioB-M_at | No | Yes | No |
| 233 | NA | NA | AFFX-r2-Ec-bioC-3_at | No | Yes | No |
| 234 | NA | NA | AFFX-r2-Ec-bioC-5_at | No | Yes | No |
| 235 | NA | NA | AFFX-ThrX-5_at | No | Yes | No |
| 236 | keratin associated | Krtap4-16 | NM_001013823_at | No | Yes | No |
| | protein 4-16 | | | | | |
| 237 | myotilin | Myot | NM_001033621_at | No | Yes | No |
| 238 | interferon induced | Ifitm6 | NM_001033632_at | No | Yes | No |
| | transmembrane protein 6 | | | | | |
| 239 | LIM domain binding 3 | Ldb3 | NM_001039075_at | No | Yes | No |
| 240 | LIM domain binding 3 | Ldb3 | NM_001039076_at | No | Yes | No |
| 241 | myosin, heavy | Myh2 | NM_001039545_at | No | Yes | No |
| | polypeptide 2, skeletal | | | | | |
| | muscle, adult | | | | | |
| 242 | angiopoietin-like 7 | Angptl7 | NM_001039554_at | No | Yes | No |
| 243 | late cornified envelope | Lce3a | NM_001039594_at | No | Yes | No |
| | 3A | | | | | |
| 244 | nucleolar and spindle | Nusap1 | NM_001042652_at | No | Yes | No |
| | associated protein 1 | | | | | |
| 245 | myomesin 1 | Myom1 | NM_001083934_at | No | Yes | No |
| 246 | myosin, heavy | Myh3 | NM_001099635_at | No | Yes | No |
| | polypeptide 3, skeletal | | | | | |
| | muscle, embryonic | | | | | |
| 247 | predicted gene 11567 | Gm11567 | NM_001101613_at | No | Yes | No |
| 248 | proteoglycan 4 | Prg4 | NM_001110146_at | No | Yes | No |
| | (megakaryocyte | | | | | |
| | stimulating factor, | | | | | |
| | articular superficial zone | | | | | |
| | protein) | | | | | |
| 249 | troponin I, skeletal, slow 1 | Tnni1 | NM_001112702_at | No | Yes | No |
| 250 | corin | Corin | NM_001122756_at | No | Yes | No |
| 251 | cytochrome P450, family | Cyp1a1 | NM_001136059_at | No | Yes | No |
| | 1, subfamily a, | | | | | |
| | polypeptide 1 | | | | | |
| 252 | enolase 3, beta muscle | Eno3 | NM_001136062_at | No | Yes | No |
| 253 | BCLl2-like 15 | Bcl2l15 | NM_001142959_at | No | Yes | No |
| 254 | BCLl2-like 15 | Bcl2l15 | NM_001142960_at | No | Yes | No |
| 255 | troponin T3, skeletal, | Tnnt3 | NM_001163664_at | No | Yes | No |
| | fast | | | | | |
| 256 | troponin T3, skeletal, | Tnnt3 | NM_001163665_at | No | Yes | No |
| | fast | | | | | |
| 257 | troponin T3, skeletal, | Tnnt3 | NM_001163666_at | No | Yes | No |
| | fast | | | | | |
| 258 | troponin T3, skeletal, | Tnnt3 | NM_001163667_at | No | Yes | No |
| | fast | | | | | |
| 259 | troponin T3, skeletal, | Tnnt3 | NM_001163668_at | No | Yes | No |
| | fast | | | | | |
| 260 | troponin T3, skeletal, | Tnnt3 | NM_001163669_at | No | Yes | No |
| | fast | | | | | |
| 261 | troponin T3, skeletal, | Tnnt3 | NM_001163670_at | No | Yes | No |
| | fast | | | | | |
| 262 | ribosomal protein L3- | Rpl3l | NM_001163945_at | No | Yes | No |
| | like | | | | | |
| 263 | myoglobin | Mb | NM_001164047_at | No | Yes | No |
| 264 | myoglobin | Mb | NM_001164048_at | No | Yes | No |
| 265 | RIKEN cDNA 1110032A04 | 1110032A04Rik | NM_001164210_at | No | Yes | No |
| | gene | | | | | |
| 266 | tropomyosin 1, alpha | Tpm1 | NM_001164248_at | No | Yes | No |
| 267 | tropomyosin 1, alpha | Tpm1 | NM_001164251_at | No | Yes | No |
| 268 | tropomyosin 1, alpha | Tpm1 | NM_001164254_at | No | Yes | No |
| 269 | tropomyosin 1, alpha | Tpm1 | NM_001164255_at | No | Yes | No |
| 270 | tropomyosin 1, alpha | Tpm1 | NM_001164256_at | No | Yes | No |
| 271 | ect2 oncogene | Ect2 | NM_001177625_at | No | Yes | No |
| 272 | ect2 oncogene | Ect2 | NM_001177626_at | No | Yes | No |
| 273 | C-type lectin domain | Clec4n | NM_001190320_at | No | Yes | No |
| | family 4, member n | | | | | |
| 274 | C-type lectin domain | Clec4n | NM_001190321_at | No | Yes | No |
| | family 4, member n | | | | | |
| 275 | cysteine and glycine-rich | Csrp3 | NM_001198841_at | No | Yes | No |
| | protein 3 | | | | | |
| 276 | predicted gene 10229 | Gm10229 | NM_001199334_at | No | Yes | No |
| 277 | aggrecan | Acan | NM_007424_at | No | Yes | No |
| 278 | ATPase, Ca++ | Atp2a1 | NM_007504_at | No | Yes | No |
| | transporting, cardiac | | | | | |
| | muscle, fast twitch 1 | | | | | |
| 279 | cyclin B2 | Ccnb2 | NM_007630_at | No | Yes | No |
| 280 | chondroadherin | Chad | NM_007689_at | No | Yes | No |
| 281 | chitinase 3-like 1 | Chi3l1 | NM_007695_at | No | Yes | No |
| 282 | creatine kinase, muscle | Ckm | NM_007710_at | No | Yes | No |
| 283 | collagen, type XI, alpha 1 | Col11a1 | NM_007729_at | No | Yes | No |
| 284 | collagen, type VIII, alpha 1 | Col8a1 | NM_007739_at | No | Yes | No |
| 285 | collagen, type IX, alpha 1 | Col9a1 | NM_007740_at | No | Yes | No |
| 286 | collagen, type I, alpha 1 | Col1a1 | NM_007742_at | No | Yes | No |
| 287 | cytochrome c oxidase, | Cox8b | NM_007751_at | No | Yes | No |
| | subunit VIIIb | | | | | |
| 288 | cytochrome P450, family | Cyp26a1 | NM_007811_at | No | Yes | No |
| | 26, subfamily a, | | | | | |
| | polypeptide 1 | | | | | |
| 289 | ect2 oncogene | Ect2 | NM_007900_at | No | Yes | No |
| 290 | enolase 3, beta muscle | Eno3 | NM_007933_at | No | Yes | No |
| 291 | fibroblast activation | Fap | NM_007986_at | No | Yes | No |
| | protein | | | | | |
| 292 | FBJ osteosarcoma | Fosb | NM_008036_at | No | Yes | No |
| | oncogene B | | | | | |
| 293 | formyl peptide receptor 2 | Fpr2 | NM_008039_at | No | Yes | No |
| 294 | G0/G1 switch gene 2 | G0s2 | NM_008059_at | No | Yes | No |
| 295 | growth differentiation | Gdf5 | NM_008109_at | No | Yes | No |
| | factor 5 | | | | | |
| 296 | interferon activated gene | Ifi204 | NM_008329_at | No | Yes | No |
| | 204 | | | | | |
| 297 | lymphotoxin B | Ltb | NM_008518_at | No | Yes | No |
| 298 | achaete-scute complex | Ascl2 | NM_008554_at | No | Yes | No |
| | homolog 2 (Drosophila) | | | | | |
| 299 | myomesin 2 | Myom2 | NM_008664_at | No | Yes | No |
| 300 | nebulin-related | Nrap | NM_008733_at | No | Yes | No |
| | anchoring protein | | | | | |
| 301 | prostaglandin I2 | Ptgis | NM_008968_at | No | Yes | No |
| | (prostacyclin) synthase | | | | | |
| 302 | brain expressed gene 1 | Bex1 | NM_009052_at | No | Yes | No |
| 303 | repetin | Rptn | NM_009100_at | No | Yes | No |
| 304 | ryanodine receptor 1, | Ryr1 | NM_009109_at | No | Yes | No |
| | skeletal muscle | | | | | |
| 305 | S100 protein, beta | S100b | NM_009115_at | No | Yes | No |
| | polypeptide, neural | | | | | |
| 306 | thyroid hormone | Thrsp | NM_009381_at | No | Yes | No |
| | responsive SPOT14 | | | | | |
| | homolog (Rattus) | | | | | |
| 307 | troponin C, cardiac/slow | Tnnc1 | NM_009393_at | No | Yes | No |
| | skeletal | | | | | |
| 308 | troponin C2, fast | Tnnc2 | NM_009394_at | No | Yes | No |
| 309 | troponin I, skeletal, fast 2 | Tnni2 | NM_009405_at | No | Yes | No |
| 310 | adiponectin, C1Q and | Adipoq | NM_009605_at | No | Yes | No |
| | collagen domain | | | | | |
| | containing | | | | | |
| 311 | actin, alpha 1, skeletal | Acta1 | NM_009606_at | No | Yes | No |
| | muscle | | | | | |
| 312 | actin, alpha, cardiac | Actc1 | NM_009608_at | No | Yes | No |
| | muscle 1 | | | | | |
| 313 | arachidonate 8- | Alox8 | NM_009661_at | No | Yes | No |
| | lipoxygenase | | | | | |
| 314 | apolipoprotein B mRNA | Apobec2 | NM_009694_at | No | Yes | No |
| | editing enzyme, catalytic | | | | | |
| | polypeptide 2 | | | | | |
| 315 | chitinase 3-like 3 | Chi3l3 | NM_009892_at | No | Yes | No |
| 316 | chemokine (C-C motif) | Ccr1 | NM_009912_at | No | Yes | No |
| | receptor 1 | | | | | |
| 317 | collagen, type IX, alpha 3 | Col9a3 | NM_009936_at | No | Yes | No |
| 318 | cytochrome c oxidase, | Cox6a2 | NM_009943_at | No | Yes | No |
| | subunit VI a, | | | | | |
| | polypeptide 2 | | | | | |
| 319 | cytochrome c oxidase, | Cox7a1 | NM_009944_at | No | Yes | No |
| | subunit VIIa 1 | | | | | |
| 320 | cytochrome P450, family | Cyp1a1 | NM_009992_at | No | Yes | No |
| | 1, subfamily a, | | | | | |
| | polypeptide 1 | | | | | |
| 321 | Fc receptor, IgE, high | Fcer1a | NM_010184_at | No | Yes | No |
| | affinity I, alpha | | | | | |
| | polypeptide | | | | | |
| 322 | fibronectin 1 | Fn1 | NM_010233_at | No | Yes | No |
| 323 | FBJ osteosarcoma | Fos | NM_010234_at | No | Yes | No |
| | oncogene | | | | | |
| 324 | frizzled homolog 9 | Fzd9 | NM_010246_at | No | Yes | No |
| | (Drosophila) | | | | | |
| 325 | keratin 86 | Krt86 | NM_010667_at | No | Yes | No |
| 326 | keratin 6B | Krt6b | NM_010669_at | No | Yes | No |
| 327 | keratin associated | Krtap6-1 | NM_010672_at | No | Yes | No |
| | protein 6-1 | | | | | |
| 328 | keratin associated | Krtap6-2 | NM_010673_at | No | Yes | No |
| | protein 6-2 | | | | | |
| 329 | keratin associated | Krtap8-1 | NM_010675_at | No | Yes | No |
| | protein 8-1 | | | | | |
| 330 | keratin associated | Krtap8-2 | NM_010676_at | No | Yes | No |
| | protein 8-2 | | | | | |
| 331 | leukocyte cell derived | Lect1 | NM_010701_at | No | Yes | No |
| | chemotaxin 1 | | | | | |
| 332 | lipase, endothelial | Lipg | NM_010720_at | No | Yes | No |
| 333 | myosin, heavy | Myh4 | NM_010855_at | No | Yes | No |
| | polypeptide 4, skeletal | | | | | |
| | muscle | | | | | |
| 334 | myosin, light | Myl2 | NM_010861_at | No | Yes | No |
| | polypeptide 2, | | | | | |
| | regulatory, cardiac, slow | | | | | |
| 335 | myocilin | Myoc | NM_010865_at | No | Yes | No |
| 336 | myomesin 1 | Myom1 | NM_010867_at | No | Yes | No |
| 337 | phosphoenolpyruvate | Pck1 | NM_011044_at | No | Yes | No |
| | carboxykinase 1, | | | | | |
| | cytosolic | | | | | |
| 338 | prostaglandin- | Ptgs2 | NM_011198_at | No | Yes | No |
| | endoperoxide synthase 2 | | | | | |
| 339 | chemokine (C-C motif) | Ccl11 | NM_011330_at | No | Yes | No |
| | ligand 11 | | | | | |
| 340 | selectin, endothelial cell | Sele | NM_011345_at | No | Yes | No |
| 341 | selectin, platelet | Selp | NM_011347_at | No | Yes | No |
| 342 | frizzled-related protein | Frzb | NM_011356_at | No | Yes | No |
| 343 | small proline-rich | Sprr2d | NM_011470_at | No | Yes | No |
| | protein 2D | | | | | |
| 344 | small proline-rich | Sprr2f | NM_011472_at | No | Yes | No |
| | protein 2F | | | | | |
| 345 | small proline-rich | Sprr2h | NM_011474_at | No | Yes | No |
| | protein 2H | | | | | |
| 346 | small proline-rich | Sprr2i | NM_011475_at | No | Yes | No |
| | protein 2I | | | | | |
| 347 | titin-cap | Tcap | NM_011540_at | No | Yes | No |
| 348 | thrombospondin 4 | Thbs4 | NM_011582_at | No | Yes | No |
| 349 | troponin T1, skeletal, | Tnnt1 | NM_011618_at | No | Yes | No |
| | slow | | | | | |
| 350 | troponin T3, skeletal, | Tnnt3 | NM_011620_at | No | Yes | No |
| | fast | | | | | |
| 351 | titin | Ttn | NM_011652_at | No | Yes | No |
| 352 | secretoglobin, family 1A, | Scgb1a1 | NM_011681_at | No | Yes | No |
| | member 1 (uteroglobin) | | | | | |
| 353 | xin actin-binding repeat | Xirp1 | NM_011724_at | No | Yes | No |
| | containing 1 | | | | | |
| 354 | Wnt inhibitory factor 1 | Wif1 | NM_011915_at | No | Yes | No |
| 355 | osteomodulin | Omd | NM_012050_at | No | Yes | No |
| 356 | actinin alpha 3 | Actn3 | NM_013456_at | No | Yes | No |
| 357 | complement factor D | Cfd | NM_013459_at | No | Yes | No |
| | (adipsin) | | | | | |
| 358 | keratin 33B | Krt33b | NM_013570_at | No | Yes | No |
| 359 | myoglobin | Mb | NM_013593_at | No | Yes | No |
| 360 | parvalbumin | Pvalb | NM_013645_at | No | Yes | No |
| 361 | S100 calcium binding | S100a8 | NM_013650_at | No | Yes | No |
| | protein A8 (calgranulin | | | | | |
| | A) | | | | | |
| 362 | transthyretin | Ttr | NM_013697_at | No | Yes | No |
| 363 | keratin associated | Krtap14 | NM_013707_at | No | Yes | No |
| | protein 14 | | | | | |
| 364 | keratin associated | Krtap15 | NM_013713_at | No | Yes | No |
| | protein 15 | | | | | |
| 365 | defensin beta 3 | Defb3 | NM_013756_at | No | Yes | No |
| 366 | cysteine and glycine-rich | Csrp3 | NM_013808_at | No | Yes | No |
| | protein 3 | | | | | |
| 367 | antigen p97 (melanoma | Mfi2 | NM_013900_at | No | Yes | No |
| | associated) identified by | | | | | |
| | monoclonal antibodies | | | | | |
| | 133.2 and 96.5 | | | | | |
| 368 | SH3-binding domain | Sh3bgr | NM_015825_at | No | Yes | No |
| | glutamic acid-rich | | | | | |
| | protein | | | | | |
| 369 | cartilage oligomeric | Comp | NM_016685_at | No | Yes | No |
| | matrix protein | | | | | |
| 370 | myosin light chain, | Mylpf | NM_016754_at | No | Yes | No |
| | phosphorylatable, fast | | | | | |
| | skeletal muscle | | | | | |
| 371 | corin | Corin | NM_016869_at | No | Yes | No |
| 372 | D site albumin promoter | Dbp | NM_016974_at | No | Yes | No |
| | binding protein | | | | | |
| 373 | lysozyme 2 | Lyz2 | NM_017372_at | No | Yes | No |
| 374 | claudin 8 | Cldn8 | NM_018778_at | No | Yes | No |
| 375 | phosphoglycerate | Pgam2 | NM_018870_at | No | Yes | No |
| | mutase 2 | | | | | |
| 376 | melanoma inhibitory | Mia1 | NM_019394_at | No | Yes | No |
| | activity 1 | | | | | |
| 377 | eosinophil-associated, | Ear5 | NM_019398_at | No | Yes | No |
| | ribonuclease A family, | | | | | |
| | member 5 | | | | | |
| 378 | chemokine (C-C motif) | Ccl24 | NM_019577_at | No | Yes | No |
| | ligand 24 | | | | | |
| 379 | C-type lectin domain | Clec4n | NM_020001_at | No | Yes | No |
| | family 4, member n | | | | | |
| 380 | resistin like alpha | Retnla | NM_020509_at | No | Yes | No |
| 381 | chemokine ( C—X—C motif) | Cxcl10 | NM_021274_at | No | Yes | No |
| | ligand 10 | | | | | |
| 382 | proteoglycan 4 | Prg4 | NM_021400_at | No | Yes | No |
| | (megakaryocyte | | | | | |
| | stimulating factor, | | | | | |
| | articular superficial zone | | | | | |
| | protein) | | | | | |
| 383 | chemokine (C-C motif) | Ccl8 | NM_021443_at | No | Yes | No |
| | ligand 8 | | | | | |
| 384 | troponin I, skeletal, slow 1 | Tnni1 | NM_021467_at | No | Yes | No |
| 385 | myozenin 2 | Myoz2 | NM_021503_at | No | Yes | No |
| 386 | keratin associated | Krtap3-1 | NM_023511_at | No | Yes | No |
| | protein 3-1 | | | | | |
| 387 | aldehyde oxidase 4 | Aox4 | NM_023631_at | No | Yes | No |
| 388 | cardiomyopathy | Cmya5 | NM_023821_at | No | Yes | No |
| | associated 5 | | | | | |
| 389 | RIKEN cDNA 1500015010 | 1500015010Rik | NM_024283_at | No | Yes | No |
| | gene | | | | | |
| 390 | small muscle protein, X- | Smpx | NM_025357_at | No | Yes | No |
| | linked | | | | | |
| 391 | ribosomal protein L3- | Rpl3l | NM_025425_at | No | Yes | No |
| | like | | | | | |
| 392 | keratin associated | Krtap3-3 | NM_025524_at | No | Yes | No |
| | protein 3-3 | | | | | |
| 393 | sarcolipin | Sln | NM_025540_at | No | Yes | No |
| 394 | WAP four-disulfide core | Wfdc2 | NM_026323_at | No | Yes | No |
| | domain 2 | | | | | |
| 395 | keratin associated | Krtap4-2 | NM_026807_at | No | Yes | No |
| | protein 4-2 | | | | | |
| 396 | membrane-spanning 4- | Ms4a6d | NM_026835_at | No | Yes | No |
| | domains, subfamily A, | | | | | |
| | member 6D | | | | | |
| 397 | keratin associated | Krtap1-5 | NM_027157_at | No | Yes | No |
| | protein 1-5 | | | | | |
| 398 | keratin 34 | Krt34 | NM_027563_at | No | Yes | No |
| 399 | keratin associated | Krtap5-2 | NM_027844_at | No | Yes | No |
| | protein 5-2 | | | | | |
| 400 | keratin 33A | Krt33a | NM_027983_at | No | Yes | No |
| 401 | titin | Ttn | NM_028004_at | No | Yes | No |
| 402 | RIKEN cDNA 3110079015 | 3110079015Rik | NM_028473_at | No | Yes | No |
| | gene | | | | | |
| 403 | keratin associated | Krtap16-7 | NM_028621_at | No | Yes | No |
| | protein 16-7 | | | | | |
| 404 | keratin associated | Krtap9-3 | NM_029351_at | No | Yes | No |
| | protein 9-3 | | | | | |
| 405 | ankyrin repeat and SOCs | Asb5 | NM_029569_at | No | Yes | No |
| | box-containing 5 | | | | | |
| 406 | triadin | Trdn | NM_029726_at | No | Yes | No |
| 407 | chemokine ( C—X—C motif) | Cxcr6 | NM_030712_at | No | Yes | No |
| | receptor 6 | | | | | |
| 408 | gasdermin C | Gsdmc | NM_031378_at | No | Yes | No |
| 409 | cysteine-rich secretory | Crispld1 | NM_031402_at | No | Yes | No |
| | protein LCCL domain | | | | | |
| | containing 1 | | | | | |
| 410 | actinin alpha 2 | Actn2 | NM_033268_at | No | Yes | No |
| 411 | leiomodin 2 (cardiac) | Lmod2 | NM_053098_at | No | Yes | No |
| 412 | keratin associated | Krtap16-8 | NM_130856_at | No | Yes | No |
| | protein 16-8 | | | | | |
| 413 | keratin associated | Krtap16-5 | NM_130857_at | No | Yes | No |
| | protein 16-5 | | | | | |
| 414 | keratin associated | Krtap16-1 | NM_130870_at | No | Yes | No |
| | protein 16-1 | | | | | |
| 415 | keratin associated | Krtap16-9 | NM_130876_at | No | Yes | No |
| | protein 16-9 | | | | | |
| 416 | CD209d antigen | Cd209d | NM_130904_at | No | Yes | No |
| 417 | cDNA sequence | AY026312 | NM_133359_at | No | Yes | No |
| | AY026312 | | | | | |
| 418 | RIKEN cDNA 1110032A04 | 1110032A04Rik | NM_133675_at | No | Yes | No |
| | gene | | | | | |
| 419 | nucleolar and spindle | Nusap1 | NM_133851_at | No | Yes | No |
| | associated protein 1 | | | | | |
| 420 | transferrin | Trf | NM_133977_at | No | Yes | No |
| 421 | aldo-keto reductase | Akr1c14 | NM_134072_at | No | Yes | No |
| | family 1, member C14 | | | | | |
| 422 | oxidized low density | Olr1 | NM_138648_at | No | Yes | No |
| | lipoprotein (lectin-like) | | | | | |
| | receptor 1 | | | | | |
| 423 | alpha-2-macroglobulin | A2m | NM_175628_at | No | Yes | No |
| 424 | keratin associated | Krtap13-1 | NM_183189_at | No | Yes | No |
| | protein 13-1 | | | | | |
| 425 | keratin associated | Krtap16-10 | NM_183296_at | No | Yes | No |
| | protein 16-10 | | | | | |
| 426 | nebulin-related | Nrap | NM_198059_at | No | Yes | No |
| | anchoring protein | | | | | |
| 427 | creatine kinase, | Ckmt2 | NM_198415_at | No | Yes | No |
| | mitochondrial 2 | | | | | |
| 428 | H19 fetal liver mRNA | H19 | NR_001592_at | No | Yes | No |
| 429 | 18S ribosomal RNA | Rn18s | NR_003278_at | No | Yes | No |
| 430 | predicted gene 10228 | Gm10228 | XM_001474297_at | No | Yes | No |
| 431 | c-C motif chemokine 8- | LOC100503254 | XM_003085741_at | No | Yes | No |
| | like | | | | | |
| 432 | predicted gene 10229 | Gm10229 | XM_003086003_at | No | Yes | No |
| 433 | predicted gene 10228 | Gm10228 | XM_003086004_at | No | Yes | No |
| 434 | keratin associated | Krtap28-13 | XM_896507_at | No | Yes | No |
| | protein 28-13 | | | | | |
| 435 | keratin associated | Krtap28-13 | XM_919110_at | No | Yes | No |
| | protein 28-13 | | | | | |
| 436 | RIKEN cDNA 2610528A11 | 2610528A11Rik | XR_105647_at | No | Yes | No |
| | gene | | | | | |
| 437 | RIKEN cDNA 2510009E07 | 2510009E07Rik | NM_001001881_at | No | No | Yes |
| | gene | | | | | |
| 438 | ring finger and FYVE | Rffl | NM_001007465_at | No | No | Yes |
| | like domain containing | | | | | |
| | protein | | | | | |
| 439 | zinc finger protein 187 | Zfp187 | NM_001013786_at | No | No | Yes |
| 440 | zinc finger prtoein 943 | Zfp943 | NM_001025373_at | No | No | Yes |
| 441 | interleukin 1 receptor- | Il1rl1 | NM_001025602_at | No | No | Yes |
| | like 1 | | | | | |
| 442 | RIKEN cDNA 2510002D24 | 2510002D24Rik | NM_001033164_at | No | No | Yes |
| | gene | | | | | |
| 443 | nuclear cap binding | Ncbp1 | NM_001033201_at | No | No | Yes |
| | protein subunit 1 | | | | | |
| 444 | family with sequence | Fam134b | NM_001034851_at | No | No | Yes |
| | similarity 134, member B | | | | | |
| 445 | DEP domain containing | Deptor | NM_001037937_at | No | No | Yes |
| | MTOR-interacting protein | | | | | |
| 446 | calcium/calmodulin- | Camk2g | NM_001039138_at | No | No | Yes |
| | dependent protein | | | | | |
| | kinase II gamma | | | | | |
| 447 | calcium/calmodulin- | Camk2g | NM_001039139_at | No | No | Yes |
| | dependent protein | | | | | |
| | kinase II gamma | | | | | |
| 448 | nucleolar and coiled- | Nolc1 | NM_001039351_at | No | No | Yes |
| | body phosphoprotein 1 | | | | | |
| 449 | nucleolar and coiled- | Nolc1 | NM_001039352_at | No | No | Yes |
| | body phosphoprotein 1 | | | | | |
| 450 | nucleolar and coiled- | Nolc1 | NM_001039353_at | No | No | Yes |
| | body phosphoprotein 1 | | | | | |
| 451 | discs, large homolog- | Dlgap4 | NM_001042487_at | No | No | Yes |
| | associated protein 4 | | | | | |
| | (Drosophila) | | | | | |
| 452 | discs, large homolog- | Dlgap4 | NM_001042488_at | No | No | Yes |
| | associated protein 4 | | | | | |
| | (Drosophila) | | | | | |
| 453 | glutaryl-Coenzyme A | Gcdh | NM_001044744_at | No | No | Yes |
| | dehydrogenase | | | | | |
| 454 | zinc finger protein 68 | Zfp68 | NM_001044747_at | No | No | Yes |
| 455 | tumor necrosis factor | Tnfrsf9 | NM_001077508_at | No | No | Yes |
| | receptor superfamily, | | | | | |
| | member 9 | | | | | |
| 456 | tumor necrosis factor | Tnfrsf9 | NM_001077509_at | No | No | Yes |
| | receptor superfamily, | | | | | |
| | member 9 | | | | | |
| 457 | opioid growth factor | Ogfrl1 | NM_001081079_at | No | No | Yes |
| | receptor-like 1 | | | | | |
| 458 | zinc finger protein 518B | Zfp518b | NM_001081144_at | No | No | Yes |
| 459 | mitochondrial ribosomal | Mrpl44 | NM_001081210_at | No | No | Yes |
| | protein L44 | | | | | |
| 460 | kelch domain containing 5 | Klhdc5 | NM_001081237_at | No | No | Yes |
| 461 | malignant fibrous | Mfhas1 | NM_001081279_at | No | No | Yes |
| | histiocytoma amplified | | | | | |
| | sequence 1 | | | | | |
| 462 | breakpoint cluster | Bcr | NM_001081412_at | No | No | Yes |
| | region | | | | | |
| 463 | phosphatidylinositol | Pigyl | NM_001082532_at | No | No | Yes |
| | glycan anchor | | | | | |
| | biosynthesis, class Y-like | | | | | |
| 464 | WW domain binding | Wbp1 | NM_001083922_at | No | No | Yes |
| | protein 1 | | | | | |
| 465 | WW domain binding | Wbp1 | NM_001083923_at | No | No | Yes |
| | protein 1 | | | | | |
| 466 | peptidyl prolyl | Ppih | NM_001110129_at | No | No | Yes |
| | isomerase H | | | | | |
| 467 | ATPase, Ca++ | Atp2a2 | NM_001110140_at | No | No | Yes |
| | transporting, cardiac | | | | | |
| | muscle, slow twitch 2 | | | | | |
| 468 | inositol polyphosphate- | Inpp5d | NM_001110192_at | No | No | Yes |
| | 5-phosphatase D | | | | | |
| 469 | inositol polyphosphate- | Inpp5d | NM_001110193_at | No | No | Yes |
| | 5-phosphatase D | | | | | |
| 470 | cytohesin 1 | Cyth1 | NM_001112699_at | No | No | Yes |
| 471 | cytohesin 1 | Cyth1 | NM_001112700_at | No | No | Yes |
| 472 | major facilitator | Mfsd4 | NM_001114662_at | No | No | Yes |
| | superfamily domain | | | | | |
| | containing 4 | | | | | |
| 473 | transformation related | Trp63 | NM_001127259_at | No | No | Yes |
| | protein 63 | | | | | |
| 474 | transformation related | Trp63 | NM_001127260_at | No | No | Yes |
| | protein 63 | | | | | |
| 475 | transformation related | Trp63 | NM_001127262_at | No | No | Yes |
| | protein 63 | | | | | |
| 476 | transformation related | Trp63 | NM_001127264_at | No | No | Yes |
| | protein 63 | | | | | |
| 477 | peroxisome proliferator | Pparg | NM_001127330_at | No | No | Yes |
| | activated receptor | | | | | |
| | gamma | | | | | |
| 478 | aldehyde dehydrogenase | Aldh7a1 | NM_001127338_at | No | No | Yes |
| | family 7, member A1 | | | | | |
| 479 | inositol polyphosphate- | Inpp5a | NM_001127363_at | No | No | Yes |
| | 5-phosphatase A | | | | | |
| 480 | ATPase type 13A3 | Atp13a3 | NM_001128094_at | No | No | Yes |
| 481 | ATPase type 13A3 | Atp13a3 | NM_001128096_at | No | No | Yes |
| 482 | oxidation resistance 1 | Oxr1 | NM_001130163_at | No | No | Yes |
| 483 | oxidation resistance 1 | Oxr1 | NM_001130164_at | No | No | Yes |
| 484 | oxidation resistance 1 | Oxr1 | NM_001130165_at | No | No | Yes |
| 485 | oxidation resistance 1 | Oxr1 | NM_001130166_at | No | No | Yes |
| 486 | solute carrier family 39 | Slc39a14 | NM_001135151_at | No | No | Yes |
| | (zinc transporter), | | | | | |
| | member 14 | | | | | |
| 487 | solute carrier family 39 | Slc39a14 | NM_001135152_at | No | No | Yes |
| | (zinc transporter), | | | | | |
| | member 14 | | | | | |
| 488 | transmembrane protein | Tmem80 | NM_001141950_at | No | No | Yes |
| | 80 | | | | | |
| 489 | RIKEN cDNA 5730494N06 | 5730494N06Rik | NM_001141971_at | No | No | Yes |
| | gene | | | | | |
| 490 | RNA binding motif | Rbm43 | NM_001141981_at | No | No | Yes |
| | protein 43 | | | | | |
| 491 | RNA binding motif | Rbm43 | NM_001141982_at | No | No | Yes |
| | protein 43 | | | | | |
| 492 | LIM domain only 2 | Lmo2 | NM_001142335_at | No | No | Yes |
| 493 | LIM domain only 2 | Lmo2 | NM_001142336_at | No | No | Yes |
| 494 | LIM domain only 2 | Lmo2 | NM_001142337_at | No | No | Yes |
| 495 | zinc finger with KRAB | Zkscan3 | NM_001145778_at | No | No | Yes |
| | and SCAN domains 3 | | | | | |
| 496 | ral guanine nucleotide | Ralgds | NM_001145834_at | No | No | Yes |
| | dissociation stimulator | | | | | |
| 497 | ral guanine nucleotide | Ralgds | NM_001145835_at | No | No | Yes |
| | dissociation stimulator | | | | | |
| 498 | ral guanine nucleotide | Ralgds | NM_001145836_at | No | No | Yes |
| | dissociation stimulator | | | | | |
| 499 | von Willebrand factor A | Vwa5a | NM_001145957_at | No | No | Yes |
| | domain containing 5A | | | | | |
| 500 | tumor necrosis factor, | Tnfaip1 | NM_001159392_at | No | No | Yes |
| | alpha-induced protein 1 | | | | | |
| | (endothelial) | | | | | |
| 501 | origin recognition | Orc3 | NM_001159563_at | No | No | Yes |
| | complex, subunit 3 | | | | | |
| 502 | sirtuin 1 (silent mating | Sirt1 | NM_001159589_at | No | No | Yes |
| | type information | | | | | |
| | regulation 2, homolog) 1 | | | | | |
| | (S. cerevisiae) | | | | | |
| 503 | sirtuin 1 (silent mating | Sirt1 | NM_001159590_at | No | No | Yes |
| | type information | | | | | |
| | regulation 2, homolog) 1 | | | | | |
| | (S. cerevisiae) | | | | | |
| 504 | phosphatidylinositol | Pigp | NM_001159616_at | No | No | Yes |
| | glycan anchor | | | | | |
| | biosynthesis, class P | | | | | |
| 505 | phosphatidylinositol | Pigp | NM_001159617_at | No | No | Yes |
| | glycan anchor | | | | | |
| | biosynthesis, class P | | | | | |
| 506 | phosphatidylinositol | Pigp | NM_001159618_at | No | No | Yes |
| | glycan anchor | | | | | |
| | biosynthesis, class P | | | | | |
| 507 | phosphatidylinositol | Pigp | NM_001159619_at | No | No | Yes |
| | glycan anchor | | | | | |
| | biosynthesis, class P | | | | | |
| 508 | poliovirus receptor- | Pvrl2 | NM_001159724_at | No | No | Yes |
| | related 2 | | | | | |
| 509 | flavin containing | Fmo5 | NM_001161763_at | No | No | Yes |
| | monooxygenase 5 | | | | | |
| 510 | flavin containing | Fmo5 | NM_001161765_at | No | No | Yes |
| | monooxygenase 5 | | | | | |
| 511 | SWI/SNF related, matrix | Smarcb1 | NM_001161853_at | No | No | Yes |
| | associated, actin | | | | | |
| | dependent regulator of | | | | | |
| | chromatin, subfamily b, | | | | | |
| | member 1 | | | | | |
| 512 | NIMA (never in mitosis | Nek3 | NM_001162947_at | No | No | Yes |
| | gene a)-related | | | | | |
| | expressed kinase 3 | | | | | |
| 513 | RIKEN cDNA 4933407C03 | 4933407C03Rik | NM_001163262_at | No | No | Yes |
| | gene | | | | | |
| 514 | putative homeodomain | Phtf1 | NM_001163467_at | No | No | Yes |
| | transcription factor 1 | | | | | |
| 515 | putative homeodomain | Phtf1 | NM_001163468_at | No | No | Yes |
| | transcription factor 1 | | | | | |
| 516 | putative homeodomain | Phtf1 | NM_001163469_at | No | No | Yes |
| | transcription factor 1 | | | | | |
| 517 | tankyrase, TRF1- | Tnks2 | NM_001163635_at | No | No | Yes |
| | interacting ankyrin- | | | | | |
| | related ADP-ribose | | | | | |
| | polymerase 2 | | | | | |
| 518 | ribosomal RNA | Rrp1b | NM_001163734_at | No | No | Yes |
| | processing 1 homolog B | | | | | |
| | (S. cerevisiae) | | | | | |
| 519 | zinc finger protein 68 | Zfp68 | NM_001163797_at | No | No | Yes |
| 520 | N-acetylglucosamine | Nagk | NM_001164187_at | No | No | Yes |
| | kinase | | | | | |
| 521 | ring finger and FYVE | Rffl | NM_001164569_at | No | No | Yes |
| | like domain containing | | | | | |
| | protein | | | | | |
| 522 | ring finger and FYVE | Rffl | NM_001164570_at | No | No | Yes |
| | like domain containing | | | | | |
| | protein | | | | | |
| 523 | SNF related kinase | Snrk | NM_001164572_at | No | No | Yes |
| 524 | coronin, actin binding | Coro2a | NM_001164804_at | No | No | Yes |
| | protein 2A | | | | | |
| 525 | leucine rich repeat (in | Lrrfip2 | NM_001164838_at | No | No | Yes |
| | FLII) interacting protein 2 | | | | | |
| 526 | armadillo repeat | Armcx1 | NM_001166377_at | No | No | Yes |
| | containing, X-linked 1 | | | | | |
| 527 | armadillo repeat | Armcx1 | NM_001166378_at | No | No | Yes |
| | containing, X-linked 1 | | | | | |
| 528 | armadillo repeat | Armcx1 | NM_001166379_at | No | No | Yes |
| | containing, X-linked 1 | | | | | |
| 529 | armadillo repeat | Armcx1 | NM_001166380_at | No | No | Yes |
| | containing, X-linked 1 | | | | | |
| 530 | mediator complex | Med23 | NM_001166416_at | No | No | Yes |
| | subunit 23 | | | | | |
| 531 | suppressor of | Suv420h1 | NM_001167885_at | No | No | Yes |
| | variegation 4-20 | | | | | |
| | homolog 1 (Drosophila) | | | | | |
| 532 | suppressor of | Suv420h1 | NM_001167886_at | No | No | Yes |
| | variegation 4-20 | | | | | |
| | homolog 1 (Drosophila) | | | | | |
| 533 | suppressor of | Suv420h1 | NM_001167887_at | No | No | Yes |
| | variegation 4-20 | | | | | |
| | homolog 1 (Drosophila) | | | | | |
| 534 | suppressor of | Suv420h1 | NM_001167889_at | No | No | Yes |
| | variegation 4-20 | | | | | |
| | homolog 1 (Drosophila) | | | | | |
| 535 | transmembrane protein | Tmem93 | NM_001168470_at | No | No | Yes |
| | 93 | | | | | |
| 536 | nuclear factor of kappa | Nfkb2 | NM_001177369_at | No | No | Yes |
| | light polypeptide gene | | | | | |
| | enhancer in B-cells 2, | | | | | |
| | p49/p100 | | | | | |
| 537 | nuclear factor of kappa | Nfkb2 | NM_001177370_at | No | No | Yes |
| | light polypeptide gene | | | | | |
| | enhancer in B-cells 2, | | | | | |
| | p49/p100 | | | | | |
| 538 | zinc finger protein 518B | Zfp518b | NM_001177902_at | No | No | Yes |
| 539 | cerebral cavernous | Ccm2 | NM_001190343_at | No | No | Yes |
| | malformation 2 homolog | | | | | |
| | (human) | | | | | |
| 540 | cerebral cavernous | Ccm2 | NM_001190344_at | No | No | Yes |
| | malformation 2 homolog | | | | | |
| | (human) | | | | | |
| 541 | Meis homeobox 1 | Meis1 | NM_001193271_at | No | No | Yes |
| 542 | breast cancer anti- | Bcar1 | NM_001198839_at | No | No | Yes |
| | estrogen resistance 1 | | | | | |
| 543 | DDB1 and CUL4 | Dcaf11 | NM_001199009_at | No | No | Yes |
| | associated factor 11 | | | | | |
| 544 | RAB guanine nucleotide | Rabgef1 | NM_001199059_at | No | No | Yes |
| | exchange factor (GEF) 1 | | | | | |
| 545 | RIKEN cDNA 4931408A02 | 4931408A02Rik | NM_001199210_at | No | No | Yes |
| | gene | | | | | |
| 546 | caveolin 1, caveolae | Cav1 | NM_007616_at | No | No | Yes |
| | protein | | | | | |
| 547 | cyclin-dependent kinase | Cdkn2c | NM_007671_at | No | No | Yes |
| | inhibitor 2C (p18, | | | | | |
| | inhibits CDK4) | | | | | |
| 548 | chromodomain helicase | Chd1 | NM_007690_at | No | No | Yes |
| | DNA binding protein 1 | | | | | |
| 549 | cytochrome c oxidase, | Cox8a | NM_007750_at | No | No | Yes |
| | subunit VIIIa | | | | | |
| 550 | enhancer of polycomb | Epc1 | NM_007935_at | No | No | Yes |
| | homolog 1 (Drosophila) | | | | | |
| 551 | myelin protein zero-like 2 | Mpzl2 | NM_007962_at | No | No | Yes |
| 552 | farnesyltransferase, CAAX | Fnta | NM_008033_at | No | No | Yes |
| | box, alpha | | | | | |
| 553 | glutaryl-Coenzyme A | Gcdh | NM_008097_at | No | No | Yes |
| | dehydrogenase | | | | | |
| 554 | glucosaminyl (N-acetyl) | Gcnt2 | NM_008105_at | No | No | Yes |
| | transferase 2, I- | | | | | |
| | branching enzyme | | | | | |
| 555 | nuclear receptor | Nr3c1 | NM_008173_at | No | No | Yes |
| | subfamily 3, group C, | | | | | |
| | member 1 | | | | | |
| 556 | hematopoietically | Hhex | NM_008245_at | No | No | Yes |
| | expressed homeobox | | | | | |
| 557 | interleukin 10 receptor, | Il10ra | NM_008348_at | No | No | Yes |
| | alpha | | | | | |
| 558 | interleukin 7 | Il7 | NM_008371_at | No | No | Yes |
| 559 | kinesin family member | Kif3a | NM_008443_at | No | No | Yes |
| | 3A | | | | | |
| 560 | LIM domain only 2 | Lmo2 | NM_008505_at | No | No | Yes |
| 561 | myeloid cell leukemia | Mcl1 | NM_008562_at | No | No | Yes |
| | sequence 1 | | | | | |
| 562 | mesenchyme homeobox 2 | Meox2 | NM_008584_at | No | No | Yes |
| 563 | metal response element | Mtf1 | NM_008636_at | No | No | Yes |
| | binding transcription | | | | | |
| | factor 1 | | | | | |
| 564 | methylmalonyl- | Mut | NM_008650_at | No | No | Yes |
| | Coenzyme A mutase | | | | | |
| 565 | protein phosphatase 1, | Ppp1r15a | NM_008654_at | No | No | Yes |
| | regulatory (inhibitor) | | | | | |
| | subunit 15A | | | | | |
| 566 | neutrophil cytosolic | Ncf4 | NM_008677_at | No | No | Yes |
| | factor 4 | | | | | |
| 567 | nitric oxide synthase 3, | Nos3 | NM_008713_at | No | No | Yes |
| | endothelial cell | | | | | |
| 568 | phosphatidylinositol | Pigf | NM_008838_at | No | No | Yes |
| | glycan anchor | | | | | |
| | biosynthesis, class F | | | | | |
| 569 | mitogen-activated | Map2k3 | NM_008928_at | No | No | Yes |
| | protein kinase kinase 3 | | | | | |
| 570 | RAD50 homolog (S. cerevisiae) | Rad50 | NM_009012_at | No | No | Yes |
| 571 | ral guanine nucleotide | Ralgds | NM_009058_at | No | No | Yes |
| | dissociation stimulator | | | | | |
| 572 | helicase-like | Hltf | NM_009210_at | No | No | Yes |
| | transcription factor | | | | | |
| 573 | serine (or cysteine) | Serpina3n | NM_009252_at | No | No | Yes |
| | peptidase inhibitor, | | | | | |
| | clade A, member 3N | | | | | |
| 574 | tumor necrosis factor, | Tnfaip1 | NM_009395_at | No | No | Yes |
| | alpha-induced protein 1 | | | | | |
| | (endothelial) | | | | | |
| 575 | tubulin, alpha 4A | Tuba4a | NM_009447_at | No | No | Yes |
| 576 | zinc finger protein 101 | Zfp101 | NM_009542_at | No | No | Yes |
| 577 | poly (ADP-ribose) | Parp2 | NM_009632_at | No | No | Yes |
| | polymerase family, | | | | | |
| | member 2 | | | | | |
| 578 | aldehyde dehydrogenase | Aldh2 | NM_009656_at | No | No | Yes |
| | 2, mitochondrial | | | | | |
| 579 | CCAAT/enhancer binding | Cebpb | NM_009883_at | No | No | Yes |
| | protein (C/EBP), beta | | | | | |
| 580 | breast cancer anti- | Bcar1 | NM_009954_at | No | No | Yes |
| | estrogen resistance 1 | | | | | |
| 581 | enoyl-Coenzyme A delta | Eci1 | NM_010023_at | No | No | Yes |
| | isomerase 1 | | | | | |
| 582 | D-dopachrome | Ddt | NM_010027_at | No | No | Yes |
| | tautomerase | | | | | |
| 583 | early growth response 2 | Egr2 | NM_010118_at | No | No | Yes |
| 584 | coagulation factor II | F2r | NM_010169_at | No | No | Yes |
| | (thrombin) receptor | | | | | |
| 585 | fibroblast growth factor | Fgf13 | NM_010200_at | No | No | Yes |
| | 13 | | | | | |
| 586 | FMS-like tyrosine kinase 1 | Flt1 | NM_010228_at | No | No | Yes |
| 587 | flavin containing | Fmo5 | NM_010232_at | No | No | Yes |
| | monooxygenase 5 | | | | | |
| 588 | glutathione transferase | Gstz1 | NM_010363_at | No | No | Yes |
| | zeta 1 | | | | | |
| | (maleylacetoacetate | | | | | |
| | isomerase) | | | | | |
| 589 | interferon gamma | Ifngr1 | NM_010511_at | No | No | Yes |
| | receptor 1 | | | | | |
| 590 | inositol polyphosphate- | Inpp5d | NM_010566_at | No | No | Yes |
| | 5-phosphatase D | | | | | |
| 591 | eukaryotic translation | Eif6 | NM_010579_at | No | No | Yes |
| | initiation factor 6 | | | | | |
| 592 | IL2-inducible T-cell | Itk | NM_010583_at | No | No | Yes |
| | kinase | | | | | |
| 593 | keratin 17 | Krt17 | NM_010663_at | No | No | Yes |
| 594 | transmembrane emp24 | Tmed1 | NM_010744_at | No | No | Yes |
| | domain containing 1 | | | | | |
| 595 | Meis homeobox 1 | Meis1 | NM_010789_at | No | No | Yes |
| 596 | mature T-cell | Mtcp1 | NM_010839_at | No | No | Yes |
| | Proliferation 1 | | | | | |
| 597 | myeloid differentiation | Myd88 | NM_010851_at | No | No | Yes |
| | primary response gene | | | | | |
| | 88 | | | | | |
| 598 | peptidyl arginine | Padi1 | NM_011059_at | No | No | Yes |
| | deiminase, type I | | | | | |
| 599 | prefoldin 2 | Pfdn2 | NM_011070_at | No | No | Yes |
| 600 | peroxisome proliferator | Pparg | NM_011146_at | No | No | Yes |
| | activated receptor | | | | | |
| | gamma | | | | | |
| 601 | cytohesin 1 | Cyth1 | NM_011180_at | No | No | Yes |
| 602 | protein tyrosine | Ptpn1 | NM_011201_at | No | No | Yes |
| | phosphatase, non- | | | | | |
| | receptor type 1 | | | | | |
| 603 | retinoblastoma binding | Rbbp6 | NM_011247_at | No | No | Yes |
| | Protein 6 | | | | | |
| 604 | SWI/SNF related, matrix | Smarcb1 | NM_011418_at | No | No | Yes |
| | associated, actin | | | | | |
| | dependent regulator of | | | | | |
| | chromatin, subfamily b, | | | | | |
| | member 1 | | | | | |
| 605 | cirrhosis, autosomal | Cirh1a | NM_011574_at | No | No | Yes |
| | recessive 1A (human) | | | | | |
| 606 | tumor necrosis factor | Tnfrsf1a | NM_011609_at | No | No | Yes |
| | receptor superfamily, | | | | | |
| | member 1a | | | | | |
| 607 | tumor necrosis factor | Tnfrsf9 | NM_011612_at | No | No | Yes |
| | receptor superfamily, | | | | | |
| | member 9 | | | | | |
| 608 | transformation related | Trp63 | NM_011641_at | No | No | Yes |
| | Protein 63 | | | | | |
| 609 | translin | Tsn | NM_011650_at | No | No | Yes |
| 610 | transient receptor | Trpv2 | NM_011706_at | No | No | Yes |
| | potential cation channel, | | | | | |
| | subfamily V, member 2 | | | | | |
| 611 | sialic acid acetylesterase | Siae | NM_011734_at | No | No | Yes |
| 612 | makorin, ring finger | Mkrn3 | NM_011746_at | No | No | Yes |
| | protein, 3 | | | | | |
| 613 | zinc finger protein 90 | Zfp90 | NM_011764_at | No | No | Yes |
| 614 | solute carrier family 30 | Slc30a4 | NM_011774_at | No | No | Yes |
| | (zinc transporter), | | | | | |
| | member 4 | | | | | |
| 615 | germ cell-less homolog 1 | Gmcl1 | NM_011818_at | No | No | Yes |
| | (Drosophila) | | | | | |
| 616 | NIMA (never in mitosis | Nek3 | NM_011848_at | No | No | Yes |
| | gene a)-related | | | | | |
| | expressed kinase 3 | | | | | |
| 617 | solute carrier family 35 | Slc35a1 | NM_011895_at | No | No | Yes |
| | (CMP-sialic acid | | | | | |
| | transporter), member 1 | | | | | |
| 618 | solute carrier family 9 | Slc9a3r1 | NM_012030_at | No | No | Yes |
| | (sodium/hydrogen | | | | | |
| | exchanger), member 3 | | | | | |
| | regulator 1 | | | | | |
| 619 | complement component | Cr1l | NM_013499_at | No | No | Yes |
| | (3b/4b) receptor 1-like | | | | | |
| 620 | putative homeodomain | Phtf1 | NM_013629_at | No | No | Yes |
| | transcription factor 1 | | | | | |
| 621 | sema domain, | Sema4d | NM_013660_at | No | No | Yes |
| | immunoglobulin domain | | | | | |
| | (Ig), transmembrane | | | | | |
| | domain (TM) and short | | | | | |
| | cytoplasmic domain, | | | | | |
| | (semaphorin) 4D | | | | | |
| 622 | polo-like kinase 3 | Plk3 | NM_013807_at | No | No | Yes |
| | (Drosophila) | | | | | |
| 623 | zinc finger protein 68 | Zfp68 | NM_013844_at | No | No | Yes |
| 624 | Unc-51 like kinase 2 (C. elegans) | Ulk2 | NM_013881_at | No | No | Yes |
| 625 | origin recognition | Orc3 | NM_015824_at | No | No | Yes |
| | complex, subunit 3 | | | | | |
| 626 | WW domain binding | Wbp1 | NM_016757_at | No | No | Yes |
| | Protein 1 | | | | | |
| 627 | arginine vasopressin | Avpr1a | NM_016847_at | No | No | Yes |
| | receptor 1A | | | | | |
| 628 | adenylate kinase 2 | Ak2 | NM_016895_at | No | No | Yes |
| 629 | mitogen-activated | Map3k14 | NM_016896_at | No | No | Yes |
| | protein kinase kinase | | | | | |
| | kinase 14 | | | | | |
| 630 | cyclin-dependent kinase- | Cdkl2 | NM_016912_at | No | No | Yes |
| | like 2 (CDC2-related | | | | | |
| | kinase) | | | | | |
| 631 | inducible T-cell co- | Icos | NM_017480_at | No | No | Yes |
| | stimulator | | | | | |
| 632 | retinoic acid induced 12 | Rai12 | NM_018740_at | No | No | Yes |
| 633 | aldo-keto reductase | Akr1e1 | NM_018859_at | No | No | Yes |
| | family 1, member E1 | | | | | |
| 634 | nuclear factor of kappa | Nfkb2 | NM_019408_at | No | No | Yes |
| | light polypeptide gene | | | | | |
| | enhancer in B-cells 2, | | | | | |
| | p49/p100 | | | | | |
| 635 | pannexin 1 | Panx1 | NM_019482_at | No | No | Yes |
| 636 | N-acetylglucosamine | Nagk | NM_019542_at | No | No | Yes |
| | kinase | | | | | |
| 637 | phosphatidylinositol | Pigp | NM_019543_at | No | No | Yes |
| | glycan anchor | | | | | |
| | biosynthesis, class P | | | | | |
| 638 | non-metastatic cells 3, | Nme3 | NM_019730_at | No | No | Yes |
| | protein expressed in | | | | | |
| 639 | thyroid hormone | Trip4 | NM_019797_at | No | No | Yes |
| | receptor interactor 4 | | | | | |
| 640 | sirtuin 1 (silent mating | Sirt1 | NM_019812_at | No | No | Yes |
| | type information | | | | | |
| | regulation 2, homolog) 1 | | | | | |
| | (S. cerevisiae) | | | | | |
| 641 | RAB guanine nucleotide | Rabgef1 | NM_019983_at | No | No | Yes |
| | exchange factor (GEF) 1 | | | | | |
| 642 | DnaJ (Hsp40) homolog, | Dnajc4 | NM_020566_at | No | No | Yes |
| | subfamily C, member 4 | | | | | |
| 643 | integrin alpha X | Itgax | NM_021334_at | No | No | Yes |
| 644 | prokineticin receptor 1 | Prokr1 | NM_021381_at | No | No | Yes |
| 645 | trafficking protein | Trappc2l | NM_021502_at | No | No | Yes |
| | particle complex 2-like | | | | | |
| 646 | general transcription | Gtf2h2 | NM_022011_at | No | No | Yes |
| | factor II H, polypeptide 2 | | | | | |
| 647 | UDP-Gal:betaGlcNAc beta | B4galt1 | NM_022305_at | No | No | Yes |
| | 1,4- | | | | | |
| | galactosyltransferase, | | | | | |
| | polypeptide 1 | | | | | |
| 648 | DNA methyltransferase 1- | Dmap1 | NM_023178_at | No | No | Yes |
| | associated protein 1 | | | | | |
| 649 | protein phosphatase 1, | Ppp1r7 | NM_023200_at | No | No | Yes |
| | regulatory (inhibitor) | | | | | |
| | subunit 7 | | | | | |
| 650 | diablo homolog | Diablo | NM_023232_at | No | No | Yes |
| | (Drosophila) | | | | | |
| 651 | NADH dehydrogenase | Ndufaf3 | NM_023247_at | No | No | Yes |
| | (ubiquinone) 1 alpha | | | | | |
| | subcomplex, assembly | | | | | |
| | factor 3 | | | | | |
| 652 | magnesium-dependent | Mdp1 | NM_023397_at | No | No | Yes |
| | phosphatase 1 | | | | | |
| 653 | melanoma associated | Mum1 | NM_023431_at | No | No | Yes |
| | antigen (mutated) 1 | | | | | |
| 654 | zinc finger with KRAB | Zkscan3 | NM_023685_at | No | No | Yes |
| | and SCAN domains 3 | | | | | |
| 655 | tripartite motif- | Trim12a | NM_023835_at | No | No | Yes |
| | containing 12A | | | | | |
| 656 | centrosomal protein 70 | Cep70 | NM_023873_at | No | No | Yes |
| 657 | glucosaminyl (N-acetyl) | Gcnt2 | NM_023887_at | No | No | Yes |
| | transferase 2, I- | | | | | |
| | branching enzyme | | | | | |
| 658 | GPN-loop GTPase 3 | Gpn3 | NM_024216_at | No | No | Yes |
| 659 | hydroxysteroid | Hsdl2 | NM_024255_at | No | No | Yes |
| | dehydrogenase like 2 | | | | | |
| 660 | FtsJ homolog 3 (E. coli) | Ftsj3 | NM_025310_at | No | No | Yes |
| 661 | abhydrolase domain | Abhd6 | NM_025341_at | No | No | Yes |
| | containing 6 | | | | | |
| 662 | ubiquitin-fold modifier | Ufc1 | NM_025388_at | No | No | Yes |
| | conjugating enzyme 1 | | | | | |
| 663 | COMM domain containing 4 | Commd4 | NM_025417_at | No | No | Yes |
| 664 | ribosomal protein L7- | Rpl7l1 | NM_025433_at | No | No | Yes |
| | like 1 | | | | | |
| 665 | nicolin 1 | Nicn1 | NM_025449_at | No | No | Yes |
| 666 | family with sequence | Fam134b | NM_025459_at | No | No | Yes |
| | similarity 134, member B | | | | | |
| 667 | transmembrane protein | Tmem208 | NM_025486_at | No | No | Yes |
| | 208 | | | | | |
| 668 | RIKEN cDNA 1810046J19 | 1810046J19Rik | NM_025559_at | No | No | Yes |
| | gene | | | | | |
| 669 | DET1 and DDB1 | Dda1 | NM_025600_at | No | No | Yes |
| | associated 1 | | | | | |
| 670 | EP300 interacting | Eid1 | NM_025613_at | No | No | Yes |
| | inhibitor of | | | | | |
| | differentiation 1 | | | | | |
| 671 | nucleoside- | Ntpcr | NM_025636_at | No | No | Yes |
| | triphosphatase, cancer- | | | | | |
| | related | | | | | |
| 672 | general transcription | Gtf3a | NM_025652_at | No | No | Yes |
| | factor III A | | | | | |
| 673 | endoplasmic reticulum | Erlec1 | NM_025745_at | No | No | Yes |
| | lectin 1 | | | | | |
| 674 | RIKEN cDNA 4933434E20 | 4933434E20Rik | NM_025762_at | No | No | Yes |
| | gene | | | | | |
| 675 | F-box protein 25 | Fbxo25 | NM_025785_at | No | No | Yes |
| 676 | C-type lectin domain | Clec14a | NM_025809_at | No | No | Yes |
| | family 14, member a | | | | | |
| 677 | TLR4 interactor with | Tril | NM_025817_at | No | No | Yes |
| | leucine-rich repeats | | | | | |
| 678 | acyl-Coenzyme A | Acad8 | NM_025862_at | No | No | Yes |
| | dehydrogenase family, | | | | | |
| | member 8 | | | | | |
| 679 | DNA segment, Chr 16, | D16Ertd472e | NM_025967_at | No | No | Yes |
| | ERATO Doi 472, | | | | | |
| | expressed | | | | | |
| 680 | zinc finger with KRAB | Zkscan6 | NM_026107_at | No | No | Yes |
| | and SCAN domains 6 | | | | | |
| 681 | polymerase (RNA) II | Polr2g | NM_026329_at | No | No | Yes |
| | (DNA directed) | | | | | |
| | polypeptide G | | | | | |
| 682 | syntaxin 17 | Stx17 | NM_026343_at | No | No | Yes |
| 683 | plakophilin 4 | Pkp4 | NM_026361_at | No | No | Yes |
| 684 | dicarbonyl L-xylulose | Dcxr | NM_026428_at | No | No | Yes |
| | reductase | | | | | |
| 685 | serine/arginine-rich | Srsf6 | NM_026499_at | No | No | Yes |
| | splicing factor 6 | | | | | |
| 686 | biphenyl hydrolase-like | Bphl | NM_026512_at | No | No | Yes |
| | (serine hydrolase, breast | | | | | |
| | epithelial mucin- | | | | | |
| | associated antigen) | | | | | |
| 687 | emopamil binding | Ebpl | NM_026598_at | No | No | Yes |
| | Protein-like | | | | | |
| 688 | intraflagellar transport | Ift80 | NM_026641_at | No | No | Yes |
| | 80 homolog | | | | | |
| | (Chlamydomonas) | | | | | |
| 689 | immature colon | Ict1 | NM_026729_at | No | No | Yes |
| | carcinoma transcript 1 | | | | | |
| 690 | zinc finger, FYVE | Zfyve21 | NM_026752_at | No | No | Yes |
| | domain containing 21 | | | | | |
| 691 | mutL homolog 1 (E. coli) | Mlh1 | NM_026810_at | No | No | Yes |
| 692 | RIKEN cDNA 1500032L24 | 1500032L24Rik | NM_026914_at | No | No | Yes |
| | gene | | | | | |
| 693 | CCR4-NOT transcription | Cnot8 | NM_026949_at | No | No | Yes |
| | complex, subunit 8 | | | | | |
| 694 | CDC42 effector protein | Cdc42ep1 | NM_027219_at | No | No | Yes |
| | (Rho GTPase binding) 1 | | | | | |
| 695 | ring finger protein 157 | Rnf157 | NM_027258_at | No | No | Yes |
| 696 | mediator complex | Med23 | NM_027347_at | No | No | Yes |
| | subunit 23 | | | | | |
| 697 | RIKEN cDNA 5730494N06 | 5730494N06Rik | NM_027478_at | No | No | Yes |
| | gene | | | | | |
| 698 | ankyrin repeat domain | Ankrd24 | NM_027480_at | No | No | Yes |
| | 24 | | | | | |
| 699 | RIKEN cDNA 4931408A02 | 4931408A02Rik | NM_027627_at | No | No | Yes |
| | gene | | | | | |
| 700 | leucine rich repeat (in | Lrrfip2 | NM_027742_at | No | No | Yes |
| | FLII) interacting protein 2 | | | | | |
| 701 | transmembrane protein | Tmem80 | NM_027797_at | No | No | Yes |
| | 80 | | | | | |
| 702 | ring finger protein 135 | Rnf135 | NM_028019_at | No | No | Yes |
| 703 | cell division cycle | Cdca4 | NM_028023_at | No | No | Yes |
| | associated 4 | | | | | |
| 704 | solute carrier family 25, | Slc25a35 | NM_028048_at | No | No | Yes |
| | member 35 | | | | | |
| 705 | carbohydrate (N- | Chst14 | NM_028117_at | No | No | Yes |
| | acetylgalactosamine 4-0) | | | | | |
| | sulfotransferase 14 | | | | | |
| 706 | coiled-coil domain | Ccdc123 | NM_028120_at | No | No | Yes |
| | containing 123 | | | | | |
| 707 | RIKEN cDNA 2210016L21 | 2210016L21Rik | NM_028211_at | No | No | Yes |
| | gene | | | | | |
| 708 | ribosomal RNA | Rrp1b | NM_028244_at | No | No | Yes |
| | processing 1 homolog B | | | | | |
| | (S. cerevisiae) | | | | | |
| 709 | transmembrane protein | Tmem55a | NM_028264_at | No | No | Yes |
| | 55A | | | | | |
| 710 | centromere protein V | Cenpv | NM_028448_at | No | No | Yes |
| 711 | chibby homolog 1 | Cby1 | NM_028634_at | No | No | Yes |
| | (Drosophila) | | | | | |
| 712 | transmembrane and | Tmtc4 | NM_028651_at | No | No | Yes |
| | tetratricopeptide repeat | | | | | |
| | containing 4 | | | | | |
| 713 | MTERF domain | Mterfd3 | NM_028832_at | No | No | Yes |
| | containing 3 | | | | | |
| 714 | RIKEN cDNA 4933407C03 | 4933407C03Rik | NM_028941_at | No | No | Yes |
| | gene | | | | | |
| 715 | RIKEN cDNA 4930444A02 | 4930444A02Rik | NM_029037_at | No | No | Yes |
| | gene | | | | | |
| 716 | TDP-glucose 4,6- | Tgds | NM_029578_at | No | No | Yes |
| | dehydratase | | | | | |
| 717 | jumonji domain | Jmjd5 | NM_029842_at | No | No | Yes |
| | containing 5 | | | | | |
| 718 | H2A histone family, | H2afv | NM_029938_at | No | No | Yes |
| | member V | | | | | |
| 719 | CDKN2A interacting | Cdkn2aipnl | NM_029976_at | No | No | Yes |
| | protein N-terminal like | | | | | |
| 720 | phosphatidylinositol | Pigh | NM_029988_at | No | No | Yes |
| | glycan anchor | | | | | |
| | biosynthesis, class H | | | | | |
| 721 | armadillo repeat | Armcx1 | NM_030066_at | No | No | Yes |
| | containing, X-linked 1 | | | | | |
| 722 | bromodomain | Brd8 | NM_030147_at | No | No | Yes |
| | containing 8 | | | | | |
| 723 | RNA binding motif | Rbm43 | NM_030243_at | No | No | Yes |
| | protein 43 | | | | | |
| 724 | NEDD4 binding protein 1 | N4bp1 | NM_030563_at | No | No | Yes |
| 725 | tripartite motif- | Trim34a | NM_030684_at | No | No | Yes |
| | containing 34A | | | | | |
| 726 | interleukin 23, alpha | Il23a | NM_031252_at | No | No | Yes |
| | subunit p19 | | | | | |
| 727 | protein kinase, AMP- | Prkab1 | NM_031869_at | No | No | Yes |
| | activated, beta 1 non- | | | | | |
| | catalytic subunit | | | | | |
| 728 | nucleoporin 62 | Nup62 | NM_053074_at | No | No | Yes |
| 729 | nucleolar and coiled- | Nolc1 | NM_053086_at | No | No | Yes |
| | body phosphoprotein 1 | | | | | |
| 730 | RIKEN cDNA 1110038F14 | 1110038F14Rik | NM_054099_at | No | No | Yes |
| | gene | | | | | |
| 731 | histamine N- | Hnmt | NM_080462_at | No | No | Yes |
| | methyltransferase | | | | | |
| 732 | oxidation resistance 1 | Oxr1 | NM_130885_at | No | No | Yes |
| 733 | glucosaminyl (N-acetyl) | Gcnt2 | NM_133219_at | No | No | Yes |
| | transferase 2, I- | | | | | |
| | branching enzyme | | | | | |
| 734 | DDB1 and CUL4 | Dcaf11 | NM_133734_at | No | No | Yes |
| | associated factor 11 | | | | | |
| 735 | SNF related kinase | Snrk | NM_133741_at | No | No | Yes |
| 736 | exonuclease 3′-5′ | Exd2 | NM_133798_at | No | No | Yes |
| | domain containing 2 | | | | | |
| 737 | ubiquitin associated | Ubac1 | NM_133835_at | No | No | Yes |
| | domain containing 1 | | | | | |
| 738 | CLP1, cleavage and | Clp1 | NM_133840_at | No | No | Yes |
| | polyadenylation factor I | | | | | |
| | subunit, homolog (S. cerevisiae) | | | | | |
| 739 | G protein-coupled | Gpr125 | NM_133911_at | No | No | Yes |
| | receptor 125 | | | | | |
| 740 | coiled-coil-helix-coiled- | Chchd4 | NM_133928_at | No | No | Yes |
| | coil-helix domain | | | | | |
| | containing 4 | | | | | |
| 741 | nuclear mitotic | Numa1 | NM_133947_at | No | No | Yes |
| | apparatus protein 1 | | | | | |
| 742 | KDEL (Lys-Asp-Glu-Leu) | Kdelr1 | NM_133950_at | No | No | Yes |
| | endoplasmic reticulum | | | | | |
| | protein retention | | | | | |
| | receptor 1 | | | | | |
| 743 | aldehyde dehydrogenase | Aldh7a1 | NM_138600_at | No | No | Yes |
| | family 7, member A1 | | | | | |
| 744 | nucleosome assembly | Nap1l3 | NM_138742_at | No | No | Yes |
| | protein 1-like 3 | | | | | |
| 745 | Rab40b, member RAS | Rab40b | NM_139147_at | No | No | Yes |
| | oncogene family | | | | | |
| 746 | fukutin | Fktn | NM_139309_at | No | No | Yes |
| 747 | SEC14-like 2 (S. cerevisiae) | Sec14l2 | NM_144520_at | No | No | Yes |
| 748 | solute carrier family 39 | Slc39a14 | NM_144808_at | No | No | Yes |
| | (zinc transporter), | | | | | |
| | member 14 | | | | | |
| 749 | HEAT repeat containing 1 | Heatr1 | NM_144835_at | No | No | Yes |
| 750 | cDNA sequence BC018507 | BC018507 | NM_144837_at | No | No | Yes |
| 751 | suppressor of | Suv420h1 | NM_144871_at | No | No | Yes |
| | variegation 4-20 | | | | | |
| | homolog 1 (Drosophila) | | | | | |
| 752 | DEP domain containing | Deptor | NM_145470_at | No | No | Yes |
| | MTOR-interacting protein | | | | | |
| 753 | EF-hand calcium binding | Efcab7 | NM_145549_at | No | No | Yes |
| | domain 7 | | | | | |
| 754 | cerebral cavernous | Ccm2 | NM_146014_at | No | No | Yes |
| | malformation 2 homolog | | | | | |
| | (human) | | | | | |
| 755 | RIKEN cDNA 3830406C13 | 3830406C13Rik | NM_146051_at | No | No | Yes |
| | gene | | | | | |
| 756 | transcription factor B1, | Tfb1m | NM_146074_at | No | No | Yes |
| | mitochondrial | | | | | |
| 757 | discs, large homolog- | Dlgap4 | NM_146128_at | No | No | Yes |
| | associated protein 4 | | | | | |
| | (Drosophila) | | | | | |
| 758 | pyrophosphatase | Ppa2 | NM_146141_at | No | No | Yes |
| | (inorganic) 2 | | | | | |
| 759 | ring finger and WD | Rfwd3 | NM_146218_at | No | No | Yes |
| | repeat domain 3 | | | | | |
| 760 | cDNA sequence BC024479 | BC024479 | NM_146222_at | No | No | Yes |
| 761 | zinc finger protein 825 | Zfp825 | NM_146231_at | No | No | Yes |
| 762 | general transcription | Gtf3c5 | NM_148928_at | No | No | Yes |
| | factor IIIC, polypeptide 5 | | | | | |
| 763 | potassium channel | Kctd11 | NM_153143_at | No | No | Yes |
| | tetramerisation domain | | | | | |
| | containing 11 | | | | | |
| 764 | family with sequence | Fam102a | NM_153560_at | No | No | Yes |
| | similarity 102, member A | | | | | |
| 765 | nucleolar complex | Noc4l | NM_153570_at | No | No | Yes |
| | associated 4 homolog (S. cerevisiae) | | | | | |
| 766 | protein-L-isoaspartate (D- | Pcmtd2 | NM_153594_at | No | No | Yes |
| | aspartate) O- | | | | | |
| | methyltransferase | | | | | |
| | domain containing 2 | | | | | |
| 767 | U2 small nuclear RNA | U2af1l4 | NM_170760_at | No | No | Yes |
| | auxiliary factor 1-like 4 | | | | | |
| 768 | F-box protein 42 | Fbxo42 | NM_172518_at | No | No | Yes |
| 769 | PQ loop repeat | Pqlc3 | NM_172574_at | No | No | Yes |
| | containing | | | | | |
| 770 | F11 receptor | F11r | NM_172647_at | No | No | Yes |
| 771 | von Willebrand factor A | Vwa5a | NM_172767_at | No | No | Yes |
| | domain containing 5A | | | | | |
| 772 | torsin A interacting | Tor1aip2 | NM_172843_at | No | No | Yes |
| | protein 2 | | | | | |
| 773 | regulator of | Rcc2 | NM_173867_at | No | No | Yes |
| | chromosome | | | | | |
| | condensation 2 | | | | | |
| 774 | cytoskeleton-associated | Ckap4 | NM_175451_at | No | No | Yes |
| | protein 4 | | | | | |
| 775 | plakophilin 4 | Pkp4 | NM_175464_at | No | No | Yes |
| 776 | WD repeat domain 43 | Wdr43 | NM_175639_at | No | No | Yes |
| 777 | RIKEN cDNA | 4930471M23Rik | NM_175675_at | No | No | Yes |
| | 4930471M23 gene | | | | | |
| 778 | RIKEN cDNA 4833442J19 | 4833442J19Rik | NM_177101_at | No | No | Yes |
| | gene | | | | | |
| 779 | cyclin-dependent kinase- | Cdkl2 | NM_177270_at | No | No | Yes |
| | like 2 (CDC2-related | | | | | |
| | kinase) | | | | | |
| 780 | HMG box domain | Hmgxb4 | NM_178017_at | No | No | Yes |
| | containing 4 | | | | | |
| 781 | RIKEN cDNA 3830406C13 | 3830406C13Rik | NM_178141_at | No | No | Yes |
| | gene | | | | | |
| 782 | calcium/calmodulin- | Camk2g | NM_178597_at | No | No | Yes |
| | dependent protein | | | | | |
| | kinase II gamma | | | | | |
| 783 | coronin, actin binding | Coro2a | NM_178893_at | No | No | Yes |
| | protein 2A | | | | | |
| 784 | inositol polyphosphate- | Inpp5a | NM_183144_at | No | No | Yes |
| | 5-phosphatase A | | | | | |
| 785 | endothelin converting | Ece1 | NM_199307_at | No | No | Yes |
| | enzyme 1 | | | | | |
| 786 | F-box protein 3 | Fbxo3 | NM_212433_at | No | No | Yes |
| 787 | RIKEN cDNA 4933431E20 | 4933431E20Rik | NR_015459_at | No | No | Yes |
| | gene | | | | | |
| 788 | RIKEN cDNA 1810058I24 | 1810058I24Rik | NR_015608_at | No | No | Yes |
| | gene | | | | | |
| 789 | RIKEN cDNA 1810058I24 | 1810058I24Rik | NR_027875_at | No | No | Yes |
| | gene | | | | | |
| 790 | retinoic acid induced 12 | Rai12 | NR_027884_at | No | No | Yes |
| 791 | retinoic acid induced 12 | Rai12 | NR_027977_at | No | No | Yes |
| 792 | magnesium-dependent | Mdp1 | NR_028316_at | No | No | Yes |
| | phosphatase 1 | | | | | |
| 793 | RNA binding motif | Rbmx | NR_029425_at | No | No | Yes |
| | protein, X chromosome | | | | | |
| 794 | DDB1 and CUL4 | Dcaf11 | NR_037572_at | No | No | Yes |
| | associated factor 11 | | | | | |

| | | log₂- | | | | | | | | | | | |
|---|---|---|---|---|---|---|---|---|---|---|---|---|---|
| | | fold | | | Kendall | | | | | | | | |
| | | change | | Corr. | Adj p | Log₂ Expression Level | | | | | | | |
| # | Adj p | (LFC) | SE | Coeff. | rank | Untreated | DBP | DEET | DMP | Acetone | DBIT | DEHM | DEP |
| 1 | 0.00091 | 1.12 | 0.77 | 0.60 | 1129.5 | 5.69 | 6.82 | 6.74 | 5.82 | 5.63 | 6.20 | 6.44 | 5.52 |
| 2 | 0.00091 | 1.04 | 0.56 | 0.69 | 289 | 5.89 | 6.93 | 6.44 | 6.47 | 7.13 | 6.75 | 7.16 | 7.18 |
| 3 | 0.00091 | 1.31 | 0.71 | 0.63 | 807 | 8.51 | 9.82 | 9.66 | 8.83 | 8.73 | 9.56 | 9.61 | 8.98 |
| 4 | 0.00091 | 1.31 | 0.71 | 0.63 | 807 | 8.51 | 9.82 | 9.66 | 8.83 | 8.73 | 9.56 | 9.61 | 8.98 |
| 5 | 0.04753 | 1.21 | 1.10 | 0.33 | 6143.5 | 6.32 | 7.53 | 8.20 | 6.08 | 6.36 | 7.34 | 7.81 | 6.10 |
| 6 | 0.00091 | 1.29 | 0.77 | 0.55 | 1825.5 | 5.62 | 6.91 | 7.42 | 6.09 | 5.62 | 6.36 | 6.33 | 7.14 |
| 7 | 0.00091 | 1.23 | 0.75 | 0.53 | 2087.5 | 5.04 | 6.26 | 6.76 | 5.32 | 5.07 | 5.73 | 5.65 | 6.55 |
| 8 | 0.00091 | 1.29 | 0.77 | 0.55 | 1825.5 | 5.62 | 6.91 | 7.42 | 6.09 | 5.62 | 6.36 | 6.33 | 7.14 |
| 9 | 0.00091 | 1.29 | 0.77 | 0.55 | 1825.5 | 5.62 | 6.91 | 7.42 | 6.09 | 5.62 | 6.36 | 6.33 | 7.14 |
| 10 | 0.00091 | 1.29 | 0.77 | 0.55 | 1825.5 | 5.62 | 6.91 | 7.42 | 6.09 | 5.62 | 6.36 | 6.33 | 7.14 |
| 11 | 0.00091 | 1.23 | 0.75 | 0.53 | 2087.5 | 5.04 | 6.26 | 6.76 | 5.32 | 5.07 | 5.73 | 5.65 | 6.55 |
| 12 | 0.00091 | 1.23 | 0.75 | 0.53 | 2087.5 | 5.04 | 6.26 | 6.76 | 5.32 | 5.07 | 5.73 | 5.65 | 6.55 |
| 13 | 0.00091 | 1.23 | 0.75 | 0.53 | 2087.5 | 5.04 | 6.26 | 6.76 | 5.32 | 5.07 | 5.73 | 5.65 | 6.55 |
| 14 | 0.00091 | 1.52 | 0.83 | 0.70 | 265.5 | 4.61 | 6.13 | 5.31 | 4.91 | 4.90 | 5.61 | 5.35 | 4.71 |
| 15 | 0.00091 | 1.52 | 0.83 | 0.70 | 265.5 | 4.61 | 6.13 | 5.31 | 4.91 | 4.90 | 5.61 | 5.35 | 4.71 |
| 16 | 0.00091 | 1.39 | 0.84 | 0.60 | 1129.5 | 6.07 | 7.45 | 7.68 | 6.50 | 6.15 | 6.54 | 6.67 | 7.54 |
| 17 | 0.00091 | 1.41 | 0.81 | 0.56 | 1683 | 8.08 | 9.48 | 9.60 | 8.80 | 8.79 | 9.40 | 9.88 | 9.32 |
| 18 | 0.00091 | 1.41 | 0.81 | 0.56 | 1683 | 8.08 | 9.48 | 9.60 | 8.80 | 8.79 | 9.40 | 9.88 | 9.32 |
| 19 | 0.00091 | 1.48 | 0.93 | 0.52 | 2218.5 | 8.13 | 9.61 | 10.04 | 8.29 | 8.22 | 8.62 | 8.65 | 8.41 |
| 20 | 0.00091 | 5.93 | 3.07 | 0.56 | 1683 | 5.18 | 11.11 | 11.87 | 8.26 | 7.12 | 11.09 | 11.17 | 8.85 |
| 21 | 0.00091 | 1.51 | 0.86 | 0.50 | 2583.5 | 7.76 | 9.27 | 9.61 | 7.91 | 7.81 | 9.29 | 9.16 | 8.77 |
| 22 | 0.00145 | 1.97 | 1.37 | 0.48 | 3075 | 6.26 | 8.24 | 8.47 | 5.99 | 7.76 | 7.90 | 7.40 | 5.30 |
| 23 | 0.00195 | 1.27 | 0.97 | 0.45 | 3644.5 | 5.62 | 6.89 | 7.75 | 5.65 | 5.76 | 6.35 | 6.72 | 6.03 |
| 24 | 0.00091 | 1.16 | 0.60 | 0.60 | 1129.5 | 6.95 | 8.10 | 8.21 | 7.33 | 6.97 | 7.55 | 7.61 | 7.92 |
| 25 | 0.00091 | 1.16 | 0.60 | 0.60 | 1129.5 | 6.95 | 8.10 | 8.21 | 7.33 | 6.97 | 7.55 | 7.61 | 7.92 |
| 26 | 0.00145 | 1.57 | 1.07 | 0.50 | 2583.5 | 6.99 | 8.56 | 8.83 | 7.55 | 6.97 | 7.74 | 8.88 | 7.51 |
| 27 | 0.00145 | 1.57 | 1.07 | 0.50 | 2583.5 | 6.99 | 8.56 | 8.83 | 7.55 | 6.97 | 7.74 | 8.88 | 7.51 |
| 28 | 0.00145 | 1.57 | 1.07 | 0.50 | 2583.5 | 6.99 | 8.56 | 8.83 | 7.55 | 6.97 | 7.74 | 8.88 | 7.51 |
| 29 | 0.00091 | 2.10 | 1.17 | 0.55 | 1779 | 5.13 | 7.22 | 7.20 | 5.77 | 4.96 | 6.24 | 6.20 | 5.79 |
| 30 | 0.00091 | 1.26 | 0.68 | 0.55 | 1825.5 | 7.66 | 8.92 | 9.06 | 8.30 | 8.52 | 8.44 | 8.53 | 8.76 |
| 31 | 0.00091 | 1.26 | 0.68 | 0.55 | 1825.5 | 7.66 | 8.92 | 9.06 | 8.30 | 8.52 | 8.44 | 8.53 | 8.76 |
| 32 | 0.00091 | 1.26 | 0.68 | 0.55 | 1825.5 | 7.66 | 8.92 | 9.06 | 8.30 | 8.52 | 8.44 | 8.53 | 8.76 |
| 33 | 0.01547 | 1.08 | 0.81 | 0.47 | 3139 | 5.46 | 6.54 | 6.10 | 5.48 | 5.65 | 6.33 | 5.87 | 5.34 |
| 34 | 0.00091 | 1.49 | 0.75 | 0.65 | 617 | 6.13 | 7.61 | 7.43 | 6.50 | 6.46 | 7.24 | 6.96 | 6.85 |
| 35 | 0.00091 | 1.49 | 0.75 | 0.65 | 617 | 6.13 | 7.61 | 7.43 | 6.50 | 6.46 | 7.24 | 6.96 | 6.85 |
| 36 | 0.00091 | 1.31 | 0.71 | 0.63 | 807 | 8.51 | 9.82 | 9.66 | 8.83 | 8.73 | 9.56 | 9.61 | 8.98 |
| 37 | 0.00091 | 1.31 | 0.71 | 0.63 | 807 | 8.51 | 9.82 | 9.66 | 8.83 | 8.73 | 9.56 | 9.61 | 8.98 |
| 38 | 0.00091 | 1.31 | 0.71 | 0.63 | 807 | 8.51 | 9.82 | 9.66 | 8.83 | 8.73 | 9.56 | 9.61 | 8.98 |
| 39 | 0.00091 | 1.29 | 0.79 | 0.60 | 1129.5 | 6.88 | 8.18 | 8.11 | 7.03 | 7.16 | 8.07 | 8.15 | 7.28 |
| 40 | 0.00091 | 1.29 | 0.78 | 0.55 | 1779 | 6.96 | 8.24 | 8.22 | 7.09 | 7.23 | 8.14 | 8.21 | 7.39 |
| 41 | 0.00091 | 1.30 | 0.79 | 0.59 | 1279.5 | 7.14 | 8.44 | 8.43 | 7.32 | 7.42 | 8.32 | 8.37 | 7.54 |
| 42 | 0.00091 | 1.28 | 0.78 | 0.58 | 1379.5 | 6.79 | 8.07 | 7.97 | 6.91 | 7.03 | 7.98 | 8.05 | 7.18 |
| 43 | 0.00091 | 1.28 | 0.78 | 0.58 | 1379.5 | 6.79 | 8.07 | 7.97 | 6.91 | 7.03 | 7.98 | 8.05 | 7.18 |
| 44 | 0.00091 | 1.30 | 0.79 | 0.59 | 1279.5 | 7.14 | 8.44 | 8.43 | 7.32 | 7.42 | 8.32 | 8.37 | 7.54 |
| 45 | 0.00091 | 1.28 | 0.78 | 0.58 | 1379.5 | 6.79 | 8.07 | 7.97 | 6.91 | 7.03 | 7.98 | 8.05 | 7.18 |
| 46 | 0.00091 | 1.30 | 0.79 | 0.59 | 1279.5 | 7.14 | 8.44 | 8.43 | 7.32 | 7.42 | 8.32 | 8.37 | 7.54 |
| 47 | 0.00091 | 1.44 | 0.78 | 0.50 | 2583.5 | 7.52 | 8.96 | 9.23 | 8.08 | 7.47 | 8.02 | 8.19 | 8.45 |
| 48 | 0.00091 | 1.44 | 0.78 | 0.50 | 2583.5 | 7.52 | 8.96 | 9.23 | 8.08 | 7.47 | 8.02 | 8.19 | 8.45 |
| 49 | 0.00091 | 1.44 | 0.78 | 0.50 | 2583.5 | 7.52 | 8.96 | 9.23 | 8.08 | 7.47 | 8.02 | 8.19 | 8.45 |
| 50 | 0.00091 | 1.80 | 1.06 | 0.53 | 2087.5 | 8.32 | 10.11 | 10.17 | 9.13 | 7.97 | 8.69 | 9.16 | 9.67 |
| 51 | 0.00091 | 1.94 | 0.96 | 0.70 | 265.5 | 6.90 | 8.84 | 8.15 | 7.49 | 7.28 | 8.60 | 8.07 | 7.95 |
| 52 | 0.00091 | 1.02 | 0.63 | 0.57 | 1585.5 | 9.34 | 10.36 | 10.64 | 9.61 | 9.55 | 9.72 | 9.91 | 9.60 |
| 53 | 0.00091 | 1.59 | 1.01 | 0.45 | 3611.5 | 6.99 | 8.58 | 9.22 | 8.18 | 7.74 | 8.14 | 7.59 | 8.70 |
| 54 | 0.00091 | 1.56 | 0.76 | 0.78 | 34.5 | 9.06 | 10.63 | 10.01 | 9.63 | 9.23 | 10.44 | 10.01 | 10.81 |
| 55 | 0.00091 | 1.22 | 0.92 | 0.44 | 3687.5 | 6.34 | 7.56 | 8.18 | 6.47 | 6.67 | 7.13 | 7.52 | 6.30 |
| 56 | 0.00091 | 1.58 | 0.84 | 0.57 | 1585.5 | 8.59 | 10.17 | 10.41 | 9.09 | 8.58 | 9.37 | 9.87 | 9.40 |
| 57 | 0.00091 | 1.26 | 0.68 | 0.55 | 1825.5 | 7.66 | 8.92 | 9.06 | 8.30 | 8.52 | 8.44 | 8.53 | 8.76 |
| 58 | 0.00091 | 1.39 | 0.79 | 0.64 | 739.5 | 8.08 | 9.48 | 9.46 | 8.81 | 8.50 | 8.40 | 8.86 | 8.74 |
| 59 | 0.00091 | 1.01 | 0.54 | 0.67 | 508.5 | 7.71 | 8.72 | 8.46 | 8.07 | 8.28 | 8.78 | 8.53 | 7.93 |
| 60 | 0.00091 | 1.11 | 0.54 | 0.73 | 158 | 6.62 | 7.73 | 7.24 | 6.96 | 6.59 | 7.36 | 7.14 | 7.26 |
| 61 | 0.00091 | 1.50 | 0.82 | 0.60 | 1129.5 | 10.65 | 12.15 | 12.16 | 11.13 | 11.19 | 12.27 | 12.17 | 12.58 |
| 62 | 0.00145 | 1.31 | 0.82 | 0.58 | 1379.5 | 7.21 | 8.52 | 8.52 | 7.95 | 7.64 | 8.38 | 8.35 | 7.63 |
| 63 | 0.00091 | 1.91 | 1.07 | 0.51 | 2453.5 | 9.45 | 11.36 | 11.76 | 10.28 | 10.28 | 11.85 | 11.37 | 10.52 |
| 64 | 0.00091 | 1.01 | 0.54 | 0.56 | 1683 | 10.46 | 11.47 | 11.40 | 10.44 | 10.06 | 11.24 | 10.72 | 11.04 |
| 65 | 0.00091 | 1.40 | 0.87 | 0.53 | 2087.5 | 6.48 | 7.88 | 8.25 | 6.81 | 6.46 | 7.59 | 7.48 | 6.97 |
| 66 | 0.00091 | 1.21 | 0.77 | 0.56 | 1683 | 7.64 | 8.85 | 9.07 | 8.09 | 7.87 | 8.83 | 9.28 | 7.90 |
| 67 | 0.00091 | 1.35 | 0.79 | 0.54 | 1962 | 4.13 | 5.48 | 5.71 | 4.20 | 4.12 | 5.01 | 4.83 | 4.52 |
| 68 | 0.00091 | 5.43 | 2.85 | 0.56 | 1683 | 4.81 | 10.24 | 10.87 | 7.36 | 6.73 | 9.36 | 10.77 | 8.29 |
| 69 | 0.00195 | 1.37 | 1.06 | 0.46 | 3470.5 | 5.17 | 6.54 | 7.06 | 5.28 | 4.99 | 5.99 | 5.98 | 5.13 |
| 70 | 0.00239 | 1.55 | 1.20 | 0.46 | 3354.5 | 3.93 | 5.48 | 5.72 | 3.86 | 4.18 | 4.59 | 5.97 | 4.38 |
| 71 | 0.00091 | 2.14 | 1.05 | 0.75 | 77.5 | 6.37 | 8.51 | 7.84 | 7.42 | 6.68 | 7.40 | 7.48 | 7.95 |
| 72 | 0.00091 | 1.57 | 1.07 | 0.52 | 2399 | 7.55 | 9.12 | 9.65 | 7.77 | 7.93 | 9.14 | 9.24 | 7.61 |
| 73 | 0.00091 | 2.73 | 1.80 | 0.52 | 2399 | 4.94 | 7.67 | 8.88 | 5.38 | 5.55 | 6.45 | 7.85 | 6.09 |
| 74 | 0.00091 | 1.15 | 0.56 | 0.75 | 103 | 6.55 | 7.70 | 7.48 | 7.07 | 7.00 | 7.15 | 7.37 | 7.13 |
| 75 | 0.00091 | 1.48 | 0.93 | 0.52 | 2218.5 | 8.13 | 9.61 | 10.04 | 8.29 | 8.22 | 8.62 | 8.65 | 8.41 |
| 76 | 0.00145 | 1.28 | 1.08 | 0.48 | 3075 | 4.19 | 5.46 | 5.76 | 4.15 | 4.30 | 4.33 | 4.82 | 4.13 |
| 77 | 0.00091 | 1.39 | 0.73 | 0.59 | 1279.5 | 8.32 | 9.71 | 9.62 | 8.59 | 8.48 | 9.56 | 9.49 | 9.75 |
| 78 | 0.00091 | 1.53 | 0.76 | 0.78 | 26 | 9.23 | 10.76 | 9.85 | 9.90 | 9.31 | 9.97 | 10.77 | 10.71 |
| 79 | 0.00408 | 1.63 | 1.12 | 0.52 | 2399 | 6.46 | 8.09 | 8.03 | 7.10 | 6.49 | 7.17 | 8.35 | 6.51 |
| 80 | 0.00091 | 1.11 | 0.55 | 0.65 | 617 | 12.12 | 13.23 | 13.18 | 12.64 | 12.42 | 12.84 | 12.79 | 12.79 |
| 81 | 0.00091 | 1.09 | 0.72 | 0.49 | 2941 | 5.61 | 6.69 | 6.94 | 5.57 | 5.50 | 6.25 | 6.04 | 5.69 |
| 82 | 0.00091 | 1.49 | 1.02 | 0.53 | 2195 | 4.79 | 6.28 | 6.28 | 4.77 | 5.03 | 5.90 | 7.04 | 4.90 |
| 83 | 0.00091 | 1.13 | 0.67 | 0.48 | 3075 | 6.46 | 7.59 | 8.09 | 6.70 | 6.32 | 7.65 | 6.74 | 7.87 |
| 84 | 0.00091 | 2.68 | 1.41 | 0.58 | 1420.5 | 5.64 | 8.32 | 8.70 | 6.49 | 6.04 | 7.19 | 8.34 | 7.51 |
| 85 | 0.00758 | 2.73 | 2.20 | 0.37 | 5293 | 5.11 | 7.84 | 9.57 | 4.59 | 4.71 | 5.91 | 6.86 | 4.61 |
| 86 | 0.00091 | 1.43 | 0.96 | 0.48 | 3075 | 5.73 | 7.16 | 8.02 | 6.27 | 6.08 | 7.32 | 6.96 | 6.60 |
| 87 | 0.01452 | 2.97 | 2.43 | 0.38 | 4931.5 | 6.59 | 9.56 | 11.32 | 8.57 | 8.85 | 9.21 | 11.39 | 7.01 |
| 88 | 0.00091 | 4.56 | 2.60 | 0.52 | 2399 | 4.11 | 8.67 | 10.03 | 5.28 | 4.54 | 6.60 | 8.54 | 5.04 |
| 89 | 0.02132 | 1.07 | 0.77 | 0.43 | 4000.5 | 4.97 | 6.04 | 6.25 | 5.43 | 5.23 | 5.57 | 5.72 | 5.15 |
| 90 | 0.00091 | 1.43 | 0.89 | 0.53 | 2087.5 | 9.41 | 10.84 | 11.13 | 9.55 | 9.45 | 10.41 | 11.38 | 10.08 |
| 91 | 0.00492 | 1.76 | 1.37 | 0.35 | 5555 | 6.48 | 8.25 | 9.01 | 6.99 | 6.14 | 7.94 | 9.37 | 6.27 |
| 92 | 0.00091 | 1.07 | 0.52 | 0.77 | 42.5 | 6.50 | 7.56 | 7.14 | 6.96 | 6.80 | 7.12 | 7.20 | 7.41 |
| 93 | 0.00091 | 1.59 | 0.88 | 0.55 | 1825.5 | 8.39 | 9.98 | 10.13 | 8.40 | 8.25 | 9.54 | 9.80 | 8.87 |
| 94 | 0.00091 | 1.94 | 1.11 | 0.55 | 1779 | 6.90 | 8.85 | 9.19 | 7.22 | 7.10 | 7.98 | 7.73 | 7.83 |
| 95 | 0.00091 | 2.10 | 1.17 | 0.55 | 1779 | 5.13 | 7.22 | 7.20 | 5.77 | 4.96 | 6.24 | 6.20 | 5.79 |
| 96 | 0.00091 | 2.30 | 1.30 | 0.58 | 1420.5 | 6.49 | 8.79 | 9.02 | 7.21 | 6.62 | 8.49 | 8.32 | 6.84 |
| 97 | 0.00091 | 1.07 | 0.66 | 0.64 | 739.5 | 4.97 | 6.04 | 5.86 | 5.27 | 5.16 | 5.81 | 5.33 | 5.27 |
| 98 | 0.00091 | 1.80 | 1.19 | 0.50 | 2718.5 | 4.19 | 5.99 | 6.82 | 4.12 | 4.29 | 5.78 | 6.87 | 5.94 |
| 99 | 0.00091 | 1.75 | 0.88 | 0.64 | 739.5 | 8.31 | 10.05 | 10.05 | 8.88 | 8.55 | 9.59 | 9.44 | 9.42 |
| 100 | 0.00091 | 1.34 | 0.92 | 0.43 | 4000.5 | 6.72 | 8.07 | 8.86 | 6.73 | 6.49 | 7.47 | 7.71 | 7.53 |
| 101 | 0.00091 | 1.31 | 0.71 | 0.63 | 807 | 8.51 | 9.82 | 9.66 | 8.83 | 8.73 | 9.56 | 9.61 | 8.98 |
| 102 | 0.00091 | 2.62 | 1.50 | 0.50 | 2718.5 | 6.19 | 8.81 | 9.63 | 6.82 | 6.34 | 7.69 | 7.33 | 7.45 |
| 103 | 0.00091 | 1.91 | 1.14 | 0.58 | 1420.5 | 5.77 | 7.68 | 7.87 | 6.02 | 6.00 | 6.57 | 6.94 | 5.97 |
| 104 | 0.00091 | 1.12 | 0.59 | 0.66 | 588.5 | 8.26 | 9.37 | 8.72 | 8.05 | 8.40 | 8.95 | 8.30 | 8.68 |
| 105 | 0.00324 | 1.32 | 0.89 | 0.51 | 2453.5 | 6.07 | 7.39 | 7.47 | 6.98 | 6.39 | 6.91 | 7.92 | 6.61 |
| 106 | 0.00091 | 1.21 | 0.77 | 0.58 | 1379.5 | 3.46 | 4.67 | 4.16 | 3.42 | 3.65 | 3.67 | 3.84 | 3.60 |
| 107 | 0.00091 | 1.11 | 0.62 | 0.67 | 508.5 | 9.64 | 10.76 | 10.14 | 10.07 | 9.31 | 9.57 | 9.61 | 10.83 |
| 108 | 0.01547 | 1.08 | 0.81 | 0.47 | 3139 | 5.46 | 6.54 | 6.10 | 5.48 | 5.65 | 6.33 | 5.87 | 5.34 |
| 109 | 0.00091 | 1.19 | 0.59 | 0.80 | 13.5 | 7.38 | 8.58 | 7.86 | 8.29 | 8.13 | 8.36 | 8.54 | 8.39 |
| 110 | 0.00091 | 1.95 | 1.02 | 0.65 | 617 | 4.90 | 6.85 | 6.71 | 5.39 | 4.94 | 6.34 | 6.96 | 6.99 |
| 111 | 0.00091 | 1.20 | 0.77 | 0.52 | 2399 | 7.58 | 8.77 | 9.32 | 7.91 | 7.87 | 8.34 | 8.42 | 8.18 |
| 112 | 0.00091 | 1.76 | 0.94 | 0.56 | 1683 | 7.08 | 8.85 | 8.83 | 7.36 | 7.16 | 8.51 | 8.94 | 8.80 |
| 113 | 0.00091 | 2.09 | 1.19 | 0.60 | 1129.5 | 8.26 | 10.34 | 10.60 | 9.03 | 8.62 | 9.44 | 9.72 | 10.92 |
| 114 | 0.00091 | 2.66 | 1.51 | 0.63 | 807 | 7.88 | 10.54 | 10.49 | 8.40 | 7.91 | 9.23 | 9.06 | 8.09 |
| 115 | 0.00091 | 1.02 | 0.51 | 0.64 | 683.5 | 9.71 | 10.73 | 10.54 | 10.00 | 9.72 | 10.82 | 10.49 | 11.04 |
| 116 | 0.00091 | 1.59 | 0.97 | 0.49 | 2941 | 7.77 | 9.37 | 9.76 | 7.95 | 8.01 | 8.19 | 8.89 | 8.63 |
| 117 | 0.00091 | 1.06 | 0.56 | 0.56 | 1683 | 8.68 | 9.74 | 9.88 | 9.04 | 8.90 | 9.93 | 9.30 | 10.08 |
| 118 | 0.00687 | 1.84 | 1.40 | 0.44 | 3927.5 | 4.93 | 6.76 | 7.42 | 4.70 | 6.22 | 6.59 | 6.23 | 4.27 |
| 119 | 0.00091 | 1.23 | 0.72 | 0.52 | 2399 | 7.36 | 8.59 | 9.11 | 7.81 | 7.48 | 8.16 | 8.38 | 8.26 |
| 120 | 0.00195 | 1.27 | 0.97 | 0.45 | 3644.5 | 5.62 | 6.89 | 7.75 | 5.65 | 5.76 | 6.35 | 6.72 | 6.03 |
| 121 | 0.00091 | 1.12 | 0.63 | 0.53 | 2087.5 | 11.46 | 12.57 | 12.82 | 11.81 | 11.56 | 12.01 | 12.07 | 11.85 |
| 122 | 0.00091 | 1.06 | 0.60 | 0.58 | 1420.5 | 6.63 | 7.68 | 7.80 | 6.70 | 6.56 | 7.18 | 7.21 | 7.07 |
| 123 | 0.00145 | 1.57 | 1.07 | 0.50 | 2583.5 | 6.99 | 8.56 | 8.83 | 7.55 | 6.97 | 7.74 | 8.88 | 7.51 |
| 124 | 0.00091 | 1.52 | 0.80 | 0.58 | 1420.5 | 5.22 | 6.73 | 6.64 | 5.29 | 5.11 | 6.05 | 5.53 | 5.65 |
| 125 | 0.00239 | 2.09 | 1.61 | 0.52 | 2218.5 | 5.29 | 7.38 | 7.68 | 5.43 | 5.30 | 5.94 | 7.41 | 4.95 |
| 126 | 0.00145 | 1.35 | 0.79 | 0.59 | 1206.5 | 4.43 | 5.78 | 5.63 | 4.60 | 5.01 | 4.75 | 4.96 | 4.71 |
| 127 | 0.00091 | 3.10 | 1.84 | 0.57 | 1585.5 | 5.37 | 8.47 | 9.13 | 6.68 | 5.49 | 7.60 | 7.18 | 5.56 |
| 128 | 0.00145 | 2.03 | 1.21 | 0.61 | 967 | 5.77 | 7.81 | 7.64 | 6.23 | 6.37 | 6.70 | 7.10 | 5.85 |
| 129 | 0.00091 | 3.23 | 1.90 | 0.54 | 1962 | 6.22 | 9.45 | 10.34 | 6.90 | 6.38 | 8.08 | 8.49 | 6.84 |
| 130 | 0.00091 | 2.84 | 1.78 | 0.52 | 2218.5 | 4.27 | 7.11 | 8.00 | 4.42 | 4.53 | 4.55 | 5.92 | 4.41 |
| 131 | 0.01263 | 1.38 | 1.01 | 0.42 | 4160.5 | 7.50 | 8.88 | 9.42 | 7.69 | 7.46 | 8.61 | 8.61 | 7.71 |
| 132 | 0.01781 | 1.01 | 0.71 | 0.49 | 2941 | 6.44 | 7.45 | 7.33 | 6.80 | 6.44 | 7.41 | 6.72 | 6.26 |
| 133 | 0.00091 | 1.52 | 0.73 | 0.81 | 7.5 | 8.48 | 10.00 | 9.59 | 9.63 | 9.15 | 9.64 | 9.81 | 10.18 |
| 134 | 0.00091 | 1.06 | 0.73 | 0.51 | 2453.5 | 6.96 | 8.02 | 8.44 | 7.17 | 7.01 | 7.55 | 7.64 | 6.95 |
| 135 | 0.03210 | 1.06 | 0.88 | 0.35 | 5555 | 5.72 | 6.78 | 6.78 | 5.55 | 6.14 | 6.28 | 6.67 | 6.21 |
| 136 | 0.00091 | 1.75 | 1.12 | 0.52 | 2218.5 | 7.05 | 8.79 | 9.41 | 7.61 | 7.21 | 8.94 | 9.52 | 7.42 |
| 137 | 0.00091 | 1.26 | 0.68 | 0.53 | 2087.5 | 9.06 | 10.32 | 10.57 | 9.54 | 9.35 | 9.60 | 9.58 | 9.89 |
| 138 | 0.04753 | 1.21 | 1.10 | 0.33 | 6143.5 | 6.32 | 7.53 | 8.20 | 6.08 | 6.36 | 7.34 | 7.81 | 6.10 |
| 139 | 0.00091 | 1.20 | 0.65 | 0.61 | 1089.5 | 5.58 | 6.78 | 6.72 | 5.74 | 5.69 | 5.73 | 6.22 | 5.55 |
| 140 | 0.00091 | 1.53 | 0.79 | 0.61 | 1089.5 | 8.86 | 10.39 | 10.41 | 9.27 | 8.87 | 10.01 | 9.79 | 10.15 |
| 141 | 0.00091 | 1.35 | 0.94 | 0.51 | 2453.5 | 6.10 | 7.45 | 8.15 | 6.26 | 6.11 | 6.81 | 6.47 | 5.93 |
| 142 | 0.00091 | 1.57 | 1.07 | 0.52 | 2399 | 7.55 | 9.12 | 9.65 | 7.77 | 7.93 | 9.14 | 9.24 | 7.61 |
| 143 | 0.00091 | 1.29 | 0.77 | 0.55 | 1825.5 | 5.62 | 6.91 | 7.42 | 6.09 | 5.62 | 6.36 | 6.33 | 7.14 |
| 144 | 0.00091 | 1.30 | 0.74 | 0.67 | 451 | 4.44 | 5.74 | 5.31 | 4.56 | 4.51 | 5.02 | 5.00 | 4.85 |
| 145 | 0.00091 | 1.34 | 0.68 | 0.64 | 739.5 | 11.40 | 12.73 | 12.77 | 11.93 | 11.79 | 12.08 | 12.25 | 12.21 |
| 146 | 0.00091 | 1.39 | 0.84 | 0.60 | 1129.5 | 6.07 | 7.45 | 7.68 | 6.50 | 6.15 | 6.54 | 6.67 | 7.54 |
| 147 | 0.00091 | 3.11 | 1.85 | 0.54 | 1962 | 5.75 | 8.86 | 9.73 | 6.42 | 5.56 | 7.46 | 7.65 | 6.11 |
| 148 | 0.00195 | 1.48 | 0.88 | 0.53 | 2087.5 | 5.65 | 7.13 | 7.32 | 6.03 | 5.88 | 6.77 | 6.93 | 6.78 |
| 149 | 0.00195 | 1.67 | 1.05 | 0.61 | 967 | 6.23 | 7.90 | 7.57 | 6.85 | 6.90 | 7.32 | 7.01 | 6.67 |
| 150 | 0.00091 | 1.51 | 0.86 | 0.50 | 2583.5 | 7.76 | 9.27 | 9.61 | 7.91 | 7.81 | 9.29 | 9.16 | 8.77 |
| 151 | 0.00091 | 1.08 | 0.56 | 0.64 | 739.5 | 6.88 | 7.96 | 7.91 | 7.09 | 7.05 | 7.24 | 7.22 | 7.93 |
| 152 | 0.00091 | 1.12 | 0.58 | 0.63 | 807 | 7.93 | 9.05 | 8.99 | 8.20 | 7.79 | 8.54 | 8.58 | 8.61 |
| 153 | 0.00091 | 2.12 | 1.10 | 0.59 | 1279.5 | 5.23 | 7.35 | 7.32 | 5.48 | 5.77 | 6.44 | 6.46 | 5.57 |
| 154 | 0.00091 | 1.03 | 0.54 | 0.62 | 926.5 | 7.76 | 8.79 | 8.59 | 8.46 | 8.34 | 8.32 | 8.69 | 8.81 |
| 155 | 0.00091 | 1.06 | 0.64 | 0.58 | 1379.5 | 4.39 | 5.45 | 5.14 | 4.32 | 5.18 | 5.44 | 4.62 | 4.53 |
| 156 | 0.00091 | 5.93 | 3.07 | 0.56 | 1683 | 5.18 | 11.11 | 11.87 | 8.26 | 7.12 | 11.09 | 11.17 | 8.85 |
| 157 | 0.00091 | 1.60 | 0.96 | 0.53 | 2087.5 | 6.54 | 8.13 | 8.41 | 6.73 | 6.25 | 7.24 | 7.24 | 7.94 |
| 158 | 0.00091 | 1.90 | 0.96 | 0.67 | 451 | 4.90 | 6.81 | 6.24 | 5.21 | 5.29 | 6.03 | 7.23 | 6.40 |
| 159 | 0.00091 | 1.73 | 0.89 | 0.65 | 617 | 5.68 | 7.42 | 7.32 | 6.15 | 5.91 | 6.74 | 6.42 | 6.77 |
| 160 | 0.00534 | 1.36 | 0.99 | 0.48 | 3075 | 7.34 | 8.70 | 9.00 | 7.84 | 7.52 | 9.05 | 8.98 | 7.12 |
| 161 | 0.00091 | 1.20 | 0.57 | 0.76 | 72 | 6.74 | 7.94 | 7.35 | 7.01 | 7.15 | 7.34 | 7.23 | 7.65 |
| 162 | 0.00091 | 1.03 | 0.73 | 0.48 | 3075 | 5.84 | 6.86 | 7.47 | 6.07 | 5.67 | 6.39 | 6.52 | 6.03 |
| 163 | 0.00449 | 1.09 | 0.84 | 0.44 | 3927.5 | 7.93 | 9.02 | 9.59 | 8.65 | 8.25 | 8.91 | 9.22 | 8.28 |
| 164 | 0.00091 | 1.44 | 0.78 | 0.50 | 2583.5 | 7.52 | 8.96 | 9.23 | 8.08 | 7.47 | 8.02 | 8.19 | 8.45 |
| 165 | 0.00091 | 1.46 | 1.20 | 0.47 | 3264 | 3.75 | 5.21 | 6.37 | 3.91 | 3.93 | 4.29 | 4.76 | 3.83 |
| 166 | 0.01692 | 1.09 | 0.83 | 0.41 | 4384 | 5.76 | 6.84 | 6.93 | 5.60 | 6.52 | 6.69 | 6.45 | 5.69 |
| 167 | 0.00091 | 2.09 | 1.09 | 0.64 | 683.5 | 7.05 | 9.14 | 9.08 | 7.80 | 7.39 | 8.37 | 8.37 | 8.16 |
| 168 | 0.00091 | 2.61 | 1.24 | 0.75 | 77.5 | 6.90 | 9.51 | 8.22 | 7.43 | 7.15 | 8.70 | 8.95 | 8.43 |
| 169 | 0.00091 | 1.51 | 1.04 | 0.50 | 2718.5 | 6.12 | 7.63 | 8.13 | 6.29 | 6.22 | 7.37 | 5.89 | 6.18 |
| 170 | 0.00091 | 1.36 | 0.80 | 0.60 | 1129.5 | 5.48 | 6.84 | 6.73 | 6.29 | 5.31 | 5.69 | 5.85 | 7.16 |
| 171 | 0.00091 | 1.47 | 0.83 | 0.52 | 2399 | 6.94 | 8.41 | 8.74 | 7.19 | 7.15 | 8.67 | 7.49 | 7.44 |
| 172 | 0.00091 | 1.22 | 0.68 | 0.66 | 588.5 | 7.08 | 8.31 | 8.18 | 7.71 | 7.17 | 8.07 | 7.84 | 7.88 |
| 173 | 0.00091 | 1.06 | 0.58 | 0.61 | 967 | 6.74 | 7.79 | 7.85 | 7.04 | 6.63 | 7.18 | 6.99 | 7.16 |
| 174 | 0.00091 | 1.35 | 0.76 | 0.56 | 1683 | 7.64 | 8.99 | 9.02 | 7.88 | 7.73 | 8.64 | 8.48 | 8.11 |
| 175 | 0.00091 | 1.80 | 1.06 | 0.53 | 2087.5 | 8.32 | 10.11 | 10.17 | 9.13 | 7.97 | 8.69 | 9.16 | 9.67 |
| 176 | 0.00091 | 1.01 | 0.52 | 0.64 | 739.5 | 5.13 | 6.14 | 6.03 | 5.28 | 5.39 | 6.06 | 5.79 | 6.07 |
| 177 | 0.00239 | 2.65 | 1.96 | 0.49 | 2941 | 6.13 | 8.78 | 9.74 | 7.24 | 7.60 | 8.80 | 9.86 | 6.76 |
| 178 | 0.00091 | 1.52 | 0.83 | 0.70 | 265.5 | 4.61 | 6.13 | 5.31 | 4.91 | 4.90 | 5.61 | 5.35 | 4.71 |
| 179 | 0.00091 | 1.81 | 0.90 | 0.63 | 807 | 8.78 | 10.59 | 10.48 | 9.35 | 9.27 | 10.52 | 10.67 | 9.93 |
| 180 | 0.00091 | 1.11 | 0.75 | 0.52 | 2218.5 | 7.18 | 8.30 | 8.63 | 7.08 | 7.03 | 7.77 | 7.87 | 6.95 |
| 181 | 0.00091 | 1.12 | 0.77 | 0.60 | 1129.5 | 5.69 | 6.82 | 6.74 | 5.82 | 5.63 | 6.20 | 6.44 | 5.52 |
| 182 | 0.00091 | 1.53 | 0.83 | 0.54 | 1962 | 8.15 | 9.68 | 9.96 | 8.58 | 8.05 | 9.25 | 9.03 | 8.85 |
| 183 | 0.00091 | 1.15 | 0.58 | 0.72 | 186 | 7.84 | 8.99 | 8.69 | 8.24 | 7.61 | 8.40 | 8.18 | 8.33 |
| 184 | 0.00091 | 1.33 | 0.68 | 0.62 | 926.5 | 7.95 | 9.28 | 9.33 | 8.73 | 8.31 | 8.85 | 8.84 | 8.88 |
| 185 | 0.00091 | 1.09 | 0.59 | 0.69 | 289 | 6.78 | 7.87 | 7.50 | 7.10 | 7.09 | 7.37 | 7.66 | 7.02 |
| 186 | 0.00091 | 1.52 | 0.77 | 0.65 | 617 | 8.64 | 10.16 | 10.10 | 9.04 | 8.76 | 10.02 | 10.11 | 9.48 |
| 187 | 0.00091 | 1.25 | 0.61 | 0.69 | 289 | 5.33 | 6.58 | 6.52 | 5.85 | 5.49 | 6.12 | 6.14 | 6.04 |
| 188 | 0.00091 | 1.41 | 0.81 | 0.56 | 1683 | 8.08 | 9.48 | 9.60 | 8.80 | 8.79 | 9.40 | 9.88 | 9.32 |
| 189 | 0.00091 | 1.00 | 0.61 | 0.40 | 4704.5 | 7.30 | 8.31 | 8.71 | 7.20 | 7.13 | 7.73 | 7.56 | 7.71 |
| 190 | 0.00091 | 3.05 | 2.24 | 0.54 | 1962 | 3.36 | 6.41 | 8.25 | 3.88 | 3.46 | 4.37 | 6.24 | 3.94 |
| 191 | 0.00145 | 1.06 | 0.70 | 0.49 | 2941 | 7.21 | 8.27 | 8.41 | 6.88 | 6.65 | 7.37 | 7.26 | 7.08 |
| 192 | 0.00091 | 2.56 | 1.36 | 0.64 | 739.5 | 6.36 | 8.92 | 8.90 | 7.26 | 6.42 | 8.08 | 8.14 | 7.76 |
| 193 | 0.00091 | 3.82 | 2.06 | 0.55 | 1825.5 | 5.27 | 9.09 | 9.78 | 6.66 | 5.90 | 8.71 | 9.40 | 8.17 |
| 194 | 0.00091 | 1.15 | 0.68 | 0.48 | 3075 | 8.54 | 9.70 | 10.02 | 8.85 | 9.00 | 9.73 | 9.79 | 9.33 |
| 195 | 0.00091 | 1.25 | 0.61 | 0.82 | 2.5 | 5.99 | 7.23 | 6.22 | 6.73 | 6.38 | 6.66 | 7.26 | 6.38 |
| 196 | 0.00091 | 1.85 | 0.90 | 0.66 | 588.5 | 9.40 | 11.24 | 11.01 | 9.84 | 9.47 | 10.88 | 10.74 | 10.47 |
| 197 | 0.00283 | 2.85 | 1.92 | 0.50 | 2718.5 | 5.95 | 8.80 | 9.08 | 5.67 | 7.64 | 8.69 | 7.93 | 4.42 |
| 198 | 0.00091 | 1.61 | 0.87 | 0.60 | 1129.5 | 6.35 | 7.96 | 7.84 | 6.70 | 6.66 | 7.75 | 7.91 | 7.28 |
| 199 | 0.00091 | 1.11 | 0.65 | 0.55 | 1825.5 | 8.06 | 9.16 | 9.48 | 8.34 | 8.16 | 8.81 | 8.53 | 8.72 |
| 200 | 0.00145 | 1.26 | 0.93 | 0.42 | 4160.5 | 5.63 | 6.88 | 7.56 | 5.52 | 5.60 | 6.65 | 6.64 | 5.79 |
| 201 | 0.00091 | 1.89 | 0.92 | 0.73 | 158 | 5.32 | 7.20 | 6.82 | 5.73 | 5.55 | 6.50 | 6.04 | 6.23 |
| 202 | 0.00091 | 1.19 | 0.64 | 0.59 | 1279.5 | 8.71 | 9.90 | 10.01 | 9.12 | 9.05 | 9.28 | 9.20 | 9.86 |
| 203 | 0.00091 | 1.42 | 0.71 | 0.68 | 394 | 8.90 | 10.32 | 10.02 | 9.52 | 9.49 | 10.09 | 9.81 | 10.18 |
| 204 | 0.00091 | 2.62 | 1.36 | 0.70 | 265.5 | 6.55 | 9.16 | 8.41 | 8.37 | 8.60 | 8.32 | 8.60 | 8.75 |
| 205 | 0.00368 | 1.20 | 0.75 | 0.59 | 1279.5 | 5.70 | 6.90 | 6.11 | 6.16 | 5.50 | 5.70 | 6.27 | 6.68 |
| 206 | 0.00091 | 1.28 | 0.78 | 0.58 | 1379.5 | 6.79 | 8.07 | 7.97 | 6.91 | 7.03 | 7.98 | 8.05 | 7.18 |
| 207 | 0.00091 | 1.49 | 0.75 | 0.65 | 617 | 6.13 | 7.61 | 7.43 | 6.50 | 6.46 | 7.24 | 6.96 | 6.85 |
| 208 | 0.00091 | 1.16 | 0.60 | 0.60 | 1129.5 | 6.95 | 8.10 | 8.21 | 7.33 | 6.97 | 7.55 | 7.61 | 7.92 |
| 209 | 0.00091 | 1.51 | 0.92 | 0.52 | 2218.5 | 6.95 | 8.46 | 8.43 | 7.42 | 7.69 | 8.12 | 8.63 | 8.05 |
| 210 | 0.00091 | 1.07 | 0.56 | 0.64 | 683.5 | 7.78 | 8.85 | 8.68 | 8.07 | 7.71 | 8.60 | 8.64 | 8.83 |
| 211 | 0.00091 | 1.41 | 0.95 | 0.55 | 1779 | 4.79 | 6.21 | 6.12 | 4.46 | 4.58 | 5.36 | 5.52 | 5.14 |
| 212 | 0.00091 | 1.51 | 1.04 | 0.50 | 2718.5 | 6.12 | 7.63 | 8.13 | 6.29 | 6.22 | 7.37 | 5.89 | 6.18 |
| 213 | 0.00091 | 1.49 | 0.75 | 0.65 | 617 | 6.13 | 7.61 | 7.43 | 6.50 | 6.46 | 7.24 | 6.96 | 6.85 |
| 214 | 0.00091 | 1.58 | 1.07 | 0.53 | 2087.5 | 7.36 | 8.94 | 9.45 | 7.60 | 7.73 | 8.95 | 9.04 | 7.40 |
| 215 | 0.00091 | 3.10 | 1.84 | 0.57 | 1585.5 | 5.37 | 8.47 | 9.13 | 6.68 | 5.49 | 7.60 | 7.18 | 5.56 |
| 216 | 0.17394 | −0.80 | 1.09 | −0.28 | 7625.5 | 10.23 | 9.43 | 10.97 | 9.12 | 10.14 | 9.58 | 10.82 | 8.68 |
| 217 | 0.05878 | −1.43 | 1.50 | −0.39 | 4836 | 12.07 | 10.64 | 12.25 | 11.1 | 11.09 | 10.91 | 12.28 | 10.33 |
| 218 | 0.15063 | −1.16 | 1.27 | −0.29 | 7339 | 11.68 | 10.52 | 12.23 | 10.38 | 11.18 | 10.85 | 12.09 | 9.73 |
| 219 | 0.81402 | −0.23 | 1.19 | −0.03 | 15885.5 | 8.40 | 8.17 | 6.61 | 8.76 | 7.89 | 7.55 | 8.85 | 9.17 |
| 220 | 0.81402 | −0.21 | 1.16 | −0.02 | 16099 | 8.48 | 8.27 | 6.72 | 8.81 | 8.01 | 7.59 | 8.89 | 9.22 |
| 221 | 0.85397 | −0.22 | 1.17 | −0.02 | 16099 | 9.14 | 8.92 | 7.43 | 9.53 | 8.66 | 8.27 | 9.51 | 9.87 |
| 222 | 0.85397 | −0.23 | 1.02 | −0.03 | 15885.5 | 10.03 | 9.80 | 8.50 | 10.36 | 9.58 | 9.27 | 10.36 | 10.67 |
| 223 | 0.81402 | −0.22 | 1.07 | −0.03 | 15885.5 | 9.65 | 9.43 | 8.06 | 10.00 | 9.20 | 8.89 | 10.01 | 10.33 |
| 224 | 0.77481 | −0.14 | 0.73 | −0.04 | 15420.5 | 11.75 | 11.61 | 10.65 | 11.97 | 11.53 | 11.20 | 12.05 | 12.26 |
| 225 | 0.81402 | −0.17 | 0.78 | −0.02 | 16099 | 10.99 | 10.82 | 9.84 | 11.25 | 10.70 | 10.39 | 11.28 | 11.52 |
| 226 | 0.11540 | 0.64 | 0.73 | 0.33 | 6143.5 | 6.17 | 6.81 | 6.68 | 6.61 | 6.58 | 6.94 | 7.31 | 6.61 |
| 227 | 0.12994 | 0.61 | 0.76 | 0.31 | 6652 | 6.02 | 6.62 | 6.44 | 6.67 | 6.23 | 6.78 | 7.23 | 6.68 |
| 228 | 0.27593 | 0.60 | 0.83 | 0.24 | 8600 | 5.83 | 6.43 | 6.37 | 6.37 | 6.17 | 6.60 | 7.03 | 6.37 |
| 229 | 0.68900 | 0.43 | 1.04 | 0.13 | 12615.5 | 5.58 | 6.01 | 4.97 | 7.07 | 4.83 | 6.17 | 6.96 | 7.14 |
| 230 | 0.89542 | −0.19 | 1.10 | 0.01 | 16758 | 8.91 | 8.72 | 7.29 | 9.33 | 8.44 | 8.10 | 9.38 | 9.65 |
| 231 | 0.89542 | −0.25 | 1.17 | −0.01 | 16646.5 | 8.59 | 8.35 | 6.82 | 9.07 | 8.07 | 7.72 | 8.98 | 9.28 |
| 232 | 0.81402 | −0.27 | 1.14 | −0.02 | 16099 | 9.00 | 8.73 | 7.28 | 9.45 | 8.49 | 8.12 | 9.36 | 9.72 |
| 233 | 0.97119 | −0.20 | 0.98 | 0.04 | 15323.5 | 10.31 | 10.11 | 8.86 | 10.67 | 9.95 | 9.56 | 10.65 | 10.93 |
| 234 | 0.77481 | −0.26 | 1.09 | −0.05 | 15120 | 10.18 | 9.92 | 8.60 | 10.59 | 9.78 | 9.37 | 10.50 | 10.92 |
| 235 | 0.39661 | 0.36 | 0.77 | 0.21 | 9615 | 5.32 | 5.68 | 4.82 | 6.61 | 4.69 | 5.78 | 6.70 | 6.52 |
| 236 | 0.27658 | 1.25 | 1.48 | 0.21 | 9615 | 4.63 | 5.88 | 6.68 | 4.22 | 6.32 | 6.40 | 5.40 | 3.85 |
| 237 | 0.37089 | −0.56 | 0.94 | −0.21 | 9762.5 | 7.74 | 7.18 | 8.78 | 7.84 | 7.91 | 7.49 | 7.21 | 5.34 |
| 238 | 0.03259 | −1.12 | 1.05 | −0.38 | 5141 | 7.33 | 6.21 | 6.55 | 6.56 | 6.51 | 6.14 | 5.93 | 6.00 |
| 239 | 0.24604 | −0.46 | 0.75 | −0.24 | 8850 | 7.28 | 6.83 | 7.94 | 7.32 | 7.41 | 7.05 | 6.68 | 5.51 |
| 240 | 0.24604 | −0.46 | 0.75 | −0.24 | 8850 | 7.28 | 6.83 | 7.94 | 7.32 | 7.41 | 7.05 | 6.68 | 5.51 |
| 241 | 0.05702 | −1.15 | 1.15 | −0.34 | 5990 | 8.29 | 7.14 | 8.96 | 7.27 | 8.17 | 7.65 | 6.98 | 5.67 |
| 242 | 0.77481 | −0.13 | 0.87 | −0.09 | 13834 | 7.60 | 7.47 | 8.74 | 7.02 | 6.99 | 7.26 | 7.39 | 6.21 |
| 243 | 0.73019 | 0.62 | 1.13 | 0.10 | 13537.5 | 4.21 | 4.83 | 5.94 | 4.09 | 4.51 | 4.67 | 6.62 | 3.86 |
| 244 | 0.00145 | −1.10 | 0.76 | −0.55 | 1734.5 | 8.25 | 7.15 | 7.82 | 8.43 | 8.50 | 7.87 | 8.41 | 7.66 |
| 245 | 0.24604 | −0.53 | 0.74 | −0.24 | 8701 | 8.01 | 7.48 | 8.33 | 7.95 | 8.06 | 7.59 | 7.25 | 6.25 |
| 246 | 0.11319 | −1.15 | 1.47 | −0.28 | 7625.5 | 6.29 | 5.15 | 6.83 | 4.67 | 6.81 | 6.06 | 5.39 | 4.56 |
| 247 | 0.10518 | 0.75 | 1.12 | 0.24 | 8955.5 | 4.65 | 5.40 | 6.42 | 4.36 | 6.19 | 5.90 | 5.32 | 4.06 |
| 248 | 0.40265 | −0.29 | 1.24 | −0.18 | 10870.5 | 5.24 | 4.95 | 7.39 | 4.28 | 4.54 | 5.15 | 5.37 | 4.01 |
| 249 | 0.26953 | −0.49 | 0.80 | −0.25 | 8382.5 | 6.11 | 5.62 | 6.51 | 5.32 | 6.22 | 6.06 | 5.69 | 5.23 |
| 250 | 0.44743 | −0.68 | 1.60 | −0.18 | 10870.5 | 6.25 | 5.57 | 8.11 | 4.90 | 5.03 | 5.54 | 5.15 | 4.26 |
| 251 | 0.19724 | 0.48 | 0.72 | 0.31 | 6825 | 7.93 | 8.41 | 7.81 | 7.62 | 7.60 | 7.61 | 7.25 | 7.67 |
| 252 | 0.05098 | −0.68 | 0.74 | −0.36 | 5388.5 | 9.16 | 8.48 | 9.86 | 9.13 | 9.21 | 8.93 | 8.77 | 7.69 |
| 253 | 0.27612 | 0.49 | 0.77 | 0.21 | 9914.5 | 5.51 | 6.00 | 5.84 | 5.38 | 5.59 | 5.93 | 5.54 | 5.43 |
| 254 | 0.30431 | 0.47 | 0.75 | 0.24 | 8955.5 | 5.53 | 6.00 | 5.92 | 5.44 | 5.55 | 5.97 | 5.58 | 5.45 |
| 255 | 0.19839 | −0.66 | 0.87 | −0.27 | 7837 | 9.70 | 9.04 | 10.45 | 9.68 | 9.68 | 9.22 | 9.18 | 7.20 |
| 256 | 0.19839 | −0.66 | 0.87 | −0.27 | 7837 | 9.70 | 9.04 | 10.45 | 9.68 | 9.68 | 9.22 | 9.18 | 7.20 |
| 257 | 0.19839 | −0.66 | 0.87 | −0.27 | 7837 | 9.70 | 9.04 | 10.45 | 9.68 | 9.68 | 9.22 | 9.18 | 7.20 |
| 258 | 0.19839 | −0.66 | 0.87 | −0.27 | 7837 | 9.70 | 9.04 | 10.45 | 9.68 | 9.68 | 9.22 | 9.18 | 7.20 |
| 259 | 0.19839 | −0.66 | 0.87 | −0.27 | 7837 | 9.70 | 9.04 | 10.45 | 9.68 | 9.68 | 9.22 | 9.18 | 7.20 |
| 260 | 0.19839 | −0.66 | 0.87 | −0.27 | 7837 | 9.70 | 9.04 | 10.45 | 9.68 | 9.68 | 9.22 | 9.18 | 7.20 |
| 261 | 0.19839 | −0.66 | 0.87 | −0.27 | 7837 | 9.70 | 9.04 | 10.45 | 9.68 | 9.68 | 9.22 | 9.18 | 7.20 |
| 262 | 0.27499 | −0.46 | 0.75 | −0.24 | 8850 | 6.05 | 5.59 | 6.83 | 6.13 | 6.04 | 5.91 | 5.68 | 4.63 |
| 263 | 0.02669 | −1.10 | 1.00 | −0.42 | 4233.5 | 10.57 | 9.47 | 11.20 | 10.09 | 10.44 | 9.96 | 9.60 | 7.88 |
| 264 | 0.02669 | −1.10 | 1.00 | −0.42 | 4233.5 | 10.57 | 9.47 | 11.20 | 10.09 | 10.44 | 9.96 | 9.60 | 7.88 |
| 265 | 0.64689 | −0.73 | 1.29 | −0.19 | 10385.5 | 5.04 | 4.32 | 6.20 | 4.51 | 4.31 | 4.49 | 4.07 | 4.25 |
| 266 | 0.33779 | −0.55 | 0.74 | −0.24 | 8850 | 10.42 | 9.86 | 10.96 | 10.29 | 10.28 | 9.97 | 10.13 | 8.93 |
| 267 | 0.33779 | −0.53 | 0.73 | −0.24 | 8850 | 10.42 | 9.89 | 10.95 | 10.31 | 10.30 | 9.99 | 10.16 | 8.95 |
| 268 | 0.33779 | −0.55 | 0.74 | −0.24 | 8850 | 10.42 | 9.86 | 10.96 | 10.29 | 10.28 | 9.97 | 10.13 | 8.93 |
| 269 | 0.33779 | −0.55 | 0.74 | −0.24 | 8850 | 10.42 | 9.86 | 10.96 | 10.29 | 10.28 | 9.97 | 10.13 | 8.93 |
| 270 | 0.33779 | −0.55 | 0.74 | −0.24 | 8850 | 10.42 | 9.86 | 10.96 | 10.29 | 10.28 | 9.97 | 10.13 | 8.93 |
| 271 | 0.00965 | −0.90 | 0.72 | −0.41 | 4323 | 6.38 | 5.48 | 6.32 | 6.41 | 6.77 | 6.25 | 6.66 | 5.49 |
| 272 | 0.00965 | −0.90 | 0.72 | −0.41 | 4323 | 6.38 | 5.48 | 6.32 | 6.41 | 6.77 | 6.25 | 6.66 | 5.49 |
| 273 | 0.01637 | 0.95 | 0.77 | 0.39 | 4773.5 | 6.45 | 7.40 | 8.03 | 6.81 | 6.56 | 7.30 | 7.31 | 7.04 |
| 274 | 0.01637 | 0.95 | 0.77 | 0.39 | 4773.5 | 6.45 | 7.40 | 8.03 | 6.81 | 6.56 | 7.30 | 7.31 | 7.04 |
| 275 | 0.33779 | −0.41 | 0.96 | −0.21 | 9762.5 | 7.42 | 7.01 | 8.76 | 7.12 | 7.65 | 7.40 | 7.28 | 5.86 |
| 276 | 0.05566 | 1.52 | 1.56 | 0.34 | 5990 | 5.89 | 7.41 | 7.88 | 6.13 | 7.78 | 7.26 | 7.44 | 4.91 |
| 277 | 0.52467 | −0.68 | 1.49 | −0.17 | 11150.5 | 7.58 | 6.90 | 8.36 | 6.17 | 6.34 | 6.71 | 6.15 | 5.10 |
| 278 | 0.56445 | −0.47 | 0.82 | −0.04 | 15614.5 | 9.73 | 9.27 | 9.93 | 10.25 | 9.46 | 9.21 | 9.33 | 7.87 |
| 279 | 0.06240 | −1.05 | 0.88 | −0.33 | 6225.5 | 7.43 | 6.37 | 7.09 | 7.57 | 7.78 | 7.18 | 7.97 | 6.68 |
| 280 | 0.44743 | −0.73 | 1.56 | −0.16 | 11614.5 | 7.95 | 7.22 | 9.45 | 5.98 | 7.03 | 7.51 | 6.64 | 5.55 |
| 281 | 0.22083 | 1.09 | 1.09 | 0.30 | 7124 | 7.17 | 8.25 | 8.84 | 8.48 | 7.90 | 8.08 | 9.66 | 8.24 |
| 282 | 0.30506 | −0.63 | 0.85 | −0.23 | 9073.5 | 9.42 | 8.79 | 9.97 | 9.59 | 9.33 | 8.95 | 8.91 | 7.36 |
| 283 | 0.60546 | −0.30 | 1.03 | −0.10 | 13337.5 | 6.16 | 5.86 | 7.36 | 5.15 | 5.50 | 5.95 | 5.70 | 5.16 |
| 284 | 0.17922 | −0.63 | 1.10 | −0.25 | 8382.5 | 6.87 | 6.24 | 8.14 | 5.85 | 6.00 | 6.34 | 6.36 | 5.54 |
| 285 | 0.22108 | −0.63 | 0.96 | −0.23 | 9073.5 | 6.12 | 5.49 | 6.92 | 5.10 | 5.57 | 5.45 | 5.29 | 5.13 |
| 286 | 0.33718 | −0.76 | 0.84 | −0.18 | 10622 | 10.24 | 9.48 | 9.20 | 10.49 | 9.57 | 9.86 | 10.45 | 11.26 |
| 287 | 0.06811 | −0.83 | 0.87 | −0.35 | 5648 | 8.10 | 7.26 | 8.94 | 7.45 | 7.91 | 7.75 | 7.29 | 5.94 |
| 288 | 0.30506 | −0.44 | 0.72 | −0.19 | 10385.5 | 4.84 | 4.41 | 5.53 | 3.96 | 4.32 | 4.34 | 4.10 | 3.88 |
| 289 | 0.00965 | −0.90 | 0.72 | −0.41 | 4323 | 6.38 | 5.48 | 6.32 | 6.41 | 6.77 | 6.25 | 6.66 | 5.49 |
| 290 | 0.05098 | −0.68 | 0.74 | −0.36 | 5388.5 | 9.16 | 8.48 | 9.86 | 9.13 | 9.21 | 8.93 | 8.77 | 7.69 |
| 291 | 0.00195 | −0.79 | 0.71 | −0.45 | 3547.5 | 7.63 | 6.84 | 8.26 | 6.71 | 7.34 | 7.17 | 6.84 | 6.83 |
| 292 | 0.52430 | −0.38 | 0.90 | −0.16 | 11614.5 | 5.88 | 5.50 | 5.78 | 6.50 | 6.10 | 5.04 | 5.26 | 6.45 |
| 293 | 0.01136 | 0.86 | 0.82 | 0.38 | 4931.5 | 3.88 | 4.74 | 5.03 | 3.89 | 4.08 | 3.99 | 4.32 | 3.90 |
| 294 | 0.45054 | −0.43 | 0.91 | −0.17 | 11150.5 | 7.88 | 7.45 | 9.05 | 7.04 | 6.87 | 7.45 | 7.24 | 7.33 |
| 295 | 1.00000 | −0.05 | 0.82 | −0.01 | 16646.5 | 5.27 | 5.22 | 6.13 | 4.61 | 4.93 | 5.16 | 4.67 | 4.63 |
| 296 | 0.01263 | 0.96 | 0.87 | 0.38 | 4931.5 | 4.33 | 5.28 | 6.06 | 4.28 | 4.50 | 5.60 | 5.39 | 3.98 |
| 297 | 0.03958 | 0.90 | 0.73 | 0.42 | 4160.5 | 5.71 | 6.60 | 6.57 | 5.92 | 6.03 | 6.27 | 6.00 | 5.45 |
| 298 | 0.00091 | −1.49 | 0.86 | −0.47 | 3194 | 5.92 | 4.44 | 4.15 | 5.94 | 6.05 | 4.84 | 5.78 | 5.95 |
| 299 | 0.22111 | −0.48 | 0.81 | −0.22 | 9376 | 6.99 | 6.51 | 8.12 | 6.94 | 7.30 | 7.02 | 6.56 | 5.35 |
| 300 | 0.52315 | −0.46 | 0.73 | −0.18 | 10870.5 | 7.57 | 7.10 | 8.15 | 7.61 | 7.75 | 7.32 | 7.16 | 6.11 |
| 301 | 0.44832 | −0.30 | 1.05 | −0.15 | 11792 | 6.52 | 6.21 | 8.03 | 5.37 | 6.12 | 6.39 | 6.15 | 5.38 |
| 302 | 0.24604 | 0.78 | 1.00 | 0.16 | 11614.5 | 5.81 | 6.59 | 7.18 | 5.28 | 6.22 | 6.05 | 5.38 | 6.49 |
| 303 | 0.39516 | −0.59 | 1.06 | −0.16 | 11614.5 | 6.97 | 6.38 | 7.94 | 6.53 | 6.67 | 6.61 | 8.09 | 5.55 |
| 304 | 0.11319 | −0.68 | 0.77 | −0.32 | 6592.5 | 7.37 | 6.69 | 7.65 | 7.11 | 7.28 | 6.86 | 6.61 | 5.41 |
| 305 | 0.20284 | −0.44 | 0.91 | −0.25 | 8382.5 | 7.44 | 7.00 | 8.56 | 6.44 | 6.76 | 7.14 | 6.85 | 6.26 |
| 306 | 0.15099 | −0.48 | 0.74 | −0.26 | 8173.5 | 7.14 | 6.66 | 8.04 | 6.30 | 5.82 | 6.58 | 6.44 | 6.36 |
| 307 | 0.17103 | −0.57 | 1.04 | −0.25 | 8382.5 | 5.64 | 5.07 | 6.01 | 4.71 | 5.80 | 5.59 | 5.14 | 4.49 |
| 308 | 0.08397 | −0.81 | 0.84 | −0.35 | 5832.5 | 11.31 | 10.50 | 11.97 | 11.23 | 11.32 | 10.80 | 10.80 | 8.93 |
| 309 | 0.24604 | −0.66 | 0.84 | −0.27 | 8044 | 10.66 | 9.99 | 11.41 | 10.70 | 10.69 | 10.18 | 10.15 | 8.39 |
| 310 | 0.00145 | −0.91 | 0.73 | −0.47 | 3317.5 | 10.64 | 9.74 | 11.29 | 10.00 | 9.54 | 9.93 | 10.62 | 9.68 |
| 311 | 0.06083 | −0.88 | 0.89 | −0.36 | 5388.5 | 10.87 | 9.99 | 11.73 | 10.75 | 10.67 | 10.40 | 10.59 | 8.70 |
| 312 | 0.93261 | −0.01 | 0.95 | 0.03 | 15885.5 | 5.62 | 5.61 | 7.36 | 5.50 | 6.39 | 6.55 | 6.00 | 4.43 |
| 313 | 0.08429 | 0.99 | 1.16 | 0.31 | 6825 | 4.61 | 5.61 | 5.94 | 5.63 | 5.58 | 5.70 | 7.86 | 5.40 |
| 314 | 0.24652 | −0.60 | 0.87 | −0.27 | 7837 | 7.98 | 7.38 | 8.73 | 7.87 | 7.96 | 7.78 | 7.58 | 5.97 |
| 315 | 0.05854 | 0.88 | 0.90 | 0.33 | 6322 | 3.98 | 4.85 | 4.87 | 3.95 | 3.60 | 4.28 | 4.88 | 3.54 |
| 316 | 0.00449 | 0.86 | 0.73 | 0.44 | 3927.5 | 3.87 | 4.73 | 4.96 | 3.87 | 3.79 | 4.64 | 4.76 | 4.01 |
| 317 | 0.31305 | −0.44 | 1.00 | −0.20 | 10120 | 6.19 | 5.75 | 6.88 | 5.16 | 5.26 | 5.59 | 5.52 | 4.93 |
| 318 | 0.12913 | −0.74 | 0.79 | −0.33 | 6398.5 | 8.22 | 7.48 | 8.55 | 7.92 | 8.11 | 7.79 | 7.65 | 6.23 |
| 319 | 0.12648 | −0.87 | 0.92 | −0.33 | 6225.5 | 7.33 | 6.45 | 8.27 | 7.04 | 7.21 | 7.04 | 6.55 | 4.97 |
| 320 | 0.19724 | 0.48 | 0.72 | 0.31 | 6825 | 7.93 | 8.41 | 7.81 | 7.62 | 7.60 | 7.61 | 7.25 | 7.67 |
| 321 | 0.24604 | 0.62 | 0.84 | 0.22 | 9376 | 4.19 | 4.81 | 5.74 | 4.20 | 4.30 | 5.41 | 5.01 | 3.88 |
| 322 | 0.17474 | −0.62 | 0.73 | −0.27 | 8044 | 10.63 | 10.01 | 11.29 | 10.14 | 10.42 | 10.88 | 10.99 | 10.42 |
| 323 | 0.17366 | 0.88 | 0.95 | 0.21 | 9615 | 9.44 | 10.32 | 10.56 | 10.12 | 9.56 | 9.58 | 9.92 | 11.08 |
| 324 | 0.68907 | −0.26 | 0.90 | −0.10 | 13646.5 | 6.43 | 6.17 | 7.52 | 5.84 | 5.78 | 6.11 | 5.90 | 5.27 |
| 325 | 0.10081 | 0.99 | 1.20 | 0.27 | 7737.5 | 4.39 | 5.38 | 5.85 | 3.74 | 6.01 | 5.54 | 5.46 | 3.47 |
| 326 | 0.33030 | 1.37 | 2.21 | 0.20 | 10207 | 6.02 | 7.39 | 8.86 | 7.10 | 7.41 | 7.29 | 9.90 | 5.74 |
| 327 | 0.09974 | 1.33 | 1.63 | 0.27 | 7737.5 | 5.04 | 6.37 | 7.13 | 5.24 | 6.89 | 6.70 | 6.63 | 4.20 |
| 328 | 0.08540 | 1.00 | 1.19 | 0.29 | 7423.5 | 4.82 | 5.82 | 6.18 | 4.90 | 6.21 | 5.83 | 5.72 | 4.35 |
| 329 | 0.17254 | 1.60 | 1.92 | 0.25 | 8475.5 | 6.48 | 8.08 | 9.10 | 6.23 | 8.26 | 8.41 | 8.20 | 4.29 |
| 330 | 0.33601 | 1.13 | 1.54 | 0.19 | 10385.5 | 4.67 | 5.80 | 6.53 | 5.18 | 6.72 | 6.28 | 6.56 | 3.72 |
| 331 | 0.44712 | −0.83 | 1.84 | −0.17 | 11150.5 | 6.49 | 5.66 | 8.14 | 4.81 | 4.89 | 5.37 | 5.46 | 3.90 |
| 332 | 0.00195 | 0.92 | 0.71 | 0.47 | 3353 | 5.27 | 6.19 | 6.87 | 5.52 | 5.12 | 5.93 | 5.96 | 5.98 |
| 333 | 0.52442 | −0.44 | 0.81 | −0.21 | 10011.5 | 10.23 | 9.79 | 11.06 | 10.63 | 10.44 | 9.96 | 10.17 | 7.80 |
| 334 | 0.02303 | −1.48 | 1.37 | −0.43 | 4074.5 | 6.38 | 4.90 | 7.14 | 4.61 | 6.41 | 6.19 | 5.51 | 4.24 |
| 335 | 0.06172 | −0.71 | 0.96 | −0.31 | 6726 | 10.68 | 9.96 | 11.60 | 9.53 | 9.70 | 10.12 | 9.77 | 8.75 |
| 336 | 0.24604 | −0.53 | 0.74 | −0.24 | 8701 | 8.01 | 7.48 | 8.33 | 7.95 | 8.06 | 7.59 | 7.25 | 6.25 |
| 337 | 0.09636 | 0.61 | 1.04 | 0.21 | 9914.5 | 5.81 | 6.42 | 8.00 | 5.46 | 5.37 | 6.32 | 6.63 | 6.51 |
| 338 | 0.00091 | 0.99 | 0.74 | 0.54 | 1962 | 4.16 | 5.15 | 5.64 | 4.60 | 4.30 | 4.93 | 5.93 | 4.64 |
| 339 | 0.01325 | 0.93 | 0.75 | 0.41 | 4384 | 6.51 | 7.43 | 7.91 | 6.79 | 6.27 | 7.23 | 6.94 | 6.35 |
| 340 | 0.00091 | 0.91 | 0.84 | 0.44 | 3687.5 | 4.03 | 4.94 | 6.10 | 4.34 | 4.19 | 4.37 | 4.77 | 4.17 |
| 341 | 0.00571 | 0.89 | 0.78 | 0.50 | 2583.5 | 5.54 | 6.43 | 7.19 | 5.71 | 5.47 | 5.72 | 6.04 | 5.72 |
| 342 | 0.15661 | −0.58 | 1.12 | −0.27 | 7837 | 6.05 | 5.47 | 7.06 | 5.08 | 5.11 | 5.30 | 5.60 | 4.72 |
| 343 | 0.52285 | 1.22 | 1.64 | 0.15 | 11879 | 6.19 | 7.41 | 9.10 | 6.57 | 6.69 | 7.52 | 9.60 | 6.04 |
| 344 | 0.51951 | 1.19 | 1.47 | 0.18 | 10870.5 | 3.69 | 4.88 | 5.97 | 3.88 | 4.02 | 4.43 | 6.82 | 3.55 |
| 345 | 0.29676 | 1.17 | 1.39 | 0.21 | 9914.5 | 6.83 | 8.00 | 9.10 | 6.93 | 7.00 | 8.24 | 9.95 | 6.44 |
| 346 | 0.43709 | 1.63 | 1.89 | 0.20 | 10071 | 4.63 | 6.25 | 8.09 | 5.17 | 5.30 | 6.29 | 8.53 | 5.12 |
| 347 | 0.08349 | −0.85 | 0.90 | −0.35 | 5832.5 | 8.78 | 7.93 | 9.42 | 8.57 | 8.65 | 8.17 | 8.07 | 6.39 |
| 348 | 0.06965 | −0.89 | 0.97 | −0.32 | 6592.5 | 7.65 | 6.76 | 8.46 | 6.36 | 7.28 | 7.46 | 7.14 | 5.96 |
| 349 | 0.05658 | −1.43 | 1.44 | −0.35 | 5648 | 7.44 | 6.01 | 7.91 | 6.03 | 7.42 | 7.01 | 6.20 | 4.91 |
| 350 | 0.19839 | −0.66 | 0.87 | −0.27 | 7837 | 9.70 | 9.04 | 10.45 | 9.68 | 9.68 | 9.22 | 9.18 | 7.20 |
| 351 | 0.11479 | −0.75 | 0.85 | −0.33 | 6398.5 | 9.58 | 8.84 | 9.74 | 9.51 | 9.73 | 8.94 | 9.01 | 7.56 |
| 352 | 0.68900 | −0.32 | 0.74 | −0.13 | 12524.5 | 7.62 | 7.30 | 6.57 | 7.32 | 6.85 | 6.63 | 6.21 | 6.80 |
| 353 | 0.04638 | 0.60 | 0.80 | 0.27 | 7737.5 | 5.20 | 5.80 | 6.82 | 6.08 | 5.23 | 5.62 | 5.67 | 4.92 |
| 354 | 0.37184 | −0.58 | 1.17 | −0.19 | 10385.5 | 9.39 | 8.81 | 10.67 | 8.10 | 8.42 | 8.83 | 8.76 | 7.61 |
| 355 | 0.29261 | −0.47 | 0.99 | −0.20 | 10067.5 | 8.19 | 7.72 | 9.66 | 7.34 | 7.57 | 7.61 | 7.76 | 6.80 |
| 356 | 0.73019 | −0.25 | 0.81 | −0.10 | 13646.5 | 7.37 | 7.12 | 7.97 | 7.86 | 7.47 | 7.10 | 7.12 | 5.60 |
| 357 | 0.02449 | −0.79 | 0.88 | −0.36 | 5388.5 | 10.10 | 9.31 | 11.11 | 8.93 | 8.93 | 9.34 | 10.29 | 8.77 |
| 358 | 0.15936 | 0.74 | 0.83 | 0.26 | 8265 | 5.81 | 6.55 | 6.77 | 5.30 | 6.79 | 6.43 | 6.14 | 5.28 |
| 359 | 0.02669 | −1.10 | 1.00 | −0.42 | 4233.5 | 10.57 | 9.47 | 11.20 | 10.09 | 10.44 | 9.96 | 9.60 | 7.88 |
| 360 | 0.21969 | −0.58 | 0.79 | −0.27 | 8044 | 9.57 | 8.99 | 10.21 | 9.90 | 9.59 | 9.23 | 9.21 | 7.46 |
| 361 | 0.33562 | 1.83 | 2.54 | 0.21 | 9914.5 | 7.20 | 9.03 | 11.43 | 8.44 | 9.40 | 9.72 | 11.62 | 7.49 |
| 362 | 0.02749 | 0.88 | 0.81 | 0.47 | 3139 | 4.38 | 5.25 | 4.28 | 5.22 | 4.47 | 4.10 | 5.09 | 6.09 |
| 363 | 0.44639 | 0.46 | 1.41 | 0.10 | 13211 | 4.79 | 5.25 | 6.49 | 4.16 | 6.43 | 5.89 | 5.75 | 3.53 |
| 364 | 0.56490 | 0.84 | 1.94 | 0.08 | 14086.5 | 5.27 | 6.12 | 8.10 | 4.87 | 7.39 | 7.26 | 6.88 | 3.43 |
| 365 | 0.05658 | 0.91 | 1.02 | 0.30 | 7028 | 3.71 | 4.62 | 5.48 | 4.18 | 4.01 | 4.07 | 6.42 | 3.71 |
| 366 | 0.33779 | −0.41 | 0.96 | −0.21 | 9762.5 | 7.42 | 7.01 | 8.76 | 7.12 | 7.65 | 7.40 | 7.28 | 5.86 |
| 367 | 0.44764 | −0.32 | 0.88 | −0.14 | 12105.5 | 6.07 | 5.76 | 6.96 | 5.19 | 5.46 | 5.69 | 5.45 | 5.03 |
| 368 | 0.01870 | −0.66 | 0.74 | −0.36 | 5388.5 | 6.24 | 5.58 | 7.06 | 6.40 | 6.14 | 6.05 | 5.84 | 5.07 |
| 369 | 0.27756 | −0.77 | 1.30 | −0.17 | 11150.5 | 6.86 | 6.08 | 8.42 | 5.54 | 6.11 | 6.50 | 6.18 | 5.42 |
| 370 | 0.04258 | −0.90 | 0.89 | −0.38 | 5141 | 10.63 | 9.73 | 11.36 | 10.48 | 10.55 | 10.15 | 10.22 | 8.26 |
| 371 | 0.44743 | −0.68 | 1.60 | −0.18 | 10870.5 | 6.25 | 5.57 | 8.11 | 4.90 | 5.03 | 5.54 | 5.15 | 4.26 |
| 372 | 0.00195 | −1.31 | 0.76 | −0.61 | 1051 | 9.43 | 8.13 | 8.66 | 9.49 | 9.85 | 8.69 | 8.89 | 9.51 |
| 373 | 0.56499 | −0.35 | 0.77 | −0.14 | 12105.5 | 10.58 | 10.23 | 11.59 | 10.38 | 10.25 | 11.06 | 11.13 | 10.10 |
| 374 | 0.00091 | −1.71 | 0.90 | −0.55 | 1887.5 | 7.05 | 5.34 | 5.32 | 6.50 | 7.16 | 5.81 | 5.48 | 5.68 |
| 375 | 0.37119 | −0.62 | 0.81 | −0.24 | 8701 | 7.91 | 7.29 | 8.82 | 7.94 | 7.96 | 7.72 | 7.55 | 6.09 |
| 376 | 0.22468 | −0.82 | 1.38 | −0.24 | 8701 | 6.18 | 5.36 | 7.41 | 4.88 | 4.92 | 5.44 | 5.09 | 4.46 |
| 377 | 0.19360 | −0.35 | 0.94 | −0.21 | 10011.5 | 7.48 | 7.13 | 9.22 | 7.32 | 6.82 | 7.49 | 7.81 | 6.21 |
| 378 | 0.01781 | 0.96 | 0.80 | 0.38 | 4931.5 | 5.82 | 6.79 | 7.25 | 5.85 | 5.71 | 6.37 | 6.49 | 5.70 |
| 379 | 0.01637 | 0.95 | 0.77 | 0.39 | 4773.5 | 6.45 | 7.40 | 8.03 | 6.81 | 6.56 | 7.30 | 7.31 | 7.04 |
| 380 | 0.73019 | −0.33 | 1.16 | −0.09 | 13834 | 7.53 | 7.20 | 8.70 | 7.33 | 7.67 | 8.16 | 8.34 | 7.54 |
| 381 | 0.00449 | 0.80 | 0.76 | 0.44 | 3687.5 | 5.31 | 6.11 | 6.94 | 5.41 | 5.24 | 5.46 | 5.83 | 5.57 |
| 382 | 0.40265 | −0.29 | 1.24 | −0.18 | 10870.5 | 5.24 | 4.95 | 7.39 | 4.28 | 4.54 | 5.15 | 5.37 | 4.01 |
| 383 | 0.85397 | −0.04 | 0.83 | −0.05 | 15120 | 9.30 | 9.25 | 10.67 | 9.47 | 8.97 | 9.78 | 10.20 | 9.07 |
| 384 | 0.26953 | −0.49 | 0.80 | −0.25 | 8382.5 | 6.11 | 5.62 | 6.51 | 5.32 | 6.22 | 6.06 | 5.69 | 5.23 |
| 385 | 0.10031 | −0.93 | 0.99 | −0.35 | 5832.5 | 6.37 | 5.44 | 6.82 | 5.50 | 6.45 | 5.88 | 5.28 | 4.33 |
| 386 | 0.04873 | 0.77 | 0.82 | 0.35 | 5752.5 | 5.81 | 6.58 | 6.99 | 5.81 | 6.93 | 6.69 | 6.55 | 5.50 |
| 387 | 0.77481 | −0.44 | 1.01 | −0.02 | 16099 | 7.81 | 7.37 | 8.52 | 7.93 | 7.50 | 7.99 | 8.47 | 6.86 |
| 388 | 0.52315 | −0.49 | 0.78 | −0.13 | 12524.5 | 7.70 | 7.21 | 7.98 | 7.73 | 7.70 | 7.12 | 7.07 | 5.65 |
| 389 | 0.30928 | −0.80 | 1.45 | −0.20 | 10120 | 6.59 | 5.80 | 8.23 | 5.17 | 5.58 | 6.07 | 5.85 | 4.91 |
| 390 | 0.11397 | −0.71 | 0.98 | −0.30 | 6936.5 | 8.33 | 7.62 | 9.28 | 8.17 | 8.48 | 8.15 | 7.75 | 5.79 |
| 391 | 0.27499 | −0.46 | 0.75 | −0.24 | 8850 | 6.05 | 5.59 | 6.83 | 6.13 | 6.04 | 5.91 | 5.68 | 4.63 |
| 392 | 0.64771 | 0.50 | 1.11 | 0.10 | 13337.5 | 6.14 | 6.63 | 7.33 | 5.71 | 7.17 | 6.94 | 6.68 | 4.77 |
| 393 | 0.30488 | −0.66 | 1.16 | −0.24 | 8850 | 6.20 | 5.55 | 7.37 | 5.40 | 6.63 | 6.08 | 5.26 | 4.02 |
| 394 | 0.64771 | −0.34 | 1.19 | −0.07 | 14538 | 5.91 | 5.57 | 7.40 | 5.02 | 5.57 | 5.56 | 5.36 | 4.64 |
| 395 | 0.10353 | 1.11 | 1.20 | 0.26 | 8265 | 3.94 | 5.04 | 5.82 | 3.57 | 5.18 | 5.14 | 4.90 | 3.62 |
| 396 | 0.01485 | 0.95 | 0.83 | 0.36 | 5465 | 6.78 | 7.73 | 8.55 | 7.12 | 6.77 | 7.91 | 8.00 | 6.95 |
| 397 | 0.19731 | 1.07 | 1.25 | 0.27 | 7737.5 | 4.57 | 5.64 | 6.07 | 5.32 | 6.21 | 5.64 | 5.98 | 4.24 |
| 398 | 0.30526 | 1.06 | 1.42 | 0.21 | 9914.5 | 5.59 | 6.65 | 7.42 | 5.49 | 7.29 | 6.99 | 6.86 | 4.71 |
| 399 | 0.52315 | 0.29 | 0.81 | 0.16 | 11367 | 3.69 | 3.98 | 3.95 | 3.37 | 4.23 | 3.99 | 4.19 | 3.32 |
| 400 | 0.11974 | 1.04 | 1.28 | 0.28 | 7482 | 5.23 | 6.27 | 7.06 | 4.85 | 6.51 | 6.41 | 6.02 | 4.46 |
| 401 | 0.11479 | −0.75 | 0.85 | −0.33 | 6398.5 | 9.58 | 8.84 | 9.74 | 9.51 | 9.73 | 8.94 | 9.01 | 7.56 |
| 402 | 0.30731 | −0.55 | 1.19 | −0.21 | 10011.5 | 7.86 | 7.31 | 8.95 | 6.65 | 6.89 | 7.42 | 7.09 | 6.11 |
| 403 | 0.15228 | 0.99 | 1.16 | 0.27 | 7737.5 | 6.26 | 7.25 | 7.70 | 6.62 | 7.61 | 7.55 | 7.47 | 5.56 |
| 404 | 0.30439 | 0.89 | 1.39 | 0.18 | 10622 | 3.79 | 4.68 | 5.62 | 3.69 | 5.49 | 5.21 | 4.78 | 3.11 |
| 405 | 0.77481 | 0.03 | 0.94 | 0.01 | 16758 | 6.31 | 6.34 | 8.08 | 6.83 | 6.49 | 6.63 | 6.34 | 4.84 |
| 406 | 0.24604 | −0.55 | 0.80 | −0.23 | 9073.5 | 7.78 | 7.23 | 8.33 | 7.93 | 7.95 | 7.28 | 7.14 | 5.65 |
| 407 | 0.07636 | 1.17 | 0.99 | 0.41 | 4384 | 5.06 | 6.24 | 6.33 | 5.61 | 5.45 | 6.61 | 5.95 | 4.78 |
| 408 | 0.40353 | 0.97 | 1.23 | 0.27 | 7737.5 | 5.21 | 6.18 | 6.64 | 5.68 | 6.35 | 6.56 | 7.23 | 5.23 |
| 409 | 0.13500 | −0.57 | 0.93 | −0.26 | 8173.5 | 5.55 | 4.97 | 6.76 | 4.80 | 5.02 | 5.08 | 5.04 | 4.38 |
| 410 | 0.11149 | −0.58 | 0.87 | −0.32 | 6592.5 | 7.44 | 6.86 | 8.32 | 7.23 | 7.58 | 7.27 | 7.02 | 5.47 |
| 411 | 0.93261 | −0.01 | 0.87 | −0.05 | 15120 | 6.04 | 6.03 | 7.43 | 6.38 | 6.56 | 6.16 | 6.14 | 4.49 |
| 412 | 0.08449 | 1.51 | 1.80 | 0.27 | 7954.5 | 5.14 | 6.65 | 7.50 | 5.19 | 7.11 | 7.05 | 6.80 | 3.85 |
| 413 | 0.26908 | 0.89 | 1.61 | 0.16 | 11367 | 4.91 | 5.79 | 6.86 | 4.90 | 6.91 | 6.57 | 6.51 | 3.48 |
| 414 | 0.60470 | 0.57 | 1.29 | 0.10 | 13537.5 | 4.28 | 4.84 | 5.49 | 4.14 | 5.97 | 5.23 | 5.83 | 3.69 |
| 415 | 0.13339 | 0.61 | 1.00 | 0.21 | 9615 | 3.73 | 4.34 | 4.45 | 4.00 | 5.06 | 4.60 | 5.07 | 3.31 |
| 416 | 0.21775 | −0.54 | 0.76 | −0.22 | 9376 | 7.35 | 6.80 | 8.28 | 7.20 | 7.19 | 7.51 | 7.74 | 6.75 |
| 417 | 0.08397 | 1.04 | 1.34 | 0.26 | 8265 | 5.38 | 6.42 | 7.25 | 5.97 | 7.24 | 6.89 | 6.74 | 4.37 |
| 418 | 0.64689 | −0.73 | 1.29 | −0.19 | 10385.5 | 5.04 | 4.32 | 6.20 | 4.51 | 4.31 | 4.49 | 4.07 | 4.25 |
| 419 | 0.00145 | −1.10 | 0.76 | −0.55 | 1734.5 | 8.25 | 7.15 | 7.82 | 8.43 | 8.50 | 7.87 | 8.41 | 7.66 |
| 420 | 0.04772 | −0.86 | 0.88 | −0.33 | 6225.5 | 9.60 | 8.74 | 10.58 | 8.72 | 8.59 | 9.23 | 9.66 | 8.44 |
| 421 | 0.00283 | −0.70 | 0.78 | −0.40 | 4630 | 5.03 | 4.34 | 6.28 | 4.48 | 4.98 | 5.16 | 4.87 | 4.63 |
| 422 | 0.00091 | 0.96 | 0.72 | 0.56 | 1683 | 3.34 | 4.30 | 4.83 | 3.41 | 3.46 | 3.86 | 3.89 | 3.61 |
| 423 | 0.27522 | −0.45 | 1.10 | −0.21 | 9762.5 | 5.53 | 5.08 | 6.93 | 4.48 | 4.86 | 5.04 | 5.20 | 4.50 |
| 424 | 0.44617 | 0.71 | 1.17 | 0.14 | 11998 | 5.38 | 6.09 | 6.39 | 4.81 | 6.53 | 6.50 | 5.85 | 4.48 |
| 425 | 0.21975 | 1.12 | 1.61 | 0.19 | 10385.5 | 4.09 | 5.21 | 6.32 | 4.44 | 6.09 | 5.67 | 5.83 | 3.34 |
| 426 | 0.52315 | −0.46 | 0.73 | −0.18 | 10870.5 | 7.57 | 7.10 | 8.15 | 7.61 | 7.75 | 7.32 | 7.16 | 6.11 |
| 427 | 0.09807 | −0.98 | 1.06 | −0.33 | 6225.5 | 7.30 | 6.33 | 8.30 | 7.01 | 7.33 | 7.01 | 6.67 | 4.79 |
| 428 | 0.08412 | −0.52 | 0.71 | −0.30 | 7220 | 6.36 | 5.83 | 6.62 | 5.90 | 5.88 | 5.95 | 5.79 | 5.28 |
| 429 | 0.09704 | −1.07 | 1.20 | −0.32 | 6592.5 | 11.45 | 10.38 | 11.98 | 10.19 | 11.03 | 10.53 | 11.82 | 9.67 |
| 430 | 0.05417 | 1.32 | 1.61 | 0.32 | 6500 | 4.18 | 5.50 | 6.14 | 4.88 | 5.88 | 5.56 | 5.35 | 3.47 |
| 431 | 0.85397 | −0.04 | 0.83 | −0.05 | 15120 | 9.30 | 9.25 | 10.67 | 9.47 | 8.97 | 9.78 | 10.20 | 9.07 |
| 432 | 0.05566 | 1.52 | 1.56 | 0.34 | 5990 | 5.89 | 7.41 | 7.88 | 6.13 | 7.78 | 7.26 | 7.44 | 4.91 |
| 433 | 0.05417 | 1.32 | 1.61 | 0.32 | 6500 | 4.18 | 5.50 | 6.14 | 4.88 | 5.88 | 5.56 | 5.35 | 3.47 |
| 434 | 0.08862 | 0.96 | 1.09 | 0.30 | 7124 | 4.63 | 5.60 | 5.91 | 3.85 | 5.74 | 5.68 | 5.37 | 3.92 |
| 435 | 0.08862 | 0.96 | 1.09 | 0.30 | 7124 | 4.63 | 5.60 | 5.91 | 3.85 | 5.74 | 5.68 | 5.37 | 3.92 |
| 436 | 0.19076 | 1.42 | 1.76 | 0.24 | 8600 | 5.05 | 6.47 | 7.37 | 5.93 | 5.58 | 6.07 | 8.05 | 5.31 |
| 437 | 0.00091 | −0.57 | 0.30 | −0.69 | 351.5 | 8.56 | 7.99 | 8.29 | 8.33 | 8.48 | 8.35 | 8.27 | 8.20 |
| 438 | 0.00091 | 0.30 | 0.16 | 0.73 | 158 | 7.75 | 8.04 | 7.79 | 7.83 | 7.89 | 7.87 | 7.67 | 7.90 |
| 439 | 0.00091 | −0.43 | 0.22 | −0.69 | 319.5 | 9.85 | 9.42 | 9.51 | 9.60 | 9.88 | 9.50 | 9.53 | 9.78 |
| 440 | 0.00091 | −0.40 | 0.21 | −0.70 | 249.5 | 8.65 | 8.24 | 8.48 | 8.56 | 8.67 | 8.36 | 8.39 | 8.37 |
| 441 | 0.00091 | 0.91 | 0.45 | 0.77 | 42.5 | 7.49 | 8.40 | 7.87 | 7.88 | 7.88 | 8.27 | 7.74 | 7.57 |
| 442 | 0.00091 | −0.49 | 0.27 | −0.69 | 319.5 | 8.30 | 7.80 | 7.86 | 8.12 | 8.17 | 7.89 | 8.02 | 8.07 |
| 443 | 0.00091 | 0.30 | 0.16 | 0.74 | 116.5 | 8.89 | 9.19 | 9.04 | 8.95 | 9.01 | 9.07 | 9.07 | 9.14 |
| 444 | 0.00091 | 0.61 | 0.31 | 0.69 | 289 | 9.94 | 10.55 | 10.40 | 10.09 | 10.01 | 10.38 | 10.04 | 9.87 |
| 445 | 0.00091 | 0.99 | 0.47 | 0.81 | 7.5 | 8.13 | 9.12 | 8.56 | 8.59 | 8.42 | 9.00 | 8.92 | 8.89 |
| 446 | 0.00091 | −0.43 | 0.24 | −0.71 | 235 | 7.84 | 7.40 | 7.59 | 7.61 | 7.85 | 7.49 | 7.55 | 7.78 |
| 447 | 0.00091 | −0.43 | 0.24 | −0.71 | 235 | 7.84 | 7.40 | 7.59 | 7.61 | 7.85 | 7.49 | 7.55 | 7.78 |
| 448 | 0.00091 | 0.71 | 0.37 | 0.74 | 116.5 | 7.38 | 8.09 | 7.78 | 7.83 | 7.45 | 7.78 | 7.98 | 8.10 |
| 449 | 0.00091 | 0.71 | 0.37 | 0.74 | 116.5 | 7.38 | 8.09 | 7.78 | 7.83 | 7.45 | 7.78 | 7.98 | 8.10 |
| 450 | 0.00091 | 0.71 | 0.37 | 0.74 | 116.5 | 7.38 | 8.09 | 7.78 | 7.83 | 7.45 | 7.78 | 7.98 | 8.10 |
| 451 | 0.00091 | 0.50 | 0.28 | 0.70 | 265.5 | 9.25 | 9.75 | 9.52 | 9.33 | 9.30 | 9.61 | 9.39 | 9.56 |
| 452 | 0.00091 | 0.50 | 0.28 | 0.70 | 265.5 | 9.25 | 9.75 | 9.52 | 9.33 | 9.30 | 9.61 | 9.39 | 9.56 |
| 453 | 0.00091 | −0.60 | 0.29 | −0.76 | 51.5 | 7.22 | 6.61 | 6.89 | 7.02 | 7.09 | 6.71 | 6.83 | 6.92 |
| 454 | 0.00091 | −0.33 | 0.18 | −0.69 | 351.5 | 8.15 | 7.82 | 8.04 | 8.10 | 8.18 | 7.97 | 8.02 | 8.03 |
| 455 | 0.00091 | 0.61 | 0.32 | 0.72 | 186 | 4.62 | 5.23 | 4.94 | 4.74 | 4.74 | 4.88 | 4.79 | 4.84 |
| 456 | 0.00091 | 0.61 | 0.32 | 0.72 | 186 | 4.62 | 5.23 | 4.94 | 4.74 | 4.74 | 4.88 | 4.79 | 4.84 |
| 457 | 0.00091 | −0.32 | 0.18 | −0.72 | 172.5 | 7.00 | 6.68 | 6.81 | 6.91 | 7.11 | 6.91 | 6.85 | 6.73 |
| 458 | 0.00091 | −0.32 | 0.18 | −0.69 | 319.5 | 5.86 | 5.54 | 5.82 | 5.70 | 5.81 | 5.62 | 5.60 | 5.67 |
| 459 | 0.00091 | −0.38 | 0.21 | −0.69 | 319.5 | 8.61 | 8.22 | 8.42 | 8.52 | 8.72 | 8.31 | 8.37 | 8.38 |
| 460 | 0.00091 | −0.75 | 0.40 | −0.72 | 172.5 | 7.11 | 6.36 | 6.86 | 6.98 | 6.86 | 6.65 | 7.04 | 6.82 |
| 461 | 0.00091 | 0.57 | 0.29 | 0.73 | 158 | 9.56 | 10.12 | 9.94 | 9.70 | 9.70 | 10.10 | 9.90 | 9.88 |
| 462 | 0.00145 | 0.30 | 0.16 | 0.70 | 265.5 | 6.81 | 7.12 | 6.93 | 6.88 | 6.80 | 7.02 | 7.04 | 6.99 |
| 463 | 0.00091 | −0.46 | 0.24 | −0.69 | 351.5 | 8.41 | 7.95 | 8.16 | 8.40 | 8.36 | 8.01 | 8.14 | 8.43 |
| 464 | 0.00091 | −0.30 | 0.16 | −0.76 | 60.5 | 7.51 | 7.21 | 7.43 | 7.32 | 7.35 | 7.40 | 7.14 | 7.14 |
| 465 | 0.00091 | −0.30 | 0.16 | −0.76 | 60.5 | 7.51 | 7.21 | 7.43 | 7.32 | 7.35 | 7.40 | 7.14 | 7.14 |
| 466 | 0.00091 | −0.48 | 0.23 | −0.77 | 47.5 | 6.46 | 5.98 | 6.24 | 6.34 | 6.27 | 5.93 | 6.39 | 6.14 |
| 467 | 0.00091 | 0.19 | 0.10 | 0.69 | 371.5 | 11.17 | 11.36 | 11.31 | 11.29 | 11.13 | 11.39 | 11.26 | 11.28 |
| 468 | 0.00091 | 0.51 | 0.27 | 0.74 | 116.5 | 5.93 | 6.44 | 6.13 | 6.06 | 6.20 | 6.39 | 6.22 | 6.46 |
| 469 | 0.00091 | 0.51 | 0.27 | 0.74 | 116.5 | 5.93 | 6.44 | 6.13 | 6.06 | 6.20 | 6.39 | 6.22 | 6.46 |
| 470 | 0.00091 | 0.24 | 0.13 | 0.69 | 289 | 6.38 | 6.62 | 6.39 | 6.53 | 6.35 | 6.52 | 6.35 | 6.63 |
| 471 | 0.00091 | 0.24 | 0.13 | 0.69 | 289 | 6.38 | 6.62 | 6.39 | 6.53 | 6.35 | 6.52 | 6.35 | 6.63 |
| 472 | 0.00091 | −0.38 | 0.22 | −0.71 | 235 | 5.13 | 4.75 | 4.97 | 4.94 | 5.00 | 4.89 | 4.83 | 4.75 |
| 473 | 0.00091 | 0.38 | 0.19 | 0.80 | 13.5 | 6.55 | 6.92 | 6.66 | 6.67 | 6.66 | 6.89 | 6.88 | 6.95 |
| 474 | 0.00091 | 0.39 | 0.20 | 0.79 | 20.5 | 6.69 | 7.08 | 6.80 | 6.83 | 6.81 | 7.04 | 7.06 | 7.07 |
| 475 | 0.00091 | 0.39 | 0.20 | 0.79 | 20.5 | 6.69 | 7.08 | 6.80 | 6.83 | 6.81 | 7.04 | 7.06 | 7.07 |
| 476 | 0.00091 | 0.38 | 0.19 | 0.80 | 13.5 | 6.55 | 6.92 | 6.66 | 6.67 | 6.66 | 6.89 | 6.88 | 6.95 |
| 477 | 0.00091 | −0.74 | 0.39 | −0.78 | 38 | 8.21 | 7.47 | 7.92 | 7.93 | 7.84 | 7.45 | 7.58 | 7.29 |
| 478 | 0.00091 | −0.26 | 0.14 | −0.71 | 235 | 6.05 | 5.78 | 5.92 | 5.90 | 6.02 | 5.85 | 6.07 | 6.06 |
| 479 | 0.00091 | 0.25 | 0.15 | 0.71 | 222.5 | 8.01 | 8.27 | 8.07 | 8.12 | 8.03 | 8.24 | 8.00 | 8.36 |
| 480 | 0.00091 | 0.46 | 0.24 | 0.69 | 289 | 7.64 | 8.10 | 8.04 | 7.76 | 7.64 | 8.07 | 7.98 | 7.79 |
| 481 | 0.00091 | 0.46 | 0.24 | 0.69 | 289 | 7.64 | 8.10 | 8.04 | 7.76 | 7.64 | 8.07 | 7.98 | 7.79 |
| 482 | 0.00091 | −0.40 | 0.21 | −0.76 | 60.5 | 8.85 | 8.45 | 8.71 | 8.76 | 8.80 | 8.59 | 8.75 | 8.55 |
| 483 | 0.00091 | −0.40 | 0.21 | −0.76 | 60.5 | 8.85 | 8.45 | 8.71 | 8.76 | 8.80 | 8.59 | 8.75 | 8.55 |
| 484 | 0.00091 | −0.40 | 0.21 | −0.76 | 60.5 | 8.85 | 8.45 | 8.71 | 8.76 | 8.80 | 8.59 | 8.75 | 8.55 |
| 485 | 0.00091 | −0.40 | 0.21 | −0.76 | 60.5 | 8.85 | 8.45 | 8.71 | 8.76 | 8.80 | 8.59 | 8.75 | 8.55 |
| 486 | 0.00091 | 0.84 | 0.42 | 0.71 | 222.5 | 5.48 | 6.31 | 5.97 | 5.58 | 5.62 | 5.85 | 6.03 | 5.81 |
| 487 | 0.00091 | 0.84 | 0.42 | 0.71 | 222.5 | 5.48 | 6.31 | 5.97 | 5.58 | 5.62 | 5.85 | 6.03 | 5.81 |
| 488 | 0.00091 | −0.93 | 0.47 | −0.69 | 319.5 | 6.85 | 5.92 | 6.05 | 6.51 | 6.66 | 5.99 | 6.09 | 6.30 |
| 489 | 0.00091 | −0.51 | 0.27 | −0.75 | 90.5 | 9.78 | 9.27 | 9.64 | 9.65 | 9.75 | 9.46 | 9.44 | 9.39 |
| 490 | 0.00091 | −0.34 | 0.20 | −0.71 | 235 | 7.66 | 7.32 | 7.51 | 7.41 | 7.74 | 7.45 | 7.37 | 7.39 |
| 491 | 0.00091 | −0.34 | 0.20 | −0.71 | 235 | 7.66 | 7.32 | 7.51 | 7.41 | 7.74 | 7.45 | 7.37 | 7.39 |
| 492 | 0.00091 | −0.55 | 0.33 | −0.76 | 60.5 | 7.96 | 7.41 | 7.72 | 7.45 | 7.65 | 7.66 | 7.57 | 7.22 |
| 493 | 0.00091 | −0.55 | 0.33 | −0.76 | 60.5 | 7.96 | 7.41 | 7.72 | 7.45 | 7.65 | 7.66 | 7.57 | 7.22 |
| 494 | 0.00091 | −0.55 | 0.33 | −0.76 | 60.5 | 7.96 | 7.41 | 7.72 | 7.45 | 7.65 | 7.66 | 7.57 | 7.22 |
| 495 | 0.00091 | −0.23 | 0.13 | −0.69 | 319.5 | 8.66 | 8.42 | 8.54 | 8.60 | 8.68 | 8.49 | 8.50 | 8.56 |
| 496 | 0.00091 | 0.63 | 0.32 | 0.78 | 26 | 8.36 | 8.99 | 8.49 | 8.67 | 8.30 | 8.66 | 8.53 | 8.93 |
| 497 | 0.00091 | 0.63 | 0.32 | 0.78 | 26 | 8.36 | 8.99 | 8.49 | 8.67 | 8.30 | 8.66 | 8.53 | 8.93 |
| 498 | 0.00091 | 0.63 | 0.32 | 0.78 | 26 | 8.36 | 8.99 | 8.49 | 8.67 | 8.30 | 8.66 | 8.53 | 8.93 |
| 499 | 0.00091 | −0.33 | 0.20 | −0.69 | 319.5 | 8.50 | 8.16 | 8.45 | 8.30 | 8.39 | 8.28 | 8.38 | 8.23 |
| 500 | 0.00091 | 0.31 | 0.17 | 0.72 | 214 | 9.16 | 9.47 | 9.26 | 9.20 | 9.13 | 9.50 | 9.28 | 9.19 |
| 501 | 0.00091 | −0.26 | 0.13 | −0.80 | 17 | 6.38 | 6.12 | 6.25 | 6.18 | 6.40 | 6.11 | 6.21 | 6.24 |
| 502 | 0.00091 | −0.40 | 0.21 | −0.69 | 351.5 | 8.63 | 8.23 | 8.49 | 8.57 | 8.77 | 8.29 | 8.66 | 8.70 |
| 503 | 0.00091 | −0.40 | 0.21 | −0.69 | 351.5 | 8.63 | 8.23 | 8.49 | 8.57 | 8.77 | 8.29 | 8.66 | 8.70 |
| 504 | 0.00091 | −0.39 | 0.20 | −0.69 | 319.5 | 10.10 | 9.71 | 10.07 | 9.96 | 10.01 | 9.72 | 9.58 | 9.74 |
| 505 | 0.00091 | −0.39 | 0.20 | −0.69 | 319.5 | 10.10 | 9.71 | 10.07 | 9.96 | 10.01 | 9.72 | 9.58 | 9.74 |
| 506 | 0.00091 | −0.39 | 0.20 | −0.69 | 319.5 | 10.10 | 9.71 | 10.07 | 9.96 | 10.01 | 9.72 | 9.58 | 9.74 |
| 507 | 0.00091 | −0.39 | 0.20 | −0.69 | 319.5 | 10.10 | 9.71 | 10.07 | 9.96 | 10.01 | 9.72 | 9.58 | 9.74 |
| 508 | 0.00091 | 0.58 | 0.29 | 0.73 | 158 | 7.63 | 8.21 | 7.86 | 7.83 | 7.54 | 8.33 | 7.70 | 7.90 |
| 509 | 0.00091 | −0.73 | 0.40 | −0.70 | 249.5 | 6.57 | 5.84 | 6.25 | 6.31 | 6.03 | 5.92 | 5.73 | 5.86 |
| 510 | 0.00091 | −0.73 | 0.40 | −0.70 | 249.5 | 6.57 | 5.84 | 6.25 | 6.31 | 6.03 | 5.92 | 5.73 | 5.86 |
| 511 | 0.00145 | −0.41 | 0.21 | −0.71 | 235 | 8.75 | 8.35 | 8.57 | 8.64 | 8.74 | 8.36 | 8.63 | 8.66 |
| 512 | 0.00091 | −0.77 | 0.40 | −0.69 | 319.5 | 6.85 | 6.08 | 6.25 | 6.37 | 7.10 | 6.31 | 6.50 | 6.65 |
| 513 | 0.00091 | 0.46 | 0.25 | 0.70 | 265.5 | 9.38 | 9.83 | 9.54 | 9.60 | 9.49 | 9.73 | 9.52 | 9.87 |
| 514 | 0.00145 | −0.36 | 0.18 | −0.72 | 194.5 | 8.70 | 8.34 | 8.54 | 8.62 | 8.74 | 8.44 | 8.57 | 8.52 |
| 515 | 0.00145 | −0.36 | 0.18 | −0.72 | 194.5 | 8.70 | 8.34 | 8.54 | 8.62 | 8.74 | 8.44 | 8.57 | 8.52 |
| 516 | 0.00145 | −0.36 | 0.18 | −0.72 | 194.5 | 8.70 | 8.34 | 8.54 | 8.62 | 8.74 | 8.44 | 8.57 | 8.52 |
| 517 | 0.00091 | 0.30 | 0.16 | 0.75 | 77.5 | 7.53 | 7.83 | 7.66 | 7.66 | 7.63 | 7.89 | 7.70 | 7.73 |
| 518 | 0.00091 | 0.64 | 0.34 | 0.75 | 103 | 7.37 | 8.01 | 7.54 | 7.48 | 7.57 | 7.61 | 7.71 | 7.90 |
| 519 | 0.00091 | −0.33 | 0.18 | −0.69 | 351.5 | 8.15 | 7.82 | 8.04 | 8.10 | 8.18 | 7.97 | 8.02 | 8.03 |
| 520 | 0.00091 | −0.52 | 0.30 | −0.73 | 141.5 | 8.78 | 8.26 | 8.51 | 8.58 | 8.70 | 8.38 | 8.53 | 8.26 |
| 521 | 0.00091 | 0.30 | 0.16 | 0.73 | 158 | 7.75 | 8.04 | 7.79 | 7.83 | 7.89 | 7.87 | 7.67 | 7.90 |
| 522 | 0.00091 | 0.30 | 0.16 | 0.73 | 158 | 7.75 | 8.04 | 7.79 | 7.83 | 7.89 | 7.87 | 7.67 | 7.90 |
| 523 | 0.00091 | 0.45 | 0.23 | 0.69 | 289 | 10.22 | 10.67 | 10.48 | 10.26 | 10.32 | 10.40 | 10.40 | 10.45 |
| 524 | 0.00091 | 0.88 | 0.48 | 0.72 | 186 | 6.14 | 7.02 | 6.32 | 6.60 | 6.60 | 6.95 | 7.00 | 6.64 |
| 525 | 0.00091 | 0.39 | 0.22 | 0.70 | 265.5 | 9.29 | 9.69 | 9.45 | 9.31 | 9.39 | 9.46 | 9.55 | 9.52 |
| 526 | 0.00091 | −0.70 | 0.38 | −0.73 | 141.5 | 7.48 | 6.78 | 7.30 | 7.19 | 7.42 | 7.05 | 7.17 | 7.28 |
| 527 | 0.00091 | −0.70 | 0.38 | −0.73 | 141.5 | 7.48 | 6.78 | 7.30 | 7.19 | 7.42 | 7.05 | 7.17 | 7.28 |
| 528 | 0.00091 | −0.70 | 0.38 | −0.73 | 141.5 | 7.48 | 6.78 | 7.30 | 7.19 | 7.42 | 7.05 | 7.17 | 7.28 |
| 529 | 0.00091 | −0.70 | 0.38 | −0.73 | 141.5 | 7.48 | 6.78 | 7.30 | 7.19 | 7.42 | 7.05 | 7.17 | 7.28 |
| 530 | 0.00091 | −0.49 | 0.25 | −0.69 | 319.5 | 8.38 | 7.89 | 8.10 | 8.29 | 8.56 | 7.94 | 8.09 | 8.46 |
| 531 | 0.00091 | −0.56 | 0.27 | −0.73 | 141.5 | 7.80 | 7.25 | 7.69 | 7.71 | 7.79 | 7.29 | 7.68 | 7.88 |
| 532 | 0.00091 | −0.56 | 0.27 | −0.73 | 141.5 | 7.80 | 7.25 | 7.69 | 7.71 | 7.79 | 7.29 | 7.68 | 7.88 |
| 533 | 0.00091 | −0.56 | 0.27 | −0.73 | 141.5 | 7.80 | 7.25 | 7.69 | 7.71 | 7.79 | 7.29 | 7.68 | 7.88 |
| 534 | 0.00091 | −0.50 | 0.30 | −0.72 | 172.5 | 4.18 | 3.67 | 3.95 | 3.84 | 3.87 | 3.79 | 4.00 | 4.07 |
| 535 | 0.00091 | 0.33 | 0.17 | 0.69 | 289 | 9.18 | 9.51 | 9.36 | 9.21 | 9.11 | 9.44 | 9.22 | 9.28 |
| 536 | 0.00091 | 0.94 | 0.49 | 0.69 | 289 | 7.32 | 8.26 | 7.95 | 7.63 | 7.31 | 7.79 | 7.81 | 7.87 |
| 537 | 0.00091 | 0.94 | 0.49 | 0.69 | 289 | 7.32 | 8.26 | 7.95 | 7.63 | 7.31 | 7.79 | 7.81 | 7.87 |
| 538 | 0.00091 | −0.32 | 0.18 | −0.69 | 319.5 | 5.86 | 5.54 | 5.82 | 5.70 | 5.81 | 5.62 | 5.60 | 5.67 |
| 539 | 0.00091 | 0.42 | 0.24 | 0.69 | 371.5 | 7.73 | 8.15 | 7.96 | 7.83 | 7.77 | 8.21 | 7.99 | 8.17 |
| 540 | 0.00091 | 0.42 | 0.24 | 0.69 | 371.5 | 7.73 | 8.15 | 7.96 | 7.83 | 7.77 | 8.21 | 7.99 | 8.17 |
| 541 | 0.00091 | −0.98 | 0.52 | −0.69 | 351.5 | 7.87 | 6.89 | 7.08 | 7.48 | 7.92 | 7.30 | 7.42 | 7.55 |
| 542 | 0.00091 | 0.53 | 0.27 | 0.75 | 103 | 7.06 | 7.58 | 7.25 | 7.13 | 7.00 | 7.04 | 7.08 | 7.45 |
| 543 | 0.00091 | −0.28 | 0.15 | −0.69 | 319.5 | 9.56 | 9.27 | 9.40 | 9.49 | 9.48 | 9.25 | 9.29 | 9.41 |
| 544 | 0.00091 | 0.46 | 0.24 | 0.76 | 72 | 9.05 | 9.52 | 9.27 | 9.29 | 9.19 | 9.44 | 9.08 | 9.42 |
| 545 | 0.00091 | 0.59 | 0.30 | 0.72 | 214 | 6.45 | 7.04 | 6.74 | 6.65 | 6.69 | 6.59 | 6.68 | 7.02 |
| 546 | 0.00091 | −0.21 | 0.12 | −0.71 | 235 | 11.56 | 11.35 | 11.49 | 11.40 | 11.43 | 11.35 | 11.55 | 11.35 |
| 547 | 0.00091 | −0.61 | 0.32 | −0.75 | 90.5 | 9.76 | 9.15 | 9.57 | 9.42 | 9.46 | 9.42 | 9.44 | 9.18 |
| 548 | 0.00091 | 0.39 | 0.20 | 0.70 | 265.5 | 7.02 | 7.41 | 7.31 | 7.13 | 7.05 | 7.42 | 7.29 | 7.49 |
| 549 | 0.00091 | −0.19 | 0.11 | −0.69 | 351.5 | 10.66 | 10.47 | 10.51 | 10.57 | 10.57 | 10.48 | 10.51 | 10.52 |
| 550 | 0.00091 | −0.42 | 0.22 | −0.70 | 249.5 | 9.39 | 8.97 | 9.22 | 9.34 | 9.57 | 8.98 | 9.21 | 9.58 |
| 551 | 0.00091 | 0.38 | 0.23 | 0.72 | 214 | 7.36 | 7.74 | 7.53 | 7.46 | 7.38 | 7.60 | 7.21 | 7.47 |
| 552 | 0.00091 | −0.35 | 0.17 | −0.76 | 60.5 | 10.83 | 10.48 | 10.63 | 10.72 | 10.78 | 10.58 | 10.67 | 10.69 |
| 553 | 0.00091 | −0.60 | 0.29 | −0.76 | 51.5 | 7.22 | 6.61 | 6.89 | 7.02 | 7.09 | 6.71 | 6.83 | 6.92 |
| 554 | 0.00091 | 0.69 | 0.35 | 0.76 | 72 | 5.38 | 6.07 | 5.70 | 5.83 | 5.54 | 6.04 | 5.66 | 5.90 |
| 555 | 0.00091 | −0.57 | 0.28 | −0.77 | 47.5 | 9.74 | 9.17 | 9.44 | 9.53 | 9.64 | 9.34 | 9.29 | 9.46 |
| 556 | 0.00091 | −0.81 | 0.43 | −0.69 | 351.5 | 7.02 | 6.21 | 6.59 | 6.97 | 7.03 | 6.61 | 6.50 | 6.54 |
| 557 | 0.00091 | 0.90 | 0.45 | 0.70 | 265.5 | 6.08 | 6.97 | 6.51 | 6.02 | 6.17 | 6.71 | 6.12 | 6.46 |
| 558 | 0.00091 | −0.81 | 0.43 | −0.76 | 60.5 | 4.71 | 3.91 | 4.55 | 4.25 | 4.58 | 4.26 | 4.35 | 3.99 |
| 559 | 0.00091 | −0.50 | 0.25 | −0.81 | 10.5 | 6.79 | 6.28 | 6.62 | 6.62 | 6.42 | 6.33 | 6.42 | 6.46 |
| 560 | 0.00091 | −0.55 | 0.33 | −0.76 | 60.5 | 7.96 | 7.41 | 7.72 | 7.45 | 7.65 | 7.66 | 7.57 | 7.22 |
| 561 | 0.00091 | 0.34 | 0.17 | 0.72 | 214 | 10.76 | 11.10 | 10.91 | 10.84 | 10.87 | 11.00 | 11.01 | 10.95 |
| 562 | 0.00091 | −0.56 | 0.31 | −0.74 | 127.5 | 7.32 | 6.76 | 7.04 | 7.07 | 7.06 | 6.97 | 6.92 | 6.75 |
| 563 | 0.00091 | 0.43 | 0.24 | 0.69 | 371.5 | 6.93 | 7.37 | 7.08 | 7.04 | 6.95 | 7.18 | 7.13 | 7.01 |
| 564 | 0.00091 | −0.49 | 0.24 | −0.78 | 38 | 8.71 | 8.22 | 8.62 | 8.54 | 8.61 | 8.25 | 8.39 | 8.45 |
| 565 | 0.00091 | 0.58 | 0.31 | 0.74 | 116.5 | 8.55 | 9.13 | 8.80 | 8.89 | 8.60 | 8.97 | 8.93 | 8.99 |
| 566 | 0.00091 | 0.63 | 0.32 | 0.72 | 186 | 6.60 | 7.23 | 6.93 | 6.76 | 6.87 | 7.08 | 6.85 | 6.96 |
| 567 | 0.00091 | 0.63 | 0.35 | 0.72 | 197 | 6.67 | 7.30 | 6.94 | 6.88 | 6.61 | 7.09 | 6.88 | 7.18 |
| 568 | 0.00091 | 0.47 | 0.25 | 0.72 | 214 | 8.19 | 8.66 | 8.24 | 8.47 | 8.27 | 8.85 | 8.52 | 8.59 |
| 569 | 0.00091 | 0.55 | 0.29 | 0.69 | 371.5 | 9.04 | 9.60 | 9.44 | 9.26 | 8.91 | 9.44 | 9.48 | 9.25 |
| 570 | 0.00091 | −0.41 | 0.22 | −0.74 | 127.5 | 7.92 | 7.52 | 7.74 | 7.84 | 8.02 | 7.74 | 7.88 | 7.89 |
| 571 | 0.00091 | 0.63 | 0.32 | 0.78 | 26 | 8.36 | 8.99 | 8.49 | 8.67 | 8.30 | 8.66 | 8.53 | 8.93 |
| 572 | 0.00091 | −0.56 | 0.27 | −0.76 | 60.5 | 8.69 | 8.14 | 8.39 | 8.59 | 8.89 | 8.20 | 8.49 | 8.67 |
| 573 | 0.00091 | 0.90 | 0.50 | 0.69 | 289 | 8.10 | 8.99 | 8.57 | 8.60 | 7.74 | 8.31 | 8.87 | 7.97 |
| 574 | 0.00091 | 0.31 | 0.17 | 0.72 | 214 | 9.16 | 9.47 | 9.26 | 9.20 | 9.13 | 9.50 | 9.28 | 9.19 |
| 575 | 0.00091 | 0.31 | 0.17 | 0.69 | 289 | 12.16 | 12.47 | 12.24 | 12.20 | 12.19 | 12.45 | 12.18 | 12.31 |
| 576 | 0.00091 | −1.28 | 0.63 | −0.75 | 90.5 | 8.40 | 7.12 | 7.70 | 8.20 | 8.61 | 7.71 | 8.11 | 8.15 |
| 577 | 0.00091 | −0.41 | 0.20 | −0.75 | 82 | 8.81 | 8.40 | 8.58 | 8.68 | 8.94 | 8.38 | 8.68 | 8.92 |
| 578 | 0.00091 | −0.31 | 0.17 | −0.72 | 204 | 10.40 | 10.10 | 10.34 | 10.25 | 10.20 | 10.22 | 10.21 | 10.19 |
| 579 | 0.00091 | 0.67 | 0.34 | 0.72 | 186 | 10.20 | 10.86 | 10.56 | 10.37 | 10.06 | 10.45 | 10.44 | 10.58 |
| 580 | 0.00091 | 0.53 | 0.27 | 0.75 | 103 | 7.06 | 7.58 | 7.25 | 7.13 | 7.00 | 7.04 | 7.08 | 7.45 |
| 581 | 0.00091 | −0.47 | 0.24 | −0.70 | 249.5 | 8.58 | 8.12 | 8.46 | 8.48 | 8.43 | 8.16 | 8.28 | 8.26 |
| 582 | 0.00091 | −0.35 | 0.20 | −0.69 | 319.5 | 9.16 | 8.81 | 9.12 | 8.96 | 9.07 | 8.93 | 8.78 | 8.87 |
| 583 | 0.00091 | 0.77 | 0.39 | 0.75 | 77.5 | 9.72 | 10.49 | 10.29 | 10.03 | 9.84 | 10.17 | 10.28 | 10.37 |
| 584 | 0.00091 | 0.48 | 0.24 | 0.69 | 371.5 | 9.14 | 9.62 | 9.38 | 9.11 | 9.39 | 9.60 | 9.07 | 9.14 |
| 585 | 0.00091 | −0.49 | 0.25 | −0.74 | 127.5 | 6.51 | 6.02 | 6.35 | 6.39 | 6.45 | 6.16 | 6.25 | 6.21 |
| 586 | 0.00091 | 0.39 | 0.22 | 0.70 | 265.5 | 5.25 | 5.65 | 5.34 | 5.47 | 5.30 | 5.44 | 5.33 | 5.45 |
| 587 | 0.00091 | −0.73 | 0.40 | −0.70 | 249.5 | 6.57 | 5.84 | 6.25 | 6.31 | 6.03 | 5.92 | 5.73 | 5.86 |
| 588 | 0.00091 | −0.58 | 0.32 | −0.70 | 249.5 | 8.34 | 7.76 | 8.10 | 7.97 | 7.63 | 7.63 | 7.72 | 7.79 |
| 589 | 0.00091 | 0.60 | 0.29 | 0.81 | 7.5 | 9.71 | 10.31 | 10.04 | 9.99 | 10.07 | 10.10 | 10.32 | 10.30 |
| 590 | 0.00091 | 0.51 | 0.27 | 0.74 | 116.5 | 5.93 | 6.44 | 6.13 | 6.06 | 6.20 | 6.39 | 6.22 | 6.46 |
| 591 | 0.00091 | 0.40 | 0.22 | 0.72 | 186 | 10.79 | 11.20 | 10.95 | 11.03 | 10.88 | 11.13 | 11.24 | 11.31 |
| 592 | 0.00091 | 0.76 | 0.37 | 0.75 | 103 | 4.35 | 5.12 | 4.64 | 4.56 | 4.55 | 4.58 | 4.49 | 4.69 |
| 593 | 0.00091 | 0.62 | 0.32 | 0.75 | 103 | 10.29 | 10.91 | 10.47 | 10.45 | 10.28 | 10.53 | 10.79 | 10.62 |
| 594 | 0.00091 | −0.42 | 0.21 | −0.69 | 351.5 | 7.66 | 7.24 | 7.55 | 7.64 | 7.49 | 7.33 | 7.51 | 7.48 |
| 595 | 0.00091 | −0.98 | 0.52 | −0.69 | 351.5 | 7.87 | 6.89 | 7.08 | 7.48 | 7.92 | 7.30 | 7.42 | 7.55 |
| 596 | 0.00091 | −0.46 | 0.23 | −0.70 | 249.5 | 8.40 | 7.93 | 8.08 | 8.08 | 8.51 | 8.18 | 8.02 | 8.13 |
| 597 | 0.00091 | 0.73 | 0.38 | 0.72 | 186 | 8.95 | 9.68 | 9.45 | 9.27 | 9.08 | 9.51 | 9.53 | 9.70 |
| 598 | 0.00091 | 0.36 | 0.18 | 0.75 | 103 | 3.97 | 4.33 | 4.06 | 4.15 | 4.45 | 4.32 | 4.17 | 4.19 |
| 599 | 0.00091 | 0.39 | 0.20 | 0.69 | 371.5 | 8.82 | 9.21 | 9.05 | 8.89 | 8.78 | 9.26 | 8.97 | 8.98 |
| 600 | 0.00091 | −0.74 | 0.39 | −0.78 | 38 | 8.21 | 7.47 | 7.92 | 7.93 | 7.84 | 7.45 | 7.58 | 7.29 |
| 601 | 0.00091 | 0.24 | 0.13 | 0.69 | 289 | 6.38 | 6.62 | 6.39 | 6.53 | 6.35 | 6.52 | 6.35 | 6.63 |
| 602 | 0.00091 | 0.57 | 0.29 | 0.72 | 186 | 9.06 | 9.63 | 9.16 | 9.20 | 9.11 | 9.51 | 9.31 | 9.42 |
| 603 | 0.00091 | 0.39 | 0.20 | 0.77 | 42.5 | 8.68 | 9.07 | 8.85 | 8.96 | 8.80 | 9.03 | 8.93 | 8.96 |
| 604 | 0.00145 | −0.41 | 0.21 | −0.71 | 235 | 8.75 | 8.35 | 8.57 | 8.64 | 8.74 | 8.36 | 8.63 | 8.66 |
| 605 | 0.00091 | 0.79 | 0.39 | 0.78 | 26 | 8.73 | 9.51 | 9.06 | 9.13 | 8.84 | 9.30 | 9.22 | 9.35 |
| 606 | 0.00091 | 0.35 | 0.18 | 0.84 | 1 | 10.11 | 10.47 | 10.21 | 10.26 | 10.06 | 10.46 | 10.42 | 10.44 |
| 607 | 0.00091 | 0.61 | 0.32 | 0.72 | 186 | 4.62 | 5.23 | 4.94 | 4.74 | 4.74 | 4.88 | 4.79 | 4.84 |
| 608 | 0.00091 | 0.38 | 0.19 | 0.80 | 13.5 | 6.55 | 6.92 | 6.66 | 6.67 | 6.66 | 6.89 | 6.88 | 6.95 |
| 609 | 0.00091 | −0.27 | 0.15 | −0.69 | 319.5 | 9.06 | 8.79 | 8.98 | 9.06 | 9.01 | 8.79 | 9.05 | 8.98 |
| 610 | 0.00091 | 0.63 | 0.31 | 0.70 | 265.5 | 6.62 | 7.25 | 6.85 | 6.66 | 6.88 | 7.03 | 6.98 | 7.17 |
| 611 | 0.00091 | −0.45 | 0.24 | −0.71 | 235 | 5.78 | 5.33 | 5.64 | 5.61 | 5.74 | 5.62 | 5.56 | 5.56 |
| 612 | 0.00091 | −0.60 | 0.33 | −0.69 | 338 | 4.30 | 3.70 | 3.91 | 4.04 | 4.36 | 3.95 | 3.90 | 3.77 |
| 613 | 0.00091 | −0.57 | 0.30 | −0.73 | 141.5 | 6.29 | 5.72 | 6.11 | 6.06 | 6.33 | 6.00 | 6.17 | 5.92 |
| 614 | 0.00091 | 0.41 | 0.20 | 0.72 | 186 | 10.45 | 10.86 | 10.78 | 10.72 | 10.51 | 10.79 | 10.65 | 10.78 |
| 615 | 0.00091 | −0.62 | 0.29 | −0.77 | 47.5 | 6.91 | 6.28 | 6.51 | 6.63 | 6.77 | 6.39 | 6.57 | 6.67 |
| 616 | 0.00091 | −0.77 | 0.40 | −0.69 | 319.5 | 6.85 | 6.08 | 6.25 | 6.37 | 7.10 | 6.31 | 6.50 | 6.65 |
| 617 | 0.00091 | −0.42 | 0.21 | −0.75 | 82 | 7.36 | 6.94 | 7.20 | 7.07 | 7.26 | 6.97 | 7.07 | 7.22 |
| 618 | 0.00091 | 0.78 | 0.39 | 0.73 | 158 | 9.16 | 9.93 | 9.48 | 9.34 | 9.07 | 9.99 | 9.36 | 9.31 |
| 619 | 0.00091 | −0.24 | 0.14 | −0.70 | 275 | 10.01 | 9.77 | 10.02 | 9.85 | 9.97 | 9.65 | 9.98 | 9.68 |
| 620 | 0.00145 | −0.36 | 0.18 | −0.72 | 194.5 | 8.70 | 8.34 | 8.54 | 8.62 | 8.74 | 8.44 | 8.57 | 8.52 |
| 621 | 0.00091 | 0.58 | 0.32 | 0.69 | 289 | 6.20 | 6.78 | 6.44 | 6.30 | 6.30 | 6.68 | 6.48 | 6.36 |
| 622 | 0.00091 | 1.00 | 0.52 | 0.74 | 116.5 | 7.72 | 8.72 | 8.10 | 7.87 | 7.71 | 8.18 | 8.01 | 8.29 |
| 623 | 0.00091 | −0.33 | 0.18 | −0.69 | 351.5 | 8.15 | 7.82 | 8.04 | 8.10 | 8.18 | 7.97 | 8.02 | 8.03 |
| 624 | 0.00091 | −0.48 | 0.25 | −0.72 | 172.5 | 7.36 | 6.88 | 7.32 | 7.21 | 7.29 | 7.20 | 7.20 | 7.26 |
| 625 | 0.00091 | −0.26 | 0.13 | −0.80 | 17 | 6.38 | 6.12 | 6.25 | 6.18 | 6.40 | 6.11 | 6.21 | 6.24 |
| 626 | 0.00091 | −0.30 | 0.16 | −0.76 | 60.5 | 7.51 | 7.21 | 7.43 | 7.32 | 7.35 | 7.40 | 7.14 | 7.14 |
| 627 | 0.00091 | −1.01 | 0.50 | −0.69 | 351.5 | 7.35 | 6.34 | 6.51 | 7.12 | 7.04 | 6.43 | 6.53 | 6.09 |
| 628 | 0.00145 | 0.51 | 0.28 | 0.69 | 371.5 | 9.05 | 9.56 | 9.19 | 9.34 | 8.91 | 9.50 | 9.30 | 9.39 |
| 629 | 0.00091 | 0.77 | 0.38 | 0.69 | 289 | 5.93 | 6.71 | 6.27 | 6.08 | 5.89 | 6.35 | 6.12 | 6.27 |
| 630 | 0.00091 | −0.62 | 0.33 | −0.69 | 351.5 | 7.30 | 6.67 | 6.88 | 7.03 | 7.18 | 6.78 | 6.89 | 7.08 |
| 631 | 0.00091 | 0.68 | 0.37 | 0.69 | 289 | 4.95 | 5.63 | 5.23 | 5.10 | 5.21 | 5.43 | 5.44 | 4.96 |
| 632 | 0.00091 | 0.38 | 0.19 | 0.74 | 116.5 | 8.94 | 9.32 | 9.22 | 9.08 | 8.97 | 9.31 | 9.06 | 9.04 |
| 633 | 0.00091 | −0.45 | 0.25 | −0.72 | 204 | 7.70 | 7.25 | 7.40 | 7.40 | 7.56 | 7.45 | 7.39 | 7.26 |
| 634 | 0.00091 | 0.94 | 0.49 | 0.69 | 289 | 7.32 | 8.26 | 7.95 | 7.63 | 7.31 | 7.79 | 7.81 | 7.87 |
| 635 | 0.00091 | 0.46 | 0.23 | 0.81 | 4.5 | 7.90 | 8.35 | 8.10 | 8.21 | 8.01 | 8.17 | 8.14 | 8.19 |
| 636 | 0.00091 | −0.52 | 0.30 | −0.73 | 141.5 | 8.78 | 8.26 | 8.51 | 8.58 | 8.70 | 8.38 | 8.53 | 8.26 |
| 637 | 0.00091 | −0.39 | 0.20 | −0.69 | 319.5 | 10.10 | 9.71 | 10.07 | 9.96 | 10.01 | 9.72 | 9.58 | 9.74 |
| 638 | 0.00091 | −0.51 | 0.25 | −0.75 | 82 | 7.83 | 7.32 | 7.51 | 7.63 | 7.65 | 7.42 | 7.32 | 7.57 |
| 639 | 0.00145 | −0.46 | 0.27 | −0.69 | 351.5 | 6.58 | 6.11 | 6.39 | 6.44 | 6.55 | 6.28 | 6.33 | 6.38 |
| 640 | 0.00091 | −0.40 | 0.21 | −0.69 | 351.5 | 8.63 | 8.23 | 8.49 | 8.57 | 8.77 | 8.29 | 8.66 | 8.70 |
| 641 | 0.00091 | 0.46 | 0.24 | 0.76 | 72 | 9.05 | 9.52 | 9.27 | 9.29 | 9.19 | 9.44 | 9.08 | 9.42 |
| 642 | 0.00091 | −0.37 | 0.19 | −0.73 | 141.5 | 8.79 | 8.42 | 8.57 | 8.56 | 8.76 | 8.52 | 8.52 | 8.55 |
| 643 | 0.00091 | 0.74 | 0.38 | 0.78 | 26 | 6.36 | 7.10 | 6.47 | 6.69 | 6.68 | 7.06 | 6.60 | 6.79 |
| 644 | 0.00091 | −0.29 | 0.17 | −0.69 | 351.5 | 3.76 | 3.47 | 3.55 | 3.55 | 3.50 | 3.63 | 3.52 | 3.58 |
| 645 | 0.00091 | −0.38 | 0.20 | −0.74 | 127.5 | 9.50 | 9.12 | 9.47 | 9.31 | 9.29 | 9.09 | 9.24 | 9.27 |
| 646 | 0.00091 | −0.61 | 0.33 | −0.72 | 204 | 7.37 | 6.76 | 7.33 | 7.05 | 7.28 | 6.77 | 7.05 | 7.03 |
| 647 | 0.00091 | 0.57 | 0.29 | 0.73 | 158 | 9.17 | 9.74 | 9.23 | 9.27 | 9.29 | 9.71 | 9.44 | 9.34 |
| 648 | 0.00091 | −0.37 | 0.20 | −0.73 | 165 | 7.42 | 7.05 | 7.27 | 7.28 | 7.40 | 6.83 | 7.09 | 7.34 |
| 649 | 0.00091 | −0.35 | 0.20 | −0.75 | 90.5 | 7.66 | 7.30 | 7.51 | 7.56 | 7.64 | 7.26 | 7.53 | 7.43 |
| 650 | 0.00091 | −0.21 | 0.12 | −0.69 | 351.5 | 8.67 | 8.46 | 8.56 | 8.62 | 8.60 | 8.44 | 8.57 | 8.56 |
| 651 | 0.00091 | −0.36 | 0.20 | −0.72 | 204 | 7.66 | 7.31 | 7.53 | 7.43 | 7.51 | 7.31 | 7.40 | 7.20 |
| 652 | 0.00091 | −0.40 | 0.21 | −0.74 | 127.5 | 8.27 | 7.87 | 8.07 | 8.16 | 8.19 | 8.03 | 8.07 | 8.05 |
| 653 | 0.00091 | −0.73 | 0.38 | −0.69 | 351.5 | 8.23 | 7.49 | 7.61 | 7.93 | 8.29 | 7.63 | 7.84 | 8.07 |
| 654 | 0.00091 | −0.23 | 0.13 | −0.69 | 319.5 | 8.66 | 8.42 | 8.54 | 8.60 | 8.68 | 8.49 | 8.50 | 8.56 |
| 655 | 0.00091 | −0.75 | 0.41 | −0.73 | 141.5 | 5.93 | 5.18 | 5.87 | 5.52 | 6.09 | 5.37 | 5.66 | 5.79 |
| 656 | 0.00091 | −0.61 | 0.32 | −0.72 | 172.5 | 5.72 | 5.10 | 5.33 | 5.64 | 5.82 | 5.24 | 5.52 | 5.66 |
| 657 | 0.00091 | 0.69 | 0.35 | 0.76 | 72 | 5.38 | 6.07 | 5.70 | 5.83 | 5.54 | 6.04 | 5.66 | 5.90 |
| 658 | 0.00091 | −0.45 | 0.21 | −0.79 | 22 | 7.66 | 7.21 | 7.41 | 7.51 | 7.66 | 7.30 | 7.43 | 7.53 |
| 659 | 0.00091 | 0.38 | 0.22 | 0.69 | 289 | 8.76 | 9.13 | 8.94 | 8.94 | 8.69 | 9.38 | 8.85 | 9.11 |
| 660 | 0.00091 | 0.58 | 0.31 | 0.72 | 186 | 8.11 | 8.69 | 8.45 | 8.49 | 8.26 | 8.68 | 8.70 | 8.70 |
| 661 | 0.00091 | 0.44 | 0.24 | 0.69 | 371.5 | 8.93 | 9.37 | 9.19 | 9.23 | 9.01 | 9.38 | 9.07 | 9.27 |
| 662 | 0.00091 | −0.51 | 0.27 | −0.72 | 172.5 | 9.85 | 9.33 | 9.75 | 9.74 | 9.70 | 9.38 | 9.80 | 9.71 |
| 663 | 0.00145 | −0.24 | 0.13 | −0.69 | 319.5 | 8.91 | 8.67 | 8.73 | 8.83 | 8.91 | 8.62 | 8.77 | 8.83 |
| 664 | 0.00091 | 0.44 | 0.23 | 0.70 | 265.5 | 8.25 | 8.69 | 8.37 | 8.46 | 8.17 | 8.63 | 8.49 | 8.31 |
| 665 | 0.00091 | −0.59 | 0.31 | −0.74 | 127.5 | 6.27 | 5.68 | 6.11 | 6.04 | 6.23 | 6.05 | 6.00 | 5.86 |
| 666 | 0.00091 | 0.73 | 0.37 | 0.71 | 222.5 | 9.28 | 10.01 | 9.82 | 9.55 | 9.40 | 9.83 | 9.45 | 9.32 |
| 667 | 0.00091 | −0.25 | 0.14 | −0.78 | 38 | 9.51 | 9.26 | 9.39 | 9.38 | 9.44 | 9.22 | 9.34 | 9.33 |
| 668 | 0.00091 | −0.33 | 0.19 | −0.69 | 351.5 | 9.70 | 9.37 | 9.58 | 9.62 | 9.51 | 9.38 | 9.39 | 9.48 |
| 669 | 0.00091 | 0.33 | 0.17 | 0.75 | 103 | 8.25 | 8.58 | 8.47 | 8.44 | 8.30 | 8.69 | 8.44 | 8.67 |
| 670 | 0.00091 | −0.46 | 0.24 | −0.77 | 47.5 | 10.33 | 9.87 | 10.19 | 10.13 | 10.24 | 10.03 | 10.18 | 10.02 |
| 671 | 0.00145 | −0.44 | 0.23 | −0.72 | 204 | 7.43 | 6.99 | 7.19 | 7.28 | 7.38 | 7.25 | 7.12 | 7.21 |
| 672 | 0.00091 | −0.76 | 0.38 | −0.77 | 47.5 | 8.58 | 7.82 | 8.28 | 8.41 | 8.49 | 8.06 | 8.47 | 8.22 |
| 673 | 0.00091 | −0.25 | 0.14 | −0.69 | 351.5 | 7.49 | 7.24 | 7.44 | 7.36 | 7.51 | 7.37 | 7.48 | 7.32 |
| 674 | 0.00091 | −0.30 | 0.15 | −0.70 | 249.5 | 8.94 | 8.64 | 8.72 | 8.84 | 8.78 | 8.66 | 8.80 | 8.67 |
| 675 | 0.00091 | −0.78 | 0.39 | −0.79 | 19 | 7.19 | 6.41 | 6.83 | 6.86 | 7.09 | 6.70 | 7.01 | 6.69 |
| 676 | 0.00091 | −0.34 | 0.19 | −0.69 | 351.5 | 4.63 | 4.30 | 4.64 | 4.47 | 4.58 | 4.51 | 4.42 | 4.38 |
| 677 | 0.00091 | −0.71 | 0.36 | −0.75 | 82 | 6.08 | 5.36 | 5.61 | 5.62 | 5.75 | 5.69 | 5.67 | 5.67 |
| 678 | 0.00145 | −0.58 | 0.31 | −0.69 | 319.5 | 6.96 | 6.37 | 6.52 | 6.77 | 7.02 | 6.68 | 6.67 | 6.66 |
| 679 | 0.00091 | 0.53 | 0.26 | 0.82 | 2.5 | 8.08 | 8.62 | 8.22 | 8.29 | 8.36 | 8.68 | 8.33 | 8.71 |
| 680 | 0.00091 | 0.60 | 0.29 | 0.75 | 103 | 6.79 | 7.39 | 7.15 | 7.00 | 6.92 | 7.25 | 7.04 | 7.17 |
| 681 | 0.00091 | −0.29 | 0.16 | −0.69 | 319.5 | 9.36 | 9.07 | 9.20 | 9.32 | 9.34 | 9.15 | 9.28 | 9.22 |
| 682 | 0.00145 | −0.27 | 0.14 | −0.72 | 172.5 | 7.20 | 6.93 | 7.01 | 7.03 | 7.06 | 7.00 | 7.00 | 7.06 |
| 683 | 0.00091 | 0.44 | 0.23 | 0.71 | 222.5 | 9.30 | 9.73 | 9.55 | 9.47 | 9.61 | 9.72 | 9.73 | 9.84 |
| 684 | 0.00091 | −0.49 | 0.25 | −0.73 | 141.5 | 8.40 | 7.90 | 8.08 | 8.14 | 8.17 | 8.05 | 8.11 | 8.07 |
| 685 | 0.00091 | −0.37 | 0.20 | −0.75 | 82 | 10.47 | 10.10 | 10.39 | 10.27 | 10.59 | 10.08 | 10.43 | 10.42 |
| 686 | 0.00145 | −0.54 | 0.29 | −0.72 | 172.5 | 8.08 | 7.54 | 7.84 | 7.97 | 8.03 | 7.79 | 7.78 | 7.70 |
| 687 | 0.00091 | −0.61 | 0.31 | −0.73 | 141.5 | 8.70 | 8.10 | 8.49 | 8.54 | 8.59 | 8.27 | 8.26 | 8.32 |
| 688 | 0.00091 | −0.37 | 0.19 | −0.72 | 204 | 5.80 | 5.43 | 5.55 | 5.61 | 5.80 | 5.64 | 5.63 | 5.61 |
| 689 | 0.00091 | −0.74 | 0.40 | −0.71 | 235 | 8.64 | 7.91 | 8.59 | 8.55 | 8.55 | 8.03 | 8.43 | 8.35 |
| 690 | 0.00091 | −0.97 | 0.48 | −0.78 | 31.5 | 8.40 | 7.43 | 7.89 | 8.05 | 8.08 | 7.73 | 7.83 | 7.80 |
| 691 | 0.00091 | −0.55 | 0.29 | −0.72 | 204 | 8.05 | 7.50 | 7.64 | 7.88 | 8.19 | 7.60 | 7.88 | 7.87 |
| 692 | 0.00091 | −0.29 | 0.16 | −0.77 | 47.5 | 10.05 | 9.76 | 9.95 | 9.91 | 9.93 | 9.81 | 9.81 | 9.86 |
| 693 | 0.00091 | −0.47 | 0.23 | −0.72 | 172.5 | 8.13 | 7.67 | 8.01 | 7.97 | 8.18 | 7.94 | 7.96 | 8.06 |
| 694 | 0.00091 | 0.33 | 0.18 | 0.69 | 289 | 8.55 | 8.88 | 8.66 | 8.59 | 8.48 | 8.84 | 8.52 | 8.66 |
| 695 | 0.00091 | 0.43 | 0.22 | 0.69 | 371.5 | 4.56 | 4.99 | 4.90 | 4.72 | 4.57 | 4.89 | 4.57 | 4.65 |
| 696 | 0.00091 | −0.49 | 0.25 | −0.69 | 319.5 | 8.38 | 7.89 | 8.10 | 8.29 | 8.56 | 7.94 | 8.09 | 8.46 |
| 697 | 0.00091 | −0.51 | 0.27 | −0.75 | 90.5 | 9.78 | 9.27 | 9.64 | 9.65 | 9.75 | 9.46 | 9.44 | 9.39 |
| 698 | 0.00239 | −0.54 | 0.29 | −0.71 | 235 | 6.59 | 6.05 | 6.32 | 6.23 | 6.64 | 6.22 | 6.22 | 6.36 |
| 699 | 0.00091 | 0.59 | 0.30 | 0.72 | 214 | 6.45 | 7.04 | 6.74 | 6.65 | 6.69 | 6.59 | 6.68 | 7.02 |
| 700 | 0.00091 | 0.39 | 0.22 | 0.70 | 265.5 | 9.29 | 9.69 | 9.45 | 9.31 | 9.39 | 9.46 | 9.55 | 9.52 |
| 701 | 0.00091 | −0.93 | 0.47 | −0.69 | 319.5 | 6.85 | 5.92 | 6.05 | 6.51 | 6.66 | 5.99 | 6.09 | 6.30 |
| 702 | 0.00091 | −0.75 | 0.38 | −0.73 | 141.5 | 6.43 | 5.68 | 5.96 | 6.28 | 6.57 | 5.96 | 6.07 | 6.26 |
| 703 | 0.00091 | 0.49 | 0.26 | 0.73 | 158 | 8.89 | 9.38 | 9.11 | 9.02 | 9.03 | 9.54 | 9.37 | 9.09 |
| 704 | 0.00091 | −0.65 | 0.36 | −0.72 | 204 | 5.50 | 4.85 | 5.37 | 5.20 | 5.28 | 5.23 | 4.92 | 5.14 |
| 705 | 0.00091 | −0.64 | 0.33 | −0.69 | 351.5 | 7.83 | 7.18 | 7.28 | 7.54 | 7.56 | 7.30 | 7.22 | 7.31 |
| 706 | 0.00091 | −0.61 | 0.30 | −0.75 | 90.5 | 5.74 | 5.13 | 5.37 | 5.62 | 5.75 | 5.26 | 5.62 | 5.67 |
| 707 | 0.00091 | −0.42 | 0.21 | −0.75 | 90.5 | 7.42 | 7.00 | 7.24 | 7.29 | 7.42 | 7.14 | 7.24 | 7.40 |
| 708 | 0.00091 | 0.64 | 0.34 | 0.75 | 103 | 7.37 | 8.01 | 7.54 | 7.48 | 7.57 | 7.61 | 7.71 | 7.90 |
| 709 | 0.00091 | −0.60 | 0.31 | −0.78 | 31.5 | 9.01 | 8.40 | 8.83 | 8.85 | 8.97 | 8.75 | 8.90 | 8.69 |
| 710 | 0.00091 | −0.33 | 0.19 | −0.70 | 249.5 | 7.69 | 7.35 | 7.55 | 7.45 | 7.57 | 7.38 | 7.57 | 7.54 |
| 711 | 0.00091 | −0.57 | 0.27 | −0.78 | 31.5 | 6.96 | 6.39 | 6.62 | 6.82 | 6.74 | 6.57 | 6.69 | 6.59 |
| 712 | 0.00091 | −0.79 | 0.40 | −0.81 | 10.5 | 7.16 | 6.37 | 6.85 | 6.76 | 7.10 | 6.43 | 6.62 | 6.78 |
| 713 | 0.00091 | −1.28 | 0.62 | −0.75 | 90.5 | 6.79 | 5.51 | 6.29 | 6.53 | 6.76 | 5.76 | 6.21 | 5.78 |
| 714 | 0.00091 | 0.46 | 0.25 | 0.70 | 265.5 | 9.38 | 9.83 | 9.54 | 9.60 | 9.49 | 9.73 | 9.52 | 9.87 |
| 715 | 0.00091 | −0.46 | 0.24 | −0.73 | 141.5 | 5.91 | 5.45 | 5.68 | 5.87 | 5.84 | 5.71 | 5.82 | 5.80 |
| 716 | 0.00145 | −0.45 | 0.23 | −0.72 | 204 | 7.72 | 7.27 | 7.48 | 7.62 | 7.62 | 7.46 | 7.58 | 7.68 |
| 717 | 0.00091 | −0.49 | 0.25 | −0.69 | 319.5 | 6.19 | 5.70 | 5.98 | 6.10 | 6.13 | 5.68 | 5.78 | 5.85 |
| 718 | 0.00091 | −0.51 | 0.27 | −0.72 | 172.5 | 10.48 | 9.98 | 10.13 | 10.38 | 10.56 | 10.22 | 10.32 | 10.29 |
| 719 | 0.00091 | 0.35 | 0.19 | 0.69 | 289 | 7.59 | 7.94 | 7.68 | 7.88 | 7.68 | 7.93 | 7.74 | 7.75 |
| 720 | 0.00091 | −0.81 | 0.47 | −0.70 | 249.5 | 7.08 | 6.28 | 6.63 | 6.64 | 7.27 | 6.69 | 6.70 | 6.43 |
| 721 | 0.00091 | −0.70 | 0.38 | −0.73 | 141.5 | 7.48 | 6.78 | 7.30 | 7.19 | 7.42 | 7.05 | 7.17 | 7.28 |
| 722 | 0.00145 | −0.50 | 0.26 | −0.69 | 319.5 | 8.00 | 7.50 | 7.84 | 7.91 | 8.09 | 7.56 | 7.80 | 8.21 |
| 723 | 0.00091 | −0.34 | 0.20 | −0.71 | 235 | 7.66 | 7.32 | 7.51 | 7.41 | 7.74 | 7.45 | 7.37 | 7.39 |
| 724 | 0.00091 | 0.69 | 0.34 | 0.74 | 116.5 | 8.33 | 9.02 | 8.79 | 8.56 | 8.46 | 9.01 | 8.80 | 8.70 |
| 725 | 0.00091 | −0.92 | 0.46 | −0.75 | 90.5 | 6.99 | 6.07 | 6.47 | 6.68 | 7.12 | 6.26 | 6.74 | 6.70 |
| 726 | 0.00091 | 0.48 | 0.25 | 0.73 | 158 | 4.30 | 4.78 | 4.50 | 4.44 | 4.33 | 4.67 | 4.51 | 4.28 |
| 727 | 0.00091 | 0.26 | 0.15 | 0.69 | 371.5 | 7.16 | 7.42 | 7.17 | 7.25 | 7.17 | 7.43 | 7.45 | 7.39 |
| 728 | 0.00091 | 0.42 | 0.23 | 0.76 | 72 | 8.43 | 8.84 | 8.50 | 8.66 | 8.53 | 8.92 | 8.62 | 8.79 |
| 729 | 0.00091 | 0.71 | 0.37 | 0.74 | 116.5 | 7.38 | 8.09 | 7.78 | 7.83 | 7.45 | 7.78 | 7.98 | 8.10 |
| 730 | 0.00091 | −0.70 | 0.35 | −0.74 | 127.5 | 7.87 | 7.17 | 7.74 | 7.75 | 7.84 | 7.12 | 7.74 | 7.68 |
| 731 | 0.00091 | −0.88 | 0.46 | −0.76 | 60.5 | 7.21 | 6.33 | 7.06 | 6.88 | 7.02 | 6.51 | 6.98 | 6.59 |
| 732 | 0.00091 | −0.40 | 0.21 | −0.76 | 60.5 | 8.85 | 8.45 | 8.71 | 8.76 | 8.80 | 8.59 | 8.75 | 8.55 |
| 733 | 0.00091 | 0.60 | 0.32 | 0.75 | 103 | 5.63 | 6.23 | 5.80 | 5.96 | 5.72 | 6.13 | 5.88 | 6.18 |
| 734 | 0.00091 | −0.28 | 0.15 | −0.69 | 319.5 | 9.56 | 9.27 | 9.40 | 9.49 | 9.48 | 9.25 | 9.29 | 9.41 |
| 735 | 0.00091 | 0.45 | 0.23 | 0.69 | 289 | 10.22 | 10.67 | 10.48 | 10.26 | 10.32 | 10.40 | 10.40 | 10.45 |
| 736 | 0.00091 | −0.38 | 0.22 | −0.69 | 319.5 | 7.28 | 6.90 | 7.07 | 6.97 | 7.35 | 7.05 | 6.86 | 7.01 |
| 737 | 0.00091 | −0.40 | 0.20 | −0.72 | 204 | 9.06 | 8.66 | 8.91 | 9.00 | 9.13 | 8.86 | 8.88 | 9.08 |
| 738 | 0.00091 | 0.40 | 0.21 | 0.75 | 103 | 7.43 | 7.82 | 7.60 | 7.68 | 7.46 | 7.84 | 7.62 | 8.01 |
| 739 | 0.00195 | −0.80 | 0.42 | −0.73 | 141.5 | 9.72 | 8.93 | 9.31 | 9.26 | 9.59 | 9.11 | 9.05 | 9.21 |
| 740 | 0.00091 | 0.63 | 0.35 | 0.69 | 371.5 | 7.56 | 8.19 | 7.99 | 7.79 | 7.64 | 8.27 | 8.04 | 7.92 |
| 741 | 0.00091 | −0.42 | 0.21 | −0.72 | 172.5 | 9.38 | 8.96 | 9.14 | 9.20 | 9.38 | 9.03 | 9.13 | 9.25 |
| 742 | 0.00091 | −0.23 | 0.12 | −0.69 | 319.5 | 10.96 | 10.74 | 10.82 | 10.92 | 11.00 | 10.94 | 10.88 | 10.86 |
| 743 | 0.00091 | −0.26 | 0.14 | −0.71 | 235 | 6.05 | 5.78 | 5.92 | 5.90 | 6.02 | 5.85 | 6.07 | 6.06 |
| 744 | 0.00091 | −0.71 | 0.37 | −0.75 | 90.5 | 4.39 | 3.67 | 4.21 | 3.93 | 4.36 | 3.78 | 3.91 | 3.92 |
| 745 | 0.00145 | −0.72 | 0.38 | −0.78 | 31.5 | 7.03 | 6.31 | 6.91 | 6.76 | 6.78 | 6.70 | 6.46 | 6.60 |
| 746 | 0.00091 | −0.58 | 0.30 | −0.72 | 172.5 | 6.60 | 6.01 | 6.43 | 6.44 | 6.56 | 6.16 | 6.33 | 6.54 |
| 747 | 0.00091 | 0.73 | 0.40 | 0.71 | 222.5 | 7.48 | 8.22 | 7.50 | 7.79 | 7.52 | 8.17 | 7.89 | 8.09 |
| 748 | 0.00091 | 0.84 | 0.42 | 0.71 | 222.5 | 5.48 | 6.31 | 5.97 | 5.58 | 5.62 | 5.85 | 6.03 | 5.81 |
| 749 | 0.00091 | 0.53 | 0.28 | 0.73 | 158 | 6.85 | 7.37 | 7.16 | 7.16 | 7.09 | 7.05 | 7.22 | 7.39 |
| 750 | 0.00091 | −0.28 | 0.13 | −0.80 | 17 | 8.79 | 8.51 | 8.67 | 8.59 | 8.90 | 8.52 | 8.70 | 8.76 |
| 751 | 0.00091 | −0.56 | 0.27 | −0.73 | 141.5 | 7.80 | 7.25 | 7.69 | 7.71 | 7.79 | 7.29 | 7.68 | 7.88 |
| 752 | 0.00091 | 0.99 | 0.47 | 0.81 | 7.5 | 8.13 | 9.12 | 8.56 | 8.59 | 8.42 | 9.00 | 8.92 | 8.89 |
| 753 | 0.00091 | −0.84 | 0.43 | −0.69 | 351.5 | 5.09 | 4.25 | 4.88 | 5.14 | 5.33 | 4.51 | 4.85 | 4.69 |
| 754 | 0.00091 | 0.42 | 0.24 | 0.69 | 371.5 | 7.73 | 8.15 | 7.96 | 7.83 | 7.77 | 8.21 | 7.99 | 8.17 |
| 755 | 0.00091 | −0.63 | 0.33 | −0.72 | 204 | 9.30 | 8.67 | 8.91 | 9.08 | 9.20 | 8.90 | 9.02 | 9.00 |
| 756 | 0.00091 | −0.88 | 0.44 | −0.70 | 249.5 | 5.97 | 5.08 | 5.50 | 5.96 | 5.94 | 5.34 | 5.60 | 5.50 |
| 757 | 0.00091 | 0.50 | 0.28 | 0.70 | 265.5 | 9.25 | 9.75 | 9.52 | 9.33 | 9.30 | 9.61 | 9.39 | 9.56 |
| 758 | 0.00145 | −0.30 | 0.15 | −0.73 | 141.5 | 8.75 | 8.45 | 8.69 | 8.63 | 8.70 | 8.47 | 8.57 | 8.49 |
| 759 | 0.00091 | −0.49 | 0.24 | −0.75 | 90.5 | 8.91 | 8.42 | 8.61 | 8.75 | 9.05 | 8.76 | 8.85 | 8.61 |
| 760 | 0.00091 | −0.42 | 0.22 | −0.72 | 172.5 | 7.27 | 6.85 | 7.01 | 6.99 | 7.34 | 6.87 | 7.18 | 7.20 |
| 761 | 0.00091 | −0.75 | 0.40 | −0.70 | 249.5 | 7.12 | 6.37 | 6.71 | 7.05 | 7.10 | 6.61 | 6.86 | 6.59 |
| 762 | 0.00091 | −0.39 | 0.20 | −0.69 | 351.5 | 7.15 | 6.76 | 6.92 | 7.13 | 7.11 | 6.75 | 6.95 | 7.07 |
| 763 | 0.00091 | 0.80 | 0.42 | 0.69 | 289 | 8.55 | 9.35 | 9.02 | 8.88 | 8.63 | 9.50 | 9.08 | 8.60 |
| 764 | 0.00091 | 0.62 | 0.31 | 0.77 | 42.5 | 8.10 | 8.71 | 8.29 | 8.31 | 8.12 | 8.34 | 8.01 | 8.29 |
| 765 | 0.00091 | 0.42 | 0.23 | 0.70 | 276 | 7.42 | 7.84 | 7.56 | 7.79 | 7.54 | 7.53 | 7.83 | 7.96 |
| 766 | 0.00091 | −0.62 | 0.31 | −0.75 | 90.5 | 8.96 | 8.34 | 8.57 | 8.76 | 9.11 | 8.65 | 8.77 | 8.68 |
| 767 | 0.00145 | −0.31 | 0.18 | −0.69 | 319.5 | 8.21 | 7.91 | 8.13 | 8.07 | 8.19 | 7.82 | 7.89 | 8.04 |
| 768 | 0.00091 | 0.63 | 0.32 | 0.75 | 103 | 6.41 | 7.04 | 6.86 | 6.77 | 6.45 | 6.92 | 6.78 | 7.05 |
| 769 | 0.00091 | −0.56 | 0.30 | −0.69 | 319.5 | 8.60 | 8.05 | 8.33 | 8.59 | 8.38 | 8.06 | 8.48 | 8.25 |
| 770 | 0.00091 | 0.52 | 0.27 | 0.71 | 222.5 | 9.35 | 9.88 | 9.52 | 9.35 | 9.27 | 9.96 | 9.54 | 9.45 |
| 771 | 0.00091 | −0.33 | 0.20 | −0.69 | 319.5 | 8.50 | 8.16 | 8.45 | 8.30 | 8.39 | 8.28 | 8.38 | 8.23 |
| 772 | 0.00091 | 0.55 | 0.27 | 0.81 | 4.5 | 8.85 | 9.40 | 9.07 | 9.10 | 9.07 | 9.53 | 9.22 | 9.48 |
| 773 | 0.00091 | 0.63 | 0.33 | 0.78 | 34.5 | 7.12 | 7.76 | 7.40 | 7.60 | 7.41 | 7.79 | 7.56 | 8.02 |
| 774 | 0.00091 | 0.66 | 0.33 | 0.69 | 371.5 | 8.33 | 8.99 | 8.63 | 8.47 | 8.41 | 9.03 | 8.83 | 8.59 |
| 775 | 0.00091 | 0.44 | 0.23 | 0.71 | 222.5 | 9.30 | 9.73 | 9.55 | 9.47 | 9.61 | 9.72 | 9.73 | 9.84 |
| 776 | 0.00091 | 0.54 | 0.27 | 0.73 | 158 | 8.59 | 9.12 | 8.95 | 8.92 | 8.84 | 8.86 | 9.05 | 9.20 |
| 777 | 0.00091 | 0.28 | 0.15 | 0.69 | 289 | 7.36 | 7.65 | 7.42 | 7.41 | 7.37 | 7.62 | 7.46 | 7.37 |
| 778 | 0.00091 | −0.68 | 0.36 | −0.69 | 319.5 | 7.02 | 6.35 | 6.67 | 6.76 | 7.16 | 6.52 | 6.74 | 6.38 |
| 779 | 0.00091 | −0.62 | 0.33 | −0.69 | 351.5 | 7.30 | 6.67 | 6.88 | 7.03 | 7.18 | 6.78 | 6.89 | 7.08 |
| 780 | 0.00091 | −0.67 | 0.35 | −0.70 | 249.5 | 7.69 | 7.02 | 7.62 | 7.69 | 7.75 | 7.15 | 7.45 | 7.60 |
| 781 | 0.00091 | −0.63 | 0.33 | −0.72 | 204 | 9.30 | 8.67 | 8.91 | 9.08 | 9.20 | 8.90 | 9.02 | 9.00 |
| 782 | 0.00091 | −0.43 | 0.24 | −0.71 | 235 | 7.84 | 7.40 | 7.59 | 7.61 | 7.85 | 7.49 | 7.55 | 7.78 |
| 783 | 0.00091 | 0.88 | 0.48 | 0.72 | 186 | 6.14 | 7.02 | 6.32 | 6.60 | 6.60 | 6.95 | 7.00 | 6.64 |
| 784 | 0.00091 | 0.25 | 0.15 | 0.71 | 222.5 | 8.01 | 8.27 | 8.07 | 8.12 | 8.03 | 8.24 | 8.00 | 8.36 |
| 785 | 0.00091 | 0.57 | 0.31 | 0.76 | 72 | 7.52 | 8.08 | 7.76 | 7.82 | 7.61 | 7.89 | 8.07 | 8.14 |
| 786 | 0.00091 | −0.23 | 0.14 | −0.72 | 172.5 | 9.25 | 9.02 | 9.13 | 9.14 | 9.30 | 9.24 | 9.04 | 8.94 |
| 787 | 0.00091 | −0.45 | 0.23 | −0.72 | 204 | 6.80 | 6.34 | 6.49 | 6.63 | 6.90 | 6.33 | 6.47 | 6.38 |
| 788 | 0.00091 | −0.32 | 0.17 | −0.69 | 319.5 | 11.39 | 11.07 | 11.14 | 11.23 | 11.33 | 11.09 | 11.16 | 11.09 |
| 789 | 0.00091 | −0.34 | 0.18 | −0.68 | 379 | 10.89 | 10.55 | 10.65 | 10.77 | 10.81 | 10.65 | 10.67 | 10.61 |
| 790 | 0.00091 | 0.38 | 0.19 | 0.74 | 116.5 | 8.94 | 9.32 | 9.22 | 9.08 | 8.97 | 9.31 | 9.06 | 9.04 |
| 791 | 0.00091 | 0.38 | 0.19 | 0.74 | 116.5 | 8.94 | 9.32 | 9.22 | 9.08 | 8.97 | 9.31 | 9.06 | 9.04 |
| 792 | 0.00091 | −0.40 | 0.21 | −0.74 | 127.5 | 8.27 | 7.87 | 8.07 | 8.16 | 8.19 | 8.03 | 8.07 | 8.05 |
| 793 | 0.00091 | −0.75 | 0.38 | −0.78 | 38 | 8.41 | 7.66 | 8.03 | 8.11 | 8.65 | 8.11 | 8.22 | 8.44 |
| 794 | 0.00091 | −0.28 | 0.15 | −0.69 | 319.5 | 9.56 | 9.27 | 9.40 | 9.49 | 9.48 | 9.25 | 9.29 | 9.41 |

Three sets of genes were selected using independent criteria that suggested biological significance based upon microarray data. The first set of genes were selected based upon log-2 fold change in DBP-4 hr vs. untreated. We took the 384 genes with the most extreme log-2 fold changes, up or down. The second set was chosen based upon the p-value for a Wilcoxon rank-sum test of differential expression between DBP-4 hr and untreated. The 384 genes with the lowest p-values were selected. Finally, the third set of genes was selected based upon the Kendall tau coefficient. This measures the strength of a linear relationship between the input variables, in this case gene expression, and the output variable of draining lymph node counts. The three gene sets were selected independently, but not surprisingly showed some overlap. The 384 genes with the most extreme tau coefficients were selected and are shown in Table 9.

TABLE 9

| | | | | | Wilcoxon | | | DBP |
|---|---|---|---|---|---|---|---|---|
| | | | | Kendall | Differ- | Un- | | Log |
| | | | | Corre- | ential | treated | DBP | Fold |
| | | | | lation | DBP vs | Ex- | Ex- | Change |
| | | | | Adj | Untreated | pres- | pres- | Versus |
| # | Gene Name | Gene Symbol | Ref Seq ID | P-value | Adj P-value | sion | sion | Untreated |
| 1 | chemokine (C-C motif) ligand 20 | Ccl20 | NM_001159738_at | 0.000719 | 0.0265 | 5.18 | 11.11 | 5.93 |
| 2 | chemokine (C-C motif) ligand 20 | Ccl20 | NM_016960_at | 0.000719 | 0.0265 | 5.18 | 11.11 | 5.93 |
| 3 | chemokine (C-X-C motif) ligand 1 | Cxcl1 | NM_008176_at | 0.002760 | 0.0265 | 4.81 | 10.24 | 5.43 |
| 4 | chemokine (C-X-C motif) ligand 2 | Cxcl2 | NM_009140_at | 0.002090 | 0.0265 | 4.11 | 8.67 | 4.56 |
| 5 | epithelial mitogen | Epgn | NM_053087_at | 0.002090 | 0.0265 | 5.27 | 9.09 | 3.82 |
| 6 | chemokine (C-C motif) ligand 2 | Ccl2 | NM_011333_at | 0.001338 | 0.0265 | 6.22 | 9.45 | 3.23 |
| 7 | chemokine (C-C motif) ligand 7 | Ccl7 | NM_013654_at | 0.001338 | 0.0265 | 5.75 | 8.86 | 3.11 |
| 8 | chemokine (C-C motif) ligand 12 | Ccl12 | NM_011331_at | 0.000978 | 0.0265 | 5.37 | 8.47 | 3.10 |
| 9 | c-C motif chemokine 12-like | LOC100504977 | XM_003085794_at | 0.000978 | 0.0265 | 5.37 | 8.47 | 3.10 |
| 10 | interleukin 6 | Il6 | NM_031168_at | 0.000842 | 0.0265 | 3.36 | 6.41 | 3.05 |
| 11 | interleukin 1 beta | Il1b | NM_008361_at | 0.003180 | 0.0265 | 4.94 | 7.67 | 2.73 |
| 12 | serine (or cysteine) peptidase inhibitor, | Serpine1 | NM_008871_at | 0.002760 | 0.0265 | 5.64 | 8.32 | 2.68 |
| | clade E, member 1 | | | | | | | |
| 13 | intercellular adhesion molecule 1 | Icam1 | NM_010493_at | 0.000189 | 0.0265 | 7.88 | 10.54 | 2.66 |
| 14 | peptide YY | Pyy | NM_145435_at | 0.000051 | 0.0265 | 6.55 | 9.16 | 2.62 |
| 15 | angiopoietin-like 4 | Angptl4 | NM_020581_at | 0.000003 | 0.0265 | 6.90 | 9.51 | 2.61 |
| 16 | B-cell leukemia/lymphoma 3 | Bcl3 | NM_033601_at | 0.000319 | 0.0265 | 6.36 | 8.92 | 2.56 |
| 17 | tumor necrosis factor alpha induced | Tnfaip6 | NM_009398_at | 0.001808 | 0.0265 | 6.49 | 8.79 | 2.30 |
| | protein 6 | | | | | | | |
| 18 | histidine decarboxylase | Hdc | NM_008230_at | 0.000062 | 0.0265 | 6.37 | 8.51 | 2.14 |
| 19 | regulator of G-protein signaling 1 | Rgs1 | NM_015811_at | 0.000444 | 0.0265 | 5.23 | 7.35 | 2.12 |
| 20 | tumor necrosis factor, alpha-induced | Tnfaip3 | NM_001166402_at | 0.001561 | 0.0265 | 5.13 | 7.22 | 2.10 |
| | protein 3 | | | | | | | |
| 21 | tumor necrosis factor, alpha-induced | Tnfaip3 | NM_009397_at | 0.001561 | 0.0265 | 5.13 | 7.22 | 2.10 |
| | protein 3 | | | | | | | |
| 22 | C-type lectin domain family 7, member a | Clec7a | NM_020008_at | 0.000319 | 0.0265 | 7.05 | 9.14 | 2.09 |
| 23 | nuclear receptor subfamily 4, group A, | Nr4a1 | NM_010444_at | 0.000159 | 0.0265 | 8.26 | 10.34 | 2.09 |
| | member 1 | | | | | | | |
| 24 | fos-like antigen 1 | Fosl1 | NM_010235_at | 0.000319 | 0.0265 | 4.90 | 6.85 | 1.95 |
| 25 | tumor necrosis factor, alpha-induced | Tnfaip2 | NM_009396_at | 0.001146 | 0.0265 | 6.90 | 8.85 | 1.94 |
| | protein 2 | | | | | | | |
| 26 | a disintegrin and metallopeptidase | Adam8 | NM_007403_at | 0.000062 | 0.0265 | 6.90 | 8.84 | 1.94 |
| | domain 8 | | | | | | | |
| 27 | cholesterol 25-hydroxylase | Ch25h | NM_009890_at | 0.003180 | 0.0265 | 5.77 | 7.68 | 1.91 |
| 28 | activity regulated cytoskeletal-associated | Arc | NM_018790_at | 0.000159 | 0.0265 | 4.90 | 6.81 | 1.90 |
| | protein | | | | | | | |
| 29 | hepatitis A virus cellular receptor 2 | Havcr2 | NM_134250_at | 0.000091 | 0.0265 | 5.32 | 7.20 | 1.89 |
| 30 | immediate early response 3 | Ier3 | NM_133662_at | 0.000719 | 0.0265 | 9.40 | 11.24 | 1.85 |
| 31 | dual specificity phosphatase 6 | Dusp6 | NM_026268_at | 0.000614 | 0.0265 | 8.78 | 10.59 | 1.81 |
| 32 | heparin-binding EGF-like growth factor | Hbegf | NM_010415_at | 0.000842 | 0.0265 | 7.08 | 8.85 | 1.76 |
| 33 | CCR4 carbon catabolite repression 4-like | Ccrn4l | NM_009834_at | 0.000319 | 0.0265 | 8.31 | 10.05 | 1.75 |
| | (S. cerevisiae) | | | | | | | |
| 34 | CD86 antigen | Cd86 | NM_019388_at | 0.000444 | 0.0265 | 5.68 | 7.42 | 1.73 |
| 35 | protease, serine, 22 | Prss22 | NM_133731_at | 0.000978 | 0.0265 | 6.35 | 7.96 | 1.61 |
| 36 | nuclear factor, interleukin 3, regulated | Nfil3 | NM_017373_at | 0.001561 | 0.0265 | 6.54 | 8.13 | 1.60 |
| 37 | cysteine rich protein 61 | Cyr61 | NM_010516_at | 0.002414 | 0.0265 | 7.77 | 9.37 | 1.59 |
| 38 | CCAAT/enhancer binding protein | Cebpd | NM_007679_at | 0.002760 | 0.0265 | 8.59 | 10.17 | 1.58 |
| | (C/EBP), delta | | | | | | | |
| 39 | B-cell translation gene 2, anti- | Btg2 | NM_007570_at | 0.000004 | 0.0265 | 9.06 | 10.63 | 1.56 |
| | proliferative | | | | | | | |
| 40 | keratin 6A | Krt6a | NM_008476_at | 0.000018 | 0.0265 | 9.23 | 10.76 | 1.53 |
| 41 | RIKEN cDNA 1810011O10 gene | 1810011O10Rik | NM_026931_at | 0.001146 | 0.0265 | 8.15 | 9.68 | 1.53 |
| 42 | zinc finger protein 36 | Zfp36 | NM_011756_at | 0.000268 | 0.0265 | 8.86 | 10.39 | 1.53 |
| 43 | solute carrier family 7 (cationic amino | Slc7a5 | NM_011404_at | 0.000062 | 0.0265 | 8.48 | 10.00 | 1.52 |
| | acid transporter, y+ system), member 5 | | | | | | | |
| 44 | solute carrier family 39 (metal ion | Slc39a8 | NM_001135149_at | 0.000075 | 0.0265 | 4.61 | 6.13 | 1.52 |
| | transporter), member 8 | | | | | | | |
| 45 | solute carrier family 39 (metal ion | Slc39a8 | NM_001135150_at | 0.000075 | 0.0265 | 4.61 | 6.13 | 1.52 |
| | transporter), member 8 | | | | | | | |
| 46 | solute carrier family 39 (metal ion | Slc39a8 | NM_026228_at | 0.000075 | 0.0265 | 4.61 | 6.13 | 1.52 |
| | transporter), member 8 | | | | | | | |
| 47 | RIKEN cDNA 1600029D21 gene | 1600029D21Rik | NM_029639_at | 0.000719 | 0.0265 | 8.64 | 10.16 | 1.52 |
| 48 | RAB20, member RAS oncogene family | Rab20 | NM_011227_at | 0.001338 | 0.0265 | 5.22 | 6.73 | 1.52 |
| 49 | early growth response 1 | Egr1 | NM_007913_at | 0.000225 | 0.0265 | 10.65 | 12.15 | 1.50 |
| 50 | AT rich interactive domain 5A (MRF1-like) | Arid5a | NM_001172205_at | 0.000225 | 0.0265 | 6.13 | 7.61 | 1.49 |
| 51 | AT rich interactive domain 5A (MRF1-like) | Arid5a | NM_001172206_at | 0.000225 | 0.0265 | 6.13 | 7.61 | 1.49 |
| 52 | AT rich interactive domain 5A (MRF1-like) | Arid5a | NM_145996_at | 0.000225 | 0.0265 | 6.13 | 7.61 | 1.49 |
| 53 | AT rich interactive domain 5A (MRF1-like) | Arid5a | NR_033310_at | 0.000225 | 0.0265 | 6.13 | 7.61 | 1.49 |
| 54 | phorbol-12-myristate-13-acetate-induced | Pmaip1 | NM_021451_at | 0.002414 | 0.0265 | 6.94 | 8.41 | 1.47 |
| | protein 1 | | | | | | | |
| 55 | phosphodiesterase 4B, cAMP specific | Pde4b | NM_001177980_at | 0.001808 | 0.0265 | 7.52 | 8.96 | 1.44 |
| 56 | phosphodiesterase 4B, cAMP specific | Pde4b | NM_001177981_at | 0.001808 | 0.0265 | 7.52 | 8.96 | 1.44 |
| 57 | phosphodiesterase 4B, cAMP specific | Pde4b | NM_001177982_at | 0.001808 | 0.0265 | 7.52 | 8.96 | 1.44 |
| 58 | phosphodiesterase 4B, cAMP specific | Pde4b | NM_019840_at | 0.001808 | 0.0265 | 7.52 | 8.96 | 1.44 |
| 59 | small proline-rich protein 1A | Sprr1a | NM_009264_at | 0.002090 | 0.0265 | 9.41 | 10.84 | 1.43 |
| 60 | CD207 antigen | Cd207 | NM_144943_at | 0.000444 | 0.0265 | 8.90 | 10.32 | 1.42 |
| 61 | a disintegrin-like and metallopeptidase | Adamts4 | NM_172845_at | 0.003180 | 0.0265 | 4.79 | 6.21 | 1.41 |
| | (reprolysin type) with thrombospondin | | | | | | | |
| | type 1 motif, 4 | | | | | | | |
| 62 | Jun-B oncogene | Junb | NM_008416_at | 0.000377 | 0.0265 | 8.32 | 9.71 | 1.39 |
| 63 | suppressor of cytokine signaling 3 | Socs3 | NM_007707_at | 0.001808 | 0.0265 | 8.08 | 9.48 | 1.39 |
| 64 | nuclear receptor subfamily 4, group A, | Nr4a2 | NM_001139509_at | 0.000614 | 0.0265 | 6.07 | 7.45 | 1.39 |
| | member 2 | | | | | | | |
| 65 | nuclear receptor subfamily 4, group A, | Nr4a2 | NM_013613_at | 0.000614 | 0.0265 | 6.07 | 7.45 | 1.39 |
| | member 2 | | | | | | | |
| 66 | G-protein coupled receptor 65 | Gpr65 | NM_008152_at | 0.000719 | 0.0265 | 4.13 | 5.48 | 1.35 |
| 67 | chemokine (C-X-C motif) ligand 16 | Cxcl16 | NM_023158_at | 0.000842 | 0.0265 | 7.64 | 8.99 | 1.35 |
| 68 | metallothionein 1 | Mt1 | NM_013602_at | 0.000377 | 0.0265 | 11.40 | 12.73 | 1.34 |
| 69 | solute carrier family 10 (sodium/bile acid | Slc10a6 | NM_029415_at | 0.000225 | 0.0265 | 7.95 | 9.28 | 1.33 |
| | cotransporter family), member 6 | | | | | | | |
| 70 | CD44 antigen | Cd44 | NM_001039150_at | 0.000268 | 0.0265 | 8.51 | 9.82 | 1.31 |
| 71 | CD44 antigen | Cd44 | NM_001039151_at | 0.000268 | 0.0265 | 8.51 | 9.82 | 1.31 |
| 72 | CD44 antigen | Cd44 | NM_001177785_at | 0.000268 | 0.0265 | 8.51 | 9.82 | 1.31 |
| 73 | CD44 antigen | Cd44 | NM_001177786_at | 0.000268 | 0.0265 | 8.51 | 9.82 | 1.31 |
| 74 | CD44 antigen | Cd44 | NM_001177787_at | 0.000268 | 0.0265 | 8.51 | 9.82 | 1.31 |
| 75 | CD44 antigen | Cd44 | NM_009851_at | 0.000268 | 0.0265 | 8.51 | 9.82 | 1.31 |
| 76 | granzyme B | Gzmb | NM_013542_at | 0.000159 | 0.0265 | 4.44 | 5.74 | 1.30 |
| 77 | spermine oxidase | Smox | NM_001177835_at | 0.000978 | 0.0265 | 7.14 | 8.44 | 1.30 |
| 78 | spermine oxidase | Smox | NM_001177838_at | 0.000978 | 0.0265 | 7.14 | 8.44 | 1.30 |
| 79 | spermine oxidase | Smox | NM_001177840_at | 0.000978 | 0.0265 | 7.14 | 8.44 | 1.30 |
| 80 | cAMP responsive element modulator | Crem | NM_001110850_at | 0.000377 | 0.0265 | 5.62 | 6.91 | 1.29 |
| 81 | cAMP responsive element modulator | Crem | NM_001110854_at | 0.000377 | 0.0265 | 5.62 | 6.91 | 1.29 |
| 82 | cAMP responsive element modulator | Crem | NM_001110855_at | 0.000377 | 0.0265 | 5.62 | 6.91 | 1.29 |
| 83 | cAMP responsive element modulator | Crem | NM_001110856_at | 0.000377 | 0.0265 | 5.62 | 6.91 | 1.29 |
| 84 | cAMP responsive element modulator | Crem | NM_013498_at | 0.000377 | 0.0265 | 5.62 | 6.91 | 1.29 |
| 85 | spermine oxidase | Smox | NM_001177833_at | 0.000719 | 0.0265 | 6.88 | 8.18 | 1.29 |
| 86 | spermine oxidase | Smox | NM_001177834_at | 0.001808 | 0.0265 | 6.96 | 8.24 | 1.29 |
| 87 | spermine oxidase | Smox | NM_001177836_at | 0.000842 | 0.0265 | 6.79 | 8.07 | 1.28 |
| 88 | spermine oxidase | Smox | NM_001177837_at | 0.000842 | 0.0265 | 6.79 | 8.07 | 1.28 |
| 89 | spermine oxidase | Smox | NM_001177839_at | 0.000842 | 0.0265 | 6.79 | 8.07 | 1.28 |
| 90 | spermine oxidase | Smox | NM_145533_at | 0.000842 | 0.0265 | 6.79 | 8.07 | 1.28 |
| 91 | basic helix-loop-helix family, member e40 | Bhlhe40 | NM_011498_at | 0.002090 | 0.0265 | 9.06 | 10.32 | 1.26 |
| 92 | suppressor of cytokine signaling 2 | Socs2 | NM_001168655_at | 0.001338 | 0.0265 | 7.66 | 8.92 | 1.26 |
| 93 | suppressor of cytokine signaling 2 | Socs2 | NM_001168656_at | 0.001338 | 0.0265 | 7.66 | 8.92 | 1.26 |
| 94 | suppressor of cytokine signaling 2 | Socs2 | NM_001168657_at | 0.001338 | 0.0265 | 7.66 | 8.92 | 1.26 |
| 95 | suppressor of cytokine signaling 2 | Socs2 | NM_007706_at | 0.001338 | 0.0265 | 7.66 | 8.92 | 1.26 |
| 96 | killer cell lectin-like receptor subfamily B | Klrb1b | NM_030599_at | 0.000075 | 0.0265 | 5.33 | 6.58 | 1.25 |
| | member 1B | | | | | | | |
| 97 | OTU domain containing 7A | Otud7a | NM_130880_at | 0.000225 | 0.0265 | 5.99 | 7.23 | 1.25 |
| 98 | cAMP responsive element modulator | Crem | NM_001110853_at | 0.000522 | 0.0265 | 5.04 | 6.26 | 1.23 |
| 99 | cAMP responsive element modulator | Crem | NM_001110857_at | 0.000522 | 0.0265 | 5.04 | 6.26 | 1.23 |
| 100 | cAMP responsive element modulator | Crem | NM_001110858_at | 0.000522 | 0.0265 | 5.04 | 6.26 | 1.23 |
| 101 | cAMP responsive element modulator | Crem | NM_001110859_at | 0.000522 | 0.0265 | 5.04 | 6.26 | 1.23 |
| 102 | prostaglandin E synthase | Ptges | NM_022415_at | 0.000091 | 0.0265 | 7.08 | 8.31 | 1.22 |
| 103 | gap junction protein, beta 2 | Gjb2 | NM_008125_at | 0.001561 | 0.0265 | 7.64 | 8.85 | 1.21 |
| 104 | receptor (calcitonin) activity modifying | Ramp3 | NM_019511_at | 0.000010 | 0.0265 | 6.74 | 7.94 | 1.20 |
| | protein 3 | | | | | | | |
| 105 | FK506 binding protein 5 | Fkbp5 | NM_010220_at | 0.000012 | 0.0265 | 7.38 | 8.58 | 1.19 |
| 106 | cytohesin 1 interacting protein | Cytip | NM_139200_at | 0.001561 | 0.0265 | 8.71 | 9.90 | 1.19 |
| 107 | solute carrier family 25 (mitochondrial | Slc25a25 | NM_001164357_at | 0.000225 | 0.0265 | 6.95 | 8.10 | 1.16 |
| | carrier, phosphate carrier), member 25 | | | | | | | |
| 108 | solute carrier family 25 (mitochondrial | Slc25a25 | NM_001164358_at | 0.000225 | 0.0265 | 6.95 | 8.10 | 1.16 |
| | carrier, phosphate carrier), member 25 | | | | | | | |
| 109 | solute carrier family 25 (mitochondrial | Slc25a25 | NM_146118_at | 0.000225 | 0.0265 | 6.95 | 8.10 | 1.16 |
| | carrier, phosphate carrier), member 25 | | | | | | | |
| 110 | ring finger protein 19B | Rnf19b | NM_029219_at | 0.000023 | 0.0265 | 7.84 | 8.99 | 1.15 |
| 111 | interleukin 7 receptor | Il7r | NM_008372_at | 0.000023 | 0.0265 | 6.55 | 7.70 | 1.15 |
| 112 | chloride intracellular channel 4 | Clic4 | NM_013885_at | 0.000225 | 0.0265 | 7.93 | 9.05 | 1.12 |
| | (mitochondrial) | | | | | | | |
| 113 | cytokine inducible SH2-containing | Cish | NM_009895_at | 0.000132 | 0.0265 | 8.26 | 9.37 | 1.12 |
| | protein | | | | | | | |
| 114 | colony stimulating factor 2 receptor, | Csf2rb | NM_007780_at | 0.000034 | 0.0265 | 6.62 | 7.73 | 1.11 |
| | beta, low-affinity (granulocyte- | | | | | | | |
| | macrophage) | | | | | | | |
| 115 | metallothionein 2 | Mt2 | NM_008630_at | 0.000319 | 0.0265 | 12.12 | 13.23 | 1.11 |
| 116 | UDP-N-acetylglucosamine | Uap1 | NM_133806_at | 0.002090 | 0.0265 | 8.06 | 9.16 | 1.11 |
| | pyrophosphorylase 1 | | | | | | | |
| 117 | ATP-binding cassette, sub-family C | Abcc3 | NM_029600_at | 0.000189 | 0.0265 | 6.78 | 7.87 | 1.09 |
| | (CFTR/MRP), member 3 | | | | | | | |
| 118 | breast cancer anti-estrogen resistance 3 | Bcar3 | NM_013867_at | 0.000091 | 0.0265 | 6.88 | 7.96 | 1.08 |
| 119 | dual specificity phosphatase 7 | Dusp7 | NM_153459_at | 0.000189 | 0.0265 | 7.78 | 8.85 | 1.07 |
| 120 | TNF receptor-associated factor 1 | Traf1 | NM_009421_at | 0.000159 | 0.0265 | 4.97 | 6.04 | 1.07 |
| 121 | stanniocalcin 1 | Stc1 | NM_009285_at | 0.000002 | 0.0265 | 6.50 | 7.56 | 1.07 |
| 122 | keratin 35 | Krt35 | NM_016880_at | 0.000614 | 0.0265 | 4.39 | 5.45 | 1.06 |
| 123 | Jun oncogene | Jun | NM_010591_at | 0.000319 | 0.0265 | 8.68 | 9.74 | 1.06 |
| 124 | plasminogen activator, urokinase | Plaur | NM_011113_at | 0.000319 | 0.0265 | 6.63 | 7.68 | 1.06 |
| | receptor | | | | | | | |
| 125 | zinc finger and BTB domain containing | Zbtb16 | NM_001033324_at | 0.000015 | 0.0650 | 5.89 | 6.93 | 1.04 |
| | 16 | | | | | | | |
| 126 | mitogen-activated protein kinase kinase | Map3k6 | NM_016693_at | 0.000614 | 0.0265 | 7.76 | 8.79 | 1.03 |
| | kinase 6 | | | | | | | |
| 127 | baculoviral IAP repeat-containing 3 | Birc3 | NM_007464_at | 0.000978 | 0.0265 | 9.34 | 10.36 | 1.02 |
| 128 | immediate early response 2 | Ier2 | NM_010499_at | 0.000159 | 0.0265 | 9.71 | 10.73 | 1.02 |
| 129 | mitogen-activated protein kinase kinase | Map3k8 | NM_007746_at | 0.000377 | 0.0265 | 7.71 | 8.72 | 1.01 |
| | kinase 8 | | | | | | | |
| 130 | fibroblast growth factor binding protein | Fgfbp1 | NM_008009_at | 0.000842 | 0.0265 | 10.46 | 11.47 | 1.01 |
| | 1 | | | | | | | |
| 131 | zinc finger protein 593 | Zfp593 | NM_024215_at | 0.000225 | 0.0265 | 5.13 | 6.14 | 1.01 |
| 132 | glycerol kinase | Gyk | NM_008194_at | 0.001338 | 0.0265 | 8.64 | 9.63 | 1.00 |
| 133 | glycerol kinase | Gyk | NM_212444_at | 0.001338 | 0.0265 | 8.64 | 9.63 | 1.00 |
| 134 | polo-like kinase 3 (Drosophila) | Plk3 | NM_013807_at | 0.000004 | 0.0265 | 7.72 | 8.72 | 1.00 |
| 135 | DEP domain containing MTOR-interacting | Deptor | NM_001037937_at | 0.000001 | 0.0265 | 8.13 | 9.12 | 0.99 |
| | protein | | | | | | | |
| 136 | DEP domain containing MTOR-interacting | Deptor | NM_145470_at | 0.000001 | 0.0265 | 8.13 | 9.12 | 0.99 |
| | protein | | | | | | | |
| 137 | eukaryotic translation initiation factor 1A | Eif1a | NM_010120_at | 0.000189 | 0.0265 | 8.42 | 9.39 | 0.97 |
| 138 | oncostatin M receptor | Osmr | NM_011019_at | 0.000522 | 0.0265 | 6.54 | 7.49 | 0.95 |
| 139 | nuclear factor of kappa light polypeptide | Nfkb2 | NM_001177369_at | 0.000159 | 0.0265 | 7.32 | 8.26 | 0.94 |
| | gene enhancer in B-cells 2, p49/p100 | | | | | | | |
| 140 | nuclear factor of kappa light polypeptide | Nfkb2 | NM_001177370_at | 0.000159 | 0.0265 | 7.32 | 8.26 | 0.94 |
| | gene enhancer in B-cells 2, p49/p100 | | | | | | | |
| 141 | nuclear factor of kappa light polypeptide | Nfkb2 | NM_019408_at | 0.000159 | 0.0265 | 7.32 | 8.26 | 0.94 |
| | gene enhancer in B-cells 2, p49/p100 | | | | | | | |
| 142 | insulin receptor substrate 2 | Irs2 | NM_001081212_at | 0.000978 | 0.0265 | 7.41 | 8.35 | 0.94 |
| 143 | G protein-coupled receptor 35 | Gpr35 | NM_001104529_at | 0.000132 | 0.0265 | 4.72 | 5.63 | 0.91 |
| 144 | G protein-coupled receptor 35 | Gpr35 | NM_022320_at | 0.000132 | 0.0265 | 4.72 | 5.63 | 0.91 |
| 145 | interleukin 1 receptor-like 1 | Il1rl1 | NM_001025602_at | 0.000028 | 0.0265 | 7.49 | 8.40 | 0.91 |
| 146 | RIKEN cDNA 5730408K05 gene | 5730408K05Rik | NR_027866_at | 0.001338 | 0.0265 | 7.24 | 8.14 | 0.91 |
| 147 | poliovirus receptor | Pvr | NM_027514_at | 0.000042 | 0.0265 | 5.82 | 6.71 | 0.90 |
| 148 | interleukin 10 receptor, alpha | Il10ra | NM_008348_at | 0.000012 | 0.0265 | 6.08 | 6.97 | 0.90 |
| 149 | ERBB receptor feedback inhibitor 1 | Errfi1 | NM_133753_at | 0.000062 | 0.0265 | 9.25 | 10.14 | 0.89 |
| 150 | aquaporin 3 | Aqp3 | NM_016689_at | 0.000189 | 0.0265 | 9.60 | 10.49 | 0.89 |
| 151 | serine/threonine kinase 17b (apoptosis- | Stk17b | NM_133810_at | 0.000978 | 0.0265 | 8.81 | 9.70 | 0.89 |
| | inducing) | | | | | | | |
| 152 | RIKEN cDNA 1810055G02 gene | 1810055G02Rik | NM_028077_at | 0.000225 | 0.0265 | 7.52 | 8.40 | 0.89 |
| 153 | coronin, actin binding protein 2A | Coro2a | NM_001164804_at | 0.000010 | 0.0265 | 6.14 | 7.02 | 0.88 |
| 154 | coronin, actin binding protein 2A | Coro2a | NM_178893_at | 0.000010 | 0.0265 | 6.14 | 7.02 | 0.88 |
| 155 | PQ loop repeat containing 1 | Pqlc1 | NM_001164420_at | 0.000051 | 0.0265 | 9.34 | 10.20 | 0.87 |
| 156 | PQ loop repeat containing 1 | Pqlc1 | NM_001164421_at | 0.000051 | 0.0265 | 9.34 | 10.20 | 0.87 |
| 157 | PQ loop repeat containing 1 | Pqlc1 | NM_001164422_at | 0.000051 | 0.0265 | 9.34 | 10.20 | 0.87 |
| 158 | PQ loop repeat containing 1 | Pqlc1 | NM_025861_at | 0.000051 | 0.0265 | 9.34 | 10.20 | 0.87 |
| 159 | solute carrier family 39 (zinc | Slc39a14 | NM_001135151_at | 0.000132 | 0.0265 | 5.48 | 6.31 | 0.84 |
| | transporter), member 14 | | | | | | | |
| 160 | solute carrier family 39 (zinc | Slc39a14 | NM_001135152_at | 0.000132 | 0.0265 | 5.48 | 6.31 | 0.84 |
| | transporter), member 14 | | | | | | | |
| 161 | solute carrier family 39 (zinc | Slc39a14 | NM_144808_at | 0.000132 | 0.0265 | 5.48 | 6.31 | 0.84 |
| | transporter), member 14 | | | | | | | |
| 162 | EF hand domain containing 2 | Efhd2 | NM_025994_at | 0.000159 | 0.0265 | 9.75 | 10.57 | 0.82 |
| 163 | Eph receptor A2 | Epha2 | NM_010139_at | 0.000225 | 0.0265 | 7.78 | 8.60 | 0.82 |
| 164 | family with sequence similarity 43, | Fam43a | NM_177632_at | 0.000842 | 0.0265 | 8.26 | 9.07 | 0.81 |
| | member A | | | | | | | |
| 165 | cysteine and glycine-rich protein 1 | Csrp1 | NM_007791_at | 0.000319 | 0.0265 | 9.08 | 9.89 | 0.81 |
| 166 | interleukin 4 receptor, alpha | Il4ra | NM_001008700_at | 0.000132 | 0.0265 | 6.66 | 7.47 | 0.81 |
| 167 | potassium channel tetramerisation | Kctd11 | NM_153143_at | 0.000268 | 0.0265 | 8.55 | 9.35 | 0.80 |
| | domain containing 11 | | | | | | | |
| 168 | cirrhosis, autosomal recessive 1A | Cirh1a | NM_011574_at | 0.000091 | 0.0265 | 8.73 | 9.51 | 0.79 |
| | (human) | | | | | | | |
| 169 | peroxisome proliferative activated | Pprc1 | NM_001081214_at | 0.000268 | 0.0265 | 8.02 | 8.81 | 0.78 |
| | receptor, gamma, coactivator-related 1 | | | | | | | |
| 170 | coagulation factor II (thrombin) receptor- | F2rl1 | NM_007974_at | 0.000132 | 0.0265 | 8.60 | 9.38 | 0.78 |
| | like 1 | | | | | | | |
| 171 | solute carrier family 9 (sodium/hydrogen | Slc9a3r1 | NM_012030_at | 0.000132 | 0.0265 | 9.16 | 9.93 | 0.78 |
| | exchanger), member 3 regulator 1 | | | | | | | |
| 172 | early growth response 2 | Egr2 | NM_010118_at | 0.000034 | 0.0265 | 9.72 | 10.49 | 0.77 |
| 173 | mitogen-activated protein kinase kinase | Map3k14 | NM_016896_at | 0.000132 | 0.0265 | 5.93 | 6.71 | 0.77 |
| | kinase 14 | | | | | | | |
| 174 | IL2-inducible T-cell kinase | Itk | NM_010583_at | 0.000008 | 0.0265 | 4.35 | 5.12 | 0.76 |
| 175 | Kruppel-like factor 10 | Klf10 | NM_013692_at | 0.000132 | 0.0265 | 9.20 | 9.95 | 0.75 |
| 176 | par-6 (partitioning defective 6) homolog | Pard6b | NM_021409_at | 0.000522 | 0.0265 | 5.79 | 6.53 | 0.74 |
| | beta (C. elegans) | | | | | | | |
| 177 | integrin alpha X | Itgax | NM_021334_at | 0.000614 | 0.0265 | 6.36 | 7.10 | 0.74 |
| 178 | RIKEN cDNA 1700017B05 gene | 1700017B05Rik | NM_028820_at | 0.000159 | 0.0265 | 6.87 | 7.60 | 0.74 |
| 179 | protein tyrosine phosphatase, non- | Ptpn22 | NM_008979_at | 0.000159 | 0.0265 | 5.97 | 6.70 | 0.73 |
| | receptor type 22 (lymphoid) | | | | | | | |
| 180 | myeloid differentiation primary response | Myd88 | NM_010851_at | 0.000132 | 0.0265 | 8.95 | 9.68 | 0.73 |
| | gene 88 | | | | | | | |
| 181 | family with sequence similarity 134, | Fam134b | NM_025459_at | 0.000008 | 0.0265 | 9.28 | 10.01 | 0.73 |
| | member B | | | | | | | |
| 182 | DEAD (Asp-Glu-Ala-Asp) box polypeptide | Ddx21 | NM_019553_at | 0.000268 | 0.0265 | 7.80 | 8.53 | 0.73 |
| | 21 | | | | | | | |
| 183 | PDZ and LIM domain 7 | Pdlim7 | NM_001114088_at | 0.000062 | 0.0265 | 6.06 | 6.79 | 0.73 |
| 184 | tripartite motif-containing 16 | Trim16 | NM_053169_at | 0.000319 | 0.0265 | 7.07 | 7.80 | 0.73 |
| 185 | potassium voltage-gated channel, Isk- | Kcne4 | NM_021342_at | 0.000522 | 0.0265 | 6.45 | 7.17 | 0.72 |
| | related subfamily, gene 4 | | | | | | | |
| 186 | purinergic receptor P2Y, G-protein | P2ry14 | NM_001008497_at | 0.000042 | 0.0265 | 5.36 | 6.08 | 0.72 |
| | coupled, 14 | | | | | | | |
| 187 | purinergic receptor P2Y, G-protein | P2ry14 | NM_133200_at | 0.000042 | 0.0265 | 5.36 | 6.08 | 0.72 |
| | coupled, 14 | | | | | | | |
| 188 | RIKEN cDNA 2310014H01 gene | 2310014H01Rik | NM_001146710_at | 0.000319 | 0.0265 | 8.25 | 8.96 | 0.71 |
| 189 | RIKEN cDNA 2310014H01 gene | 2310014H01Rik | NM_001146711_at | 0.000319 | 0.0265 | 8.25 | 8.96 | 0.71 |
| 190 | RIKEN cDNA 2310014H01 gene | 2310014H01Rik | NM_175242_at | 0.000319 | 0.0265 | 8.25 | 8.96 | 0.71 |
| 191 | psoriasis susceptibility 1 candidate 2 | Psors1c2 | NM_020576_at | 0.000268 | 0.0265 | 8.54 | 9.24 | 0.70 |
| | (human) | | | | | | | |
| 192 | cytotoxic T lymphocyte-associated protein | Ctla2a | NM_001145799_at | 0.000522 | 0.0265 | 7.97 | 8.67 | 0.70 |
| | 2 alpha | | | | | | | |
| 193 | cytotoxic T lymphocyte-associated protein | Ctla2a | NM_007796_at | 0.000522 | 0.0265 | 7.97 | 8.67 | 0.70 |
| | 2 alpha | | | | | | | |
| 194 | UDP-GlcNAc: betaGal beta-1,3-N- | B3gnt2 | NM_001169114_at | 0.000444 | 0.0265 | 7.44 | 8.14 | 0.69 |
| | acetylglucosaminyltransferase 2 | | | | | | | |
| 195 | UDP-GlcNAc: betaGal beta-1,3-N- | B3gnt2 | NM_016888_at | 0.000444 | 0.0265 | 7.44 | 8.14 | 0.69 |
| | acetylglucosaminyltransferase 2 | | | | | | | |
| 196 | NEDD4 binding protein 1 | N4bp1 | NM_030563_at | 0.000006 | 0.0265 | 8.33 | 9.02 | 0.69 |
| 197 | glucosaminyl (N-acetyl) transferase 2, I- | Gcnt2 | NM_008105_at | 0.000189 | 0.0265 | 5.38 | 6.07 | 0.69 |
| | branching enzyme | | | | | | | |
| 198 | glucosaminyl (N-acetyl) transferase 2, I- | Gcnt2 | NM_023887_at | 0.000189 | 0.0265 | 5.38 | 6.07 | 0.69 |
| | branching enzyme | | | | | | | |
| 199 | inducible T-cell co-stimulator | Icos | NM_017480_at | 0.000189 | 0.0265 | 4.95 | 5.63 | 0.68 |
| 200 | glutamine fructose-6-phosphate | Gfpt2 | NM_013529_at | 0.000319 | 0.0265 | 6.97 | 7.64 | 0.67 |
| | transaminase 2 | | | | | | | |
| 201 | cytotoxic T lymphocyte-associated protein | Ctla2b | NM_001145801_at | 0.000377 | 0.0265 | 7.45 | 8.12 | 0.67 |
| | 2 beta | | | | | | | |
| 202 | cytotoxic T lymphocyte-associated protein | Ctla2b | NM_007797_at | 0.000377 | 0.0265 | 7.45 | 8.12 | 0.67 |
| | 2 beta | | | | | | | |
| 203 | predicted gene, Gm16516 | Gm16516 | NR_027800_at | 0.000225 | 0.0265 | 6.31 | 6.97 | 0.66 |
| 204 | palmdelphin | Palmd | NM_023245_at | 0.000075 | 0.0265 | 8.48 | 9.12 | 0.65 |
| 205 | ribosomal RNA processing 1 homolog B | Rrp1b | NM_001163734_at | 0.000110 | 0.0265 | 7.37 | 8.01 | 0.64 |
| | (S. cerevisiae) | | | | | | | |
| 206 | ribosomal RNA processing 1 homolog B | Rrp1b | NM_028244_at | 0.000110 | 0.0265 | 7.37 | 8.01 | 0.64 |
| | (S. cerevisiae) | | | | | | | |
| 207 | regulator of chromosome condensation 2 | Rcc2 | NM_173867_at | 0.000159 | 0.0265 | 7.12 | 7.76 | 0.63 |
| 208 | nitric oxide synthase 3, endothelial cell | Nos3 | NM_008713_at | 0.000046 | 0.0650 | 6.67 | 7.30 | 0.63 |
| 209 | transient receptor potential cation | Trpv2 | NM_011706_at | 0.000225 | 0.0265 | 6.62 | 7.25 | 0.63 |
| | channel, subfamily V, member 2 | | | | | | | |
| 210 | coiled-coil-helix-coiled-coil-helix domain | Chchd4 | NM_133928_at | 0.000189 | 0.0265 | 7.56 | 8.19 | 0.63 |
| | containing 4 | | | | | | | |
| 211 | RIKEN cDNA 3110043O21 gene | 3110043O21Rik | NM_001081343_at | 0.000132 | 0.0265 | 7.73 | 8.36 | 0.63 |
| 212 | ral guanine nucleotide dissociation | Ralgds | NM_001145834_at | 0.000062 | 0.0265 | 8.36 | 8.99 | 0.63 |
| | stimulator | | | | | | | |
| 213 | ral guanine nucleotide dissociation | Ralgds | NM_001145835_at | 0.000062 | 0.0265 | 8.36 | 8.99 | 0.63 |
| | stimulator | | | | | | | |
| 214 | ral guanine nucleotide dissociation | Ralgds | NM_001145836_at | 0.000062 | 0.0265 | 8.36 | 8.99 | 0.63 |
| | stimulator | | | | | | | |
| 215 | ral guanine nucleotide dissociation | Ralgds | NM_009058_at | 0.000062 | 0.0265 | 8.36 | 8.99 | 0.63 |
| | stimulator | | | | | | | |
| 216 | F-box protein 42 | Fbxo42 | NM_172518_at | 0.000010 | 0.0265 | 6.41 | 7.04 | 0.63 |
| 217 | family with sequence similarity 102, | Fam102a | NM_153560_at | 0.000132 | 0.0265 | 8.10 | 8.71 | 0.62 |
| | member A | | | | | | | |
| 218 | tumor necrosis factor receptor | Tnfrsf9 | NM_001077508_at | 0.000042 | 0.0650 | 4.62 | 5.23 | 0.61 |
| | superfamily, member 9 | | | | | | | |
| 219 | tumor necrosis factor receptor | Tnfrsf9 | NM_001077509_at | 0.000042 | 0.0650 | 4.62 | 5.23 | 0.61 |
| | superfamily, member 9 | | | | | | | |
| 220 | tumor necrosis factor receptor | Tnfrsf9 | NM_011612_at | 0.000042 | 0.0650 | 4.62 | 5.23 | 0.61 |
| | superfamily, member 9 | | | | | | | |
| 221 | TGFB-induced factor homeobox 1 | Tgif1 | NM_001164074_at | 0.000189 | 0.0265 | 7.60 | 8.20 | 0.60 |
| 222 | TGFB-induced factor homeobox 1 | Tgif1 | NM_001164075_at | 0.000189 | 0.0265 | 7.60 | 8.20 | 0.60 |
| 223 | TGFB-induced factor homeobox 1 | Tgif1 | NM_001164076_at | 0.000189 | 0.0265 | 7.60 | 8.20 | 0.60 |
| 224 | TGFB-induced factor homeobox 1 | Tgif1 | NM_001164077_at | 0.000189 | 0.0265 | 7.60 | 8.20 | 0.60 |
| 225 | TGFB-induced factor homeobox 1 | Tgif1 | NM_009372_at | 0.000189 | 0.0265 | 7.60 | 8.20 | 0.60 |
| 226 | zinc finger with KRAB and SCAN domains | Zkscan6 | NM_026107_at | 0.000034 | 0.0265 | 6.79 | 7.39 | 0.60 |
| | 6 | | | | | | | |
| 227 | glucosaminyl (N-acetyl) transferase 2, I- | Gcnt2 | NM_133219_at | 0.000159 | 0.0265 | 5.63 | 6.23 | 0.60 |
| | branching enzyme | | | | | | | |
| 228 | interleukin 2 receptor, gamma chain | Il2rg | NM_013563_at | 0.000225 | 0.0265 | 6.80 | 7.40 | 0.60 |
| 229 | RIKEN cDNA 4931408A02 gene | 4931408A02Rik | NM_001199210_at | 0.000377 | 0.0265 | 6.45 | 7.04 | 0.59 |
| 230 | RIKEN cDNA 4931408A02 gene | 4931408A02Rik | NM_027627_at | 0.000377 | 0.0265 | 6.45 | 7.04 | 0.59 |
| 231 | protein phosphatase 1, regulatory | Ppp1r15a | NM_008654_at | 0.000012 | 0.0265 | 8.55 | 9.13 | 0.58 |
| | (inhibitor) subunit 15A | | | | | | | |
| 232 | poliovirus receptor-related 2 | Pvrl2 | NM_001159724_at | 0.000110 | 0.0265 | 7.63 | 8.21 | 0.58 |
| 233 | endothelin converting enzyme 1 | Ece1 | NM_199307_at | 0.000189 | 0.0265 | 7.52 | 8.08 | 0.57 |
| 234 | breast cancer anti-estrogen resistance 1 | Bcar1 | NM_001198839_at | 0.000042 | 0.0265 | 7.06 | 7.58 | 0.53 |
| 235 | breast cancer anti-estrogen resistance 1 | Bcar1 | NM_009954_at | 0.000042 | 0.0265 | 7.06 | 7.58 | 0.53 |
| 236 | HEAT repeat containing 1 | Heatr1 | NM_144835_at | 0.000051 | 0.0265 | 6.85 | 7.37 | 0.53 |
| 237 | inositol polyphosphate-5-phosphatase D | Inpp5d | NM_001110192_at | 0.000034 | 0.0265 | 5.93 | 6.44 | 0.51 |
| 238 | inositol polyphosphate-5-phosphatase D | Inpp5d | NM_001110193_at | 0.000034 | 0.0265 | 5.93 | 6.44 | 0.51 |
| 239 | inositol polyphosphate-5-phosphatase D | Inpp5d | NM_010566_at | 0.000034 | 0.0265 | 5.93 | 6.44 | 0.51 |
| 240 | zinc finger protein 52 | Zfp52 | NM_144515_at | 0.000444 | 0.0265 | 5.81 | 6.32 | 0.51 |
| 241 | interleukin 23, alpha subunit p19 | Il23a | NM_031252_at | 0.000268 | 0.0265 | 4.30 | 4.78 | 0.48 |
| 242 | RIKEN cDNA E430018J23 gene | E430018J23Rik | NM_198011_at | 0.000978 | 0.0265 | 4.36 | 4.83 | 0.47 |
| 243 | zinc finger protein 764-like | LOC100044339 | XM_001472017_at | 0.000978 | 0.0265 | 4.36 | 4.83 | 0.47 |
| 244 | 5-hydroxytryptamine (serotonin) receptor | Htr2b | NM_008311_at | 0.000319 | 0.0650 | 3.56 | 4.01 | 0.45 |
| | 2B | | | | | | | |
| 245 | ring finger protein 157 | Rnf157 | NM_027258_at | 0.000319 | 0.0265 | 4.56 | 4.99 | 0.43 |
| 246 | peptidyl arginine deiminase, type I | Padi1 | NM_011059_at | 0.000062 | 0.0265 | 3.97 | 4.33 | 0.36 |
| 247 | prokineticin receptor 1 | Prokr1 | NM_021381_at | 0.000051 | 0.0265 | 3.76 | 3.47 | −0.29 |
| 248 | zinc finger protein 2 | Zfp2 | NM_001044697_at | 0.000091 | 0.0265 | 3.68 | 3.36 | −0.32 |
| 249 | zinc finger protein 2 | Zfp2 | NM_001044698_at | 0.000091 | 0.0265 | 3.68 | 3.36 | −0.32 |
| 250 | zinc finger protein 2 | Zfp2 | NM_001044699_at | 0.000091 | 0.0265 | 3.68 | 3.36 | −0.32 |
| 251 | zinc finger protein 2 | Zfp2 | NM_001044700_at | 0.000091 | 0.0265 | 3.68 | 3.36 | −0.32 |
| 252 | Moloney leukemia virus 10 | Mov10 | NM_001163440_at | 0.000159 | 0.0265 | 4.82 | 4.43 | −0.39 |
| 253 | Moloney leukemia virus 10 | Mov10 | NM_001163441_at | 0.000159 | 0.0265 | 4.82 | 4.43 | −0.39 |
| 254 | Moloney leukemia virus 10 | Mov10 | NM_008619_at | 0.000159 | 0.0265 | 4.82 | 4.43 | −0.39 |
| 255 | zinc finger protein 30 | Zfp30 | NM_013705_at | 0.000268 | 0.0265 | 5.51 | 5.10 | −0.42 |
| 256 | DiGeorge syndrome critical region gene | Dgcr14 | NM_001081633_at | 0.000042 | 0.0265 | 5.95 | 5.52 | −0.42 |
| | 14 | | | | | | | |
| 257 | DiGeorge syndrome critical region gene | Dgcr14 | NM_022408_at | 0.000042 | 0.0265 | 5.95 | 5.52 | −0.42 |
| | 14 | | | | | | | |
| 258 | sialic acid acetylesterase | Siae | NM_011734_at | 0.000225 | 0.0265 | 5.78 | 5.33 | −0.45 |
| 259 | RIKEN cDNA 4933431E20 gene | 4933431E20Rik | NR_015459_at | 0.000018 | 0.0265 | 6.80 | 6.34 | −0.45 |
| 260 | RIKEN cDNA 1700030K09 gene | 1700030K09Rik | NM_028170_at | 0.000377 | 0.0265 | 5.97 | 5.51 | −0.46 |
| 261 | RIKEN cDNA 4930444A02 gene | 4930444A02Rik | NM_029037_at | 0.000010 | 0.0265 | 5.91 | 5.45 | −0.46 |
| 262 | rotatin | Rttn | NM_175542_at | 0.000132 | 0.0265 | 6.39 | 5.92 | −0.46 |
| 263 | sine oculis-related homeobox 5 homolog | Six5 | NM_011383_at | 0.000444 | 0.0265 | 5.45 | 4.98 | −0.47 |
| | (Drosophila) | | | | | | | |
| 264 | peptidyl prolyl isomerase H | Ppih | NM_001110129_at | 0.000018 | 0.0265 | 6.46 | 5.98 | −0.48 |
| 265 | tetraspanin 17 | Tspan17 | NM_028841_at | 0.000062 | 0.0265 | 7.33 | 6.85 | −0.48 |
| 266 | DNA-binding protein RFXANK-like | LOC100048268 | XM_003086949_at | 0.000051 | 0.0265 | 5.82 | 5.33 | −0.49 |
| 267 | jumonji domain containing 5 | Jmjd5 | NM_029842_at | 0.000075 | 0.0265 | 6.19 | 5.70 | −0.49 |
| 268 | fibroblast growth factor 13 | Fgf13 | NM_010200_at | 0.000051 | 0.0265 | 6.51 | 6.02 | −0.49 |
| 269 | kinesin family member 3A | Kif3a | NM_008443_at | 0.000159 | 0.0265 | 6.79 | 6.28 | −0.50 |
| 270 | non-metastatic cells 3, protein expressed | Nme3 | NM_019730_at | 0.000008 | 0.0265 | 7.83 | 7.32 | −0.51 |
| | in | | | | | | | |
| 271 | septin 4 | 4-Sep | NM_011129_at | 0.000132 | 0.0265 | 6.68 | 6.16 | −0.51 |
| 272 | CTF18, chromosome transmission fidelity | Chtf18 | NM_145409_at | 0.000842 | 0.0265 | 4.83 | 4.30 | −0.53 |
| | factor 18 homolog (S. cerevisiae) | | | | | | | |
| 273 | transmembrane protein 101 | Tmem101 | NM_029649_at | 0.000444 | 0.0265 | 6.98 | 6.44 | −0.54 |
| 274 | nicotinamide nucleotide | Nmnat1 | NM_133435_at | 0.000719 | 0.0265 | 5.11 | 4.57 | −0.54 |
| | adenylyltransferase 1 | | | | | | | |
| 275 | interleukin 17 receptor B | Il17rb | NM_019583_at | 0.000034 | 0.0650 | 6.31 | 5.76 | −0.55 |
| 276 | poly (ADP-ribose) polymerase family, | Parp16 | NM_177460_at | 0.000132 | 0.0265 | 6.57 | 6.03 | −0.55 |
| | member 16 | | | | | | | |
| 277 | mutL homolog 1 (E. coli) | Mlh1 | NM_026810_at | 0.000004 | 0.0265 | 8.05 | 7.50 | −0.55 |
| 278 | WD repeat domain 8 | Wdr8 | NM_021499_at | 0.000225 | 0.0265 | 6.24 | 5.69 | −0.55 |
| 279 | helicase-like transcription factor | Hltf | NM_009210_at | 0.000042 | 0.0265 | 8.69 | 8.14 | −0.56 |
| 280 | dual specificity phosphatase 19 | Dusp19 | NM_024438_at | 0.000268 | 0.0265 | 6.72 | 6.16 | −0.56 |
| 281 | suppressor of variegation 4-20 homolog 1 | Suv420h1 | NM_001167885_at | 0.000028 | 0.0265 | 7.80 | 7.25 | −0.56 |
| | (Drosophila) | | | | | | | |
| 282 | suppressor of variegation 4-20 homolog 1 | Suv420h1 | NM_001167886_at | 0.000028 | 0.0265 | 7.80 | 7.25 | −0.56 |
| | (Drosophila) | | | | | | | |
| 283 | suppressor of variegation 4-20 homolog 1 | Suv420h1 | NM_001167887_at | 0.000028 | 0.0265 | 7.80 | 7.25 | −0.56 |
| | (Drosophila) | | | | | | | |
| 284 | suppressor of variegation 4-20 homolog 1 | Suv420h1 | NM_144871_at | 0.000028 | 0.0265 | 7.80 | 7.25 | −0.56 |
| | (Drosophila) | | | | | | | |
| 285 | mesenchyme homeobox 2 | Meox2 | NM_008584_at | 0.000034 | 0.0265 | 7.32 | 6.76 | −0.56 |
| 286 | centrosomal protein 152 | Cep152 | NM_001081091_at | 0.000225 | 0.0265 | 6.64 | 6.07 | −0.57 |
| 287 | chibby homolog 1 (Drosophila) | Cby1 | NM_028634_at | 0.000042 | 0.0265 | 6.96 | 6.39 | −0.57 |
| 288 | glutathione transferase zeta 1 | Gstz1 | NM_010363_at | 0.000189 | 0.0265 | 8.34 | 7.76 | −0.58 |
| | (maleylacetoacetate isomerase) | | | | | | | |
| 289 | zinc finger protein 386 (Kruppel-like) | Zfp386 | NM_001004066_at | 0.000051 | 0.0265 | 7.72 | 7.14 | −0.58 |
| 290 | zinc finger protein 386 (Kruppel-like) | Zfp386 | NM_019565_at | 0.000051 | 0.0265 | 7.72 | 7.14 | −0.58 |
| 291 | RAD1 homolog (S. pombe) | Rad1 | NM_011232_at | 0.000159 | 0.0265 | 7.31 | 6.73 | −0.58 |
| 292 | fukutin | Fktn | NM_139309_at | 0.000444 | 0.0265 | 6.60 | 6.01 | −0.58 |
| 293 | RIKEN cDNA 1110032A03 gene | 1110032A03Rik | NM_023483_at | 0.000075 | 0.0265 | 7.94 | 7.35 | −0.58 |
| 294 | zinc finger protein 61 | Zfp61 | NM_009561_at | 0.000268 | 0.0265 | 6.39 | 5.81 | −0.59 |
| 295 | isovaleryl coenzyme A dehydrogenase | Ivd | NM_019826_at | 0.000225 | 0.0265 | 8.32 | 7.73 | −0.59 |
| 296 | zinc finger protein 810 | Zfp810 | NM_145612_at | 0.000075 | 0.0265 | 6.99 | 6.40 | −0.59 |
| 297 | makorin, ring finger protein, 3 | Mkrn3 | NM_011746_at | 0.000025 | 0.0265 | 4.30 | 3.70 | −0.60 |
| 298 | transmembrane protein 55A | Tmem55a | NM_028264_at | 0.000012 | 0.0265 | 9.01 | 8.40 | −0.60 |
| 299 | methionine sulfoxide reductase B2 | Msrb2 | NM_029619_at | 0.000719 | 0.0265 | 5.53 | 4.92 | −0.60 |
| 300 | glutaryl-Coenzyme A dehydrogenase | Gcdh | NM_001044744_at | 0.000100 | 0.0265 | 7.22 | 6.61 | −0.60 |
| 301 | glutaryl-Coenzyme A dehydrogenase | Gcdh | NM_008097_at | 0.000100 | 0.0265 | 7.22 | 6.61 | −0.60 |
| 302 | emopamil binding protein-like | Ebpl | NM_026598_at | 0.000015 | 0.0265 | 8.70 | 8.10 | −0.61 |
| 303 | ATP synthase, H+ transporting, | Atp5s | NM_026536_at | 0.000028 | 0.0650 | 7.31 | 6.70 | −0.61 |
| | mitochondrial F0 complex, subunit s | | | | | | | |
| 304 | SNF2 histone linker PHD RING helicase | Shprh | NM_001077707_at | 0.000189 | 0.0265 | 8.20 | 7.59 | −0.61 |
| 305 | coiled-coil domain containing 123 | Ccdc123 | NM_028120_at | 0.000051 | 0.0265 | 5.74 | 5.13 | −0.61 |
| 306 | centrosomal protein 70 | Cep70 | NM_023873_at | 0.000010 | 0.0265 | 5.72 | 5.10 | −0.61 |
| 307 | DEAH (Asp-Glu-Ala-Asp/His) box | Dhx57 | NM_001163759_at | 0.000319 | 0.0265 | 7.45 | 6.83 | −0.62 |
| | polypeptide 57 | | | | | | | |
| 308 | DEAH (Asp-Glu-Ala-Asp/His) box | Dhx57 | NM_198942_at | 0.000319 | 0.0265 | 7.45 | 6.83 | −0.62 |
| | polypeptide 57 | | | | | | | |
| 309 | protein-L-isoaspartate (D-aspartate) O- | Pcmtd2 | NM_153594_at | 0.000042 | 0.0265 | 8.96 | 8.34 | −0.62 |
| | methyltransferase domain containing 2 | | | | | | | |
| 310 | germ cell-less homolog 1 (Drosophila) | Gmcl1 | NM_011818_at | 0.000010 | 0.0265 | 6.91 | 6.28 | −0.62 |
| 311 | RIKEN cDNA 3830406C13 gene | 3830406C13Rik | NM_146051_at | 0.000091 | 0.0265 | 9.30 | 8.67 | −0.63 |
| 312 | RIKEN cDNA 3830406C13 gene | 3830406C13Rik | NM_178141_at | 0.000091 | 0.0265 | 9.30 | 8.67 | −0.63 |
| 313 | family with sequence similarity 161, | Fam161a | NM_028672_at | 0.000110 | 0.0650 | 5.50 | 4.87 | −0.63 |
| | member A | | | | | | | |
| 314 | carbohydrate (N-acetylgalactosamine 4-0) | Chst14 | NM_028117_at | 0.000015 | 0.0265 | 7.83 | 7.18 | −0.64 |
| | sulfotransferase 14 | | | | | | | |
| 315 | solute carrier family 25, member 35 | Slc25a35 | NM_028048_at | 0.000110 | 0.0265 | 5.50 | 4.85 | −0.65 |
| 316 | HMG box domain containing 4 | Hmgxb4 | NM_178017_at | 0.000028 | 0.0265 | 7.69 | 7.02 | −0.67 |
| 317 | RIKEN cDNA 4833442J19 gene | 4833442J19Rik | NM_177101_at | 0.000091 | 0.0650 | 7.02 | 6.35 | −0.68 |
| 318 | poly (ADP-ribose) polymerase family, | Parp12 | NM_172893_at | 0.000444 | 0.0265 | 8.23 | 7.56 | −0.68 |
| | member 12 | | | | | | | |
| 319 | RIKEN cDNA 1110038F14 gene | 1110038F14Rik | NM_054099_at | 0.000034 | 0.0265 | 7.87 | 7.17 | −0.70 |
| 320 | armadillo repeat containing, X-linked 1 | Armcx1 | NM_001166377_at | 0.000012 | 0.0650 | 7.48 | 6.78 | −0.70 |
| 321 | armadillo repeat containing, X-linked 1 | Armcx1 | NM_001166378_at | 0.000012 | 0.0650 | 7.48 | 6.78 | −0.70 |
| 322 | armadillo repeat containing, X-linked 1 | Armcx1 | NM_001166379_at | 0.000012 | 0.0650 | 7.48 | 6.78 | −0.70 |
| 323 | armadillo repeat containing, X-linked 1 | Armcx1 | NM_001166380_at | 0.000012 | 0.0650 | 7.48 | 6.78 | −0.70 |
| 324 | armadillo repeat containing, X-linked 1 | Armcx1 | NM_030066_at | 0.000012 | 0.0650 | 7.48 | 6.78 | −0.70 |
| 325 | RIKEN cDNA 2810055F11 gene | 2810055F11Rik | NM_026038_at | 0.000225 | 0.0265 | 6.85 | 6.14 | −0.71 |
| 326 | nucleosome assembly protein 1-like 3 | Nap1l3 | NM_138742_at | 0.000091 | 0.0265 | 4.39 | 3.67 | −0.71 |
| 327 | TLR4 interactor with leucine-rich repeats | Tril | NM_025817_at | 0.000023 | 0.0265 | 6.08 | 5.36 | −0.71 |
| 328 | apelin receptor | Aplnr | NM_011784_at | 0.000842 | 0.0265 | 6.57 | 5.85 | −0.72 |
| 329 | Rab40b, member RAS oncogene family | Rab40b | NM_139147_at | 0.000268 | 0.0650 | 7.03 | 6.31 | −0.72 |
| 330 | flavin containing monooxygenase 5 | Fmo5 | NM_001161763_at | 0.000062 | 0.1164 | 6.57 | 5.84 | −0.73 |
| 331 | flavin containing monooxygenase 5 | Fmo5 | NM_001161765_at | 0.000062 | 0.1164 | 6.57 | 5.84 | −0.73 |
| 332 | flavin containing monooxygenase 5 | Fmo5 | NM_010232_at | 0.000062 | 0.1164 | 6.57 | 5.84 | −0.73 |
| 333 | melanoma associated antigen (mutated) 1 | Mum1 | NM_023431_at | 0.000018 | 0.0265 | 8.23 | 7.49 | −0.73 |
| 334 | immature colon carcinoma transcript 1 | Ict1 | NM_026729_at | 0.000268 | 0.0265 | 8.64 | 7.91 | −0.74 |
| 335 | peroxisome proliferator activated | Pparg | NM_001127330_at | 0.000008 | 0.0265 | 8.21 | 7.47 | −0.74 |
| | receptor gamma | | | | | | | |
| 336 | peroxisome proliferator activated | Pparg | NM_011146_at | 0.000008 | 0.0265 | 8.21 | 7.47 | −0.74 |
| | receptor gamma | | | | | | | |
| 337 | ring finger protein 135 | Rnf135 | NM_028019_at | 0.000002 | 0.0265 | 6.43 | 5.68 | −0.75 |
| 338 | tripartite motif-containing 12A | Trim12a | NM_023835_at | 0.000719 | 0.0265 | 5.93 | 5.18 | −0.75 |
| 339 | kelch domain containing 5 | Klhdc5 | NM_001081237_at | 0.000132 | 0.0265 | 7.11 | 6.36 | −0.75 |
| 340 | zinc finger protein 825 | Zfp825 | NM_146231_at | 0.000023 | 0.0265 | 7.12 | 6.37 | −0.75 |
| 341 | RNA binding motif protein, X | Rbmx | NR_029425_at | 0.000023 | 0.0265 | 8.41 | 7.66 | −0.75 |
| | chromosome | | | | | | | |
| 342 | RIKEN cDNA 2610039C10 gene | 2610039C10Rik | NM_025642_at | 0.000522 | 0.0265 | 8.43 | 7.67 | −0.75 |
| 343 | general transcription factor III A | Gtf3a | NM_025652_at | 0.000091 | 0.0265 | 8.58 | 7.82 | −0.76 |
| 344 | tripartite motif-containing 59 | Trim59 | NM_025863_at | 0.000042 | 0.0265 | 8.28 | 7.52 | −0.76 |
| 345 | NIMA (never in mitosis gene a)-related | Nek3 | NM_001162947_at | 0.000034 | 0.0265 | 6.85 | 6.08 | −0.77 |
| | expressed kinase 3 | | | | | | | |
| 346 | NIMA (never in mitosis gene a)-related | Nek3 | NM_011848_at | 0.000034 | 0.0265 | 6.85 | 6.08 | −0.77 |
| | expressed kinase 3 | | | | | | | |
| 347 | zinc finger protein 40 | Zfp40 | NM_009555_at | 0.000159 | 0.0265 | 5.20 | 4.43 | −0.77 |
| 348 | F-box protein 25 | Fbxo25 | NM_025785_at | 0.000025 | 0.0265 | 7.19 | 6.41 | −0.78 |
| 349 | homeobox A2 | Hoxa2 | NM_010451_at | 0.000319 | 0.0265 | 6.44 | 5.66 | −0.78 |
| 350 | transmembrane and tetratricopeptide | Tmtc4 | NM_028651_at | 0.000075 | 0.0265 | 7.16 | 6.37 | −0.79 |
| | repeat containing 4 | | | | | | | |
| 351 | frizzled homolog 2 (Drosophila) | Fzd2 | NM_020510_at | 0.000189 | 0.0265 | 8.26 | 7.47 | −0.79 |
| 352 | collagen, type IV, alpha 5 | Col4a5 | NM_001163155_at | 0.000719 | 0.0265 | 7.82 | 7.02 | −0.80 |
| 353 | collagen, type IV, alpha 5 | Col4a5 | NM_007736_at | 0.000719 | 0.0265 | 7.82 | 7.02 | −0.80 |
| 354 | interleukin 7 | Il7 | NM_008371_at | 0.000159 | 0.0265 | 4.71 | 3.91 | −0.81 |
| 355 | phosphatidylinositol glycan anchor | Pigh | NM_029988_at | 0.000319 | 0.0265 | 7.08 | 6.28 | −0.81 |
| | biosynthesis, class H | | | | | | | |
| 356 | hematopoietically expressed homeobox | Hhex | NM_008245_at | 0.000051 | 0.0265 | 7.02 | 6.21 | −0.81 |
| 357 | zinc finger and BTB domain containing 8a | Zbtb8a | NM_028603_at | 0.000225 | 0.0265 | 6.41 | 5.59 | −0.82 |
| 358 | zinc finger protein 503 | Zfp503 | NM_145459_at | 0.000319 | 0.0265 | 9.51 | 8.69 | −0.82 |
| 359 | EF-hand calcium binding domain 7 | Efcab7 | NM_145549_at | 0.000377 | 0.0265 | 5.09 | 4.25 | −0.84 |
| 360 | potassium inwardly-rectifying channel, | Kcnj8 | NM_008428_at | 0.000719 | 0.0265 | 7.83 | 6.98 | −0.85 |
| | subfamily J, member 8 | | | | | | | |
| 361 | family with sequence similarity 64, | Fam64a | NM_144526_at | 0.002414 | 0.0265 | 5.46 | 4.61 | −0.85 |
| | member A | | | | | | | |
| 362 | histamine N-methyltransferase | Hnmt | NM_080462_at | 0.000189 | 0.0265 | 7.21 | 6.33 | −0.88 |
| 363 | transcription factor B1, mitochondrial | Tfb1m | NM_146074_at | 0.000268 | 0.0265 | 5.97 | 5.08 | −0.88 |
| 364 | methyltransferase like 7A1 | Mettl7a1 | NM_027334_at | 0.000159 | 0.0265 | 10.32 | 9.42 | −0.90 |
| 365 | tetratricopeptide repeat domain 30B | Ttc30b | NM_028235_at | 0.000051 | 0.0265 | 7.76 | 6.86 | −0.91 |
| 366 | cysteinyl leukotriene receptor 1 | Cysltr1 | NM_021476_at | 0.000319 | 0.0265 | 6.99 | 6.07 | −0.91 |
| 367 | asp (abnormal spindle)-like, | Aspm | NM_009791_at | 0.000978 | 0.0265 | 6.64 | 5.73 | −0.91 |
| | microcephaly associated (Drosophila) | | | | | | | |
| 368 | tripartite motif-containing 34A | Trim34a | NM_030684_at | 0.000110 | 0.0265 | 6.99 | 6.07 | −0.92 |
| 369 | ubiquitin-conjugating enzyme E2T | Ube2t | NM_026024_at | 0.000719 | 0.0265 | 5.85 | 4.93 | −0.92 |
| | (putative) | | | | | | | |
| 370 | transmembrane protein 80 | Tmem80 | NM_001141950_at | 0.000062 | 0.0265 | 6.85 | 5.92 | −0.93 |
| 371 | transmembrane protein 80 | Tmem80 | NM_027797_at | 0.000062 | 0.0265 | 6.85 | 5.92 | −0.93 |
| 372 | zinc finger, FYVE domain containing 21 | Zfyve21 | NM_026752_at | 0.000010 | 0.0265 | 8.40 | 7.43 | −0.97 |
| 373 | Meis homeobox 1 | Meis1 | NM_001193271_at | 0.000062 | 0.1164 | 7.87 | 6.89 | −0.98 |
| 374 | Meis homeobox 1 | Meis1 | NM_010789_at | 0.000062 | 0.1164 | 7.87 | 6.89 | −0.98 |
| 375 | RIKEN cDNA 5730494M16 gene | 5730494M16Rik | NM_001004361_at | 0.000377 | 0.0650 | 6.17 | 5.17 | −1.00 |
| 376 | RIKEN cDNA 5730494M16 gene | 5730494M16Rik | NM_001142697_at | 0.000377 | 0.0650 | 6.17 | 5.17 | −1.00 |
| 377 | arginine vasopressin receptor 1A | Avpr1a | NM_016847_at | 0.000015 | 0.0265 | 7.35 | 6.34 | −1.01 |
| 378 | helicase-like transcription factor | Hltf | NM_144959_at | 0.000842 | 0.0265 | 6.20 | 5.16 | −1.04 |
| 379 | transcription factor AP-2 beta | Tcfap2b | NM_001025305_at | 0.000614 | 0.0265 | 9.37 | 8.23 | −1.13 |
| 380 | transcription factor AP-2 beta | Tcfap2b | NM_009334_at | 0.000614 | 0.0265 | 9.37 | 8.23 | −1.13 |
| 381 | MTERF domain containing 3 | Mterfd3 | NM_028832_at | 0.000042 | 0.0265 | 6.79 | 5.51 | −1.28 |
| 382 | zinc finger protein 101 | Zfp101 | NM_009542_at | 0.000006 | 0.0265 | 8.40 | 7.12 | −1.28 |
| 383 | sclerostin domain containing 1 | Sostdc1 | NM_025312_at | 0.000189 | 0.0265 | 9.83 | 8.51 | −1.32 |
| 384 | claudin 8 | Cldn8 | NM_018778_at | 0.000268 | 0.0650 | 7.05 | 5.34 | −1.71 |

### Example 7

### Correlation of Regulated Genes with Known Pathways

Both DBP up-regulated genes and down-regulated genes were analyzed for their involvement in known cellular pathways in mouse. Publicly available websites such as the NCBI browser available on the internet at ncbi.nlm.nih.gov and the Ensembl Genome Browser on the internet at ensembl.org may be used to correlate genes with various cellular pathways. The pathways are ranked by correlation and are shown in Tables 10 and 11.

TABLE 10
Correlation of Cellular Pathways with Upregulated Genes

| | p (geo- | Statis- | | | |
|---|---|---|---|---|---|
| | metric | tical | P | Q | Set |
| Pathway | mean) | mean | value | value | size |
| Signaling in | 8.6E−05 | 3.61 | 2.6E−24 | 1.2E−21 | 210 |
| Immune system | | | | | |
| GPCR downstream | 2.6E−03 | 2.78 | 2.3E−15 | 5.6E−13 | 346 |
| signaling | | | | | |
| Signaling by GPCR | 3.8E−03 | 2.65 | 3.7E−14 | 6.0E−12 | 359 |
| GPCR ligand | 4.5E−03 | 2.61 | 1.2E−13 | 1.2E−11 | 214 |
| binding | | | | | |
| Class A/1 | 4.4E−03 | 2.61 | 1.3E−13 | 1.2E−11 | 174 |
| (Rhodopsin-like | | | | | |
| receptors) | | | | | |
| Peptide ligand- | 6.5E−03 | 2.49 | 2.1E−12 | 1.7E−10 | 102 |
| binding receptors | | | | | |
| G alpha (i) | 6.4E−03 | 2.47 | 2.8E−12 | 1.9E−10 | 123 |
| signalling events | | | | | |
| Toll Receptor | 6.8E−03 | 2.47 | 3.2E−12 | 1.9E−10 | 60 |
| Cascades | | | | | |
| Chemokine | 1.3E−02 | 2.32 | 2.2E−10 | 1.2E−08 | 29 |
| receptors bind | | | | | |
| chemokines | | | | | |
| Toll Like Receptor | 1.3E−02 | 2.24 | 2.6E−10 | 1.2E−08 | 45 |
| 2 Cascade | | | | | |
| Toll Like Receptor | 1.3E−02 | 2.24 | 2.6E−10 | 1.2E−08 | 45 |
| TLR6:TLR2 | | | | | |
| Cascade | | | | | |
| Toll Like Receptor | 1.3E−02 | 2.22 | 3.6E−10 | 1.4E−08 | 47 |
| 4 (TLR4) Cascade | | | | | |
| TRAF6 mediated | 1.5E−02 | 2.16 | 1.0E−09 | 3.3E−08 | 48 |
| induction of NFkB | | | | | |
| and MAP kinases | | | | | |
| upon TLR7/8 or 9 | | | | | |
| activation | | | | | |
| MyD88 dependent | 1.5E−02 | 2.16 | 1.0E−09 | 3.3E−08 | 48 |
| cascade initiated on | | | | | |
| endosome | | | | | |
| Toll Like Receptor | 1.5E−02 | 2.16 | 1.0E−09 | 3.3E−08 | 48 |
| 7/8 (TLR7/8) | | | | | |
| Cascade | | | | | |
| Toll Like Receptor | 1.6E−02 | 2.12 | 1.8E−09 | 5.5E−08 | 48 |
| 3 (TLR3) Cascade | | | | | |
| TCR signaling | 1.6E−02 | 2.11 | 2.4E−09 | 6.7E−08 | 42 |
| NFkB and MAP | 1.9E−02 | 2.07 | 4.6E−09 | 1.2E−07 | 42 |
| kinases activation | | | | | |
| mediated by TLR4 | | | | | |
| signaling repertoire | | | | | |
| MyD88- | 2.1E−02 | 2.03 | 8.9E−09 | 2.1E−07 | 44 |
| independent | | | | | |
| cascade initiated on | | | | | |
| plasma membrane | | | | | |
| MyD88:Mal | 2.1E−02 | 2.02 | 1.0E−08 | 2.1E−07 | 43 |
| cascade initiated on | | | | | |
| plasma membrane | | | | | |
| Toll Like Receptor | 2.1E−02 | 2.02 | 1.0E−08 | 2.1E−07 | 43 |
| TLR1:TLR2 | | | | | |
| Cascade | | | | | |
| MyD88 cascade | 2.1E−02 | 2.02 | 1.0E−08 | 2.1E−07 | 43 |
| initiated on plasma | | | | | |
| membrane | | | | | |
| Toll Like Receptor | 2.1E−02 | 2.02 | 1.0E−08 | 2.1E−07 | 43 |
| 10 (TLR10) | | | | | |
| Cascade | | | | | |
| Toll Like Receptor | 2.1E−02 | 2.02 | 1.0E−08 | 2.1E−07 | 43 |
| 5 (TLR5) Cascade | | | | | |
| Toll Like Receptor | 2.3E−02 | 1.99 | 1.5E−08 | 2.8E−07 | 50 |
| 9 (TLR9) Cascade | | | | | |
| Innate Immunity | 1.8E−02 | 1.97 | 1.7E−08 | 3.2E−07 | 80 |
| Signaling | | | | | |
| TRAF6 Mediated | 2.4E−02 | 1.98 | 1.8E−08 | 3.2E−07 | 43 |
| Induction of | | | | | |
| proinflammatory | | | | | |
| cytokines | | | | | |
| Activated TLR4 | 2.3E−02 | 1.98 | 1.9E−08 | 3.3E−07 | 45 |
| signalling | | | | | |
| Immunoregulatory | 2.0E−02 | 1.92 | 5.5E−08 | 9.1E−07 | 42 |
| interactions | | | | | |
| between a | | | | | |
| Lymphoid and a | | | | | |
| non-Lymphoid cell | | | | | |
| Costimulation by | 3.0E−02 | 1.86 | 1.1E−07 | 1.7E−06 | 44 |
| the CD28 family | | | | | |
| Transport of | 3.2E−02 | 1.82 | 1.6E−07 | 2.5E−06 | 69 |
| inorganic | | | | | |
| cations/anions and | | | | | |
| amino | | | | | |
| acids/oligopeptides | | | | | |
| SLC-mediated | 3.1E−02 | 1.78 | 2.6E−07 | 3.9E−06 | 143 |
| transmembrane | | | | | |
| transport | | | | | |
| Amino acid and | 3.5E−02 | 1.80 | 2.7E−07 | 3.9E−06 | 36 |
| oligopeptide SLC | | | | | |
| transporters | | | | | |
| Downstream TCR | 3.6E−02 | 1.80 | 3.4E−07 | 4.9E−06 | 27 |
| signaling | | | | | |
| p75NTR signals via | 4.3E−02 | 1.78 | 9.3E−07 | 1.3E−05 | 12 |
| NF-kB | | | | | |
| CD28 co- | 5.9E−02 | 1.57 | 6.4E−06 | 8.6E−05 | 27 |
| stimulation | | | | | |
| TAK1 activates | 5.7E−02 | 1.60 | 6.7E−06 | 8.8E−05 | 15 |
| NFkB by | | | | | |
| phosphorylation | | | | | |
| and activation of | | | | | |
| IKKs complex | | | | | |
| Transmembrane | 5.2E−02 | 1.52 | 8.9E−06 | 1.1E−04 | 174 |
| transport of small | | | | | |
| molecules | | | | | |
| Viral | 6.5E−02 | 1.52 | 1.6E−05 | 2.0E−04 | 17 |
| dsRNA:TLR3:TRIF | | | | | |
| Complex Activates | | | | | |
| RIP1 | | | | | |
| Generation of | 5.3E−02 | 1.51 | 1.8E−05 | 2.2E−04 | 20 |
| second messenger | | | | | |
| molecules | | | | | |
| MAPK targets/ | 6.7E−02 | 1.50 | 1.9E−05 | 2.2E−04 | 20 |
| Nuclear events | | | | | |
| mediated by MAP | | | | | |
| kinases | | | | | |
| Death Receptor | 7.2E−02 | 1.52 | 2.3E−05 | 2.6E−04 | 11 |
| Signalling | | | | | |
| Extrinsic Pathway | 7.2E−02 | 1.52 | 2.3E−05 | 2.6E−04 | 11 |
| for Apoptosis | | | | | |
| G alpha (s) | 7.3E−02 | 1.37 | 6.3E−05 | 6.9E−04 | 75 |
| signalling events | | | | | |
| MAP kinase | 8.4E−02 | 1.38 | 6.5E−05 | 7.0E−04 | 27 |
| activation in TLR | | | | | |
| cascade | | | | | |
| CTLA4 inhibitory | 8.7E−02 | 1.40 | 7.6E−05 | 8.0E−04 | 11 |
| signaling | | | | | |
| Integrin cell surface | 8.0E−02 | 1.32 | 1.0E−04 | 1.1E−03 | 76 |
| interactions | | | | | |
| Hemostasis | 6.9E−02 | 1.31 | 1.1E−04 | 1.1E−03 | 261 |
| RIG-I/MDA5 | 7.7E−02 | 1.35 | 1.1E−04 | 1.1E−03 | 19 |
| mediated induction | | | | | |
| of IFN-alpha/beta | | | | | |
| pathways | | | | | |
| Sema4D induced | 8.6E−02 | 1.35 | 1.2E−04 | 1.2E−03 | 15 |
| cell migration and | | | | | |
| growth-cone | | | | | |
| collapse | | | | | |
| Amino acid | 9.6E−02 | 1.31 | 1.4E−04 | 1.3E−03 | 24 |
| transport across the | | | | | |
| plasma membrane | | | | | |
| Sema4D in | 9.2E−02 | 1.30 | 1.6E−04 | 1.5E−03 | 20 |
| semaphorin | | | | | |
| signaling | | | | | |
| Nuclear Events | 9.8E−02 | 1.31 | 1.8E−04 | 1.7E−03 | 13 |
| (kinase and | | | | | |
| transcription factor | | | | | |
| activation) | | | | | |
| Cell junction | 9.1E−02 | 1.26 | 2.0E−04 | 1.8E−03 | 58 |
| organization | | | | | |
| CD28 dependent | 1.0E−01 | 1.28 | 2.2E−04 | 1.9E−03 | 17 |
| PI3K/Akt signaling | | | | | |
| CD28 dependent | 1.1E−01 | 1.27 | 2.7E−04 | 2.3E−03 | 12 |
| Vav1 pathway | | | | | |
| ERK/MAPK targets | 1.1E−01 | 1.28 | 2.8E−04 | 2.4E−03 | 10 |
| Chaperonin- | 1.0E−01 | 1.22 | 3.6E−04 | 3.0E−03 | 21 |
| mediated protein | | | | | |
| folding | | | | | |
| Cooperation of | 1.1E−01 | 1.21 | 4.3E−04 | 3.6E−03 | 20 |
| Prefoldin and | | | | | |
| TriC/CCT in actin | | | | | |
| and tubulin folding | | | | | |
| Prefoldin mediated | 1.1E−01 | 1.19 | 5.0E−04 | 4.1E−03 | 19 |
| transfer of substrate | | | | | |
| to CCT/TriC | | | | | |
| Transport of | 1.2E−01 | 1.15 | 6.3E−04 | 5.0E−03 | 39 |
| glucose and other | | | | | |
| sugars, bile salts | | | | | |
| and organic acids, | | | | | |
| metal ions and | | | | | |
| amine compounds | | | | | |
| Cytosolic tRNA | 8.4E−02 | 1.17 | 7.0E−04 | 5.4E−03 | 25 |
| aminoacylation | | | | | |
| Interleukin-2 | 1.3E−01 | 1.10 | 1.0E−03 | 8.1E−03 | 26 |
| signaling | | | | | |
| Cell-cell junction | 1.3E−01 | 1.09 | 1.2E−03 | 9.1E−03 | 31 |
| organization | | | | | |
| Interleukin receptor | 1.4E−01 | 1.08 | 1.3E−03 | 9.6E−03 | 22 |
| SHC signaling | | | | | |
| Interleukin-3, 5 and | 1.4E−01 | 1.08 | 1.3E−03 | 9.6E−03 | 22 |
| GM-CSF signaling | | | | | |
| Cell surface | 1.1E−01 | 1.05 | 1.6E−03 | 1.1E−02 | 77 |
| interactions at the | | | | | |
| vascular wall | | | | | |
| Signaling by | 1.3E−01 | 1.05 | 1.6E−03 | 1.1E−02 | 36 |
| Interleukins | | | | | |
| PD-1 signaling | 1.4E−01 | 1.07 | 1.7E−03 | 1.2E−02 | 14 |
| Semaphorin | 1.4E−01 | 1.03 | 2.0E−03 | 1.4E−02 | 53 |
| interactions | | | | | |
| Protein folding | 1.6E−01 | 0.97 | 3.4E−03 | 2.3E−02 | 26 |
| Signalling by NGF | 1.4E−01 | 0.89 | 6.1E−03 | 4.1E−02 | 184 |
| G alpha (q) | 1.9E−01 | 0.86 | 7.8E−03 | 5.2E−02 | 120 |
| signalling events | | | | | |
| GPVI-mediated | 1.8E−01 | 0.85 | 8.9E−03 | 5.8E−02 | 24 |
| activation cascade | | | | | |
| Mus musculus: | 2.0E−01 | 0.86 | 9.0E−03 | 5.8E−02 | 11 |
| Post-chaperonin | | | | | |
| tubulin folding | | | | | |
| pathway | | | | | |
| Unfolded Protein | 1.9E−01 | 0.81 | 1.1E−02 | 7.1E−02 | 50 |
| Response | | | | | |
| Basigin interactions | 1.9E−01 | 0.82 | 1.1E−02 | 7.1E−02 | 25 |
| NGF signalling via | 1.6E−01 | 0.81 | 1.1E−02 | 7.1E−02 | 106 |
| TRKA from the | | | | | |
| plasma membrane | | | | | |
| Adherens junctions | 2.0E−01 | 0.80 | 1.3E−02 | 7.6E−02 | 21 |
| interactions | | | | | |
| tRNA | 1.5E−01 | 0.81 | 1.3E−02 | 7.6E−02 | 33 |
| Aminoacylation | | | | | |
| Phosphorylation of | 1.9E−01 | 0.81 | 1.3E−02 | 7.9E−02 | 11 |
| CD3 and TCR zeta | | | | | |
| chains | | | | | |

TABLE 11
Correlation of Cellular Pathways with Downregulated Genes

| | p (geo- | Statis- | | | |
|---|---|---|---|---|---|
| | metric | tical | P | Q | Set |
| Pathway | mean) | mean | value | value | size |
| Cell Cycle, Mitotic | 2.9E−05 | −3.8E+00 | 4.7E−26 | 2.3E−23 | 237 |
| DNA Replication | 4.7E−04 | −3.0E+00 | 6.6E−17 | 1.4E−14 | 157 |
| DNA strand elongation | 8.1E−04 | −3.1E+00 | 8.9E−17 | 1.4E−14 | 29 |
| Telomere C-strand | 2.0E−03 | −2.9E+00 | 1.4E−14 | 1.7E−12 | 22 |
| (Lagging Strand) | | | | | |
| Synthesis | | | | | |
| DNA Repair | 2.4E−03 | −2.7E+00 | 4.0E−14 | 3.9E−12 | 85 |
| Extension of Telomeres | 2.4E−03 | −2.8E+00 | 4.9E−14 | 4.0E−12 | 23 |
| Telomere Maintenance | 2.8E−03 | −2.7E+00 | 8.8E−14 | 5.3E−12 | 31 |
| Chromosome | 2.8E−03 | −2.7E+00 | 8.8E−14 | 5.3E−12 | 31 |
| Maintenance | | | | | |
| Lagging Strand | 3.8E−03 | −2.7E+00 | 6.1E−13 | 3.3E−11 | 20 |
| Synthesis | | | | | |
| Mitotic M-M/G1 phases | 1.8E−03 | −2.5E+00 | 1.0E−12 | 4.9E−11 | 139 |
| Gap-filling DNA | 5.4E−03 | −2.6E+00 | 3.6E−12 | 1.5E−10 | 16 |
| repair synthesis and | | | | | |
| ligation in GG-NER | | | | | |
| Gap-filling DNA | 5.4E−03 | −2.6E+00 | 3.6E−12 | 1.5E−10 | 16 |
| repair synthesis and | | | | | |
| ligation in TC-NER | | | | | |
| Global Genomic | 6.7E−03 | −2.5E+00 | 7.4E−12 | 2.8E−10 | 33 |
| NER (GG-NER) | | | | | |
| Synthesis of DNA | 6.3E−03 | −2.4E+00 | 1.9E−11 | 6.2E−10 | 80 |
| Respiratory electron | 3.6E−03 | −2.4E+00 | 2.0E−11 | 6.2E−10 | 77 |
| transport, ATP | | | | | |
| synthesis by | | | | | |
| chemiosmotic coupling, | | | | | |
| and heat production by | | | | | |
| uncoupling proteins. | | | | | |
| Repair synthesis of | 7.4E−03 | −2.5E+00 | 2.2E−11 | 6.2E−10 | 15 |
| patch ~27-30 bases | | | | | |
| long by DNA | | | | | |
| polymerase | | | | | |
| Repair synthesis for | 7.4E−03 | −2.5E+00 | 2.2E−11 | 6.2E−10 | 15 |
| gap-filling by DNA | | | | | |
| polymerase in TC-NER | | | | | |
| Cell Cycle Checkpoints | 7.0E−03 | −2.3E+00 | 3.2E−11 | 8.5E−10 | 96 |
| Respiratory electron | 4.1E−03 | −2.4E+00 | 3.6E−11 | 9.3E−10 | 64 |
| transport | | | | | |
| G2/M Transition | 1.1E−02 | −2.3E+00 | 1.5E−10 | 3.7E−09 | 53 |
| Mitotic G2-G2/M phases | 1.1E−02 | −2.3E+00 | 1.8E−10 | 4.3E−09 | 56 |
| G2/M Checkpoints | 8.6E−03 | −2.3E+00 | 2.1E−10 | 4.7E−09 | 40 |
| Nucleotide Excision | 1.2E−02 | −2.2E+00 | 4.2E−10 | 8.9E−09 | 44 |
| Repair | | | | | |
| Processive synthesis | 1.1E−02 | −2.3E+00 | 7.2E−10 | 1.5E−08 | 15 |
| on the lagging strand | | | | | |
| S Phase | 1.0E−02 | −2.2E+00 | 7.9E−10 | 1.5E−08 | 93 |
| Removal of the Flap | 1.5E−02 | −2.2E+00 | 4.2E−09 | 7.8E−08 | 14 |
| Intermediate | | | | | |
| Transcription-coupled | 1.6E−02 | −2.1E+00 | 4.9E−09 | 8.8E−08 | 39 |
| NER (TC-NER) | | | | | |
| Centrosome maturation | 2.0E−02 | −2.0E+00 | 7.5E−09 | 1.3E−07 | 44 |
| Recruitment of | 2.0E−02 | −2.0E+00 | 7.5E−09 | 1.3E−07 | 44 |
| mitotic centrosome | | | | | |
| proteins and complexes | | | | | |
| Processive synthesis | 2.0E−02 | −2.2E+00 | 1.7E−08 | 2.7E−07 | 11 |
| on the C-strand of the | | | | | |
| telomere | | | | | |
| Activation of the pre- | 1.5E−02 | −2.0E+00 | 2.2E−08 | 3.5E−07 | 29 |
| replicative complex | | | | | |
| Activation of ATR | 2.0E−02 | −2.0E+00 | 2.5E−08 | 3.9E−07 | 34 |
| in response to replication | | | | | |
| stress | | | | | |
| DNA Replication Pre- | 2.1E−02 | −1.9E+00 | 6.9E−08 | 9.9E−07 | 66 |
| Initiation | | | | | |
| M/G1 Transition | 2.1E−02 | −1.9E+00 | 6.9E−08 | 9.9E−07 | 66 |
| Double-Strand Break | 2.1E−02 | −2.0E+00 | 7.4E−08 | 1.0E−06 | 20 |
| Repair | | | | | |
| Removal of the Flap | 2.7E−02 | −2.0E+00 | 1.0E−07 | 1.4E−06 | 10 |
| Intermediate from | | | | | |
| the C-strand | | | | | |
| Homologous | 2.8E−02 | −1.9E+00 | 2.2E−07 | 2.8E−06 | 15 |
| recombination repair | | | | | |
| of replication- | | | | | |
| independent double- | | | | | |
| strand breaks | | | | | |
| Homologous | 2.8E−02 | −1.9E+00 | 2.2E−07 | 2.8E−06 | 15 |
| Recombination Repair | | | | | |
| Mitochondrial Fatty | 3.5E−02 | −1.9E+00 | 3.2E−07 | 3.8E−06 | 13 |
| Acid Beta-Oxidation | | | | | |
| Polymerase switching | 3.1E−02 | −1.9E+00 | 3.3E−07 | 3.8E−06 | 13 |
| Leading Strand | 3.1E−02 | −1.9E+00 | 3.3E−07 | 3.8E−06 | 13 |
| Synthesis | | | | | |
| Polymerase switching | 3.1E−02 | −1.9E+00 | 3.3E−07 | 3.8E−06 | 13 |
| on the C-strand of the | | | | | |
| telomere | | | | | |
| Striated Muscle | 3.1E−03 | −2.0E+00 | 5.4E−07 | 6.0E−06 | 26 |
| Contraction | | | | | |
| Mitotic G1-G1/S phases | 2.7E−02 | −1.7E+00 | 9.3E−07 | 1.0E−05 | 93 |
| Branched-chain amino | 4.4E−02 | −1.7E+00 | 1.1E−06 | 1.2E−05 | 17 |
| acid catabolism | | | | | |
| M Phase | 1.8E−02 | −1.7E+00 | 1.2E−06 | 1.3E−05 | 73 |
| G1/S Transition | 3.0E−02 | −1.7E+00 | 1.5E−06 | 1.5E−05 | 83 |
| APC/C-mediated | 4.0E−02 | −1.6E+00 | 6.7E−06 | 6.6E−05 | 63 |
| degradation of cell | | | | | |
| cycle proteins | | | | | |
| Regulation of mitotic | 4.0E−02 | −1.6E+00 | 6.7E−06 | 6.6E−05 | 63 |
| cell cycle | | | | | |
| Loss of Nlp from | 5.6E−02 | −1.6E+00 | 7.3E−06 | 7.1E−05 | 39 |
| mitotic centrosomes | | | | | |
| Formation of incision | 5.9E−02 | −1.6E+00 | 8.3E−06 | 7.8E−05 | 21 |
| complex in GG-NER | | | | | |
| Dual incision reaction | 5.9E−02 | −1.6E+00 | 8.3E−06 | 7.8E−05 | 21 |
| in GG-NER | | | | | |
| Mitotic Prometaphase | 2.6E−02 | −1.5E+00 | 9.3E−06 | 8.5E−05 | 70 |
| Activation of APC/C | 4.8E−02 | −1.5E+00 | 1.6E−05 | 1.4E−04 | 55 |
| and APC/C:Cdc20 | | | | | |
| mediated degradation | | | | | |
| of mitotic proteins | | | | | |
| Meiotic | 6.0E−02 | −1.5E+00 | 1.7E−05 | 1.5E−04 | 22 |
| Recombination (mouse) | | | | | |
| Muscle contraction | 8.6E−03 | −1.5E+00 | 1.9E−05 | 1.7E−04 | 41 |
| Regulation of APC/C | 5.2E−02 | −1.5E+00 | 2.3E−05 | 1.9E−04 | 59 |
| activators between | | | | | |
| G1/S and early anaphase | | | | | |
| NCAM1 interactions | 5.1E−02 | −1.5E+00 | 2.8E−05 | 2.3E−04 | 29 |
| APC/C:Cdc20 | 5.4E−02 | −1.4E+00 | 3.1E−05 | 2.5E−04 | 54 |
| mediated degradation | | | | | |
| of mitotic proteins | | | | | |
| Phosphorylation of | 7.3E−02 | −1.4E+00 | 5.4E−05 | 4.4E−04 | 14 |
| the APC/C | | | | | |
| APC/C:Cdc20 | 7.6E−02 | −1.4E+00 | 7.9E−05 | 6.3E−04 | 14 |
| mediated degradation | | | | | |
| of Cyclin B | | | | | |
| Glycogen breakdown | 7.4E−02 | −1.4E+00 | 8.6E−05 | 6.7E−04 | 14 |
| (glycogenolysis) | | | | | |
| Inactivation of APC/C | 8.7E−02 | −1.3E+00 | 1.3E−04 | 9.6E−04 | 15 |
| via direct inhibition | | | | | |
| of the APC/C complex | | | | | |
| Inhibition of the | 8.7E−02 | −1.3E+00 | 1.3E−04 | 9.6E−04 | 15 |
| proteolytic activity | | | | | |
| of APC/C required for | | | | | |
| the onset of anaphase | | | | | |
| by mitotic spindle | | | | | |
| checkpoint components | | | | | |
| APC-Cdc20 mediated | 8.7E−02 | −1.3E+00 | 1.3E−04 | 9.6E−04 | 15 |
| degradation of Nek2A | | | | | |
| Cdc20:Phospho- | 7.1E−02 | −1.3E+00 | 1.4E−04 | 1.0E−03 | 52 |
| APC/C mediated | | | | | |
| degradation of Cyclin A | | | | | |
| NCAM signaling for | 7.6E−02 | −1.3E+00 | 1.5E−04 | 1.1E−03 | 52 |
| neurite out-growth | | | | | |
| APC/C:Cdh1 mediated | 7.4E−02 | −1.3E+00 | 1.8E−04 | 1.3E−03 | 54 |
| degradation of Cdc20 | | | | | |
| and other | | | | | |
| APC/C:Cdh1 targeted | | | | | |
| proteins in late | | | | | |
| mitosis/early G1 | | | | | |
| Mitotic Spindle | 9.3E−02 | −1.3E+00 | 1.8E−04 | 1.3E−03 | 16 |
| Checkpoint | | | | | |
| Glucuronidation | 9.7E−02 | −1.3E+00 | 2.9E−04 | 2.0E−03 | 10 |
| Cyclin A/B1 | 9.7E−02 | −1.3E+00 | 3.4E−04 | 2.3E−03 | 11 |
| associated events | | | | | |
| during G2/M transition | | | | | |
| Conversion from | 1.2E−01 | −1.2E+00 | 7.6E−04 | 5.1E−03 | 13 |
| APC/C:Cdc20 to | | | | | |
| APC/C:Cdh1 in late | | | | | |
| anaphase | | | | | |
| Citric acid cycle (TCA | 1.1E−01 | −1.2E+00 | 9.1E−04 | 6.0E−03 | 17 |
| cycle) | | | | | |
| APC/C:Cdc20 | 1.0E−01 | −1.1E+00 | 9.2E−04 | 6.0E−03 | 49 |
| mediated degradation | | | | | |
| of Securin | | | | | |
| Synthesis of bile acids | 1.3E−01 | −1.1E+00 | 9.3E−04 | 6.0E−03 | 22 |
| and bile salts via | | | | | |
| 7alpha-hydroxycholesterol | | | | | |
| Assembly of the pre- | 1.0E−01 | −1.1E+00 | 9.8E−04 | 6.2E−03 | 51 |
| replicative complex | | | | | |
| Metabolism of water- | 1.2E−01 | −1.1E+00 | 1.1E−03 | 7.2E−03 | 31 |
| soluble vitamins and | | | | | |
| cofactors | | | | | |
| Metabolism of porphyrins | 1.3E−01 | −1.1E+00 | 1.3E−03 | 8.0E−03 | 21 |
| Resolution of Abasic | 1.4E−01 | −1.1E+00 | 1.5E−03 | 9.2E−03 | 15 |
| Sites (AP sites) | | | | | |
| Base Excision Repair | 1.4E−01 | −1.1E+00 | 1.5E−03 | 9.2E−03 | 15 |
| Removal of DNA | 1.4E−01 | −1.1E+00 | 1.8E−03 | 1.1E−02 | 13 |
| patch containing | | | | | |
| abasic residue | | | | | |
| Resolution of AP sites | 1.4E−01 | −1.1E+00 | 1.8E−03 | 1.1E−02 | 13 |
| via the multiple- | | | | | |
| nucleotide patch | | | | | |
| replacement pathway | | | | | |
| Autodegradation of | 1.2E−01 | −1.0E+00 | 1.9E−03 | 1.1E−02 | 47 |
| Cdh1 by Cdh1:APC/C | | | | | |
| Heme degradation | 1.3E−01 | −1.1E+00 | 2.0E−03 | 1.1E−02 | 12 |
| Regulation of DNA | 1.3E−01 | −1.0E+00 | 2.1E−03 | 1.2E−02 | 57 |
| replication | | | | | |
| Removal of licensing | 1.3E−01 | −1.0E+00 | 2.1E−03 | 1.2E−02 | 57 |
| factors from origins | | | | | |
| Phase II conjugation | 1.5E−01 | −1.0E+00 | 2.1E−03 | 1.2E−02 | 44 |
| Transcriptional | 1.5E−01 | −1.0E+00 | 2.2E−03 | 1.2E−02 | 39 |
| Regulation of White | | | | | |
| Adipocyte | | | | | |
| Differentiation | | | | | |
| CDO in myogenesis | 1.5E−01 | −1.0E+00 | 2.2E−03 | 1.2E−02 | 11 |
| 1327149: Mus | 1.5E−01 | −1.0E+00 | 2.2E−03 | 1.2E−02 | 11 |
| musculus: Myogenesis | | | | | |
| Transcriptional | 1.5E−01 | −9.8E−01 | 3.0E−03 | 1.6E−02 | 40 |
| Regulation of Adipocyte | | | | | |
| Differentiation in 3T3- | | | | | |
| L1 Pre-adipocytes | | | | | |
| Synthesis of bile acids | 1.5E−01 | −9.8E−01 | 3.5E−03 | 1.9E−02 | 17 |
| and bile salts via 24- | | | | | |
| hydroxycholesterol | | | | | |
| RNA Polymerase III | 1.6E−01 | −9.6E−01 | 3.9E−03 | 2.0E−02 | 18 |
| Transcription Initiation | | | | | |
| RNA Polymerase III | 1.6E−01 | −9.6E−01 | 3.9E−03 | 2.0E−02 | 18 |
| Transcription | | | | | |
| RNA Polymerase III | 1.6E−01 | −9.6E−01 | 3.9E−03 | 2.0E−02 | 18 |
| Abortive And | | | | | |
| Retractive Initiation | | | | | |
| RNA Polymerase I, RNA | 1.6E−01 | −8.9E−01 | 6.3E−03 | 3.2E−02 | 42 |
| Polymerase III, and | | | | | |
| Mitochondrial | | | | | |
| Transcription | | | | | |
| Orc1 removal from | 1.6E−01 | −8.8E−01 | 7.1E−03 | 3.5E−02 | 55 |
| chromatin | | | | | |
| Switching of origins | 1.6E−01 | −8.8E−01 | 7.1E−03 | 3.5E−02 | 55 |
| to a post-replicative | | | | | |
| state | | | | | |
| Metabolism of | 1.9E−01 | −8.6E−01 | 8.0E−03 | 3.9E−02 | 37 |
| vitamins and cofactors | | | | | |
| Glucose metabolism | 1.2E−01 | −8.6E−01 | 8.1E−03 | 3.9E−02 | 48 |
| Formation of ATP by | 1.7E−01 | −8.9E−01 | 9.5E−03 | 4.6E−02 | 10 |
| chemiosmotic coupling | | | | | |
| Fanconi Anemia pathway | 1.9E−01 | −8.4E−01 | 1.1E−02 | 5.1E−02 | 14 |
| Biological oxidations | 1.9E−01 | −7.7E−01 | 1.5E−02 | 7.0E−02 | 97 |
| E2F mediated regulation | 1.9E−01 | −7.8E−01 | 1.6E−02 | 7.3E−02 | 16 |
| of DNA replication | | | | | |
| CDT1 association with | 1.9E−01 | −7.3E−01 | 2.1E−02 | 9.8E−02 | 45 |
| the CDC6:ORC:origin | | | | | |
| complex | | | | | |

Referring to Table 10, immune response signaling is the most relevant up-regulated pathway, followed by G protein coupled receptors that includes the chemokine receptors used by the dendritic cells to navigate migration from the skin to the draining lymph node. Referring to Table 11, the down-regulated genes are predominantly cell cycle genes.

The analysis considers all levels of gene expression changes in full thickness skin induced by topical DBP and indicates the biological pathways that are involved, with a corresponding p value for each pathway. This analysis validates that gene expression in full thickness skin induced by topical DBP corresponds to dendritic cell activation in the skin. Previously, we demonstrated that activation of this phenomenon can be used as an effective vaccine adjuvant (see U.S. Pat. Nos. 6,210,672 and 7,229,621).

### Example 8

### Seasonal Influenza Vaccine used with and without Topical Adjuvants

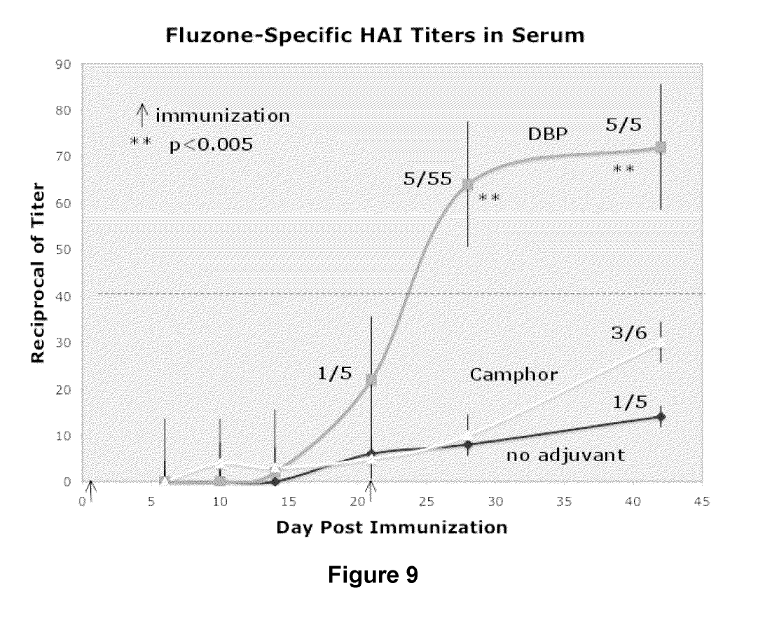

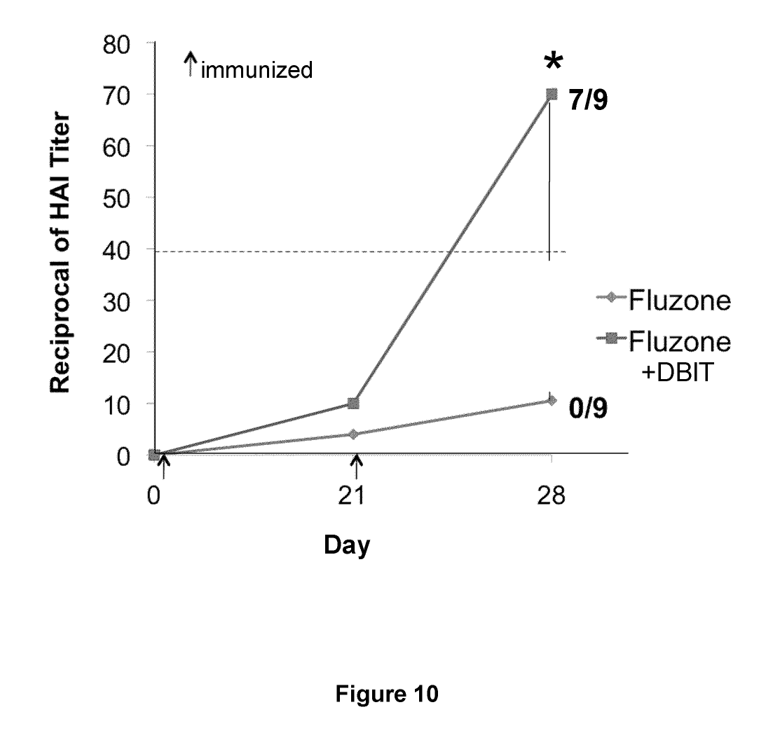

The human seasonal influenza vaccine, Fluzone, was used to immunize C57BL/6 mice with and without topical adjuvants. Groups of 5 to 9 mice each were shaved on the abdominal skin on day −1 and the following day immunized intradermally on one side of the abdomen with 50 microliters of Fluzone using a 300 microliter insulin syringe with a 28 gauge needle. Some of the mice were also treated topically over the immunization site with camphor dissolved in acetone, DBP, or dibutyl L tartrate (DBlT). Separate groups of mice were bled on days 7, 10, 14 or 21 and tested for hemagglutination inhibition (HAI) titers to Fluzone using the chicken red blood cell protocol described by the World Health Organization (WHO). Other groups of mice were reimmunized in the same way on the opposite side of the abdomen on day 21 and bled on day 28 or day 42. The data are shown in FIGS. 9 and 10. An HAI titer of 1:40 or greater is considered to be protective in human clinical trials of seasonal influenza vaccines.

FIG. 9 shows that the dose of influenza vaccine (Fluzone) used was sub-optimal for inducing a protective response, defined by the WHO as a hemagglutination inhibition (HAI) titer of 1:40 or higher. The number of individual mice that achieved a titer of 1:40 or better is shown as the fractional response. When that same dose of fluzone was accompanied by topical camphor, the HAI titer was improved. When the same dose of Fluzone was accompanied by topical DBP, the HAI response was majorly improved (p<0.005). FIG. 10 shows the same kind of experiment using the same dose of Fluzone with and without topical DBT. Topical DBT had an adjuvant effect almost but not quite as good as DBT had (FIG. 9). In other words, the adjuvant activity of camphor DBT and DBP fall in the same order as the three topical molecules have in FIG. 8, where the number of activated dendritic cells in the draining lymph node is plotted. Our algorithms that analyze gene expression induced in the skin accurately predict the number of activated dendritic cells that will be found in the skin draining lymph node after topical application of the small lipophilic molecules. Therefore, the gene expression profile will predict the relative efficacy of a topical vaccine adjuvant.

Table 12 shows data for DBP and dibutyl L tartrate (DBlT).

TABLE 12
Activation of Murine Skin Dendritic Cells by DBP vs. DBlT

| Treatment | DC/LN (SD) | MFI CD86 |
|---|---|---|
| none (n = 17) | 6,859 (3,881) | 178 (50) |
| DBP (n = 15) | 145,255 (75,019) | 618 (95) |
| DBlT (n = 18) | 101,764 (67,477) | 634 (126) |

It is understood that the examples and embodiments described herein are for illustrative purposes only and that various modifications or changes in light thereof will be suggested to persons skilled in the art and are to be included within the spirit and purview of this application and scope of any appended claims. All figures, tables, and appendices, as well as publications, patents, and patent applications, cited herein are hereby incorporated by reference in their entirety for all purposes.

What is claimed is:

1. A method of identifying a candidate adjuvant capable of activating dendritic cells, the method comprising:
a) measuring expression level of a plurality of genes in skin of an animal prior to exposure to a test compound, wherein the plurality of genes are known to be upregulated or downregulated in the skin of the animal in response to topical application of dibutyl phthalate (DBP) to skin of said animal;
b) exposing skin of an animal of the same species to the test compound;
c) measuring expression level of the plurality of genes in the skin of the animal after exposure to the test compound; and
d) comparing expression level of the plurality of genes measured in steps (a) and (c), wherein an increase or decrease in expression level of the plurality of genes following exposure to the test compound by a pre-determined change in expression level indicates that the test compound is capable of activating dendritic cells.

2. The method of claim 1, wherein said pre-determined change in expression level is selected from the group consisting of: (a) an increase by a factor of at least 2; and (b) a decrease by a factor of at least 2.

3. The method of claim 1, wherein said plurality of genes is selected from the group of genes listed in Tables 1-5 and in Table 8, Nos. 1-215.

4. The method of claim 1, wherein said plurality of genes are significantly upregulated by Toll-like Receptor (TLR) stimulation of dendritic cells.

5. The method of claim 1, wherein said plurality of genes comprise early response gene(s).

6. The method of claim 1, wherein an increase in gene expression is measured using a weighted average.

7. The method of claim 1, wherein gene expression is measured using an array comprising a substrate and a plurality of polynucleotide probes affixed to the substrate.

8. The method of claim 7, wherein the array comprises a plurality of polynucleotide probes that are specifically complementary to said plurality of genes.

9. A method of identifying a candidate immunological adjuvant capable of activating dendritic cells, the method comprising:
(a) identifying genes whose expression levels are upregulated or downregulated in skin of an animal in response to topical application of DBP and DBP analogs;
(b) quantifying the levels of activated dendritic cells in draining lymph nodes of said animal in response to topical application of DBP and DBP analogs;
(c) determining a model of activity of the DBP and DBP analogs, wherein a level of activated dendritic cells in draining lymph nodes measured in response to topical application of DBP and DBP analogs is correlated with the genes whose expression levels are upregulated or downregulated in response to topical application of DBP and DBP analogs; and
(d) determining whether a test compound is a candidate immunological adjuvant capable of activating dendritic cells on the basis of whether topical application of the test compound results in upregulation or downregulation of genes comparable to genes that show upregulation or downregulation in response to DBP or a DBP analog that leads to increased levels of activated dendritic cells in draining lymph nodes of said animal.

10. The method of claim 9, wherein said model of activity of the DBP and DBP analogs is selected from the group consisting of Bayesian additive regression trees (BART), multivariate adaptive regression splines (MARS), gradient-boosted generalized linear models (GLMs), and bagged generalized linear models.

11. The method of claim 9, wherein said genes whose expression levels are upregulated or downregulated are selected from the group of genes listed in:
(a) Table 8, Nos. 216-436 determined by the Wilcoxin model and the genes determined by the Wilcoxin model that are in common with genes listed as Nos. 1-215 of Table 8;
(b) Table 8, Nos. 437-794 determined by the Kendall model and the genes determined by the Kendall genes that are in common with genes listed as Nos. 1-215 of Table 8; and
(c) Table 9.

12. An array comprising:
(a) a solid support; and
(b) a plurality of polynucleotide probes immobilized on said solid support, wherein the plurality of polynucleotide probes are capable of hybridizing to at least 10 genes listed in Tables 1-5 and Table 8, optionally including one or more control probes.

13. The array of claim 12, wherein said probe array is a microarray.

14. The array of claim 12, wherein said plurality of polynucleotide probes is capable of hybridizing to at least 10 of the 384 genes listed in Table 9.

15. A kit comprising the array of claim 12 and instructions for test compound screening and quantification of gene expression using the microarray.

16. A method of monitoring the efficacy of a candidate adjuvant compound in a subject comprising:
a) measuring baseline expression of a plurality of genes known to be upregulated or downregulated in skin in response to topical application of DBP;
c) topically applying to the skin of said subject the candidate adjuvant compound,
d) measuring the expression of the plurality of genes after exposure of the to the candidate adjuvant compound, and
e) comparing expression levels of the plurality of genes before and after exposure to the candidate adjuvant compound, wherein a change in expression of any of the one or more of the plurality of genes by at least two-fold following exposure to the candidate adjuvant compound indicates that the compound is an effective adjuvant.

17. The method of claim 16, wherein said one or more genes are early response gene(s).

18. The method of claim 16, wherein said plurality of genes are significantly upregulated by Toll-like Receptor (TLR) stimulation.

19. The method of claim 16, wherein an expression level of said plurality of genes is known to be increased in activated dendritic cells.

20. A composition comprising:
a lipophilic molecule having a molecular weight of less than 500 daltons that induces dendritic cell migration and modulates expression level of genes in skin cells, wherein at least 20% of genes whose expression level is increased or decreased by at least 2-fold by DBP are also increased or decreased, respectively, by at least 2-fold by said lipophilic molecule, wherein the lipophilic molecule is not DBP, and
a pharmaceutically acceptable carrier.

21. A vaccine comprising an antigen and a lipophilic molecule of less than 500 daltons, wherein the molecule induces dendritic cell migration and modulates expression level of genes in skin cells, wherein at least 20% of genes whose expression level is increased or decreased by at least 2-fold by DBP are also increased or decreased, respectively, by at least 2-fold by said lipophilic molecule, wherein the molecule is not DBP.

22. A method of inducing an immune response in a subject comprising administering the vaccine of claim 21 to said subject.
